(19) United States
(12) Patent Application Publication
(10) Pub. No.: US 2026/0085368 A1
(43) Pub. Date: Mar. 26, 2026

(54) SYSTEMS AND METHODS FOR DIAGNOSING A DISEASE OR A CONDITION

(71) Applicants: Icahn School of Medicine at Mount Sinai, New York, NY (US); The Trustees of Princeton University, Princeton, NJ (US); The Simons Foundation, Inc., New York, NY (US)

(72) Inventors: Stuart C. Sealfon, New York, NY (US); Olga G. Troyanskaya, Princeton, NJ (US); Zijun Zhang, New York, NY (US)

(21) Appl. No.: 19/108,135

(22) PCT Filed: Sep. 1, 2023
(86) PCT No.: PCT/US2023/073359

§ 371 Date: Feb. 28, 2025

Related U.S. Application Data
(60) Provisional application No. 63/403,687, filed on Sep. 2, 2022.

(51) Int. Cl. C12Q 1/70; C12Q 1/6874; G16B 30/10; G16B 40/20

(52) U.S. Cl. CPC C12Q 1/701; C12Q 1/6874; G16B 30/10; G16B 40/20

(57) ABSTRACT

An infection status of a subject is determined using sequence reads from a biological sample of the subject. For each respective alternative splicing (AS) event in a plurality of AS events, there is determined (i) a corresponding first abundance metric of the AS event in the biological sample based on a mapping of each sequence read to one or more reference splice junctions in a plurality of reference splice junctions, and (ii) a corresponding second abundance metric of the AS event in the biological sample based on a mapping of each respective sequence read of the plurality of sequence reads to one or more reference isoforms in a plurality of reference isoforms. Each AS event corresponds to a locus in a reference genome. The first and second abundance metrics for each AS event are inputted into a model to obtain a predicted infection status of the subject as model output.

### CROSS REFERENCE TO RELATED APPLICATIONS

This Application claims priority to U.S. Provisional Patent Application Ser. No. 63/403,687, entitled “Systems and Methods for Diagnosing a Disease or a Condition,” filed Sep. 2, 2022, which is hereby incorporated by reference in its entirety for all purposes.

### STATEMENT REGARDING FEDERALLY SPONSORED RESEARCH OR DEVELOPMENT

This invention was made with government support under N6600119C4022, awarded by the Defense Advanced Research Projects Agency (DARPA), by 9700130 awarded by Defense Health Agency through the Naval Medical Research Center, by R01 GM071966 awarded by the National Institute of Health (NIH). The government has certain rights in the invention.

### TECHNICAL FIELD

This specification describes using various computational tools to diagnose an infection status of a subject.

### BACKGROUND

Host-based response assays (HRAs) are being developed for diagnosis of infectious disease. See Buonsenso et al., 2022, “Transcript host-RNA signatures to discriminate bacterial and viral infections in febrile children,” Pediatr. Res., 91, pg. 454-463; Galtung et al., 2022, “Prospective validation of a transcriptomic severity classifier among patients with suspected acute infection and sepsis in the emergency department,” Eur. J. Emerg. Med., 29, pg. 357-365, each of which is hereby incorporated by reference in its entirety for all purposes. Compared with conventional pathogen-based nucleic acid amplification tests (NAATs), HRAs target transcriptional alterations in the host blood instead of pathogenic materials. During the early window of infection, NAATs are known to have relatively high false negative rate, while HRAs are shown to be sensitive as early as twelve hours after viral challenge. See Kucirka et al., 2020, “Variation in false-negative rate of reverse transcriptase polymerase chain reaction-based SARS-CoV-2 tests by time since exposure,” Ann. Intern. Med., 173, pg. 262-267; Huang et al., 2011, “Temporal dynamics of host molecular responses differentiate symptomatic and asymptomatic influenza a infection,” PLoS Genet., 7, e1002234, each of which is hereby incorporated by reference in its entirety for all purposes. This characteristic of HRAs has been leveraged to develop early detection of severe acute respiratory syndrome coronavirus 2 (SARS-CoV-2) infections. See Cappuccio et al., 2022, “Earlier detection of SARSCoV-2 infection by blood RNA signature microfluidics assay,” Clin. Transl. Discov., 2, e47, which is hereby incorporated by reference in its entirety for all purposes. Similarly, HRAs have been developed where traditional NAATs have failed, such as discriminating bacterial versus viral infections and early detection of respiratory viral infection. See Buonsenso et al., 2022; Tsalik et al., 2021, “Discriminating bacterial and viral infection using a rapid host gene expression test,” Crit. Care Med., 49, pg. 1651-1663; McClain et al., 2021, “A blood-based host gene expression assay for early detection of respiratory viral infection: an index-cluster prospective cohort study,” Lancet Infect. Dis., 21, pg. 396-404, each of which is hereby incorporated by reference in its entirety for all purposes.

However, existing HRAs are restricted to gene expression signatures with unsatisfactory normalization and platform stability. Platform stability is important for effective biomarker assays in clinics because biomarkers are typically discovered from genome-scale experimental platforms (e.g., Illumina RNA sequencing [RNA-seq]) but are implemented as targeted profiling assays (e.g., microfluidic devices) for portable and scalable clinical use. Without suitable normalization gene expression signatures are subject to platform-specific bias and technical noises. Moreover, as illustrated in FIG. 4B, models based on such gene expression signatures have unsatisfactory performance.

Given the above-background, what is needed in the art are HRAs with improved normalization and platform stability.

### SUMMARY

Advantageously, the present disclosure provides HRAs with improved normalization and platform stability by making use of RNA alternative splicing (AS) in HRAs. During AS, an exon (or part of an exon) in the pre-mRNA is selectively included or excluded from the final mRNA product. See Park et al., 2018, “The expanding landscape of alternative splicing variation in human populations,” Am. J. Hum. Genet., 102, pg. 11-26, which is hereby incorporated by reference in its entirety for all purposes. In the present disclosure AS events are quantified by the ratio between the exon-inclusion isoforms and exon-skipping isoforms. In this way, AS events provide normalization and assay platform stability advantages compared with gene expression for diagnostic HRAs. By measuring the AS event as a ratio, the inclusion level of an exon normalizes to a proportion between 0 and 1 platform-specific bias and technical noises cancel out in the ratio because they equivalently affect exon-inclusion and -skipping isoforms. Accordingly, the systems and methods of the present disclosure provide a framework (e.g., a computational framework) for AS diagnostic biomarkers.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

One aspect of the present disclosure provides a method for determining an infection status of a test subject. The method is performed at a computer system comprising one or more processors and a memory storing at least one program for execution by the one or more processors.

There is obtained, in electronic form, a plurality of sequence reads from a biological sample of the test subject. In some embodiments, the plurality of sequence reads comprises at least 10,000 RNA sequence reads.

In some such embodiments, the biological sample is whole blood.

In some embodiments, the biological sample comprises a plurality of mRNA molecules and the obtaining the plurality of sequence reads further comprises sequencing the plurality of mRNA molecules using RNA sequencing. In some such embodiments, all or a portion of the plurality of mRNA molecules is derived from the test subject.

In some embodiments, the biological sample further comprises nucleic acid molecules derived from a pathogen.

In some such embodiments, the infection status of the test subject is for a SARS-CoV-2 infection.

In some embodiments, the infection status of the test subject is for a bacterial infection, a viral infection, a fungal infection, a parasitic infection, sepsis, tuberculosis, a respiratory infection, a gastrointestinal infection, a urinary tract infection, or a combination thereof.

In some embodiments, the infection status of the test subject is for an influenza infection, a human immunodeficiency viral infection, a COVID-19 infection, or a combination thereof.

In some embodiments, the plurality of sequence reads comprises at least 100,000, at least 1×10⁶, or at least 1×10⁷ sequence reads.

A determination is made, for each respective alternative splicing event in a plurality of alternative splicing events of. (i) a corresponding first abundance metric of the respective alternative splicing event in the biological sample, optionally based on a mapping of each respective sequence read in the plurality of sequence reads to one or more reference splice junctions in a plurality of reference splice junctions, and (ii), optionally, a corresponding second abundance metric of the respective alternative splicing event in the biological sample optionally based on a mapping of each respective sequence read in the plurality of sequence reads to one or more reference isoforms in a plurality of reference isoforms, where each respective alternative splicing event in the plurality of alternative splicing events corresponds to a respective locus in a plurality of loci in a reference genome for the species of the test subject, and the plurality of alternative splicing events comprises at least 10 alternative splicing events.

In some embodiments, each respective alternative splicing event in the plurality of alternative splicing events is a skipped exon, an alternative 5′ splice site, an alternative 3′ splice site, or a retained intron in the respective locus in the plurality of loci that corresponds to the respective alternative splicing event.

In some embodiments, the plurality of alternative splicing events comprises at least 10, at least 20, at least 50, at least 100, or at least 500 alternative splicing events. In some embodiments, the plurality of alternative splicing events is no more than 2000, no more than 1000, no more than 500, no more than 100, or no more than 50 alternative splicing events. In some embodiments, the plurality of alternative splicing events consists of from 100 alternative splicing events to 600 alternative splicing events. In some embodiments, the plurality of alternative splicing events consists of from 10 alternative splicing events to 50 alternative splicing events.

In some such embodiments, each respective locus in the plurality of loci is a gene in a plurality of genes.

In some embodiments, the plurality of alternative splicing events is for determining an infection status of a SARS-CoV-2 infection, and the plurality of genes comprises one or more of IGLL5, LST1, GALNS, EPSTI1, LILRB2, RIN2, PALM2AKAP2, HMGN2, TUBA8, SNHG32, KIF22, ATP6V0B, SESN3, LRRK, U91328.1, IQSEC1, RPS3A, KY, PHOSPHO1, RILP, MRPS22, and ZFYVE26.

In some embodiments, the plurality of alternative splicing events is selected from the group consisting of: skipped exon IGLL5, retained intron LST1, skipped exon GALNS, skipped exon EPSTI1, retained intron LILRB2, skipped exon RIN2, skipped exon PALM2AKAP2, retained intron HMGN2, alternative 5′ splice site TUBA8, skipped exon SNHG32, alternative 3′ splice site KIF22, alternative 5′ splice site ATP6V0B, skipped exon SESN3, alternative 3′ splice site LST1, alternative 3′ splice site LRRK, skipped exon U91328.1, alternative 3′ splice site IQSEC1, skipped exon RPS3A, alternative 5′ splice site KY, alternative 3′ splice site PHOSPHO1, skipped exon RILP, retained intron MRPS22, skipped exon ZFYVE26, skipped exon PHOSPHO1, and alternative 3′ splice site LILRB2.

In some embodiments, for a respective alternative splicing event in the plurality of alternative splicing events, the first abundance metric is a percent or proportion spliced in metric determined according to the equation:

$$\frac{\frac{incl usion count}{2}}{\frac{incl usion count}{2} + skip count}$$

where inclusion count is a count of inclusion splice junctions for a first intervening sequence corresponding to the respective alternative splicing event, each respective inclusion splice junction for the first intervening sequence comprising a first nucleic acid sequence for a 5′ or a 3′ end of the first intervening sequence and a second nucleic acid sequence for an adjoining sequence that is 5′ or 3′ of the first intervening sequence, and skip count is a count of exclusion splice junctions for the first intervening sequence corresponding to the respective alternative splicing event, each respective exclusion splice junction excluding all or a portion of the first intervening sequence.

In some embodiments, the determination further comprises aligning each respective sequence read in the plurality of sequence reads to a first reference sequence comprising the plurality of reference splice junctions. In some such embodiments, the first reference sequence is a reference human genome.

In some such embodiments, the first abundance metric is determined using an RNA sequencing mapping algorithm.

In some such embodiments, for a respective alternative splicing event in the plurality of alternative splicing events, the second abundance metric is a percent or proportion spliced in metric determined according to the equation:

$$\frac{\sum inclusion isoform TPM}{\sum all relevant isoform TPM}$$

where inclusion isoform TPM is a count of transcript isoforms in the biological sample comprising a first intervening sequence corresponding to the respective alternative splicing event, measured in transcripts per million, and all relevant isoform TPM is a count of transcript isoforms spanning the first intervening sequence corresponding to the respective alternative splicing event, measured in transcripts per million.

In some embodiments, the determination further comprises aligning each respective sequence read in the plurality of sequence reads to a second reference sequence comprising the plurality of reference isoforms. In some such embodiments, the second reference sequence is a reference human transcriptome and the aligning comprises a pseudo-alignment.

In some embodiments, the second abundance metric is determined using a differential splicing analysis algorithm.

In some embodiments, responsive to inputting the corresponding first abundance metric and/or the corresponding second abundance metric for each respective alternative splicing event in the plurality of alternative splicing events into a first model, a predicted infection status of the test subject as output from the first model is received. In some embodiments, the first model is a logistic regression model. In some embodiments, the first model is selected from the group consisting of: a neural network, a support vector machine, a Naive Bayes model, a nearest neighbor model, a boosted trees model, a random forest model, a decision tree, or a clustering model.

In some embodiments, the infection status of the test subject is a likelihood that the test subject has an infection. In some embodiments, the infection status of the test subject is a likelihood that the test subject is pre-infection, first-infection, mid-infection, or post-infection. In some embodiments, the infection status of the test subject is a binary indication as to whether or not test subject has an infection. In some such embodiments, the infection status of the test subject is a binary indication as to whether or not the test subject has a pre-infection, a first-infection, a mid-infection, or a post-infection.

Another aspect of the present disclosure provides a method of selecting a plurality of alternative splicing events. A first plurality of training samples is obtained. Each respective training sample in the first plurality of training samples (i) corresponds to a respective training subject in a first plurality of training subjects and (ii) comprises a corresponding infection status of the respective training subject. Each respective training sample in a first subset of the first plurality of training samples comprises a first infection status. Each respective training sample in a second subset of the first plurality of training samples comprises a second infection status. In some such embodiments, the first infection status is negative for infection and the second infection status is positive for infection.

In some embodiments, the first plurality of training subjects comprises a first subset of healthy subjects and a second subset of disease subjects. In some such embodiments, each respective training sample in the second subset of training samples is obtained from the second subset of disease subjects. In some embodiments, for each respective training sample in the second subset of training samples, the corresponding infection status is selected from the group consisting of pre-infection, first-infection, mid-infection, and post-infection. In some embodiments, each respective training sample in the first subset of training samples is obtained from the first subset of healthy subjects or the second subset of disease subjects.

In some embodiments, for each respective training sample in the first plurality of training samples, the corresponding infection status is determined by polymerase chain reaction, immunoglobulin G antibody testing, or immunoglobulin M antibody testing.

In some embodiments, in accordance with this aspect of the present disclosure, there is determined, for each respective training sample in the first plurality of training samples, for each respective candidate event in a plurality of candidate events, at least a first abundance metric of the respective candidate event in the respective training sample, thereby obtaining at least a plurality of first abundance metrics for the first plurality of training samples. In some such embodiments, for each respective training sample in the first plurality of training samples, for each respective candidate event in the plurality of candidate events, the corresponding first abundance metric is determined based on a mapping of each respective sequence read, in a plurality of sequence reads for the respective training sample, to one or more reference splice junctions in a plurality of reference splice junctions.

In some embodiments, in accordance with this aspect of the presented disclosure there is received, responsive to inputting at least the plurality of first abundance metrics into a second model, for each respective candidate event in the plurality of candidate events, (i) a corresponding coefficient of effect between the respective candidate event and the corresponding infection status of each respective training sample in the first plurality of training samples and, optionally, (ii) a measure of significance for the corresponding coefficient of effect.

In some such embodiments, the measure of significance is a false discovery rate.

In some such embodiments, for each respective candidate event in the plurality of candidate events, the corresponding coefficient of effect is determined as regression coefficient 3, according to the equation:

$$logit ( {ψ}_{i j l} ) = {μ}_{I} + α {Sex}_{j} + β Diseas {e}_{j} + {P}_{i j} + {δ}_{i} 1 ( {ψ}_{i j l} ∈ {Ψ}_{JCT} ) + {\sum }_{k} {γ}_{k} P {C}_{k j} + {ε}_{i j}$$

where, ψᵢⱼₗ is an inclusion level for alternative splicing event i in the RNA-seq sample j measured by approach l, l is the first abundance metric or the second abundance metric, wherein the first abundance metric comprises exon-exon splice junction counts and the second abundance metric comprises isoform ratios, μ_{I} is a baseline inclusion level for alternative splicing event i, Sexⱼ is an annotated sex for sample j with regression coefficient α, Diseaseⱼ is an annotated disease stage for sample j with regression coefficient β, Pᵢⱼ is a random effect for sample j to account for covariance among multiple RNA sequencing samples derived from the same subject, δᵢ quantifies a difference between measurement approaches for alternative splicing event i if ψᵢⱼₗ is measured by counting exon-exon splice junctions ψᵢⱼₗ∈Ψ_{JCT} as compared to isoform ratios, 1(⋅) is an indicator function, and γₖ is a coefficient for each of k principal components for sample j PCₖⱼ.

In some embodiments, the second model is a regression model and the corresponding coefficient of effect is a regression coefficient. In some such embodiments, the regression model is a linear mixed model.

In some such embodiments, for each respective training sample in the first plurality of training samples, for each respective candidate event in the plurality of candidate events, this aspect of the present disclosure further determines a second abundance metric of the respective candidate event in the respective training sample, thereby obtaining a plurality of second abundance metrics for the first plurality of training samples. The receiving further comprises inputting the plurality of second abundance metrics, with the plurality of first abundance metrics, into the second model.

An evaluation, for each respective candidate event in the plurality of candidate events, of the (i) corresponding coefficient of effect or (ii) measure of significance against one or more selection criteria is made, thereby selecting the plurality of alternative splicing events. In some such embodiments, the one or more selection criteria comprises a threshold false discovery rate of less than 0.05, less than 0.01, or less than 0.001.

Another aspect of the present disclosure provides a method of filtering a plurality of alternative splicing events using a forward selection procedure. A ranked sequence of alternative splicing events is obtained, for example, by ranking the above described plurality of alternative splicing events by their respective coefficients of effect.

A filtered subset of alternative splicing events is initialized with the highest ranked alternative splicing event in the ranked sequence of alternative splicing events. Then a plurality of iterations is performed. Each respective iteration in the plurality of iterations comprises, for each respective alternative splicing event that is the next highest ranked alternative splicing event in the ranked sequence of alternative splicing events: obtaining a respective evaluation set of alternative splicing events comprising the respective alternative splicing event and the filtered subset of alternative splicing events, for each respective validation subject in a plurality of validation subjects: (i) for each respective alternative splicing event in the evaluation set of alternative splicing events, determining at least a corresponding first abundance metric for the respective alternative splicing event in a biological sample of the respective validation subject, and (ii) receiving, responsive to inputting the corresponding first abundance metric for each respective alternative splicing event in the evaluation set of alternative splicing events into the a model, a predicted infection status of the respective validation subject as output from a first model.

The predicted infection status for each respective validation subject in the plurality of validation subjects is used to determine a corresponding evaluation metric for the respective evaluation set of alternative splicing events.

When the corresponding evaluation metric satisfies a filtering criterion, the respective alternative splicing event is added to the filtered subset of alternative splicing events and a subsequent iteration in the plurality of iterations is performed.

When the corresponding evaluation metric fails to satisfy the filtering criterion, the plurality of iterations is ended thereby obtaining the filtered subset of alternative splicing events.

In some such embodiments, for each respective validation subject in the plurality of validation subjects, for each respective alternative splicing event in the evaluation set of alternative splicing events: determine a corresponding second abundance metric for the respective alternative splicing event in the biological sample of the respective validation subject. The receiving further comprises inputting the corresponding second abundance metric, with the corresponding first abundance metric, into the first model.

In some such embodiments, for a respective iteration in the plurality of iterations: the filtering criterion is satisfied when the corresponding evaluation metric exceeds an evaluation metric for the iteration immediately prior to the respective iteration, and the filtering criterion is not satisfied when the corresponding evaluation metric does not exceed the evaluation metric for the iteration immediately prior to the respective iteration. In some such embodiments, the evaluation metric is selected from the group consisting of accuracy, positive percent agreement, and negative percent agreement.

Another aspect of the present disclosure provides a method of training the first model by a procedure. In accordance with this aspect of the present disclosure, for each respective training sample in a second plurality of training samples, for each respective alternative splicing event in the plurality of alternative splicing events, at least a third abundance metric of the respective alternative splicing event in the respective training sample.

In some such embodiments, for each respective training sample in the second plurality of training samples, for each respective alternative splicing event in the plurality of alternative splicing events, the corresponding third abundance metric is determined based on a mapping of each respective sequence read, in a plurality of sequence reads for the respective training sample, to one or more reference splice junctions in a plurality of reference splice junctions.

In some such embodiments, each respective training sample in the second plurality of training samples (i) corresponds to a respective training subject in a second plurality of training subjects and (ii) comprises a corresponding measured infection status. Each respective training sample in a first subset of the second plurality of training samples comprises a first measured infection status. Each respective training sample in a second subset of the second plurality of training samples comprises a second measured infection status.

In some embodiments, for each respective training sample in the second plurality of training samples, the corresponding measured infection status is determined by polymerase chain reaction, immunoglobulin G antibody testing, or immunoglobulin M antibody testing.

In some embodiments, the second plurality of training subjects comprises a first subset of healthy subjects and a second subset of disease subjects.

In some embodiments, the first measured infection status is negative for infection, and the second measured infection status is positive for infection. Each respective training sample in the second subset of training samples is obtained from the second subset of disease subjects.

In some embodiments, the first measured infection status is selected from the group consisting of pre-infection, first-infection, mid-infection, and post-infection.

In accordance with this aspect of the present disclosure there is received, for each respective training sample in the second plurality of training samples, responsive to inputting at least the first abundance metric of each respective alternative splicing event in the plurality of alternative splicing events into the first model, a corresponding predicted infection status of the respective training sample as output from the first model. The first model comprises a plurality of parameters.

In some embodiments, for each respective training sample in the second plurality of training samples, for each respective alternative splicing event in the plurality of alternative splicing events: determine a fourth abundance metric of the respective alternative splicing event in the respective training sample. In such embodiments, the receiving further comprises inputting the fourth abundance metric of each respective alternative splicing event in the plurality of alternative splicing events into the first model.

In accordance with this aspect of the present disclosure, a respective difference is applied to a loss function to obtain a respective output of the loss function. The respective difference is between, for each respective training sample in the second plurality of training samples, (i) the corresponding predicted infection status and (ii) the corresponding measured infection status. The respective output of the loss function is used to adjust one or more parameters in the plurality of parameters of the first model.

### INCORPORATION BY REFERENCE

All publications, patents, and patent applications mentioned in this specification are herein incorporated by reference in their entireties for all purposes to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an exemplary system topology including a computer system, in accordance with an exemplary embodiment of the present disclosure.

FIG. 2A illustrates an overview of analysis workflow to develop robust alternative splicing diagnostic biomarkers, which depicts input data for a framework of the present disclosure included cohort metadata and whole-blood RNA-seq data to develop alternative splicing (AS)-based diagnostic markers, in accordance with an exemplary embodiment of the present disclosure.

FIG. 2B illustrates an overview of analysis workflow to develop robust alternative splicing diagnostic biomarkers, which depicts data processed in two steps: AS levels were quantified by two statistical approaches; and differential analysis was performed by a regression framework, allowing for cross-sectional and longitudinal experimental designs, in accordance with an exemplary embodiment of the present disclosure.

FIG. 2C illustrates an overview of analysis workflow to develop robust alternative splicing diagnostic biomarkers, which depicts classification of disease versus controls using AS-based biomarkers, in which PSI values for AS events were used as features to train a machine-learning classifier, which were evaluated in independent cohorts, in accordance with an exemplary embodiment of the present disclosure.

FIG. 2D illustrates an overview of analysis workflow to develop robust alternative splicing diagnostic biomarkers, which depicts performance of a forward selection to select non-redundant AS signatures, in which the optimized, small-footprint signature set was then implemented in microfluidic devices for diagnostic testing, in accordance with an exemplary embodiment of the present disclosure.

FIG. 2E illustrates an overview of analysis workflow to develop robust alternative splicing diagnostic biomarkers, which depicts functional analysis of AS variations on the exon and gene levels, in which the blue band represents data processing and the green band represents signature development and analysis, in accordance with an exemplary embodiment of the present disclosure.

FIG. 3A illustrates an overview of longitudinal analysis of differential AS events in SARS-CoV-2 infection, which depicts an experimental design for the COVID-19 Health Response for Marines (CHARM) cohort, in which whole-blood specimens from subjects were collected longitudinally and sequenced by RNA-seq. controls were PCR-negative (PCR−) samples with no positive antibody tests, and in which the first sample with a PCR+ test for a given subject was labeled as first, whereby PCR− RNA-seq samples within 2 weeks before first for these subjects were labeled as pre-infection, in which PCR+ samples after the first sample were labeled as mid, while RNA-seq samples from infected subjects that turned PCR− were labeled as post-infection (post), in accordance with an exemplary embodiment of the present disclosure.

FIG. 3B illustrates an overview of longitudinal analysis of differential AS events in SARS-CoV-2 infection, which depicts definitions of the four AS events analyzed and the number of statistically significant (FDR<0.05) events identified for each disease stage, in accordance with an exemplary embodiment of the present disclosure.

FIG. 4A illustrates an overview of biomarkers of alternative splicing (AS) events cross-cohort predictive modeling, which depicts a comparison of AS and gene expression classification area under receive operating characteristic (AUROC) on the Duke cohort using varying numbers of features, in accordance with an exemplary embodiment of the present disclosure.

FIG. 4B illustrates an overview of biomarkers of alternative splicing (AS) events cross-cohort predictive modeling, which depicts a comparison of the best-performing DAS and DEG signatures (CHARM DAS and DEG) against five public transcriptome signatures on the Duke cohort, in accordance with an exemplary embodiment of the present disclosure.

FIG. 4C illustrates an overview of biomarkers of alternative splicing (AS) events cross-cohort predictive modeling, which depicts an AUROC plot for the cross-cohort SARS-CoV-2 infection classifier, in which the color bar represents the predicted scores, in accordance with an exemplary embodiment of the present disclosure.

FIG. 4D illustrates an overview of biomarkers of alternative splicing (AS) events cross-cohort predictive modeling, which depicts cross-validation predictions for control samples identified an infected subject that was mislabeled as healthy control despite serial PCR tests, in accordance with an exemplary embodiment of the present disclosure.

FIG. 4E illustrates an overview of biomarkers of alternative splicing (AS) events cross-cohort predictive modeling, which depicts PCA of AS biomarkers measured by microfluidic devices in a third independent clinical cohort, in accordance with an exemplary embodiment of the present disclosure.

FIG. 4F illustrates an overview of biomarkers of alternative splicing (AS) events cross-cohort predictive modeling, which depicts PCA of gene expression biomarkers measured by microfluidic devices generated by Cappuccio et al., 2020, in which decision boundaries are fitted by a support vector machine with a linear kernel, and in which PPA is positive percent agreement, and NPA is negative percent agreement, in accordance with an exemplary embodiment of the present disclosure.

FIG. 5A illustrates an overview of functional analysis for DAS genes, which depicts whole-blood tissue-specific gene network enrichment of genes undergoing DAS, in which representative Gene Ontology terms for each module are shown and listed in Table 5, in accordance with an exemplary embodiment of the present disclosure.

FIG. 5B illustrates an overview of functional analysis for DAS genes, which depicts the percentage of genes undergoing DAS in three representative GO terms along the temporal course of disease progression, in accordance with an exemplary embodiment of the present disclosure.

FIG. 5C illustrates an overview of functional analysis for DAS genes, which depicts the estimated coefficients represent an unbiased disease-induced AS difference between disease progression stages and healthy controls, in which the average of coefficients for upregulated and downregulated SE events are depicted, and in which horizontal bars are 95% bootstrapping confidence intervals, in accordance with an exemplary embodiment of the present disclosure.

FIGS. 5D and 5E collectively illustrate an overview of functional analysis for DAS genes, which depicts volcano plots for enrichment of proteins domains annotated by Pfam (FIG. 5D) and RBP binding sites identified by ENCODE eCLIP (FIG. 5E), in which p values were calculated from Fisher's exact test, and in which vertical dashed lines represents cut off at fold change >, and horizontal dashed lines represents FDR<0.05, in accordance with an exemplary embodiment of the present disclosure.

FIGS. 6A and 6B collectively represent hierarchical clustering of First vs Control samples using differentially spliced events identified in skipped exon events, in which PSI values were measured by two popular but technically distinct ways, based on exon-exon junction counts (Junction) and inclusion/exclusion transcript ratios (Transcript), respectively, to quantify the inclusion level of an alternative splicing event, and in which PSI values were Z-score transformed, in accordance with an exemplary embodiment of the present disclosure.

FIG. 7A illustrates an overview of blood AS biomarker evaluations, which depicts predicted infection risk scores for labeled control subjects across time points, in which predictions were made using 10-fold cross-validation and time points were adjusted for initial enrollment time (as time 0) for different subjects, in accordance with an exemplary embodiment of the present disclosure.

FIG. 7B illustrates an overview of blood AS biomarker evaluations, which depicts comparison of predicted infection risk scores for PCR-negative, seronegative control samples with and without symptoms, in accordance with an exemplary embodiment of the present disclosure.

FIG. 7C illustrates an overview of blood AS biomarker evaluations, which depicts accuracy of the Duke cohort as a function of forward selection steps, in accordance with an exemplary embodiment of the present disclosure.

FIG. 7D illustrates an overview of blood AS biomarker evaluations, which depicts accuracies computed by down-sampling of the gene expression biomarker assay to match the sample size of the alternative splicing biomarker assay, in which P-value was calculated as the frequency of down-sampled gene expression assays with accuracy equal or greater than the observed accuracy from the alternative splicing assay, in accordance with an exemplary embodiment of the present disclosure.

FIG. 7E illustrates an overview of blood AS biomarker evaluations, which depicts probe-based PSI values normalized by alternative splicing are significantly more stable than the raw probe values, in which each dot represented the within-group, between subject variance, an d in which statistical significance was determined by two-sided Mann-Whitney U test, P-values were 0.004 (PSI vs Raw in Healthy), 0.062 (PSI vs Cappuccio et al., 2020, in Healthy), 3.8e-4 (PSI vs Raw in COVID), and 0.084 (PSI vs Cappuccio et al., 2020 in COVID), in accordance with an exemplary embodiment of the present disclosure.

FIGS. 7F and 7G collectively illustrate an overview of blood AS biomarker evaluations, which depicts two representative AS signatures with SARS-CoV-2 induced DAS changes, in which skipped exon event (Exon 2) of gene GALNS (FIG. 7F), and retained intron event of gene LST1 (FIG. 7G), consistent across cohorts upon SARS-CoV-2 infection, in accordance with an exemplary embodiment of the present disclosure.

FIG. 8A illustrates an overview of a functional interpretation of AS biomarkers, which depicts a tree map showing the clustering of significantly-enriched GO terms of DAS genes, in accordance with an exemplary embodiment of the present disclosure.

FIG. 8B illustrates an overview of a functional interpretation of AS biomarkers, which depicts temporal dynamics of the other types of AS events, in which dots are average of estimated coefficients and error bars are 95% bootstrapping confidence intervals, in accordance with an exemplary embodiment of the present disclosure.

FIGS. 9A, 9B, 9C, and 9D collectively illustrate representation in the principal component space of PSI values measured by junction counts vs isoform ratios in the CHARM RNA-seq data, in which when quantified by isoform ratios, samples were unbiased by plate numbers but had higher variance (FIGS. 9C and 9D); when quantified by junction counts, samples showed biased distributions by plate number but smaller variances (FIGS. 9A and 9B), in which the methods of the present disclosure combined the information from both types of measurements to improve the statistical power and identify measurement-agnostic splicing changes.

FIGS. 10A, 10B, 10C, 10D, 10E, and 10F provide a flow chart of an example method for determining an infection status of a test subject, in which dashed boxes represent optional elements in the flow chart, in accordance with some embodiments.

FIGS. 11A, 11B, 11C, 11D, 11E, 11F, 11G, 11H, 11, 11J, 11K, 11L, 11M, 11N, 11O, 11P, 11Q, 11R, 11S, 11T, 11U, 11V, 11W, 11X, and 11Y illustrate Representative Gene Ontology terms for module M1 of FIG. 5A in accordance with an embodiment of the present disclosure.

FIGS. 12A, 12B, 12C, 12D, 12E, 12F, 12G, 12H, 12I, 12J, 12K, 12L, 12M, 12N, 12O, 12P, 12Q, 12R, 12S, 12T, 12U, 12V, 12W, 12X, and 12Y illustrate Representative Gene Ontology terms for module M2 of FIG. 5A in accordance with an embodiment of the present disclosure.

FIGS. 13A, 13B, 13C, 13D, 13E, 13F, 13G, 13H, 13I, 13J, 13K, 13L, 13M, 13N, 13O, 13P, 13Q, 13R, 13S, 13T, 13U, 13V, 13W, 13X, and 13Y illustrate representative Gene Ontology terms for module M3 of FIG. 5A in accordance with an embodiment of the present disclosure.

FIGS. 14A, 14B, 14C, 14D, 14E, 14F, 14G, 14H, 14I, 14J, 14K, 14L, 14M, 14N, 14O, 14P, 14Q, 14R, 14S, and 14T illustrate Representative Gene Ontology terms for module M4 of FIG. 5A in accordance with an embodiment of the present disclosure.

FIGS. 15A, 15B, 15C, 15D, and 15E provide a flow chart of an example method for selecting a plurality of alternative splicing events, in which dashed boxes represent optional elements in the flow chart, in accordance with one embodiment of the present disclosure.

FIGS. 16A and 16B provide a flow chart of a method of filtering a plurality of alternative splicing events using a forward selection procedure, in which dashed boxes represent optional elements in the flow chart, in accordance with one embodiment of the present disclosure.

FIGS. 17A, 17B, and 17C provide a flow chart of a method of model training, in which dashed boxes represent optional elements in the flow chart, in accordance with one embodiment of the present disclosure.

### DETAILED DESCRIPTION

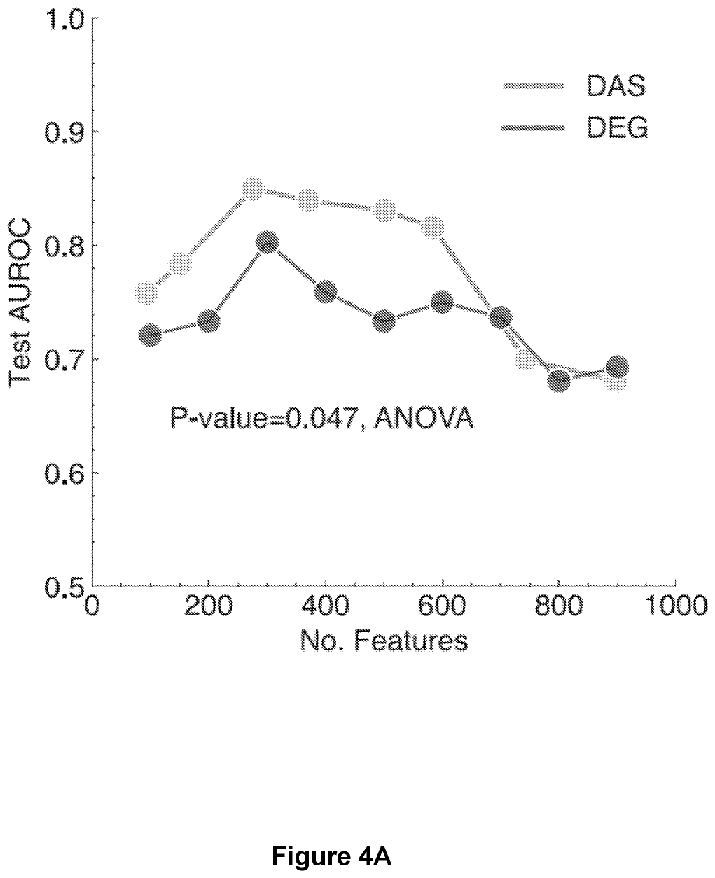

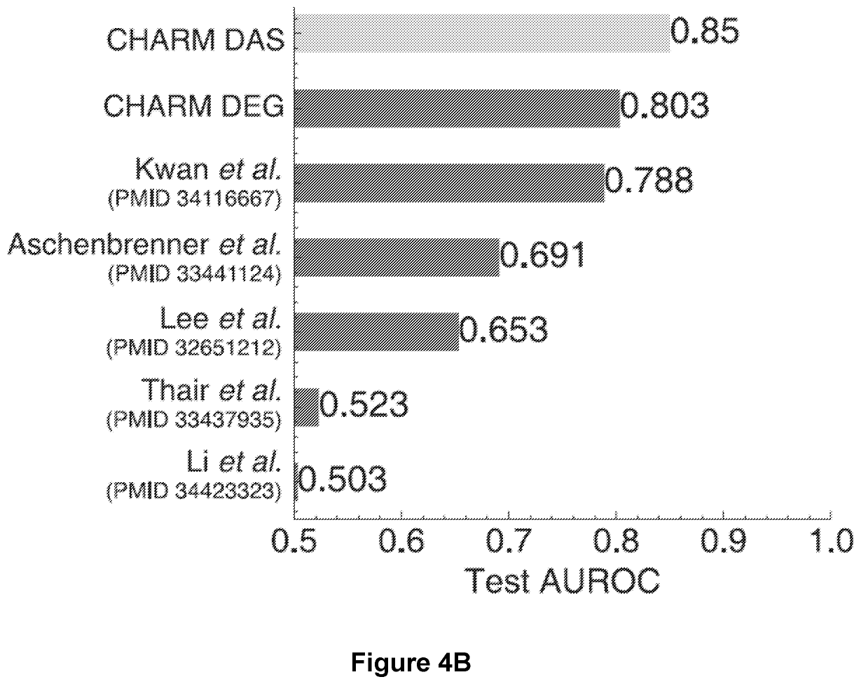

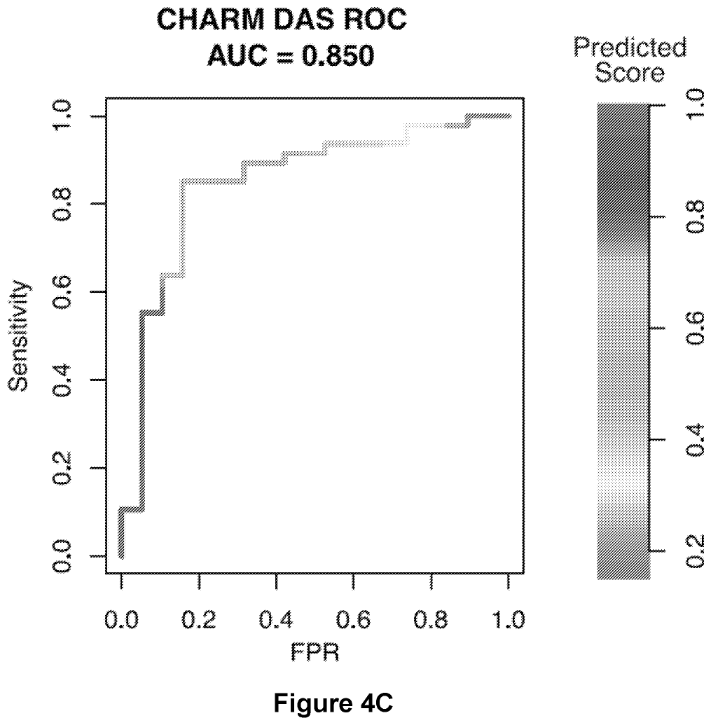

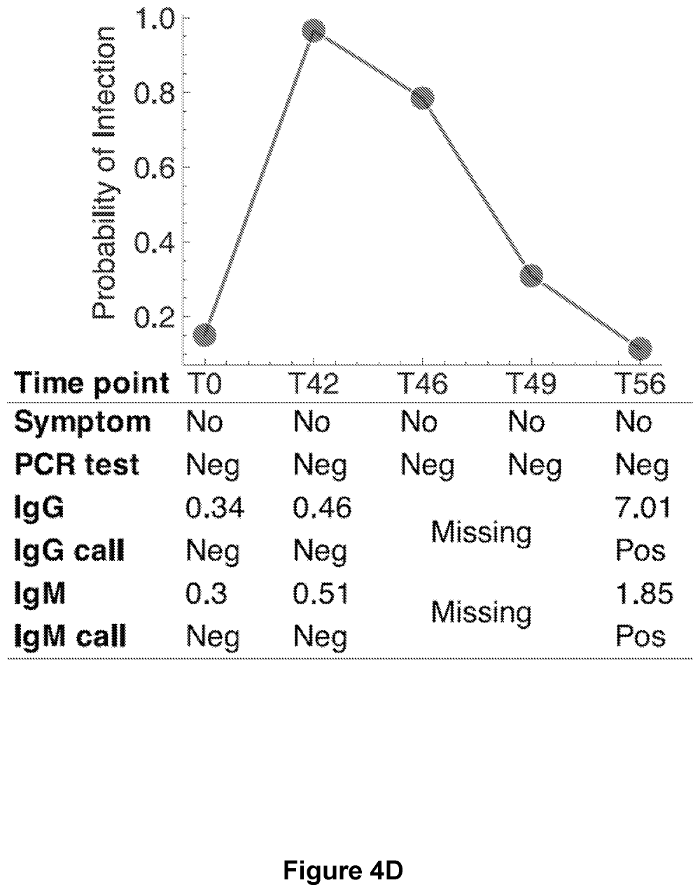

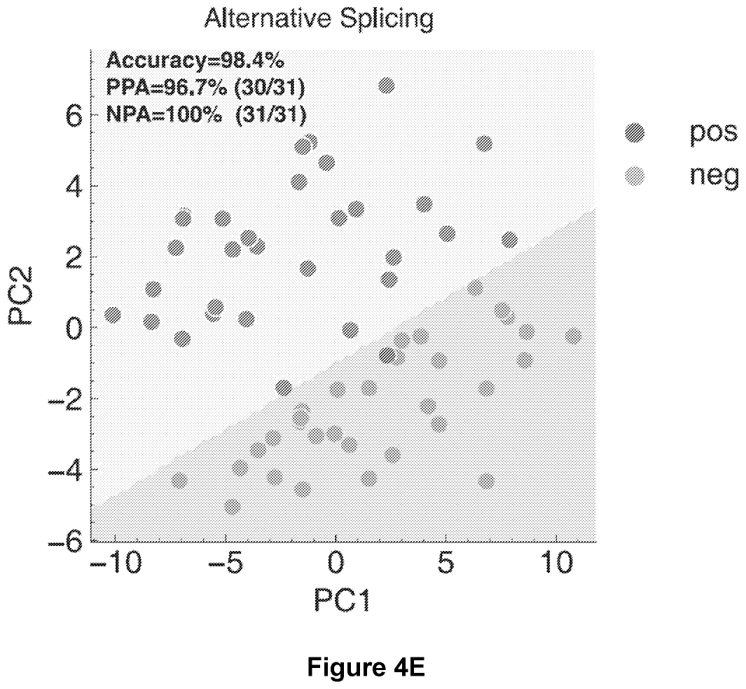

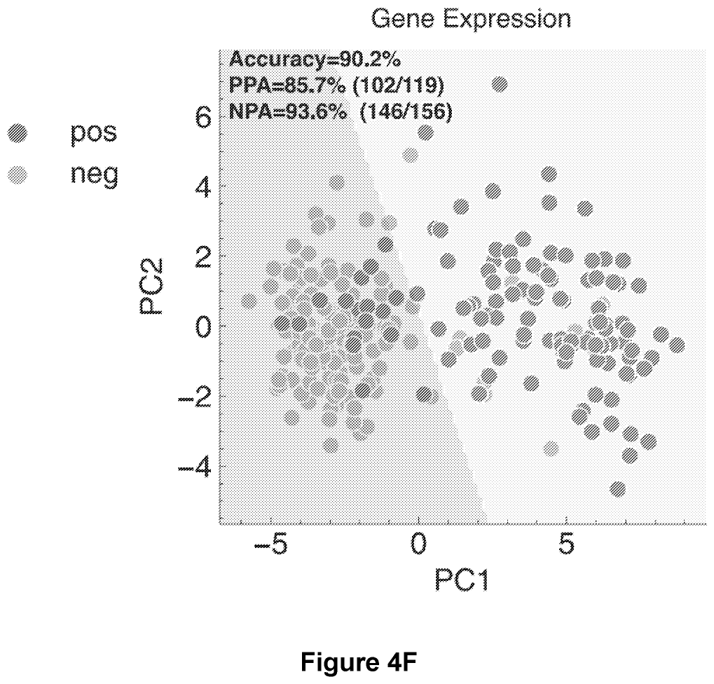

The implementations described herein provide various technical solutions for determining the status of a disease, condition, or infection in a test subject. As noted in the background, existing host-based response assays are restricted to gene expression signatures with unsatisfactory normalization and platform stability. Moreover, as illustrated in FIG. 4B, models based on such gene expression signatures have unsatisfactory performance. The claimed invention addresses these technical problems.

The claimed invention addresses these technical problems by making novel use of classifiers trained on differential alternative splicing (AS) events. During AS, an exon (or part of an exon) in the pre-mRNA is selectively included or excluded from the final mRNA product, and an AS event is quantified by the ratio between the exon-inclusion isoforms and exon-skipping isoforms. As such, AS events normalization and assay platform stability are advantageous as compared with gene expression for diagnostic host-based response assays. To this end, Example 4 identifies, using absence or presence of Covid-19 infection as a proof of principle, that AS events (e.g., those in Table 4) that are differential between case (have infection) and control (do not have infection) across different cohorts that have profoundly different demographics and other covariates, showing that AS events have cross-cohort stability.

The claimed invention further addresses the technical problems identified above for conventional host-based response assays based on gene expression by improving model performance. FIG. 4B illustrates how a model based on AS markers, when trained in the same manner as models trained on gene expression based data and tested on that same cohort as such gene based models, outperforms such gene based models. More particularly, as described in Examples 2, 4 and 9, a classifier trained on alternative splicing events that differentiate in abundance between case (infected with Covid-19) and control (not infected in Covid-19) in a cohort, labeled CHARM DAS in FIG. 4B, outperformed by a large margin five other classifiers that were each trained on published gene expression signatures for Covid-19. The DAS classifier achieved a testing AUROC of 0.85 on the same cohort that was used for the five other classifiers, demonstrating the superiority of AS signatures compared with previously published gene expression signatures.

Thus, using Covid-19 as an example, the present disclosure demonstrates that the use of AS events as markers in a model in host-response assays overcomes existing problems with conventional host-based response assays.

Advantageously, the present disclosure further provides various systems and methods for diagnosing a disease or a condition.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” means within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. All numerical values within the detailed description herein are modified by “about” the indicated value, and consider experimental error and variations that would be expected by a person having ordinary skill in the art. The term “about” can have the meaning as commonly understood by one of ordinary skill in the art. In some embodiments, the term “about” refers to ±10%. In some embodiments, the term “about” refers to ±5%.

As used herein, the term “subject,” “training subject,” or “test subject” refers to any living or non-living organism, including but not limited to a human (e.g., a male human, female human, fetus, pregnant female, child, or the like) and/or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. The terms “subject” and “patient” are used interchangeably herein and can refer to a human or non-human animal who is known to have, or potentially has, a medical condition or disorder, such as, e.g., kidney disease. In some embodiments, a subject is a “normal” or “control” subject, e.g., a subject that is not known to have a medical condition or disorder. In some embodiments, a subject is a male or female of any stage (e.g., a man, a woman, or a child).

A subject from whom an image and/or biopsy is obtained using any of the methods or systems described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20,21,22,23,24,25,26,27,28,29,30,31,32,33,34,35,36,37,38,39,40,41,42,43,44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old).

As used herein, the terms “control,” “healthy,” and “normal” describe a subject and/or an image from a subject that does not have a particular condition (e.g., kidney disease), has a baseline condition (e.g., prior to onset of the particular condition), or is otherwise healthy. In an example, a method as disclosed herein can be performed to diagnose a renal disease and/or a kidney graft failure in a subject having a renal disease using a trained model, where the model is trained using one or more training images obtained from the subject prior to the onset of the condition (e.g., at an earlier time point), or from a different, healthy subject. A control image can be obtained from a control subject, or from a database.

The term “normalize” as used herein means transforming a value or a set of values to a common frame of reference for comparison purposes. For example, when one or more pixel values corresponding to one or more pixels in a respective image are “normalized” to a predetermined statistic (e.g., a mean and/or standard deviation of one or more pixel values across one or more images), the pixel values of the respective pixels are compared to the respective statistic so that the amount by which the pixel values differ from the statistic can be determined.

As used interchangeably herein, the terms “classifier”, “model” and “machine learning model” refers to a machine learning model or algorithm. In some embodiments, such a model is a supervised machine learning model. Nonlimiting examples of supervised learning models include, but are not limited to, logistic regression models, neural networks, support vector machines, Naive Bayes algorithms, nearest neighbor models, random forest models, decision tree models, boosted trees models, multinomial logistic regression, linear models, linear regression, GradientBoosting, mixture models, hidden Markov models, Gaussian NB models, linear discriminant analysis, or any combinations thereof. In some embodiments, a machine learning model is a multinomial classifier. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier).

Neural networks.

In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in performing the methods disclosed herein. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

### Support Vector Machines.

In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms.

In some embodiments, the model is a Naive Bayes algorithm. Naïve Bayes classifiers suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

### Nearest Neighbor Algorithms.

In some embodiments, a model is a nearest neighbor algorithm. Nearest neighbor models can be memory-based and include no model to be fit. For nearest neighbors, given a query point x₀ (a first image), the k training points x₍ᵣ₎, r, . . . , k (here the training images) closest in distance to x₀ are identified and then the point x₀ is classified using the k nearest neighbors. In some embodiments, the distance to these neighbors is a function of the values of a discriminating set. In some embodiments, Euclidean distance in feature space is used to determine distance as d₍ᵢ₎=∥x₍ᵢ₎−x₍ₒ₎∥. Typically, when the nearest neighbor algorithm is used, the value data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

### Random Forest, Decision Tree, and Boosted Tree Algorithms.

In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

### Linear Discriminant Analysis Algorithms.

Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (e.g., a linear classifier) in some embodiments of the present disclosure.

### Mixture Model and Hidden Markov Model.

In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

### Clustering.

In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in a training dataset. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

### Ensembles of Models and Boosting.

In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

The term “classification” can refer to any number(s) or other characters(s) that are associated with a particular property of a sample. For example, a “+” symbol (or the word “positive”) can signify that a sample is classified as having a desired outcome or characteristic, whereas a “−” symbol (or the word “negative”) can signify that a sample is classified as having an undesired outcome or characteristic. In another example, the term “classification” refers to a respective outcome or characteristic (e.g., high risk, medium risk, low risk). In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff value refers to a value above which results are excluded. In some embodiments, a threshold value is a value above or below which a particular classification applies. Either of these terms can be used in either of these contexts.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that vary in size from tens to hundreds to thousands of base pairs. Illumina parallel sequencing can provide sequence reads vary to a lesser extent (e.g., where most sequence reads are of a length of about 200 bp or less). A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes (e.g., in hybridization arrays or capture probes) or amplification techniques, such as the polymerase chain reaction (PCR) or linear amplification using a single primer or isothermal amplification.

As disclosed herein, the terms “sequencing,” “sequence determination,” and the like refer generally to any and all biochemical processes that may be used to determine the order of biological macromolecules such as nucleic acids or proteins. For example, sequencing data can include all or a portion of the nucleotide bases in a nucleic acid molecule such as a DNA fragment.

Several aspects are described below with reference to example applications for illustration. Numerous specific details, relationships, and methods are set forth to provide a full understanding of the features described herein. The features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

### Example Systems

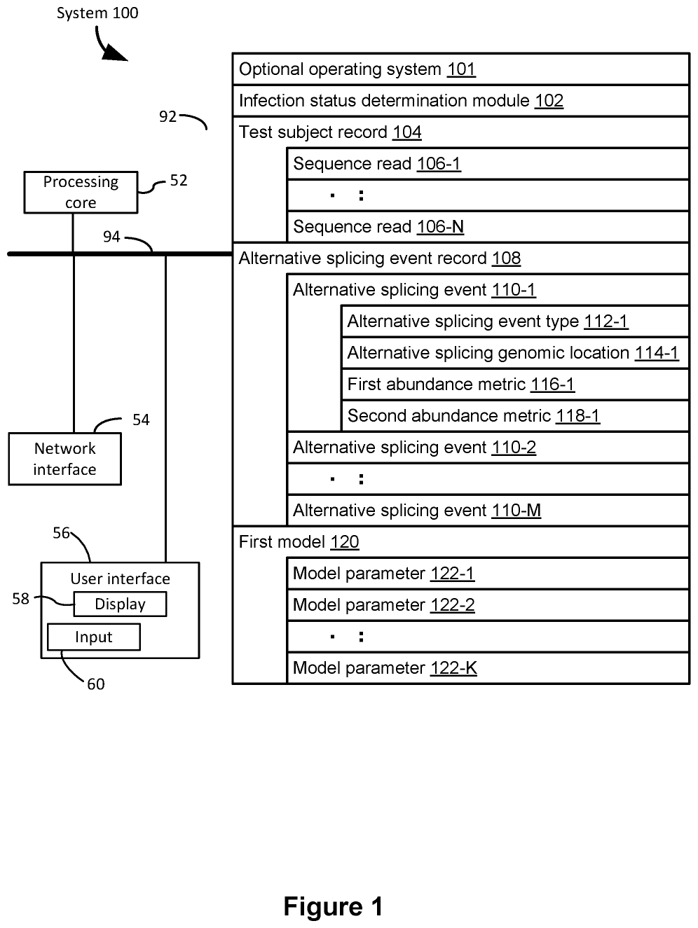

FIG. 1 illustrates a computer system 100 for determining an infection status of a test subject. In typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies is possible for the computer system 100 and all such topologies are within the scope of the present disclosure.

Turning to FIG. 1 with the foregoing in mind, the computer system 100 comprises one or more processing units (CPUs) 52, a network or other communications interface 54, a user interface 56 (e.g., including an optional display 58 and optional input 60 (e.g. keyboard or other form of input device), a memory 92 (e.g., random access memory, persistent memory, or combination thereof), and one or more communication busses 94 for interconnecting the aforementioned components. To the extent that components of memory 92 are not persistent, data in memory 92 can be seamlessly shared with non-volatile memory (not shown) or portions of memory 92 that are non-volatile/persistent using known computing techniques such as caching. Memory 92 can include mass storage that is remotely located with respect to the central processing unit(s) 52. In other words, some data stored in memory 92 may in fact be hosted on computers that are external to computer system 100 but that can be electronically accessed by the computer system 100 over an Internet, intranet, or other form of network or electronic cable using network interface 54. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 101 that includes procedures for handling various basic system services;
an infection status determination module 102 for determining an infection status of a test subject;
a test subject record comprising, in electronic form, sequence information for each RNA sequence read 106 (106-1, . . . , 106-N) in a plurality of RNA sequence reads from a biological sample of the test subject, where N is a positive integer of 2 or greater;
an alternative splicing event record 108 comprising: for each respective alternative splicing event 110 in a plurality of alternative splicing events (110-1, . . . , 110-2, . . . , 110-N) an indication of the alternative splicing event type 112, an indication of the alternative splicing event genome location 114, a value for a first abundance metric 116, and a value for a second abundance metric 118, where M is an integer greater than 2);
and
a first model 120 comprising a plurality of parameters 122-1, . . . , 122-K, where W is a positive integer of 2 or greater.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 optionally stores a subset of the modules and data structures identified above. Furthermore, in some embodiments the memory 92 stores additional modules and data structures not described above.

### Example Methods

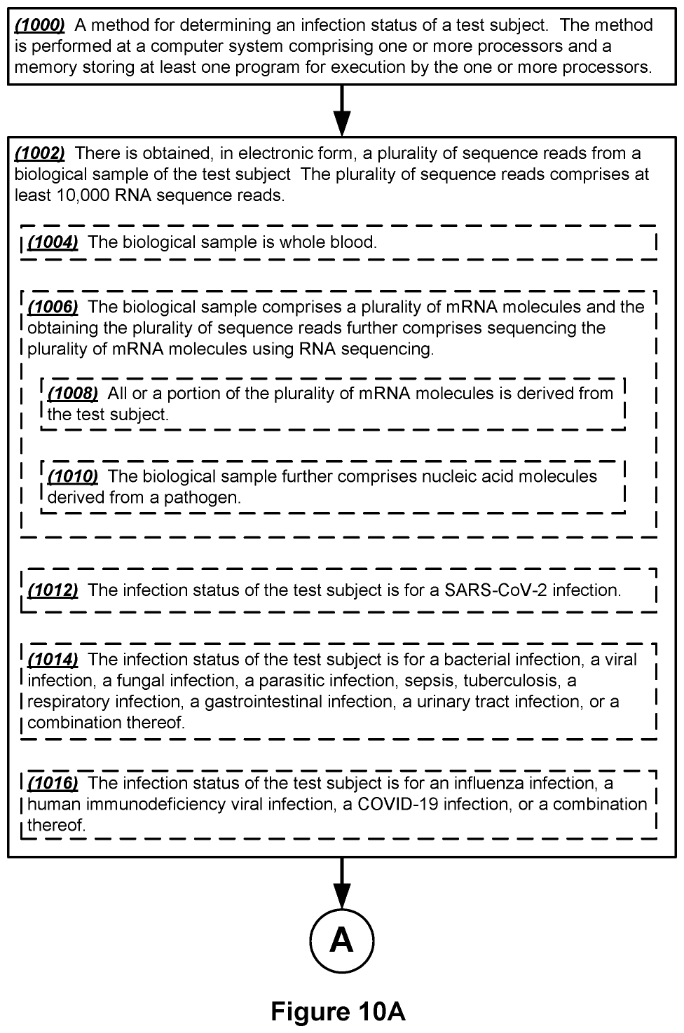

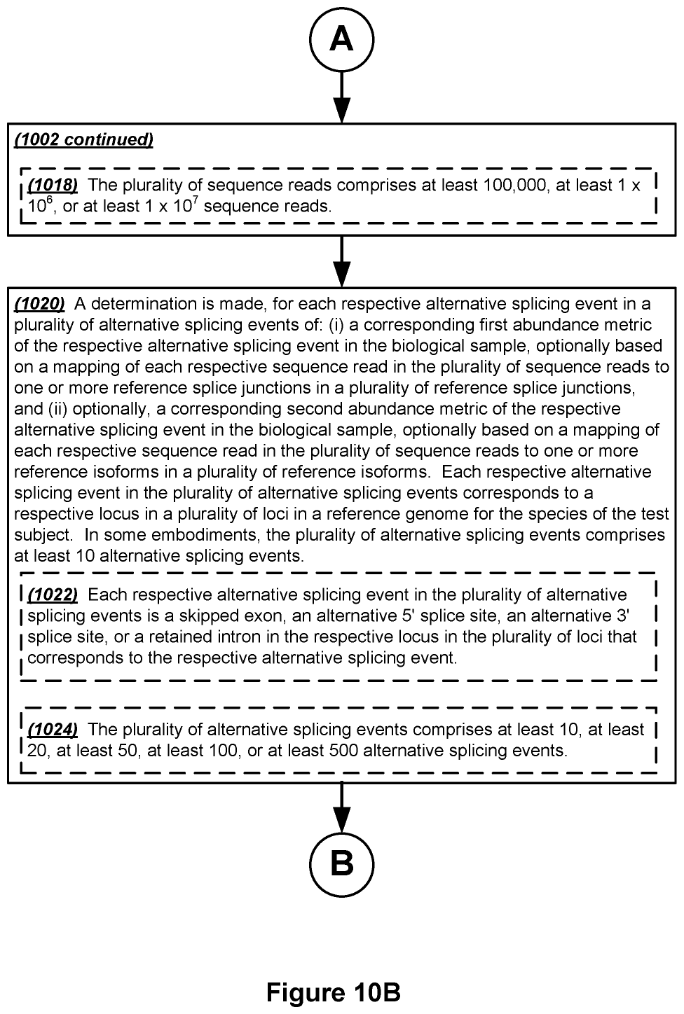

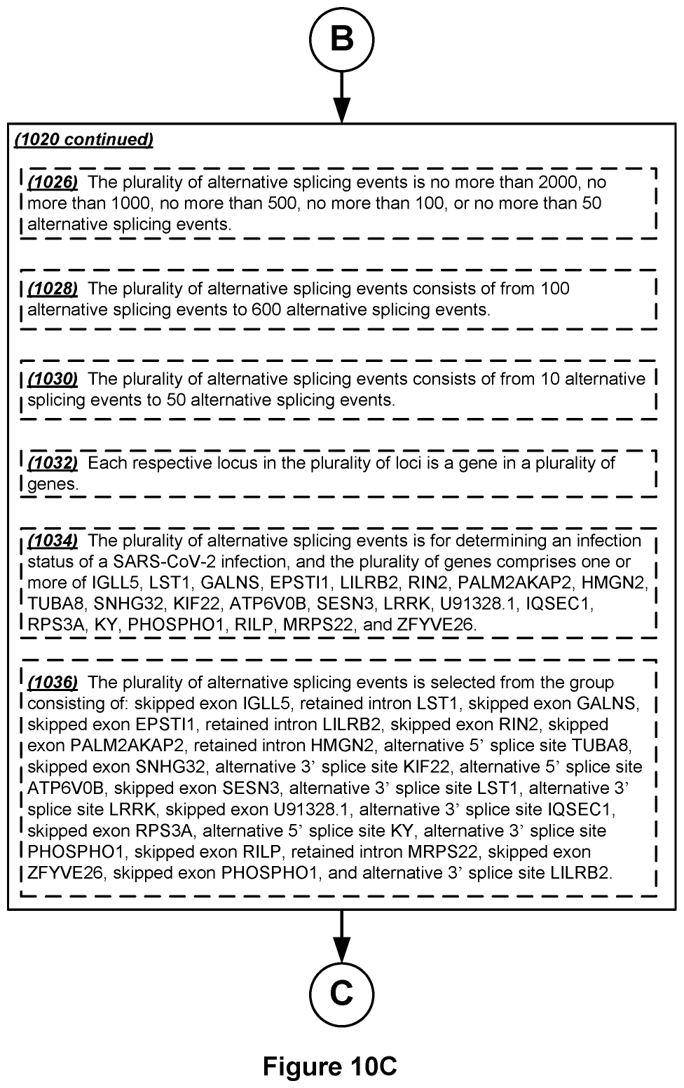

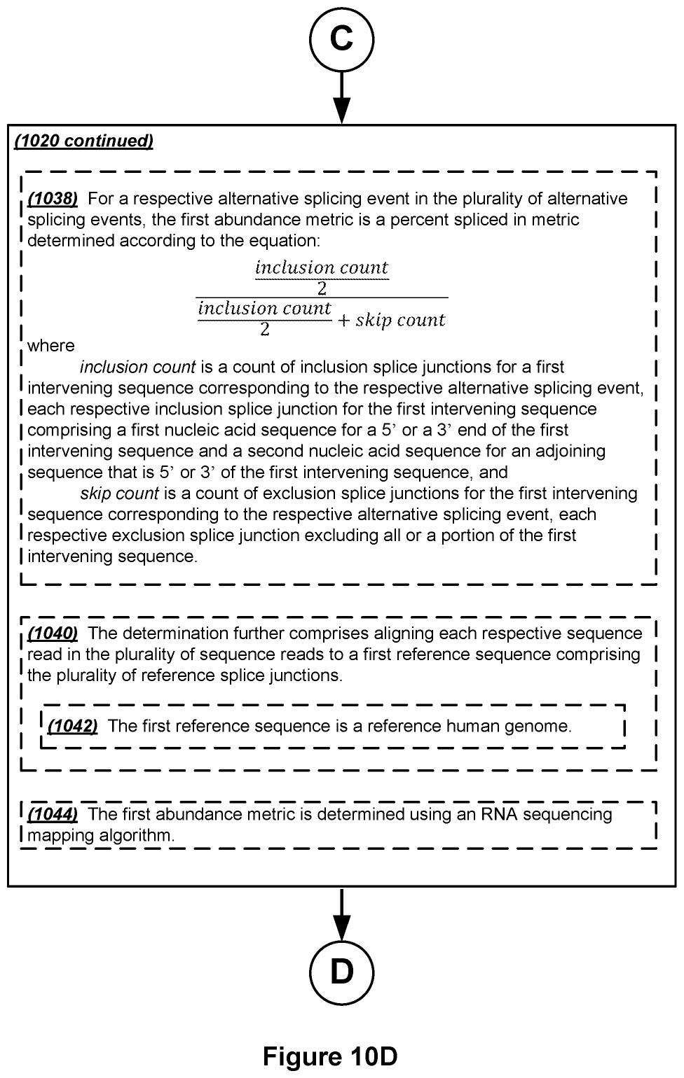

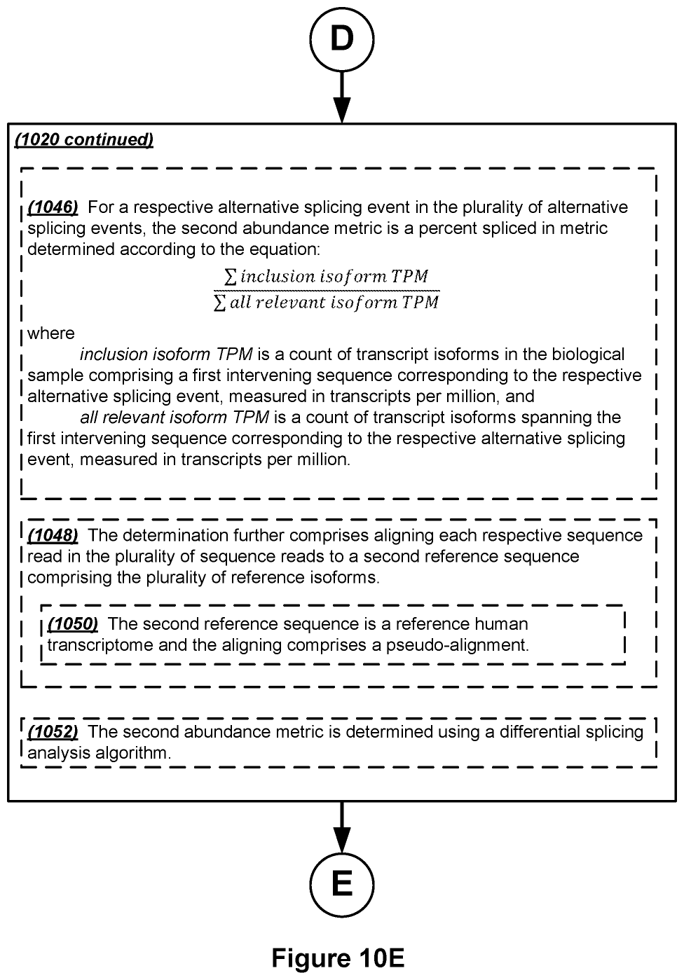

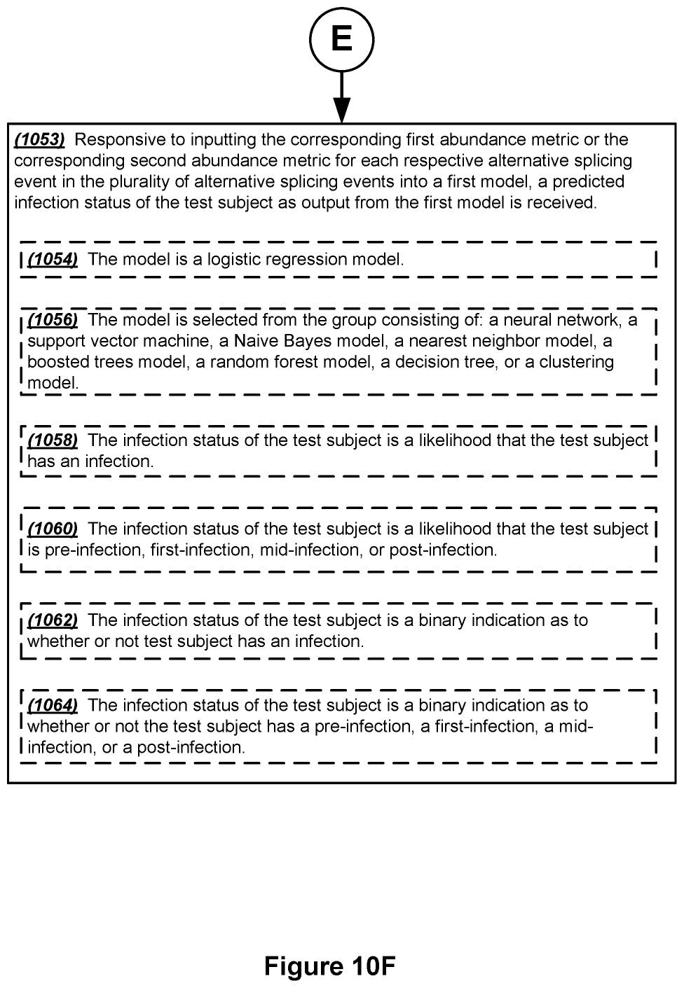

Now that an exemplary system of the present disclosure has been described, exemplary methods for determining an infection status of a test subject are disclosed in conjunction with FIGS. 10, 15, 16, and 17.

Referring to block 1000 of FIG. 10A, in some embodiments, a method for determining an infection status of a test subject is provided. The method is performed at a computer system comprising one or more processors and a memory storing at least one program for execution by the one or more processors.

Referring to block 1002, in some embodiments, there is obtained, in electronic form, a plurality of sequence reads from a biological sample of the test subject. In some embodiments, the plurality of sequence reads comprises at least 10,000 RNA sequence reads.

In some embodiments, the plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the plurality of sequence reads comprises at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, or more sequence reads. In some embodiments, the plurality of sequence reads consists of no more than 5×10⁷, no more than 1×10⁷, no more than 5×10⁶, no more than 4×10⁶, no more than 3×10⁶, no more than 2×10⁶, no more than 1×10⁶, no more than 500,000, no more than 100,000, no more than 50,000, no more than 30,000, no more than 20,000, no more than 10,000, no more than 9000, no more than 8000, no more than 7000, no more than 6000, no more than 5000, no more than 4000, no more than 3000, no more than 2000, no more than 1000, or less sequence reads.

In some embodiments, the plurality of sequence reads consists of between 1000 to 5000, from 1000 to 10,000, from 2000 to 20,000, from 5000 to 50,000, from 10,000 to 100,000, from 100,000 to 500,000 from 10,000 to 500,000, from 500,000 to 1 million, from 1 million to 30 million, from 30 million to 80 million, or from 10 million to 500 million sequence reads. In some embodiments, the plurality of sequence reads falls within another range starting no lower than 1000 sequence reads and ending no higher than 1×109 sequence reads.

Referring to block 1004, in some embodiments, the biological sample consists of whole blood. In some embodiments, the biological sample comprises blood, whole blood, plasma, serum, urine, cerebrospinal fluid, fecal, saliva, sweat, tears, pleural fluid, pericardial fluid, or peritoneal fluid from the test subject. In some embodiments, the biological sample consists of blood, whole blood, plasma, serum, urine, cerebrospinal fluid, fecal, saliva, sweat, tears, pleural fluid, pericardial fluid, or peritoneal fluid from the test subject.

Referring to block 1006, in some embodiments, the biological sample comprises a plurality of mRNA molecules and the obtaining the plurality of sequence reads further comprises sequencing the plurality of mRNA molecules using RNA sequencing. In some embodiments, the RNA sequencing is sequenced by Illumina high-throughput sequencing with paired-end reads. In some embodiments, the RNA sequencing is whole genome sequencing at an average depth of at least 5 million, 10 million, 15, million, 20 million, 25 million, 30 million, 35 million, 40 million, or 45 million.

In some embodiments, the RNA sequencing is Illumina RNA-seq.). In some embodiments the RNA sequencing is whole-blood RNA-seq. RNA-Seq (RNA sequencing) is sequences the transcriptome of a cell or tissue at a specific point in time. The transcriptome refers to the complete set of RNA molecules, including messenger RNA (mRNA), non-coding RNA, and other functional RNAs, present in a cell or tissue. For RNA-seq, RNA is isolated from the cells of the biological sample. This RNA can include both coding RNAs (mRNAs) that carry the genetic information to produce proteins, and non-coding RNAs that have various regulatory functions. The isolated RNA is converted into a sequencing library. This involves several steps, including reverse transcription of the RNA into complementary DNA (cDNA), adapter ligation, and amplification of the cDNA. The resulting library contains fragments of cDNA representing the original RNA molecules. The library is then subjected to high-throughput sequencing using platforms such as Illumina or Ion Torrent. During sequencing, the cDNA fragments are read, and the sequences of the individual fragments are generated.

Referring to block 1008, in some embodiments, all or a portion of the plurality of mRNA molecules is derived from the test subject.

Referring to block 1010, in some embodiments, the biological sample further comprises nucleic acid molecules derived from a pathogen.

Referring to block 1012, in some embodiments, the infection status of the test subject is for a SARS-CoV-2 infection.

Referring to block 1014, in some embodiments, the infection status of the test subject is for a bacterial infection, a viral infection, a fungal infection, a parasitic infection, sepsis, tuberculosis, a respiratory infection, a gastrointestinal infection, a urinary tract infection, or a combination thereof.

Referring to block 1016, in some embodiments, the infection status of the test subject is for an influenza infection, a human immunodeficiency viral infection, a COVID-19 infection, or a combination thereof.

Referring to block 1018, in some embodiments, the plurality of sequence reads comprises at least 100,000, at least 1×10⁶, or at least 1×10⁷ sequence reads.

Referring to block 1020, a determination is made, for each respective alternative splicing event 110 in a plurality of alternative splicing (AS) events (110-1, 110-2, . . . , 110-M) of a corresponding first abundance metric 116 of the respective alternative splicing event in the biological sample. In some embodiments the corresponding first abundance metric 116 uses data from on a mapping of each respective sequence read in the plurality of sequence reads to one or more reference splice junctions in a plurality of reference splice junctions. In some embodiments, a determination is also made, for each respective alternative splicing event 110 in the plurality of alternative splicing (AS) events (110-1, 110-2, . . . , 110-M) of a corresponding second abundance metric 118 of the respective alternative splicing event in the biological sample. In some embodiments the second abundance matrix makes use of a mapping of each respective sequence read in the plurality of sequence reads to one or more reference isoforms in a plurality of reference isoforms.

Each respective alternative splicing event in the plurality of alternative splicing events corresponds to a respective locus 114 in a plurality of loci in a reference genome for the species of the test subject. In some embodiments the reference genome is a human genome assembly, such as GRCh38/hg38. In some embodiments the reference genome is a human genome assembly available in on-line genome browser hosted by the National Center for Biotechnology Information (“NCBI”) or the University of California, Santa Cruz (UCSC). In some embodiments the reference genome is a human genome assembly is NCBI build 34 (UCSC equivalent: hg16), NCBI build 35 (UCSC equivalent: hg17), NCBI build 36.1 (UCSC equivalent: hg18), or GRCh37 (UCSC equivalent: hg19).

In some embodiments, the plurality of alternative splicing events comprises at least 10 alternative splicing events.

Using AS events as HRA markers is well grounded biologically. Almost all multi-exon human genes undergo AS, significantly diversifying the human transcriptome and proteome. Within infected cells, viral infection often disrupts host AS; evidence of hijacked host splicing machinery has been identified for Zika virus, human cytomegalovirus (HCMV), and SARS-CoV-2. See 9. Thompson et al., 2020, “Viral-induced alternative splicing of host genes promotes influenza replication,” Elife, 9, e55500; Banerjee et al., 2020, “SARS-CoV-2 disrupts splicing, translation, and protein trafficking to suppress host defenses,” Cell, 183, pg. 1325-1339.e21; Zhou et al., 2018, “Characterization of viral RNA splicing using whole-transcriptome datasets from host species,” Sci. Rep., 8, pg. 3273, each of which is hereby incorporated by reference in its entirety for all purposes. On the other hand, within human immune cells, a widespread splicing program switch has been observed upon external stimuli, including differential splicing of cell surface markers as well as transcriptional and RNA regulators, underlining the functional importance of splicing modulation during immune activation and response. See Martinez et al., 2013, “Control of alternative splicing in immune responses: many regulators, many predictions, much still to learn, “Immunol. Rev., 253, 216-236; Schaub et al., 2017, “Splicing in immune cells-mechanistic insights and emerging topics,” Int. Immunol., 29, pg. 173-181, each of which is hereby incorporated by reference in its entirety for all purposes.

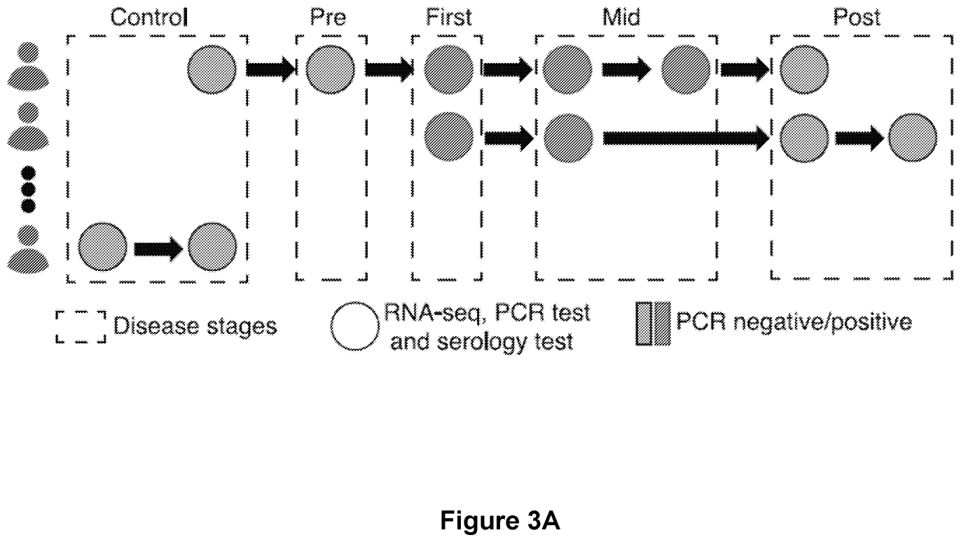

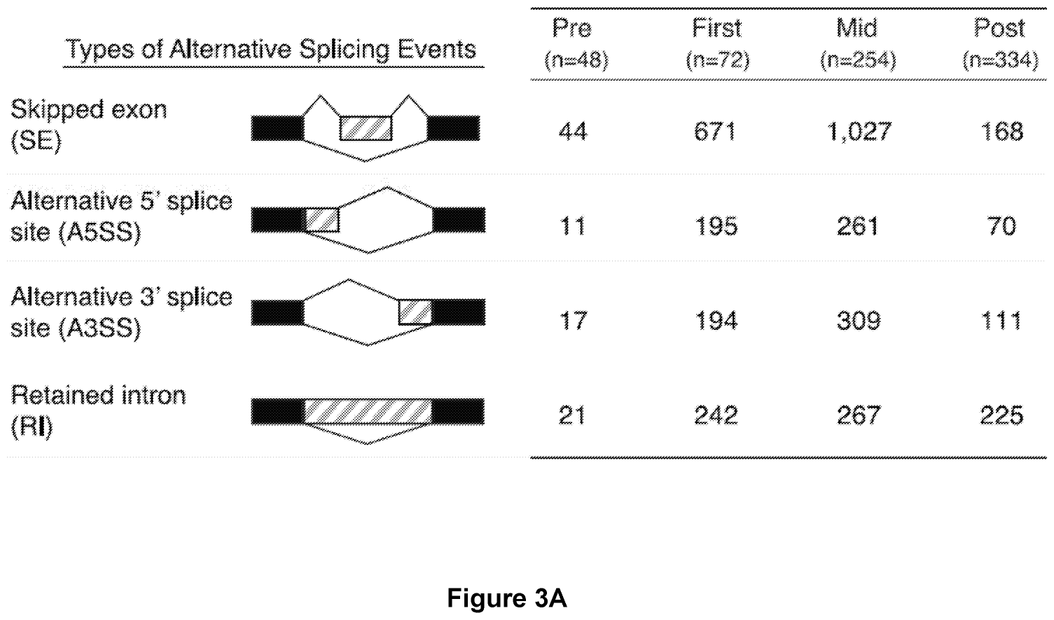

Referring to block 1022, in some embodiments, each respective alternative splicing event 110 in the plurality of alternative splicing events is one of plurality of alternative splicing event types 112. In some embodiments, is each respective alternative splicing event 110 in the plurality of alternative splicing events is a skipped exon, an alternative 5′ splice site, an alternative 3′ splice site, or a retained intron in the respective locus 114 in the plurality of loci that corresponds to the respective alternative splicing event 110. The skipped exon, alternative 5′ splice site, alternative 3′ splice site, and retained intron alternative splicing event types are defined in FIG. 3A.

Referring to block 1024, in some embodiments, the plurality of alternative splicing events comprises at least 10, at least 20, at least 50, at least 100, or at least 500 alternative splicing events. In some embodiments, the plurality of alternative splicing events consists of between 10 and 1000, between 20 and 500, between 50 and 300, between 100 and 500, or between 500 and 2000 alternative splicing events. Referring to block 1026, in some embodiments, the plurality of alternative splicing events is no more than 2000, no more than 1000, no more than 500, no more than 100, or no more than 50 alternative splicing events.

Referring to block 1028, in some embodiments, the plurality of alternative splicing events consists of from 100 alternative splicing events to 600 alternative splicing events.

Referring to block 1030, in some embodiments, the plurality of alternative splicing events consists of from 10 alternative splicing events to 50 alternative splicing events.

In some embodiments each alternative splicing events maps to a different genomic location in a reference genome. In some embodiments more than one alternative splicing event in the plurality of alternative splicing event can map the same genomic location in a reference genome.

Referring to block 1032, in some embodiments, each respective locus 114 in the plurality of loci is a gene in a plurality of genes.

Referring to block 1034, in some embodiments, the plurality of alternative splicing events is for determining an infection status of a SARS-CoV-2 infection, and the plurality of genes comprises one or more of IGLL5, LST1, GALNS, EPSTI1, LILRB2, RIN2, PALM2AKAP2, HMGN2, TUBA8, SNHG32, KIF22, ATP6V0B, SESN3, LRRK, U91328.1, IQSEC1, RPS3A, KY, PHOSPHO1, RILP, MRPS22, and ZFYVE26.

Referring to block 1036, in some embodiments, the plurality of alternative splicing events is selected from the group consisting of: skipped exon IGLL5, retained intron LST1, skipped exon GALNS, skipped exon EPSTI1, retained intron LILRB2, skipped exon RIN2, skipped exon PALM2AKAP2, retained intron HMGN2, alternative 5′ splice site TUBA8, skipped exon SNHG32, alternative 3′ splice site KIF22, alternative 5′ splice site ATP6V0B, skipped exon SESN3, alternative 3′ splice site LST1, alternative 3′ splice site LRRK, skipped exon U91328.1, alternative 3′ splice site IQSEC1, skipped exon RPS3A, alternative 5′ splice site KY, alternative 3′ splice site PHOSPHO1, skipped exon RILP, retained intron MRPS22, skipped exon ZFYVE26, skipped exon PHOSPHO1, and alternative 3′ splice site LILRB2.

In some embodiments, the plurality of alternative splicing events comprises 2, 3, 4, 5, 6, 7, 8, 9, 10, 11, 12, 13, 14, 15, 16, 17, 18, 19, 20, 21, 22, 23, 24, 25, 26, or all 27 of the alternative splicing events (rows) listed in Table 2 of Example 3. In some embodiments, the plurality of alternative splicing events consists of 2, 3, 4, 5, 6, 7, 8, 9, 10, 11, 12, 13, 14, 15, 16, 17, 18, 19, 20, 21, 22, 23, 24, 25, 26, or all 27 of the alternative splicing events listed in Table 2 of Example 3. In some embodiments, the plurality of alternative splicing events comprises 2, 3, 4, 5, 6, 7, 8, 9, 10, 11, 12, 13, 14, 15, 16, 17, 18, 19, 20, 21, 22, 23, 24, 25, 26, or all 27 of the alternative splicing events listed in Table 2 of Example 3 as well as 1 or more, 2 or more, 3 or more, 4 or more, 5 or more, 6 or more, 7 or more, 8 or more, 9 or more, or 10 or more alternative splicing events not listed in Table 2.

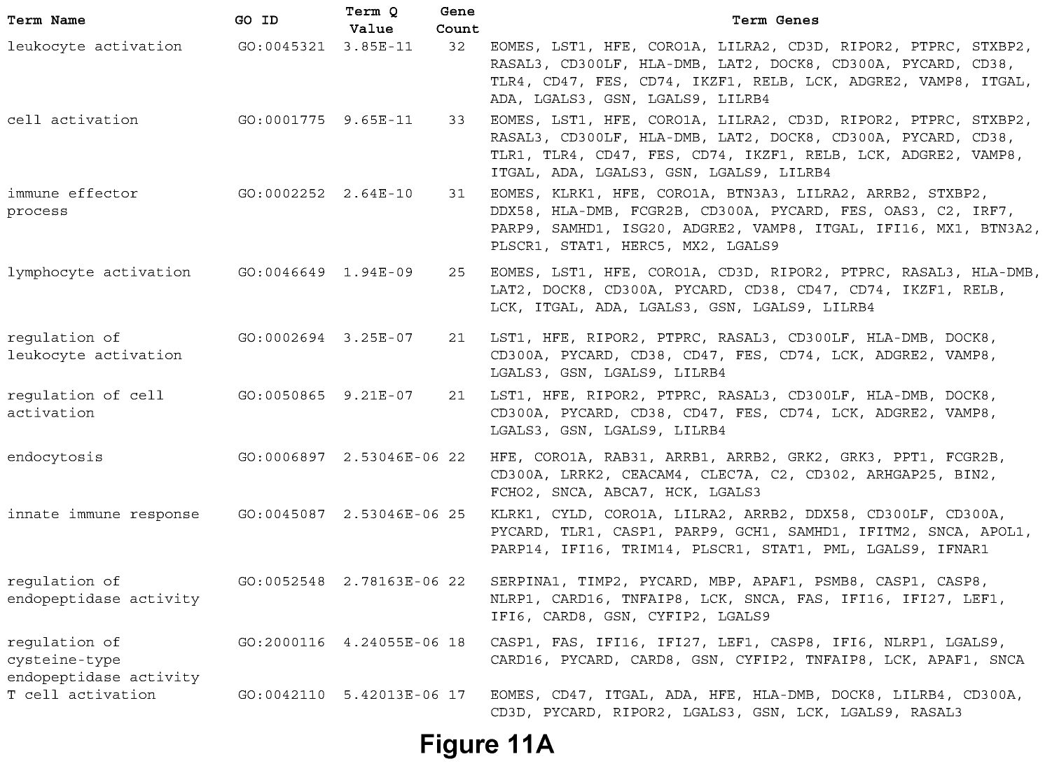

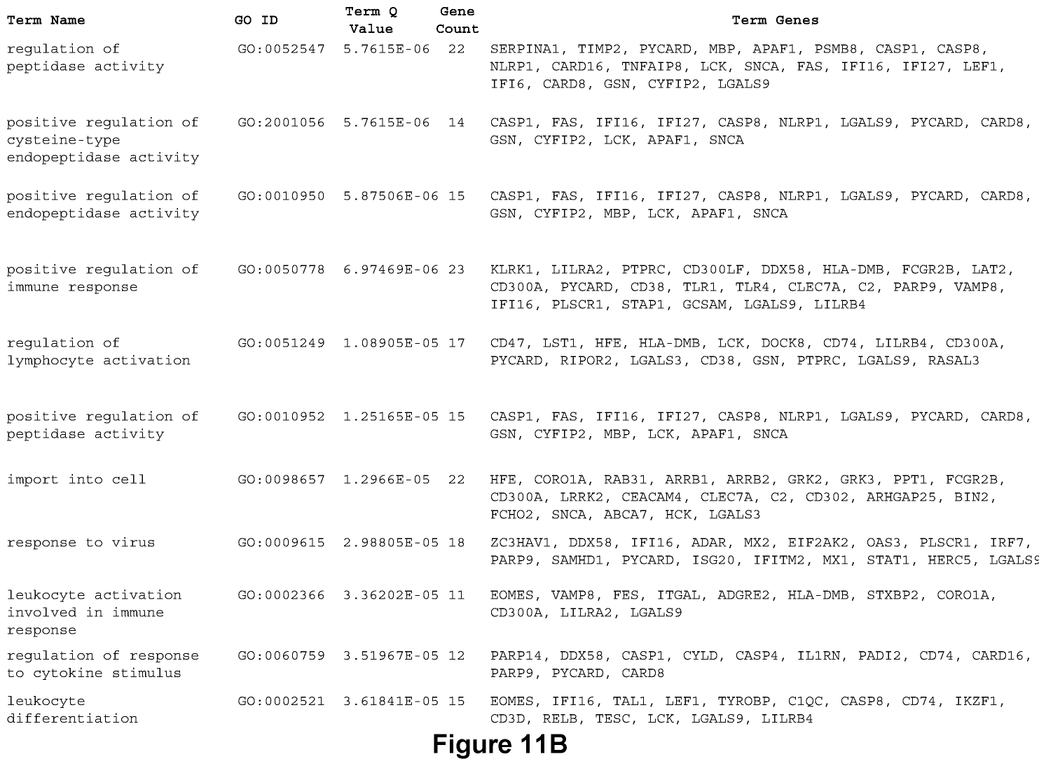

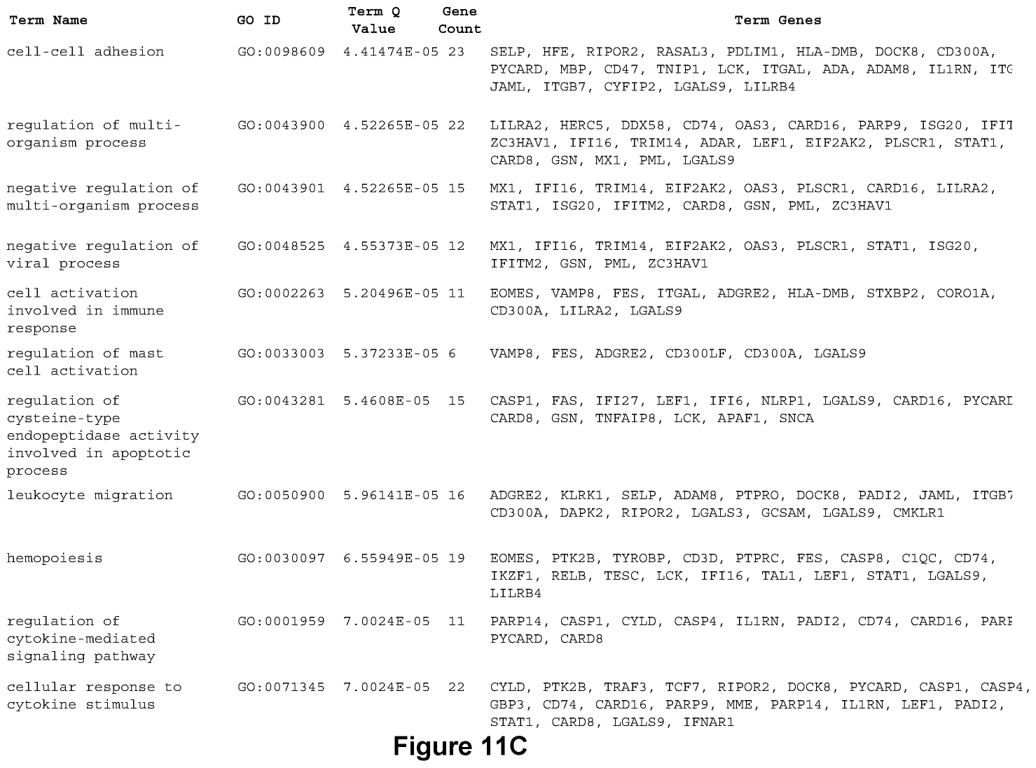

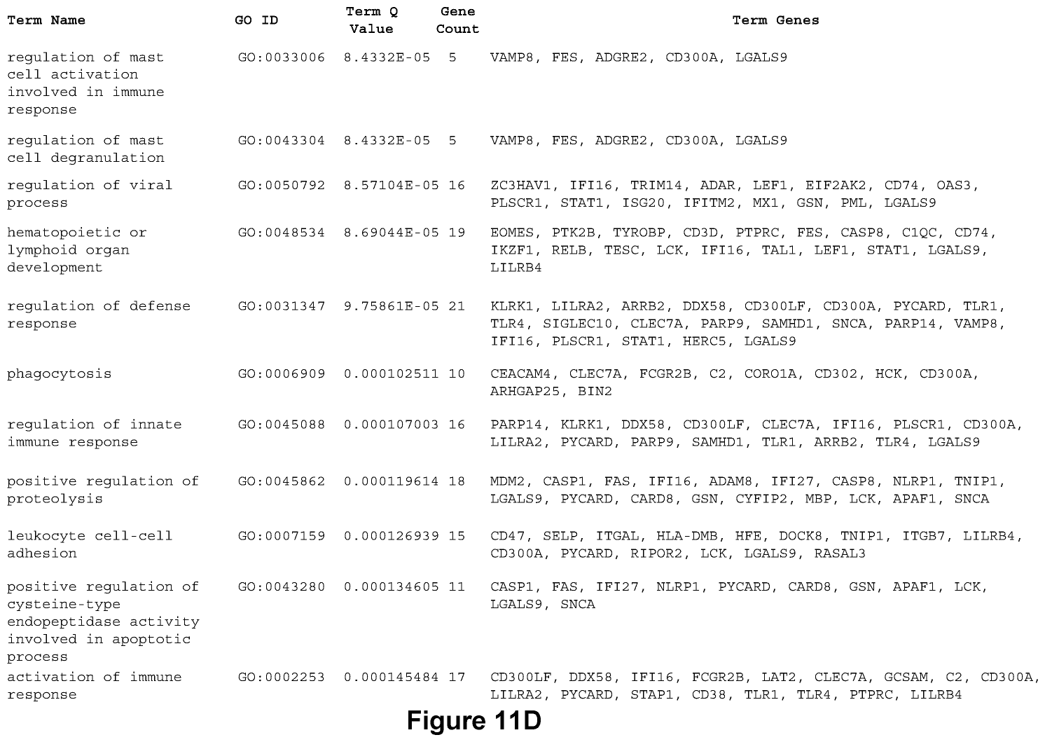

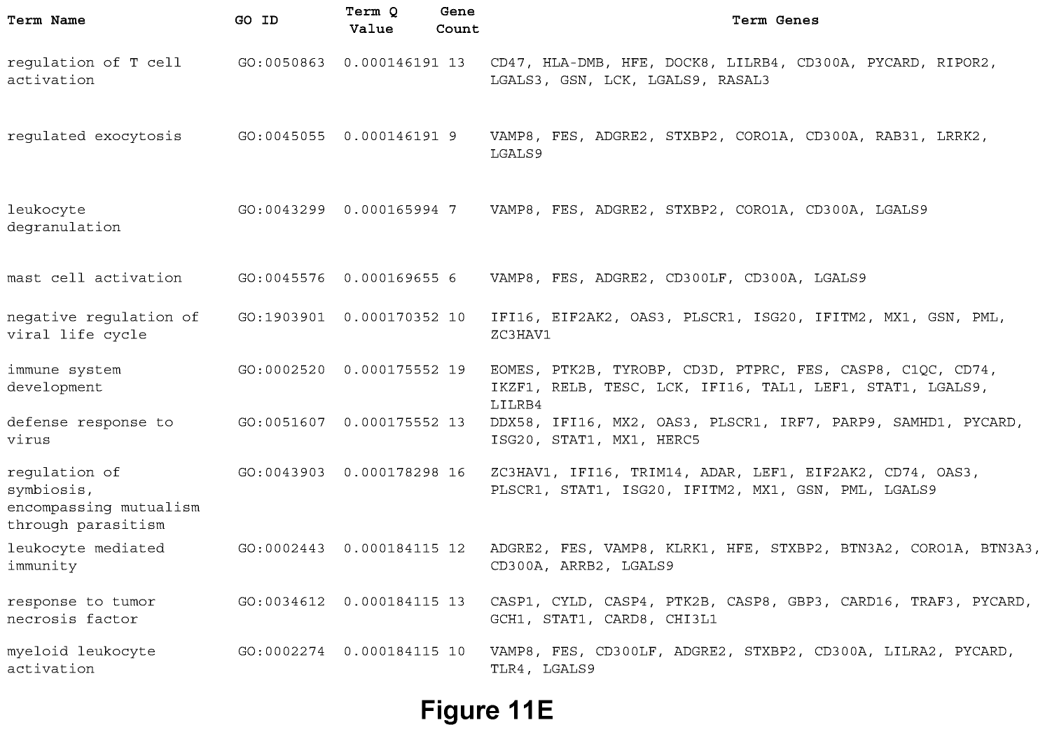

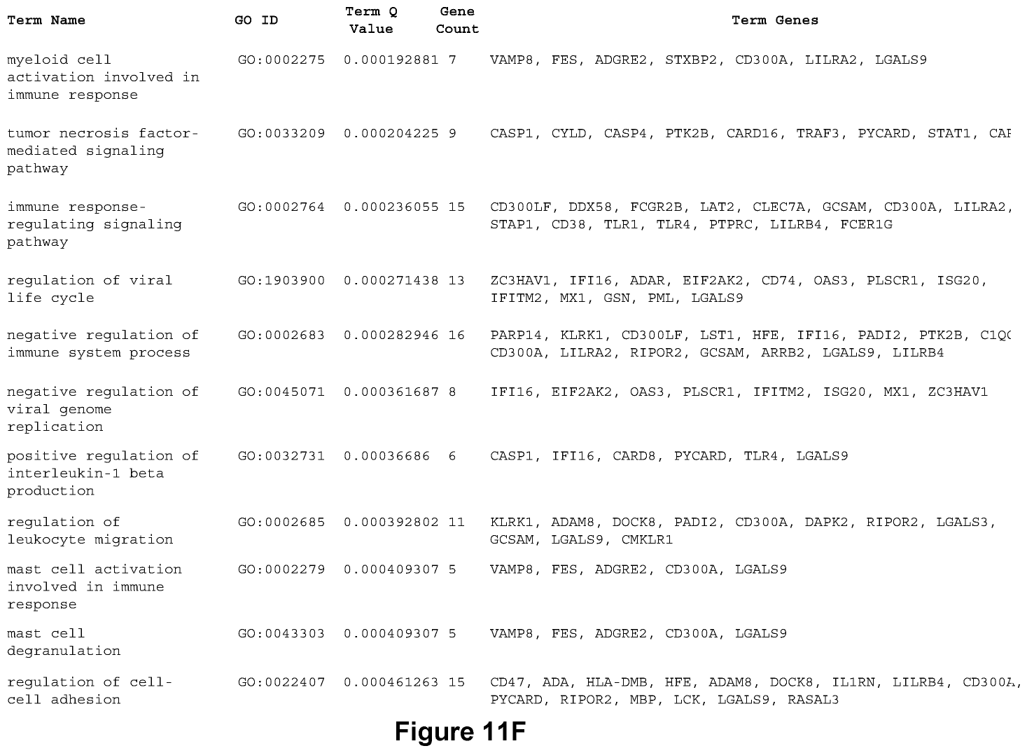

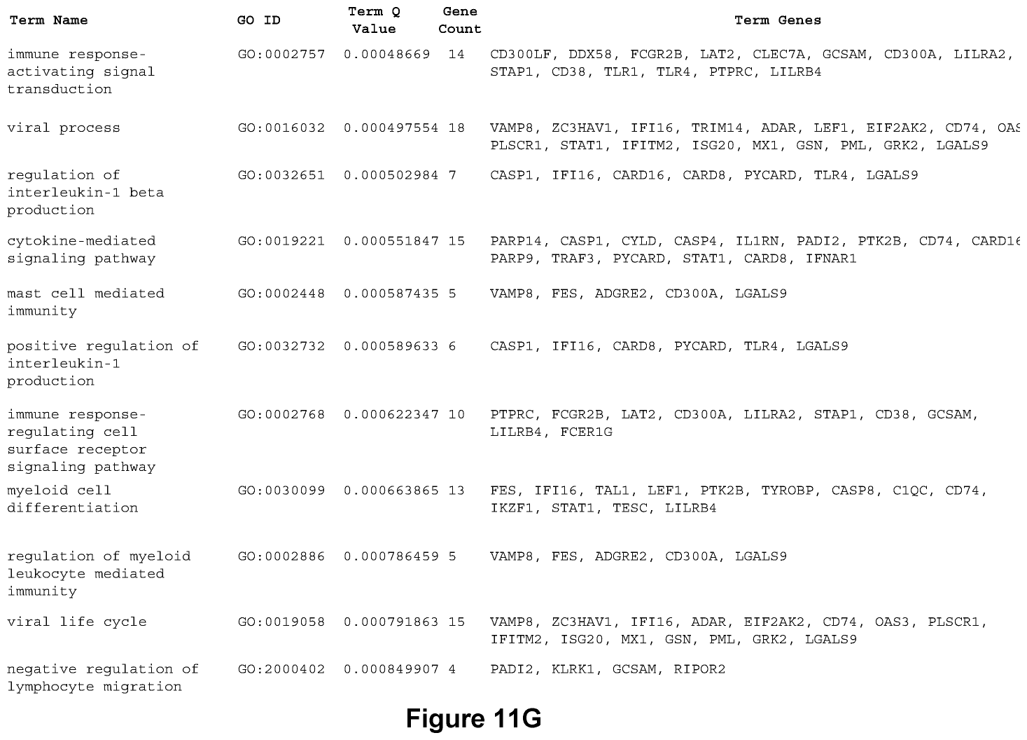

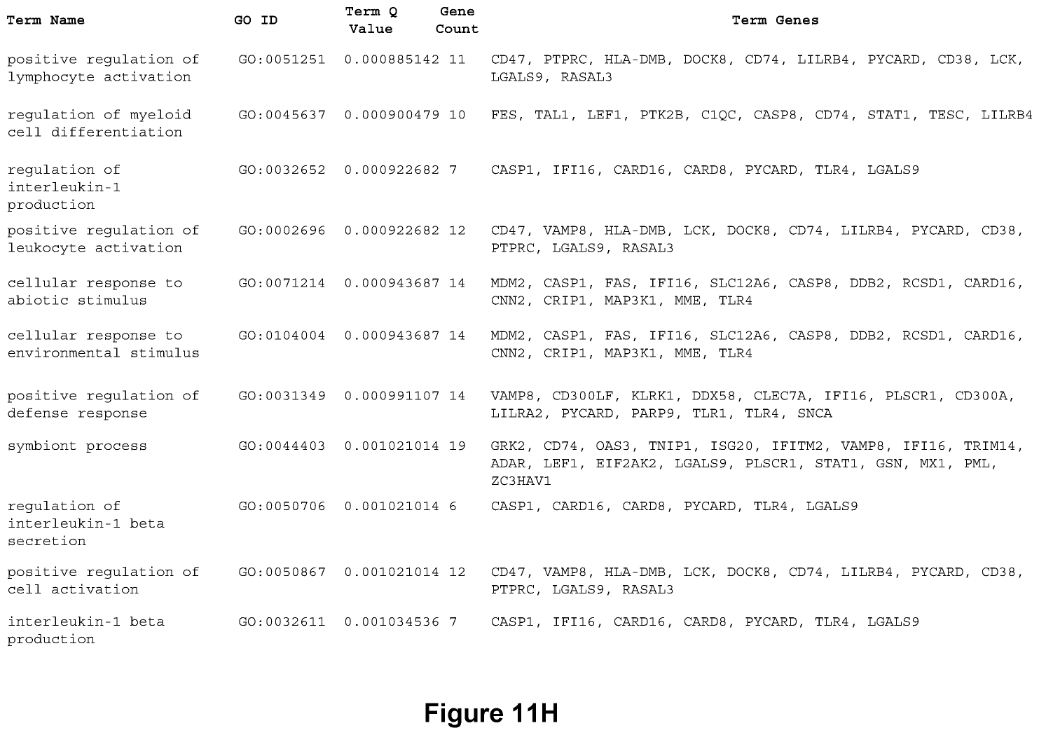

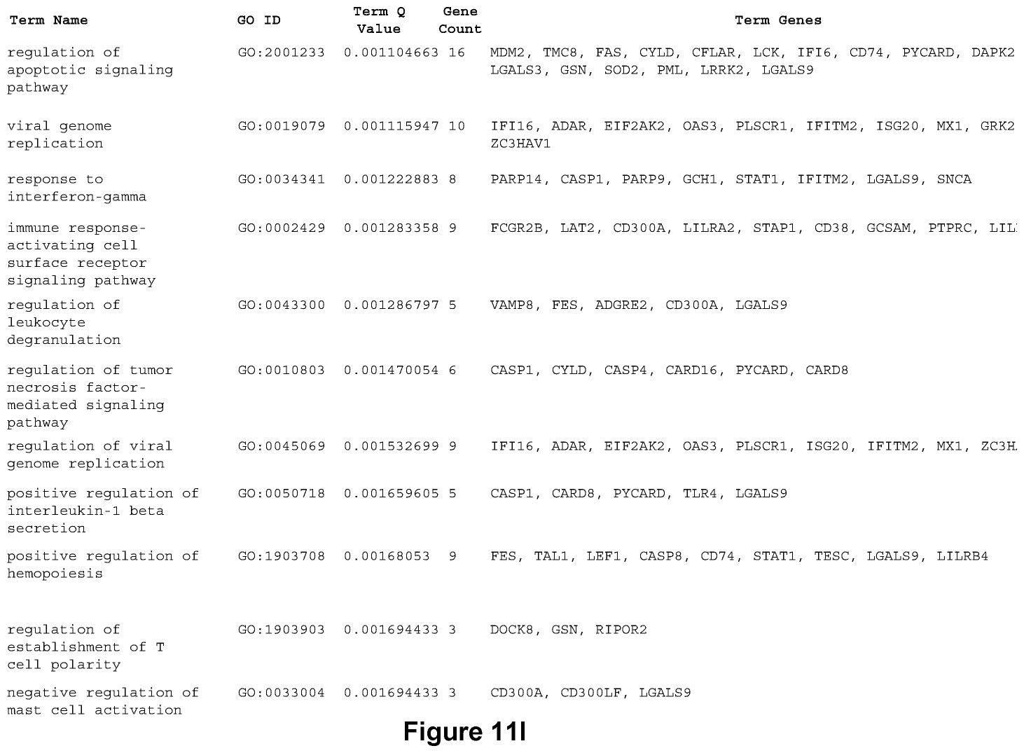

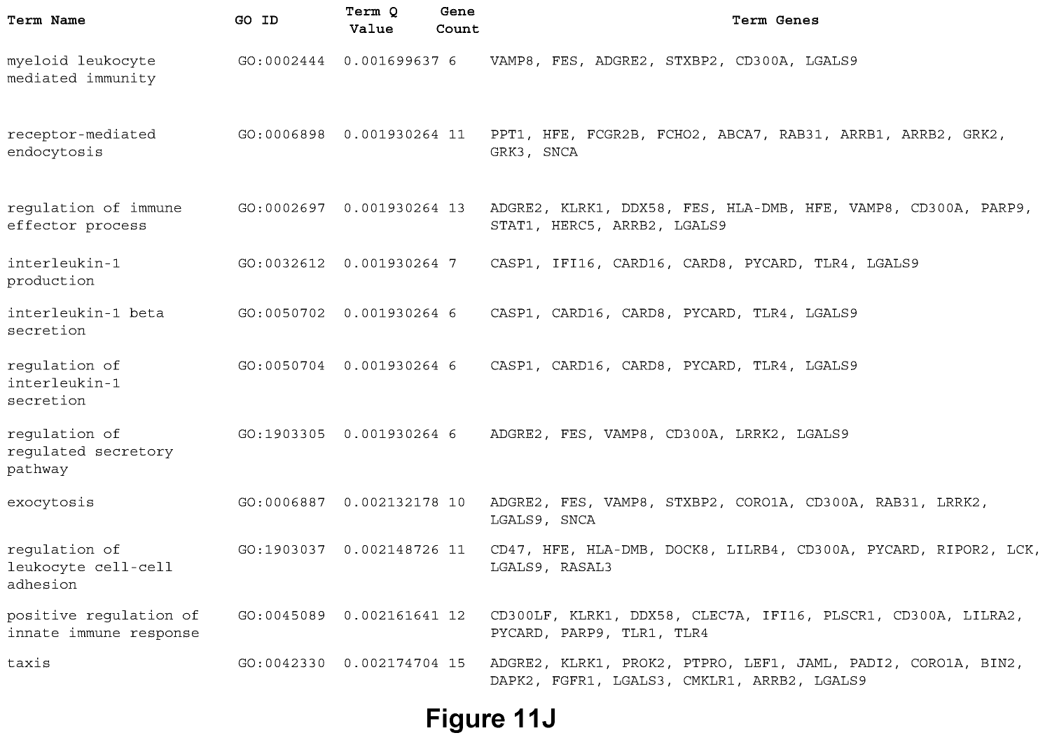

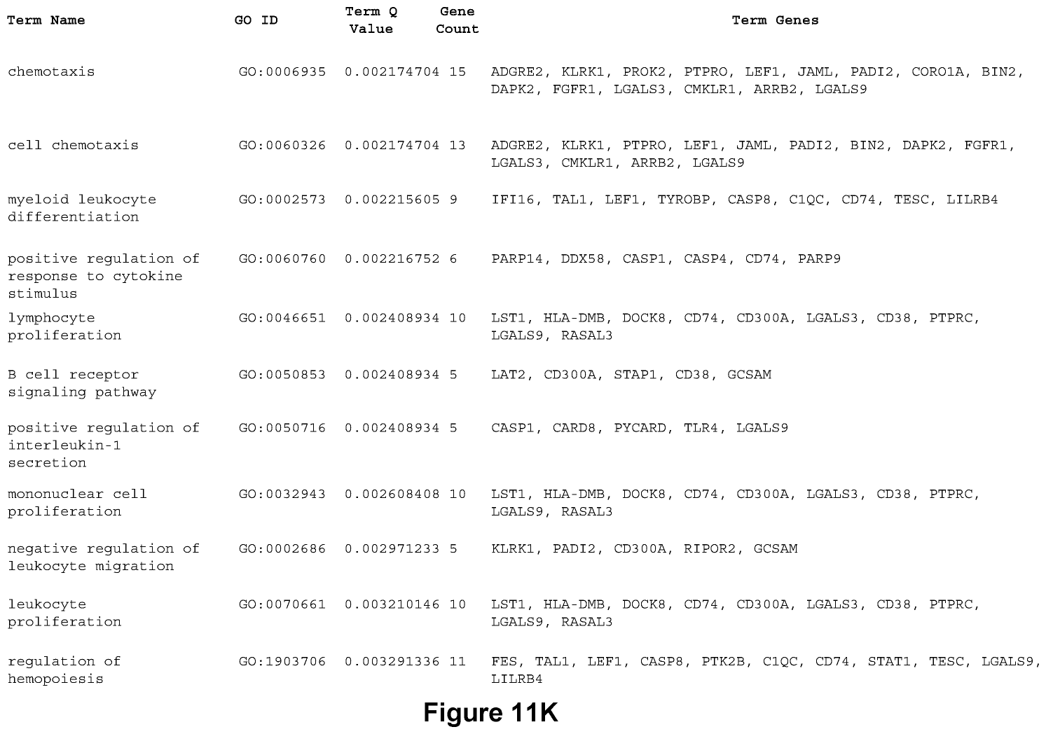

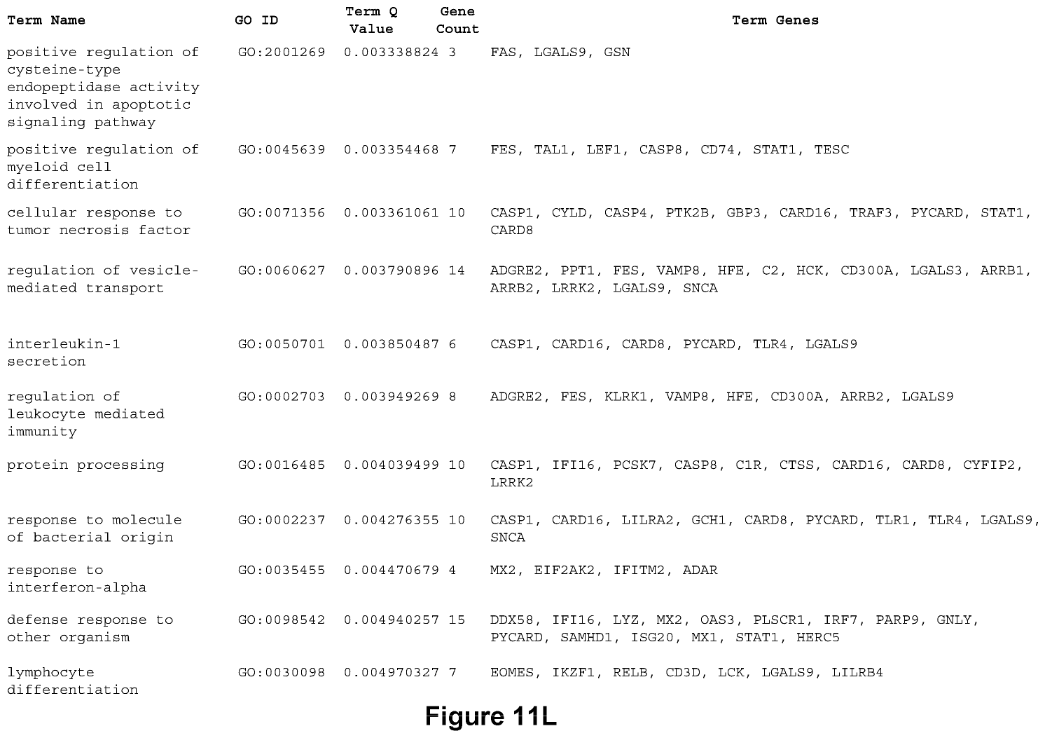

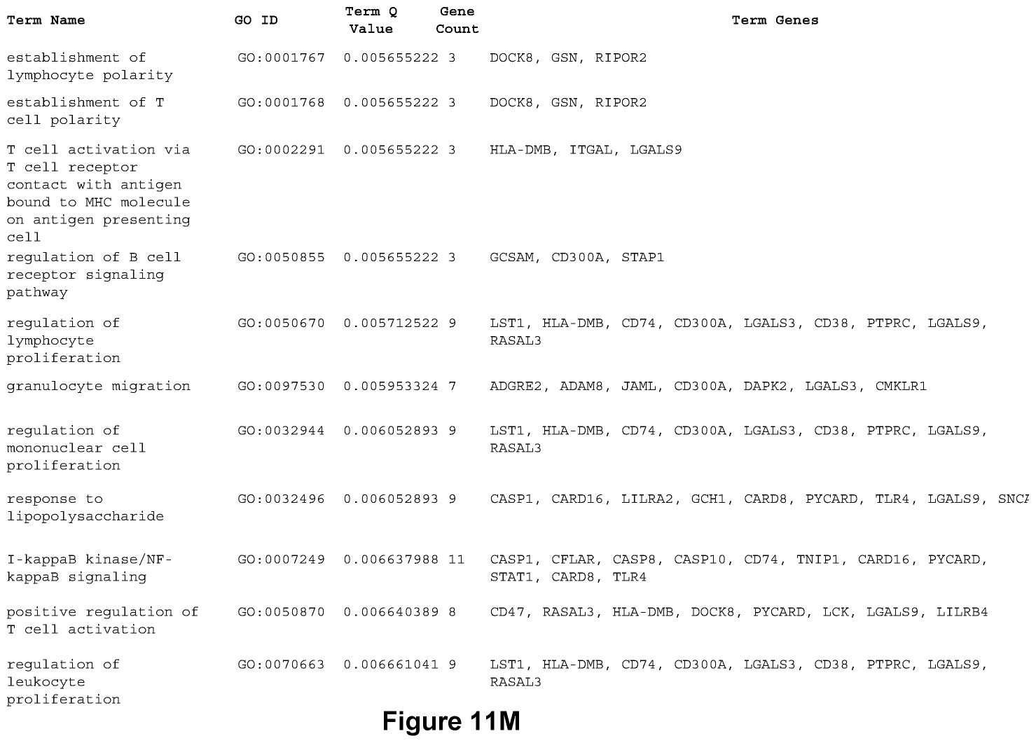

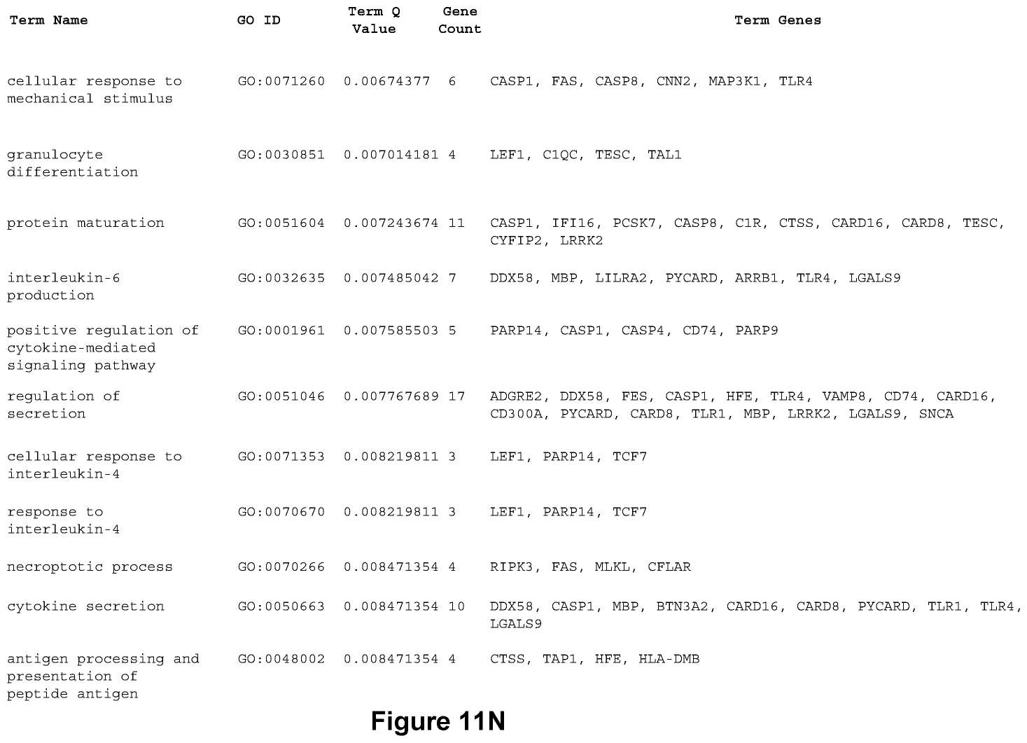

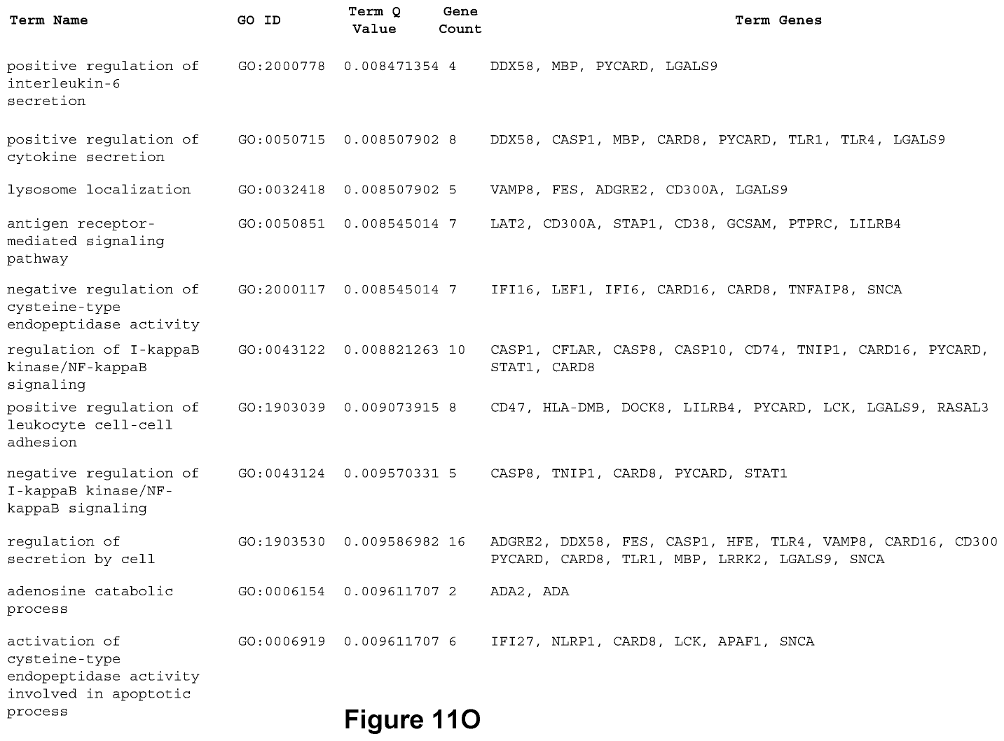

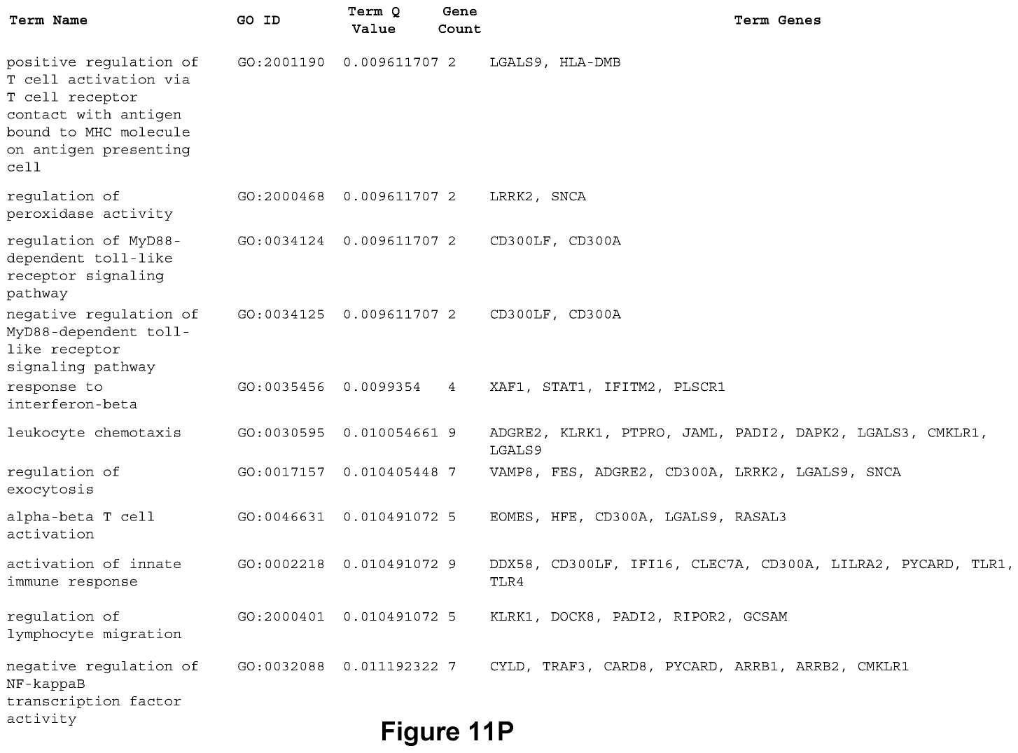

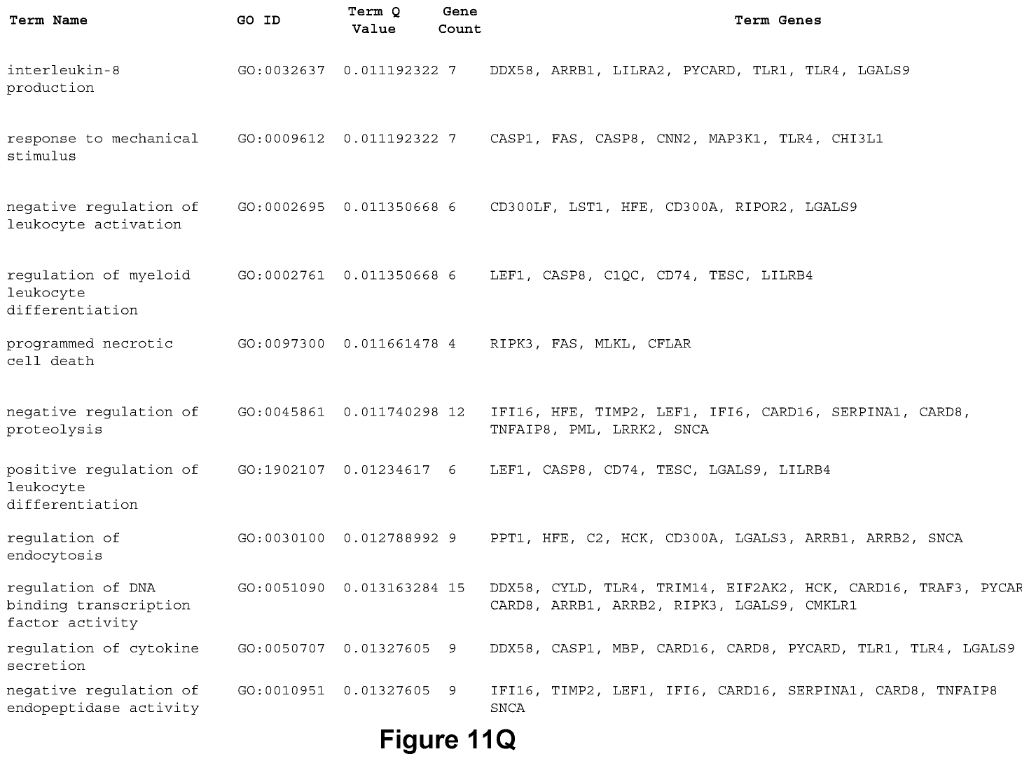

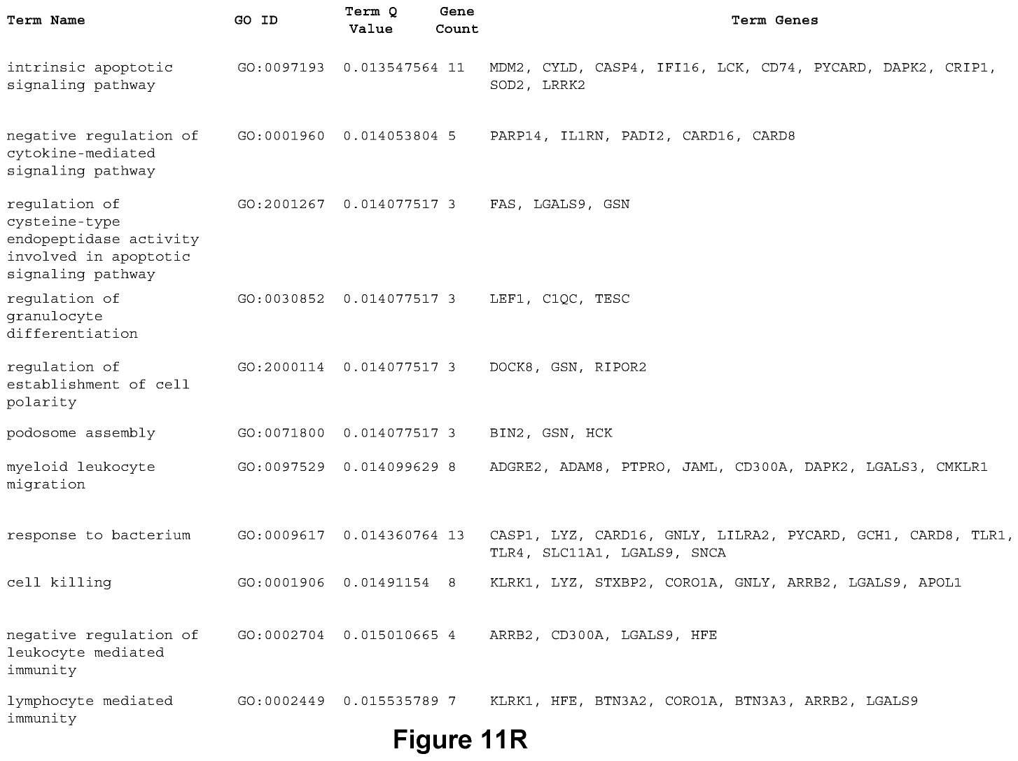

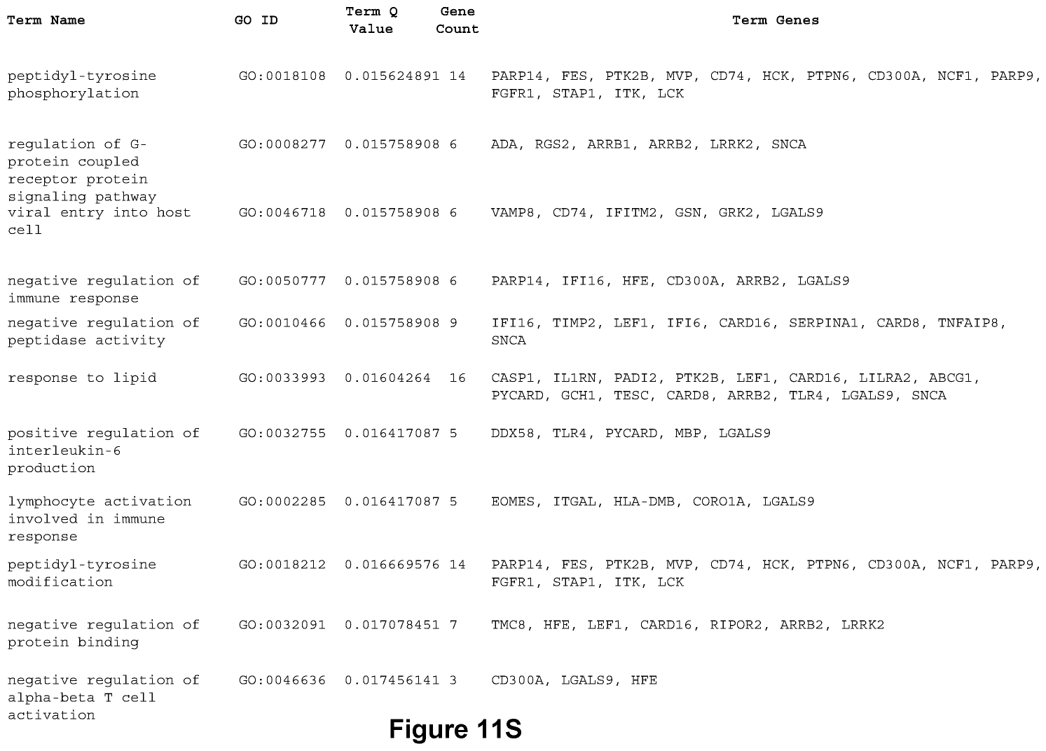

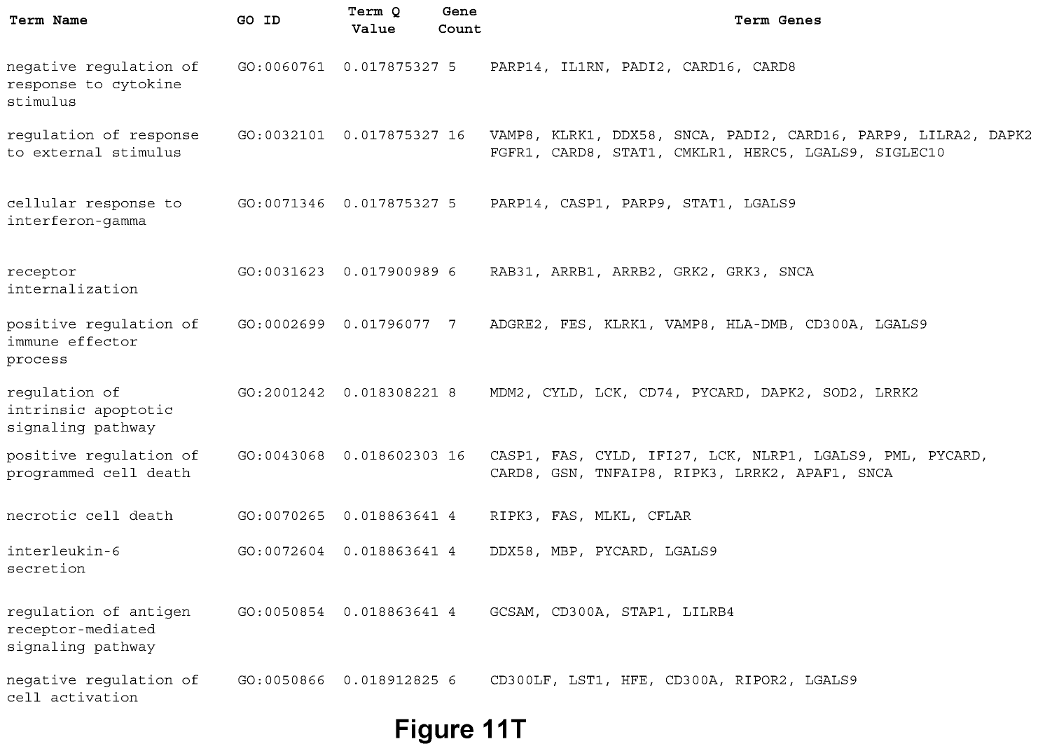

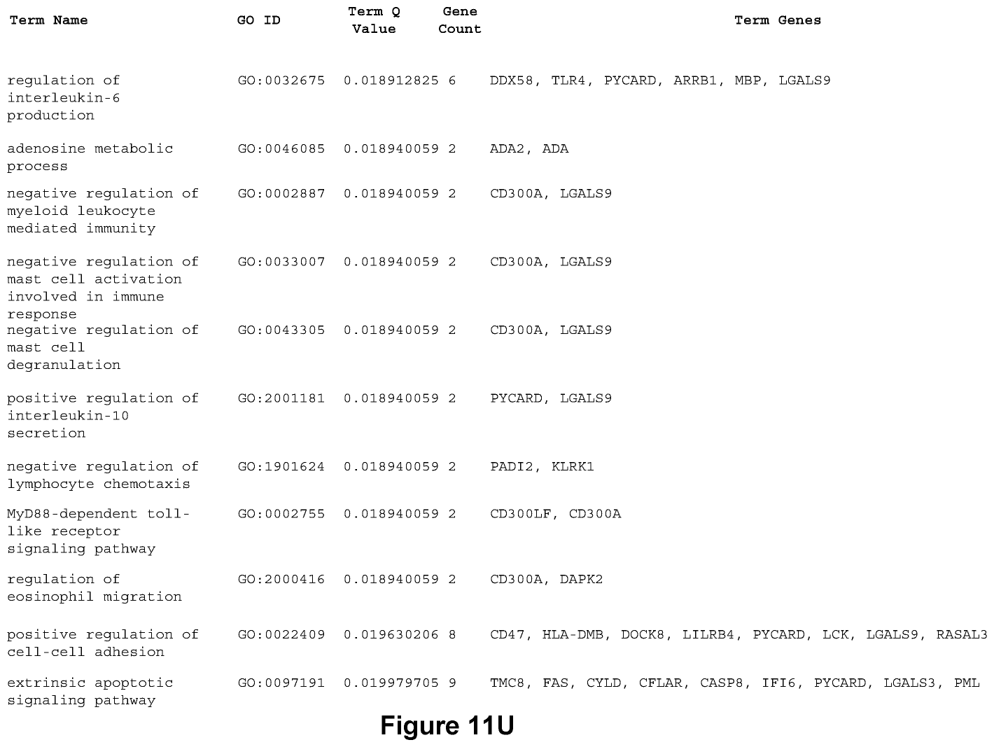

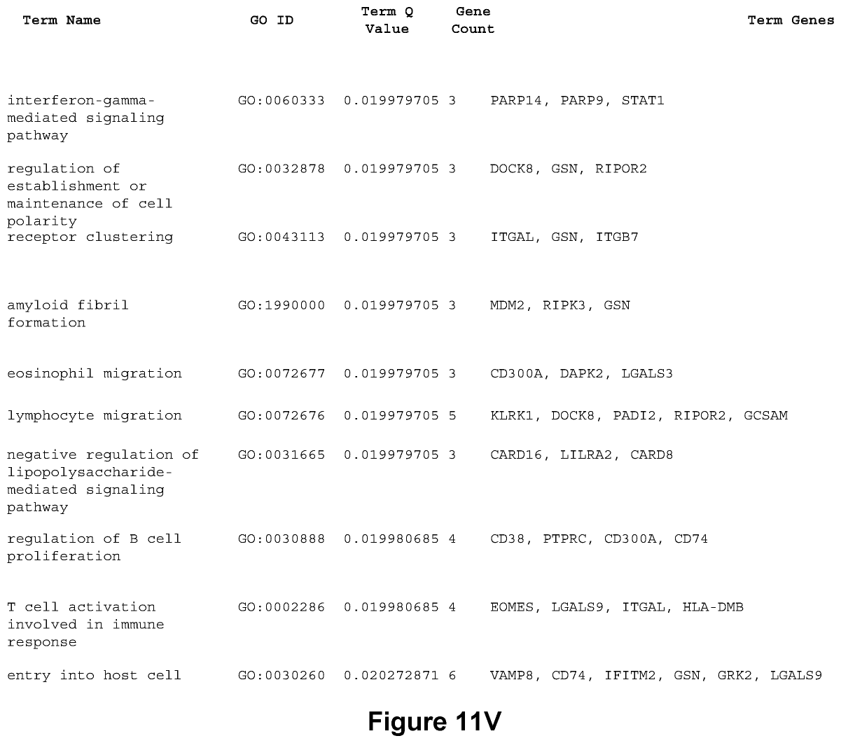

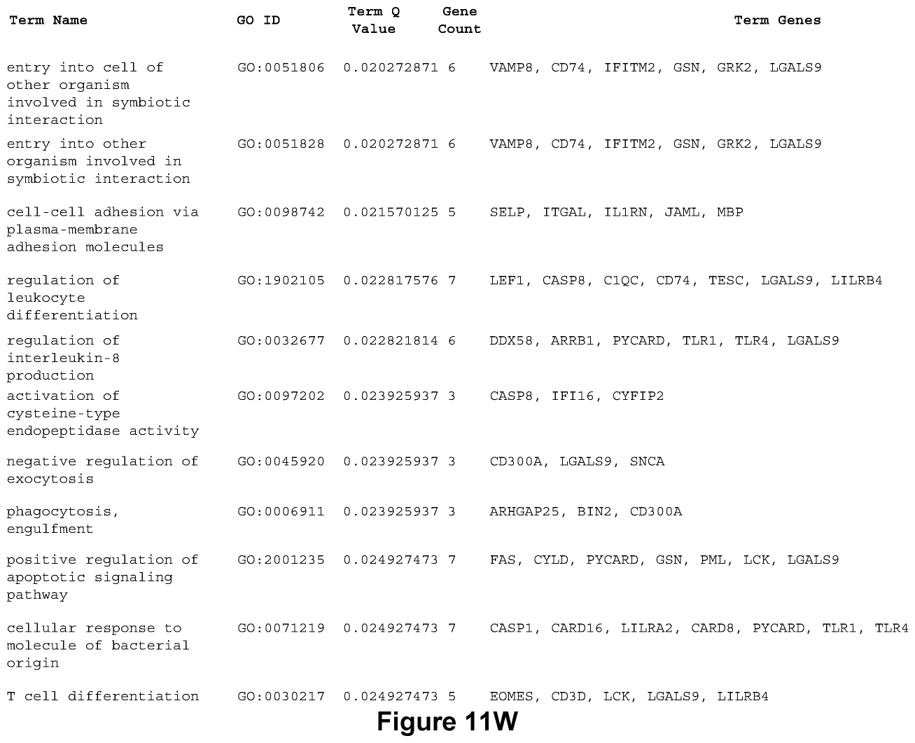

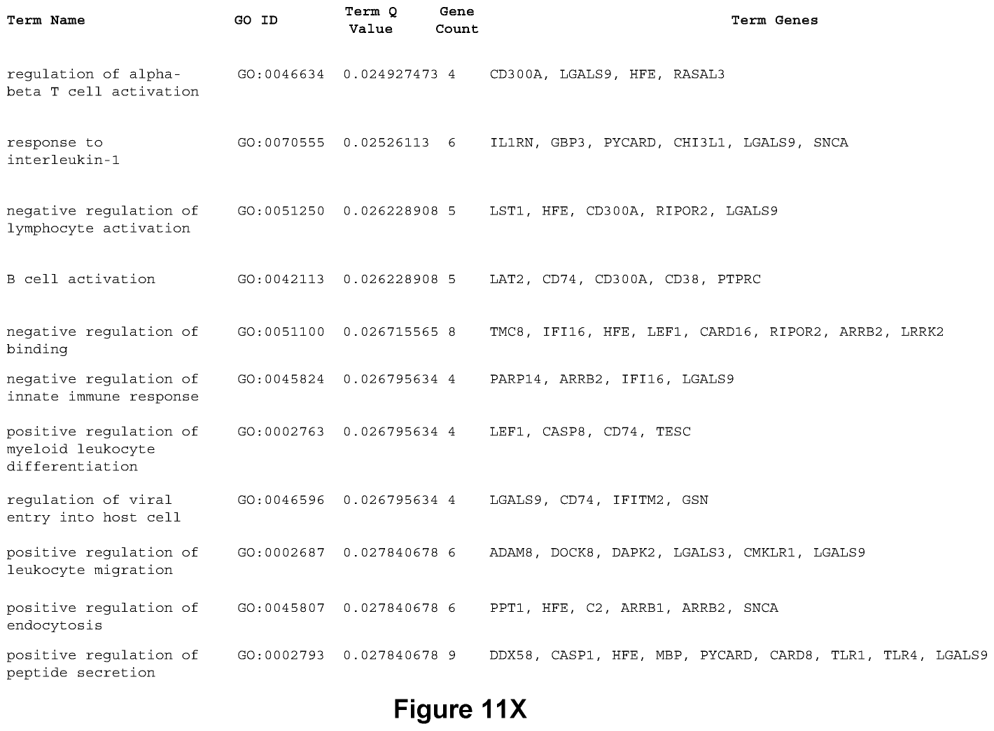

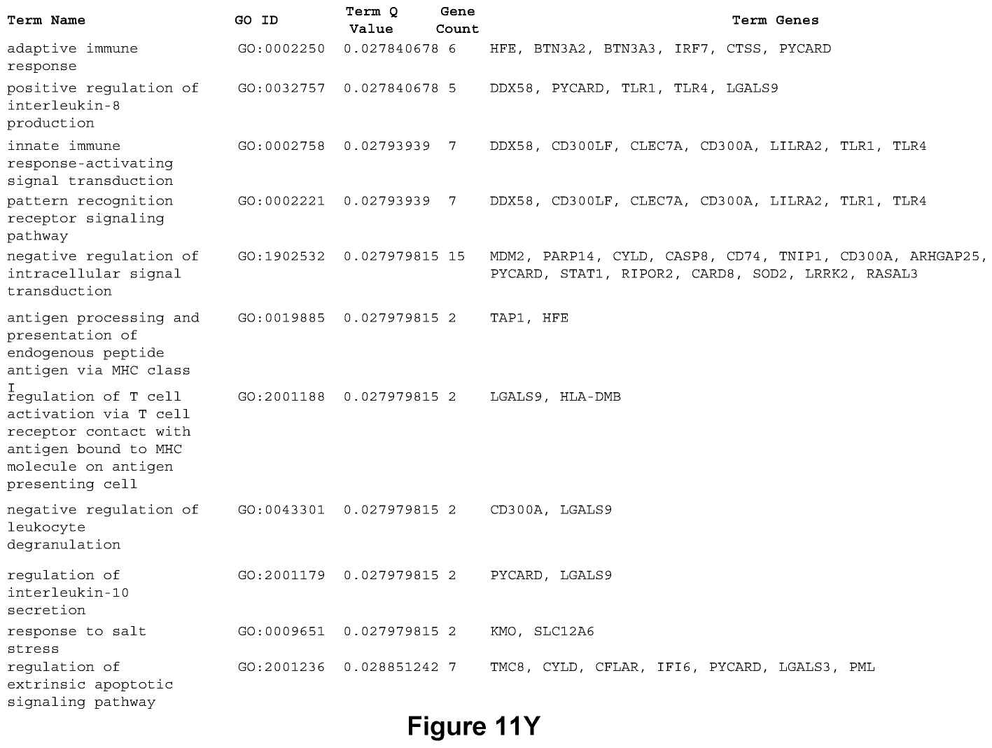

In some embodiments, the plurality of alternative splicing events comprises 2, 3, 4, 5, 6, 7, 8, 9, 10, 11, 12, 13, 14, 15, 16, 17, 18, 19, 20, 21, 22, 23, 24, 25, 26, or all of the alternative splicing events (rows) listed in Table 4 described below in conjunction with FIG. 11. In some embodiments, the plurality of alternative splicing events consists of 2, 3, 4, 5, 6, 7, 8,9, 10, 11, 12, 13, 14, 15, 16, 17, 18, 19, 20, 21, 22, 23, 24, 25, 26, 27, 28, 29, 30, between 25 and 50, or all of the alternative splicing events listed in Table 4. In some embodiments, the plurality of alternative splicing events comprises 2, 3, 4, 5, 6, 7, 8, 9, 10, 11, 12, 13, 14, 15, 16, 17, 18, 19, 20, 21, 22, 23, 24, 25, 26, or all 27 of the alternative splicing events listed in Table 4 as well as 1 or more, 2 or more, 3 or more, 4 or more, 5 or more, 6 or more, 7 or more, 8 or more, 9 or more, or 10 or more alternative splicing events not listed in Table 4. In some embodiments, the plurality of alternative splicing events comprises 2, 3, 4, 5, 6, 7, 8, 9, 10, 11, 12, 13, 14, 15, 16, 17, 18, 19, 20, 21, 22, 23, 24, 25, 26, or all 27 of the alternative splicing events listed in Table 4 as well as 1 or more, 2 or more, 3 or more, 4 or more, 5 or more, 6 or more, 7 or more, 8 or more, 9 or more, or 10 or more alternative splicing events not listed in Table 2.

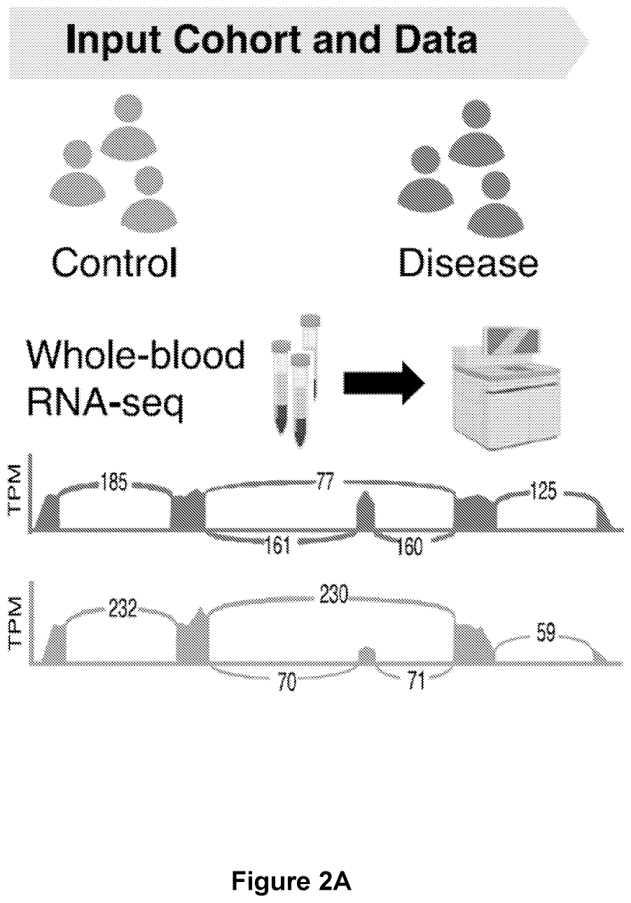

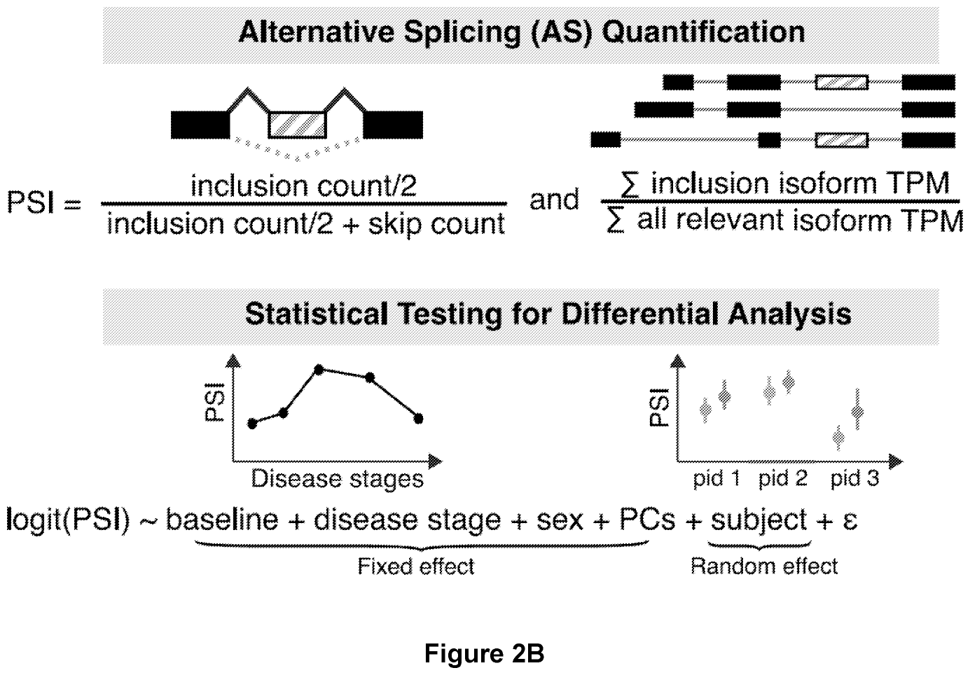

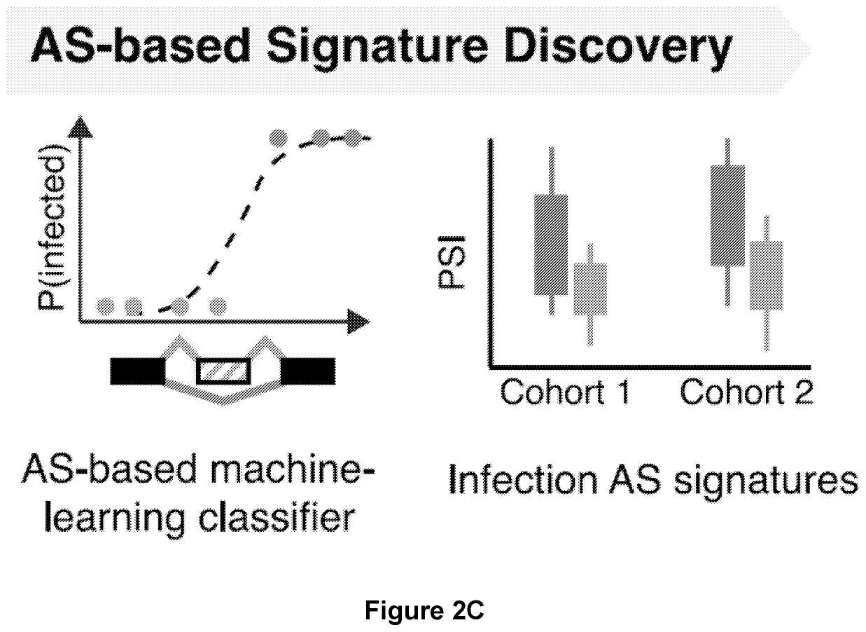

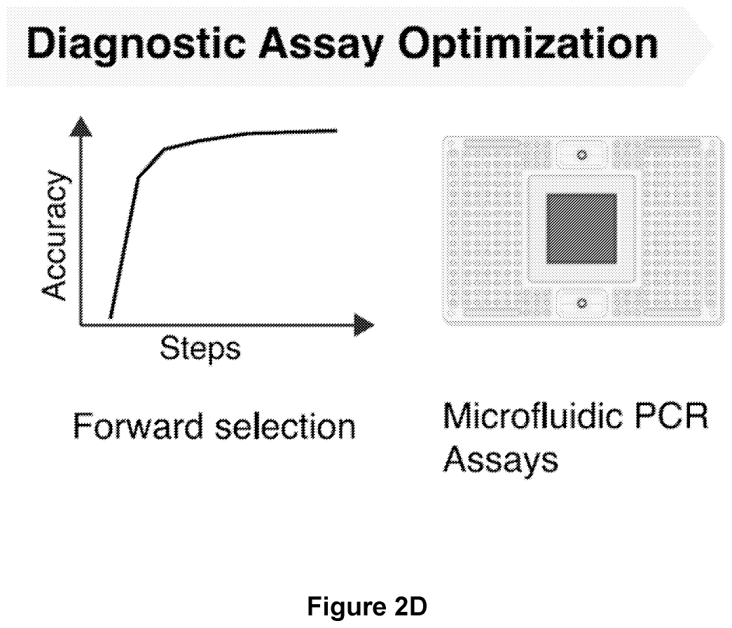

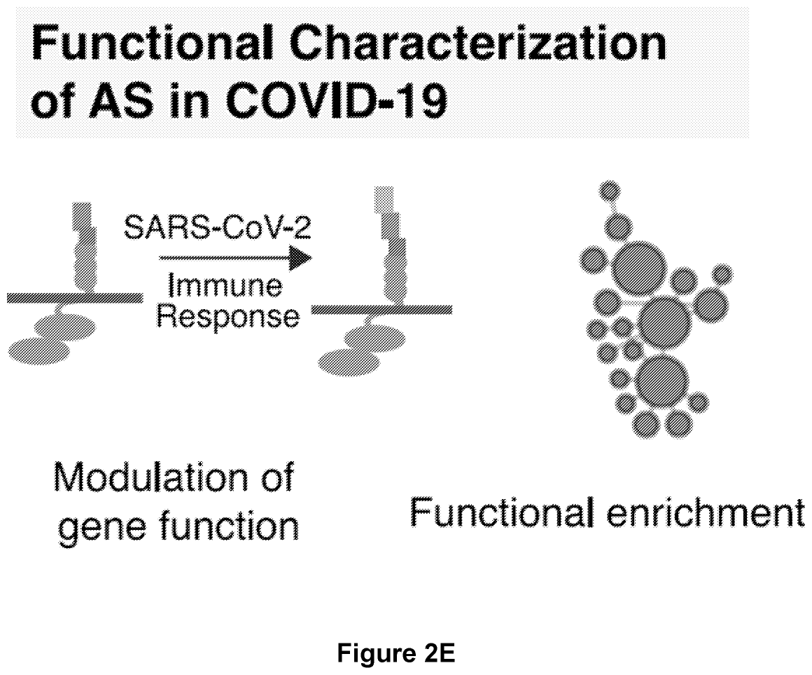

Referring to block 1038, in some embodiments, for a respective alternative splicing event in the plurality of alternative splicing events, the first abundance metric 116 is a percent or proportion spliced in metric determined according to the equation:

$$\frac{\frac{incl usion count}{2}}{\frac{incl usion count}{2} + skip count}$$

where inclusion count is a count of inclusion splice junctions for a first intervening sequence corresponding to the respective alternative splicing event, each respective inclusion splice junction for the first intervening sequence comprising a first nucleic acid sequence for a 5′ or a 3′ end of the first intervening sequence and a second nucleic acid sequence for an adjoining sequence that is 5′ or 3′ of the first intervening sequence, and skip count is a count of exclusion splice junctions for the first intervening sequence corresponding to the respective alternative splicing event, each respective exclusion splice junction excluding all or a portion of the first intervening sequence. This is illustrated in the upper left hand portion of FIG. 2B.

In some alternative embodiments, the first abundance metric 116 is a percent or proportion spliced in metric determined according to the equation:

$$\frac{inclusionn count}{i n c l usion count + skip count}$$

in accordance with Zhang et al., 2019, “Deep-learning augmented RNA-seq analysis of transcript splicing” Nat Methods. 16(4): 307-310, which is hereby incorporated by reference, where inclusion count and skip count are as defined above.

In some alternative embodiments, the first abundance metric 116 is a percent or proportion spliced in metric determined according to the equation:

$$\frac{\frac{i n c l usion count}{Q}}{\frac{i n c l usion count}{Q} + skip count}$$

where inclusion count and skip count are as defined above, and Q is a real value other than zero.

In some alternative embodiments, the first abundance metric 116 is a percent or proportion spliced in metric determined according to the equation:

$$func [ \frac{\frac{i n c l usion count}{Q}}{\frac{i n c l usion count}{Q} - skip count} ]$$

where inclusion count and skip count are as defined above, Q is a real value other than zero (e.g., 1, 2, 3.2, 1.375, etc.) and func is a mathematical operator such as log₂, log₁₀, natural log, etc.

Referring to block 1040, in some embodiments, the determination further comprises aligning each respective sequence read in the plurality of sequence reads to a first reference sequence comprising the plurality of reference splice junctions. Referring to block 1042, in some such embodiments, the first reference sequence is a reference human genome. Referring to block 1044, in some embodiments, the first abundance metric is determined using an RNA sequencing mapping algorithm. In some embodiments, the RNA sequencing mapping algorithm is Spliced Transcripts Alignment to a Reference (STAR v2.7.4), which is used to align the sequence reads to a reference human genome such as hg38 genome build with Gencode v34 index. See Dobin et al., 2013, “STAR: ultrafast universal RNA-seq aligner,” Bioinformatics, 29, pg. 15-21; and Harrow et al., 2006, “GENCODE: producing a reference annotation for ENCODE,” Genome Biol., 7(1), pg. S4.1-S4.9, each of which is hereby incorporated by reference in its entirety for all purposes. Such alignment in accordance with one embodiment of the present disclosure is described in Example 5 below.

In some embodiments, the RNA sequencing mapping algorithm is TopHat2. TopHat2 maps RNA-seq reads to a reference genome while taking into account splicing events and junctions. See, Pertea et al., 2013, “TopHat2: accurate alignment of transcriptomes in the presence of insertions, deletions and gene fusions,” Genome Biol. 14(4):R36, which is hereby incorporated by reference.

In some embodiments, the RNA sequencing mapping algorithm is HISAT2 (Hierarchical Indexing for Spliced Alignment of Transcripts). HISAT2 aligns RNA-seq reads to a reference genome using an indexing strategy that speeds up the mapping process. See, Paggi et al., 2019, “Graph-based genome alignment and genotyping with HISAT2 and HISAT-genotype,” Nat. Biotechnol. 37(8): 907, which is hereby incorporated by reference.

In some embodiments, the RNA sequencing mapping algorithm is Bowtie or Bowtie2. While initially developed for DNA sequencing, Bowtie and Bowtie2 can also be used for RNA-seq read alignment. For this and other aligners that may be used for alignment of the sequence reads of the present disclosure, see Friedel, 2012, “A comprehensive evaluation of alignment algorithms in the context of RNA-seq,” PLoS One 7(12):e52403, which is hereby incorporated by reference.

In some embodiments, the RNA sequencing mapping algorithm is based on a BWA (Burrows-Wheeler Aligner). Like Bowtie, BWA is primarily designed for DNA alignment, but it can also be used for RNA-seq data. It is based on the Burrows-Wheeler transform to align sequence reads. See, for example, Durbin, 2009, “Fast and accurate short read alignment with Burrows-Wheeler transform,” Bioinformatics 25(14):1754-60, which is hereby incorporated by reference.

In some embodiments, the RNA sequencing mapping algorithm is Subread. The Subread aligner an approach called the “seed-and-vote” algorithm to map reads accurately while considering alternative splicing events. See, Smyth and Shi, 2013, “The Subread aligner: fast, and accurate and scalable read mapping by seed-and-vote,” Nucleic Acids Res. 41(10):e108, which is hereby incorporated by reference.

In some embodiments, the RNA sequencing mapping algorithm is Kallisto. Kallisto is a pseudo-alignment-based method that quantifies transcript abundances without explicitly aligning reads to the genome. See Bray et al., 2016, “Near-optimal probabilistic RNA-seq quantification,” Nat. Biotechnol., 34, pg. 525-527, which is hereby incorporated by reference in its entirety for all purposes.

In some embodiments, the RNA sequencing mapping algorithm is GMAP (Genomic Mapping and Alignment Program). GMAP is an alignment tool specifically designed for aligning RNA-seq reads to complex genomes, handling both spliced and non-spliced alignments. See Wu and Watanabe, 2005, “GMAP: a genomic mapping and alignment program for mRNA and EST sequences,” Bioinformatics 21(9):1859-75, which is hereby incorporated by reference.

Referring to block 1046, in some embodiments, for a respective alternative splicing event in the plurality of alternative splicing events, the second abundance metric 118 is a percent or proportion spliced in metric determined according to the equation:

$$\frac{\sum inclusion isoform TPM}{\sum all relevant isoform TPM}$$

where inclusion isoform TPM is a count of transcript isoforms in the biological sample comprising a first intervening sequence corresponding to the respective alternative splicing event, measured in transcripts per million, and all relevant isoform TPM is a count of transcript isoforms spanning the first intervening sequence corresponding to the respective alternative splicing event, measured in transcripts per million. This is illustrated in the upper right hand portion of FIG. 2B. In more detail, an alternative splicing event is the representation of a local variation of the exon-intron structure that is defined by the transcripts that cover that genetic region. In some embodiments it is represented in binary form, e.g. inclusion and skipping of a cassette exon. Accordingly, an alternative splicing event is characterized in terms of the sets of transcripts that describe either form of the alternative splicing event, which can be denoted as F₁ and F₂. For instance, for the exon cassette event, skipped exon (SE), F₁ can represent the transcripts that include the exon, whereas F₂ can represent the transcripts that skip the exon. Alternatively, for the exon cassette event, skipped exon (SE), F₂ can represent the transcripts that include the exon, whereas F₁ can represent the transcripts that skip the exon. The inclusion value, proportion spliced-in (PSI), or percent or proportion spliced-in when scaled between 0 and 100%, that is used for the second abundance metric of this alternative splicing event is defined as the ratio of the abundance of transcripts that include one form of the event, F₁, over the abundance of the transcripts that contain either form of the event [2]. That is, given the abundances for the transcripts isoforms in transcript per million units (TPM) [3], which can be denoted as TPMₖ, the second abundance metric in some embodiments is calculated as:

$$second abandance event = \frac{{\sum }_{k ∈ {F}_{1}} {TPM}_{k}}{{\sum }_{j ∈ {F}_{1} ⋃ {F}_{2}} {TPM}_{j}}$$

More information on computation of PSI values using this form of equation is found in Trincado et al., 2018, “SUPPA2: fast, accurate, and uncertainty-aware differential splicing analysis across multiple conditions,” Genome Biol. 19(1) 40, which is hereby incorporated by reference.

In some alternative embodiments, the second abundance metric 118 is a percent or proportion spliced in metric determined according to the equation:

$$func [ \frac{{\sum }_{k ∈ {F}_{1}} {TPM}_{k}}{{\sum }_{j ∈ {F}_{1} ⋃ {F}_{2}} {TPM}_{j}} ]$$

and func is a mathematical operator such as log 2, log₁₀, natural log, etc.

Referring to block 1048, in some embodiments, the determination further comprises aligning each respective sequence read in the plurality of sequence reads to a second reference sequence comprising the plurality of reference isoforms. Referring to block 1050, in some embodiments, the second reference sequence is a reference human transcriptome and the aligning comprises a pseudo-alignment. For instance, in some embodiments, the sequence reads are used to quantify gene expression levels using kallisto (v0.46.0)36 which pseudo-aligns RNA-seq reads to Gencode v34 transcripts.

Referring to block 1052, in some embodiments, the second abundance metric is determined using a differential splicing analysis algorithm.

Referring to block 1053, responsive to inputting the corresponding first abundance metric or the corresponding second abundance metric for each respective alternative splicing event in the plurality of alternative splicing events into a first model, a predicted infection status of the test subject as output from the first model is received.

In some embodiments in accordance with block 1053, only the corresponding first abundance metric for each respective alternative splicing event in the plurality of alternative splicing events is inputted into the first model to obtain the predicted infection status of the test subject as output from the first model.

In some embodiments in accordance with block 1053, only the corresponding second abundance metric for each respective alternative splicing event in the plurality of alternative splicing events is inputted into the first model to obtain the predicted infection status of the test subject as output from the first model.

In some embodiments in accordance with block 1053, both the corresponding first abundance metric and the corresponding second abundance metric for each respective alternative splicing event in the plurality of alternative splicing events is inputted into the first model to obtain the predicted infection status of the test subject as output from the first model.

In some embodiments, the corresponding first abundance metric and the corresponding second abundance metric are summed together for block 1053 prior to inputting them into the first model. In some embodiments, the corresponding first abundance metric and the corresponding second abundance metric are combined together according to the equation:

$${W}_{1} * {PSI}_{1} + {W}_{2} * {PSI}_{2}$$

prior to inputting them into the first model, where W₁ is a first weight, PSI₁ is the first abundance metric, W₂ is a second weight, and PSI₂ is the second abundance metric. In some such embodiments W₁ and W₂ are both 1. In some such embodiments W₁ and W₂ are both real positive numbers but are not equal to each other. In some embodiments W₁ and W₂ are used to give more weight to one of the abundance metrics than the other.

In some embodiments, the first abundance metric and the second abundance metric for a respective alternative splicing event in the plurality of alternative splicing events are mathematically combined prior to inputting the mathematical combination into the first model. For instance, embodiments in which they are summed together, in weighted or unweighted form, have been described above. In some embodiments, the first and second abundance metrics for a respective alternative splicing event in the plurality of alternative splicing events are averaged together prior to inputting them into the first model.

In some embodiments, the first abundance metric and the second abundance metric for a respective alternative splicing event in the plurality of alternative splicing events are not mathematically combined prior to inputting the mathematical combination into the first model. That is, the first abundance metric and the second abundance metric for a respective alternative splicing event in the plurality of alternative splicing events are each inputted into the model.

In some embodiments, more than two different abundance metrics for each respective alternative splicing event are inputted into the first model. For instance, in some embodiments, 3, 4, 5, 6, or 7 different abundance metrics for each respective alternative splicing event are inputted into the first model. In some such embodiments, the abundance metrics for a respective alternative splicing event are mathematically combined (e.g., summed, averaged, etc.) prior to inputting them into the first model. In alternative embodiments, the abundance metrics for a respective alternative splicing event are each independent inputted into the first model.

In some embodiments, the first abundance metric and the second abundance metric for each respective alternative splicing event in the plurality of alternative splicing events, where the plurality of alternative splicing events is more then 10, more than 25, or more than 50 alternative splicing events is inputted into the first model in accordance with block 1053.

Referring to block 1054, in some embodiments, the model is a logistic regression model.

Referring to block 1056, in some embodiments, the model is selected from the group consisting of. a neural network, a support vector machine, a Naive Bayes model, a nearest neighbor model, a boosted trees model, a random forest model, a decision tree, or a clustering model.

Referring to block 1058, in some embodiments, the infection status of the test subject is a likelihood that the test subject has an infection.

Referring to block 1060, in some embodiments, the infection status of the test subject is a likelihood that the test subject is pre-infection, first-infection, mid-infection, or post-infection.

Referring to block 1062, in some embodiments, the infection status of the test subject is a binary indication as to whether or not test subject has an infection.

Referring to block 1064, in some embodiments, the infection status of the test subject is a binary indication as to whether or not the test subject has a pre-infection, a first-infection, a mid-infection, or a post-infection.

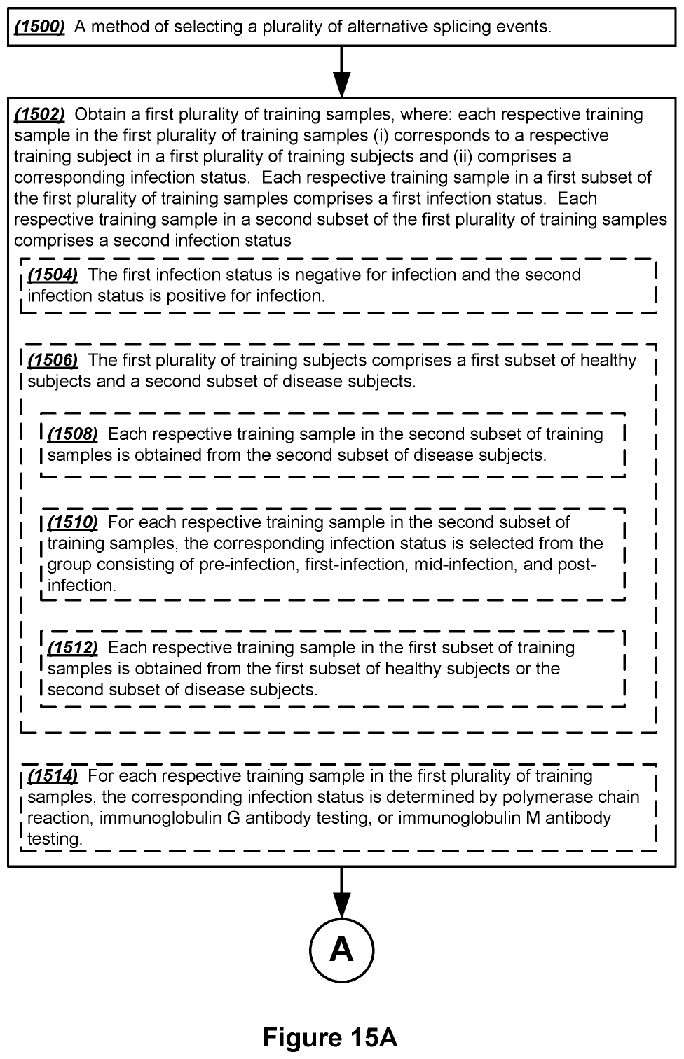

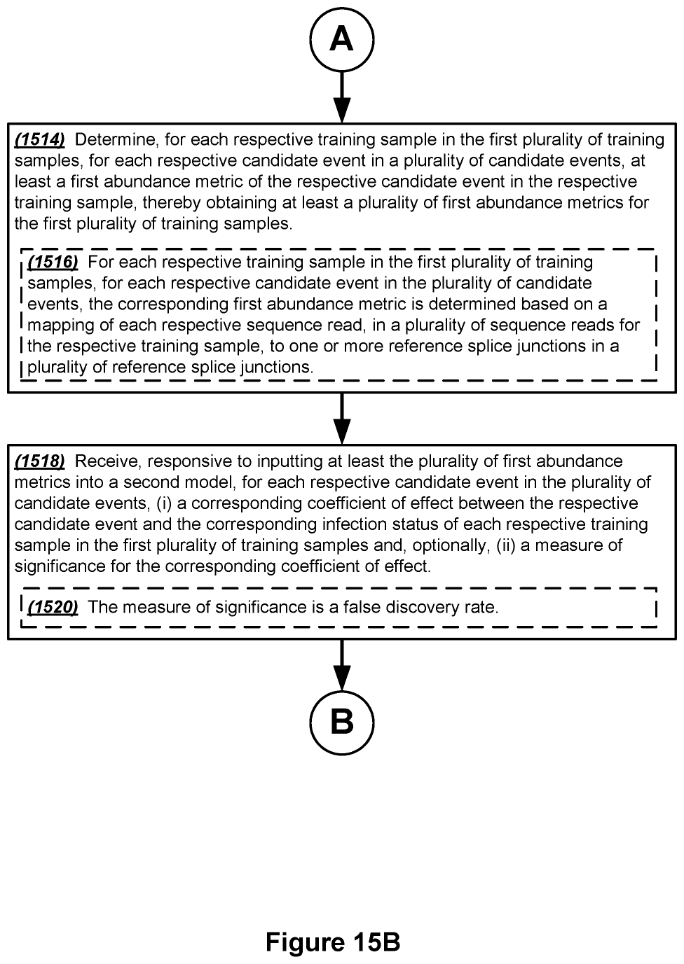

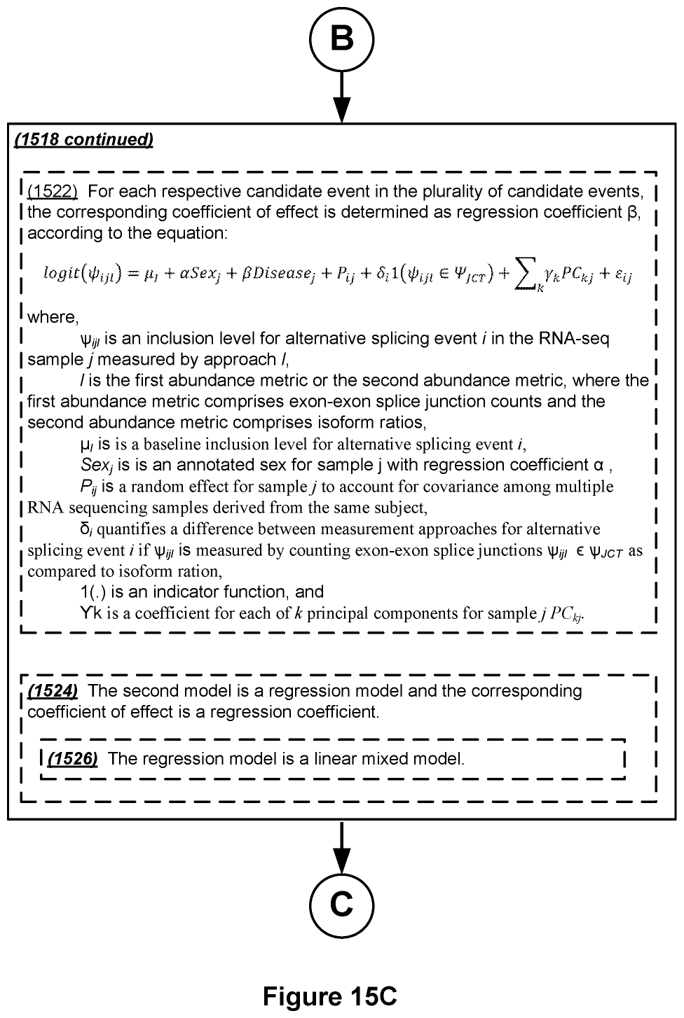

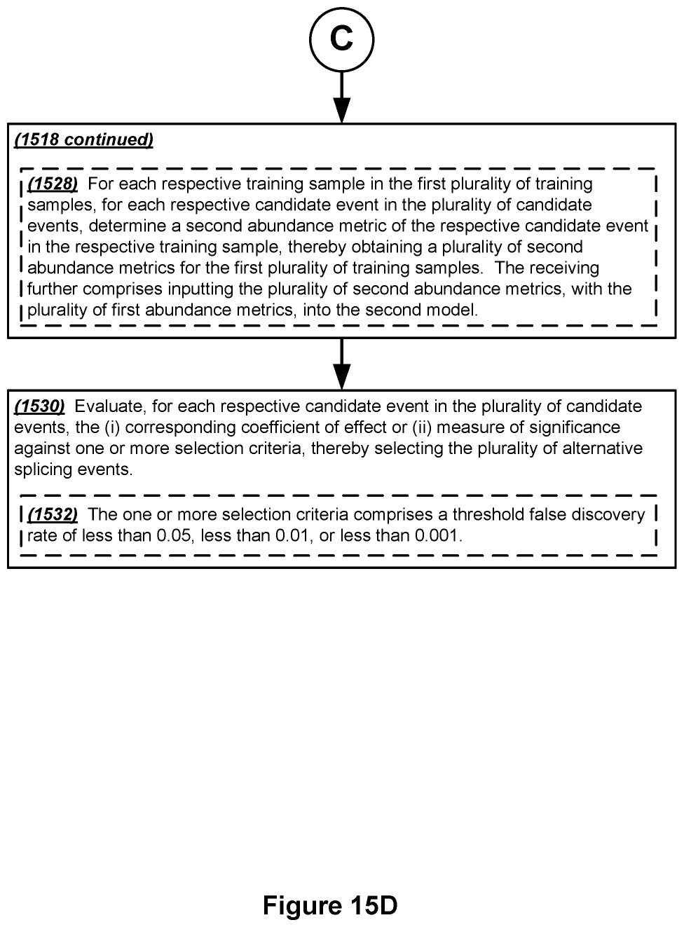

Referring to block 1500 of FIG. 15A, in some embodiments, a method of selecting a plurality of alternative splicing events is provided. Referring to block 1502, in some such embodiments, a first plurality of training samples is obtained. Each respective training sample in the first plurality of training samples (i) corresponds to a respective training subject in a first plurality of training subjects and (ii) comprises a corresponding infection status. Each respective training sample in a first subset of the first plurality of training samples comprises a first infection status. Each respective training sample in a second subset of the first plurality of training samples comprises a second infection status. In some embodiments, the first plurality of training samples comprises 100 or more training samples, 200 or more training samples, 300 or more training samples, or 400 or more training samples.

Referring to block 1504, in some embodiments, the first infection status is negative for infection and the second infection status is positive for infection.

Referring to block 1506, in some embodiments, the first plurality of training subjects comprises a first subset of healthy subjects and a second subset of disease subjects. Referring to block 1508, in some such embodiments, each respective training sample in the second subset of training samples is obtained from the second subset of disease subjects.

Referring to block 1510, in some embodiments, for each respective training sample in the second subset of training samples, the corresponding infection status is selected from the group consisting of pre-infection, first-infection, mid-infection, and post-infection. Example 4 provides an illustration of such groupings.

Referring to block 1512, in some embodiments, each respective training sample in the first subset of training samples is obtained from the first subset of healthy subjects or the second subset of disease subjects. In some embodiments, the diseased subjects are infected with SARS-CoV-2. In some embodiments, the diseased subjects have a bacterial infection, a viral infection, a fungal infection, a parasitic infection, sepsis, tuberculosis, a respiratory infection, a gastrointestinal infection, a urinary tract infection, or a combination thereof. In some embodiments, the diseased subjects have an influenza infection, a human immunodeficiency viral infection, a COVID-19 infection, or a combination thereof.

Referring to block 1514, in some embodiments, for each respective training sample in the first plurality of training samples, the corresponding infection status is determined by polymerase chain reaction, immunoglobulin G antibody testing, or immunoglobulin M antibody testing. Example 2 provides an illustration of such an embodiment.

Referring to block 1514, in some embodiments, there is determined, for each respective training sample in the first plurality of training samples, for each respective candidate event in a plurality of candidate events, at least a first abundance metric of the respective candidate event in the respective training sample, thereby obtaining at least a plurality of first abundance metrics for the first plurality of training samples. In some embodiments, this first abundance metric is any of the first abundance metrics disclosed above in block 1038. In some embodiments, this first abundance metric is any of the second abundance metrics disclosed above in block 1046.

In some embodiments, the plurality of candidate AS events comprises 100 or more candidate AS events, 200 or more AS candidate events, 300 or more candidate AS events, 400 or more candidate AS events, or 500 or more candidate AS events.

Referring to block 1516, in some embodiments, for each respective training sample in the first plurality of training samples, for each respective candidate event in the plurality of candidate events, the corresponding first abundance metric is determined based on a mapping of each respective sequence read, in a plurality of sequence reads for the respective training sample, to one or more reference splice junctions in a plurality of reference splice junctions.

Referring to block 1518, in some embodiments, there is received, responsive to inputting at least the plurality of first abundance metrics into a second model, for each respective candidate event in the plurality of candidate events, (i) a corresponding coefficient of effect between the respective candidate event and the corresponding infection status of each respective training sample in the first plurality of training samples and (ii) a measure of significance for the corresponding coefficient of effect. Referring to block 1520, in some such embodiments, the measure of significance is a false discovery rate.

Referring to block 1522, in some embodiments, for each respective candidate event in the plurality of candidate events, the corresponding coefficient of effect is determined as regression coefficient β, according to the equation:

$$logit ( {ψ}_{i j l} ) = {μ}_{I} + α {Sex}_{j} + β Diseas {e}_{j} + {P}_{i j} + {δ}_{i} 1 ( {ψ}_{i j l} ∈ {Ψ}_{JCT} ) + {\sum }_{k} {γ}_{k} P {C}_{k j} + {ε}_{i j}$$

where ψᵢⱼₗ is an inclusion level for alternative splicing event i in the RNA-seq sample j measured by approach l, l is the first abundance metric or the second abundance metric, wherein the first abundance metric comprises exon-exon splice junction counts and the second abundance metric comprises isoform ratios, μ_{I} is a baseline inclusion level for alternative splicing event i, Sexⱼ is an annotated sex for sample j with regression coefficient α, Diseaseⱼ is an annotated disease stage for sample j with regression coefficient β, Pᵢⱼ is a random effect for sample j to account for covariance among multiple RNA sequencing samples derived from the same subject, δᵢ quantifies a difference between measurement approaches for alternative splicing event i if ψᵢⱼₗ is measured by counting exon-exon splice junctions ψᵢⱼₗ∈Ψ_{JCT} as compared to isoform ratios, 1(⋅) is an indicator function, and γₖ is a coefficient for each of k principal components for sample j PCₖⱼ.

Referring to block 1524, in some embodiments, the second model is a regression model and the corresponding coefficient of effect is a regression coefficient. Referring to block 1526, in some such embodiments, the regression model is a linear mixed model.

Referring to block 1528, in some embodiments, for each respective training sample in the first plurality of training samples, for each respective candidate event in the plurality of candidate events, a second abundance metric of the respective candidate event in the respective training sample is also determined, thereby obtaining a plurality of second abundance metrics for the first plurality of training samples. In such embodiments, the receiving step of block 1518 further comprises inputting the plurality of second abundance metrics, with the plurality of first abundance metrics, into the second model.

Referring to block 1530, in some embodiments, there is evaluated, for each respective candidate event in the plurality of candidate events, the (i) corresponding coefficient of effect or (ii) measure of significance against one or more selection criteria, thereby selecting the plurality of alternative splicing events. Referring to block 1532 of FIG. 15D, in some such embodiments, the one or more selection criteria comprises a threshold false discovery rate of less than 0.05, less than 0.01, or less than 0.001.

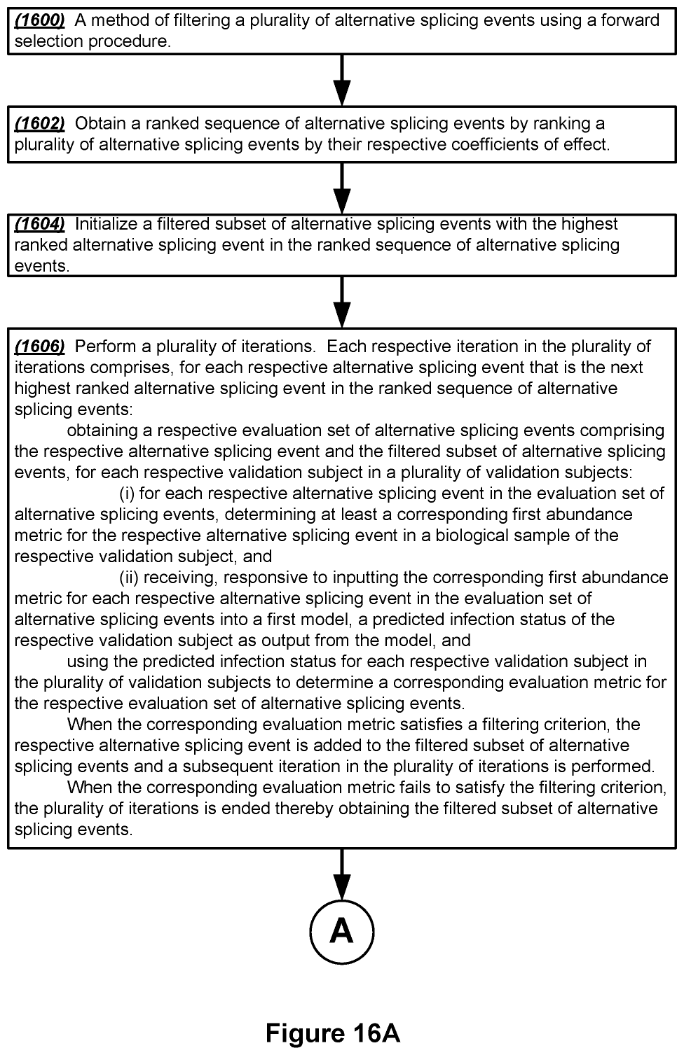

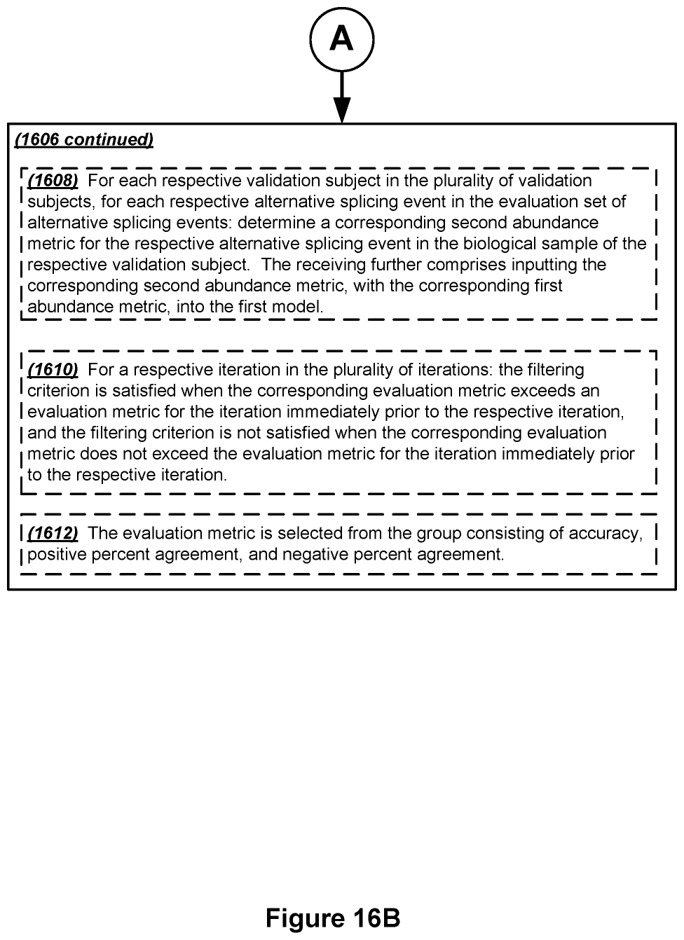

Referring to block 1600 of FIG. 16A another aspect of the present disclosure provides a method of filtering a plurality of alternative splicing events using a forward selection procedure.

Referring to block 1602, in some embodiments, a ranked sequence of alternative splicing events is obtained. For instance, in some embodiments the ranked sequence of alternative splicing events is obtained by ranking the above-described plurality of alternative splicing events by their respective coefficients of effect. In some embodiments, the ranked sequence of alternative splicing events comprises 100 or more AS events, 200 or more AS events, 300 or more AS events, 400 or more AS events, or 500 or more AS events.

Referring to block 1604, a filtered subset of alternative splicing events is initialized with the highest ranked alternative splicing event in the ranked sequence of alternative splicing events.

Referring to block 1606, a plurality of iterations is performed. Each respective iteration in the plurality of iterations comprises, for each respective alternative splicing event that is the next highest ranked alternative splicing event in the ranked sequence of alternative splicing events: obtaining a respective evaluation set of alternative splicing events comprising the respective alternative splicing event and the filtered subset of alternative splicing events, for each respective validation subject in a plurality of validation subjects: (i) for each respective alternative splicing event in the evaluation set of alternative splicing events, determining at least a corresponding first abundance metric for the respective alternative splicing event in a biological sample of the respective validation subject, and (ii) receiving, responsive to inputting the corresponding first abundance metric for each respective alternative splicing event in the evaluation set of alternative splicing events into the a model, a predicted infection status of the respective validation subject as output from a first model. The predicted infection status for each respective validation subject in the plurality of validation subjects is used to determine a corresponding evaluation metric for the respective evaluation set of alternative splicing events. When the corresponding evaluation metric satisfies a filtering criterion, the respective alternative splicing event is added to the filtered subset of alternative splicing events and a subsequent iteration in the plurality of iterations is performed. When the corresponding evaluation metric fails to satisfy the filtering criterion, the plurality of iterations is ended thereby obtaining the filtered subset of alternative splicing events.

Referring to block 1608 of FIG. 16B, in some embodiments, for each respective validation subject in the plurality of validation subjects, for each respective alternative splicing event in the evaluation set of alternative splicing events: a corresponding second abundance metric for the respective alternative splicing event in the biological sample of the respective validation subject is determined and the receiving (ii) of block 1606 further comprises inputting the corresponding second abundance metric, with the corresponding first abundance metric, into the first model.

Referring to block 1610, in some embodiments, for a respective iteration in the plurality of iterations: the filtering criterion is satisfied when the corresponding evaluation metric exceeds an evaluation metric for the iteration immediately prior to the respective iteration, and the filtering criterion is not satisfied when the corresponding evaluation metric does not exceed the evaluation metric for the iteration immediately prior to the respective iteration. Referring to block 1612, in some embodiments of block 1610, the evaluation metric is selected from the group consisting of accuracy, positive percent agreement, and negative percent agreement.

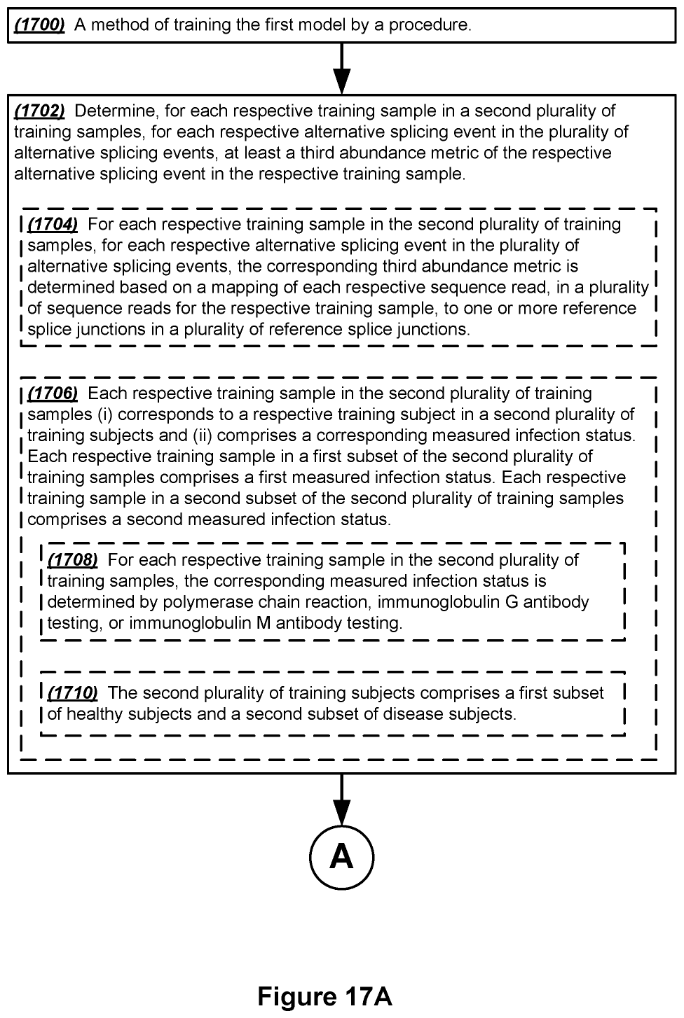

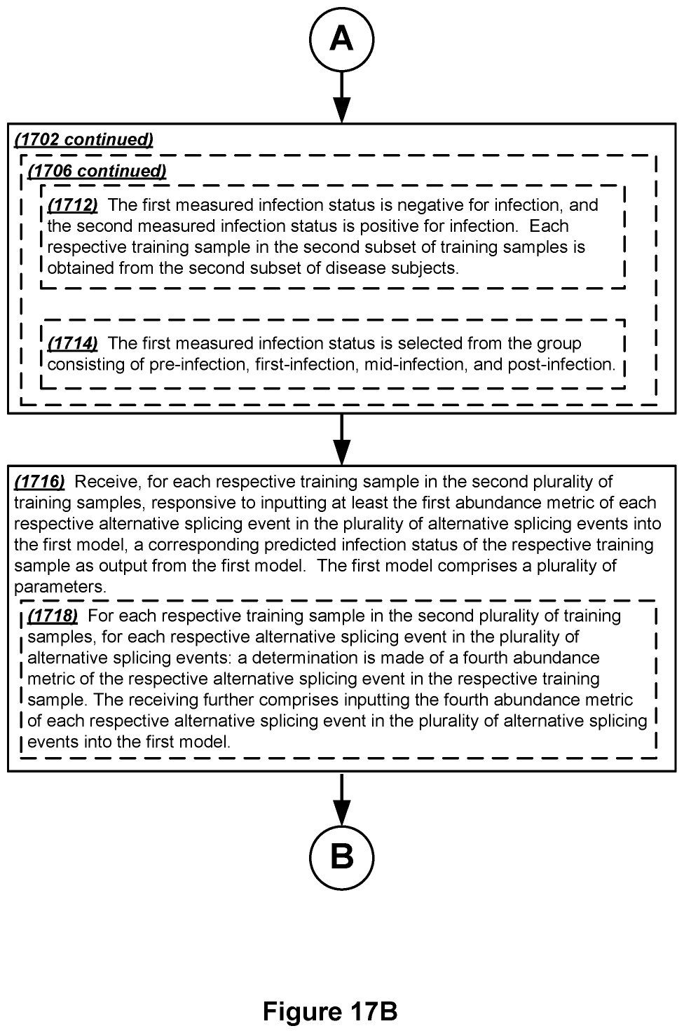

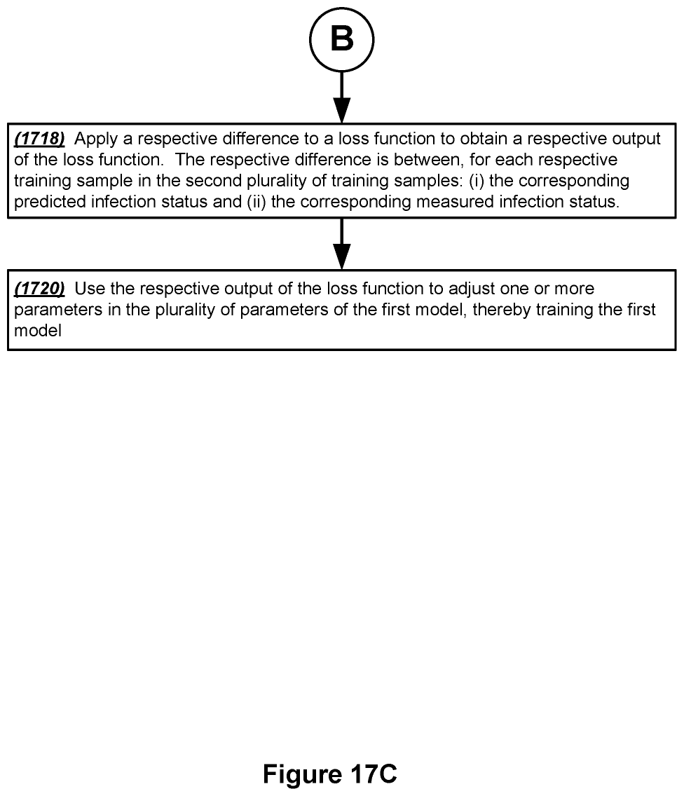

Referring to block 1700 of FIG. 17A another aspect of the present disclosure provides a method of training a model (e.g., the first model) by a procedure. Referring to block 1702, a determination is made, for each respective training sample in a second plurality of training samples, for each respective alternative splicing event in the plurality of alternative splicing events, of at least a third abundance metric of the respective alternative splicing event in the respective training sample. In some embodiments the third abundance metric is any of the first abundance metrics described in block 1038. In some embodiments the third abundance metric is any of the second abundance metrics described in block 1046.

Referring to block 1704, in some embodiments, for each respective training sample in the second plurality of training samples, for each respective alternative splicing event in the plurality of alternative splicing events, the corresponding third abundance metric is determined based on a mapping of each respective sequence read, in a plurality of sequence reads for the respective training sample, to one or more reference splice junctions in a plurality of reference splice junctions.

Referring to block 1706, in some embodiments, each respective training sample in the second plurality of training samples (i) corresponds to a respective training subject in a second plurality of training subjects and (ii) comprises a corresponding measured infection status. Each respective training sample in a first subset of the second plurality of training samples comprises a first measured infection status. Each respective training sample in a second subset of the second plurality of training samples comprises a second measured infection status.

In some embodiments, the second plurality of training samples comprises 100 or more training samples, 200 or more training samples, 300 or more training samples, or 400 or more training samples.

Referring to block 1708, in some embodiments, for each respective training sample in the second plurality of training samples, the corresponding measured infection status is determined by polymerase chain reaction, immunoglobulin G antibody testing, or immunoglobulin M antibody testing.

Referring to block 1710, in some embodiments, the second plurality of training subjects comprises a first subset of healthy subjects and a second subset of disease subjects. In some embodiments, the diseased subjects have a bacterial infection, a viral infection, a fungal infection, a parasitic infection, sepsis, tuberculosis, a respiratory infection, a gastrointestinal infection, a urinary tract infection, or a combination thereof. In some embodiments, the diseased subjects have an influenza infection, a human immunodeficiency viral infection, a COVID-19 infection, or a combination thereof.

Referring to block 1712, in some embodiments, the first measured infection status is negative for infection, and the second measured infection status is positive for infection. Each respective training sample in the second subset of training samples is obtained from the second subset of disease subjects.

Referring to block 1714, in some embodiments, the first measured infection status is selected from the group consisting of pre-infection, first-infection, mid-infection, and post-infection.

Referring to block 1716, for each respective training sample in the second plurality of training samples, responsive to inputting at least the first abundance metric of each respective alternative splicing event in the plurality of alternative splicing events into the first model, a corresponding predicted infection status of the respective training sample as is obtained as output from the first model. The first model comprises a plurality of parameters. In some embodiments, the model is a logistic regression model. In some embodiments, the model is selected from the group consisting of. a neural network, a support vector machine, a Naive Bayes model, a nearest neighbor model, a boosted trees model, a random forest model, a decision tree, or a clustering model.

Referring to block 1718, in some embodiments in accordance with block 1716, for each respective training sample in the second plurality of training samples, for each respective alternative splicing event in the plurality of alternative splicing events: a determination is made of a fourth abundance metric of the respective alternative splicing event in the respective training sample. In such embodiments, the receiving of block 1716 further comprises inputting the fourth abundance metric of each respective alternative splicing event in the plurality of alternative splicing events into the first model. In some embodiments the fourth abundance metric is any of the first abundance metrics described in block 1038 provided it is not the same as the third abundance metric. In some embodiments the fourth abundance metric is any of the second abundance metrics described in block 1046 provided it is not the same as the third abundance metric.

Referring to block 1718, in some embodiments, a respective difference is applied to a loss function to obtain a respective output of the loss function. The respective difference is between, for each respective training sample in the second plurality of training samples, (i) the corresponding predicted infection status and (ii) the corresponding measured infection status.

Referring to block 1720, the respective output of the loss function is used to adjust one or more parameters in the plurality of parameters of the first model, thereby training the first model. In some embodiments this adjustment of the one or more parameters is in the form of regression, optionally with L1 or L2 constraints. In some embodiments, this adjustment of the one or more parameters is through back-propagated through the parameters of the first model. In some such embodiments, the parameters of the first model are adjusted in such back-propagation. In an exemplary embodiment, the first model is trained against the errors in the activity class assignments (e.g., “has disease” or “does not have disease”) made by the first model, in view of the actual class assignment that is known for each training subject, by stochastic gradient descent with the AdaDelta adaptive learning method (Zeiler, 2012 “ADADELTA: an adaptive learning rate method,’” CoRR, vol. abs/1212.5701, which is hereby incorporated by reference), and the back propagation algorithm provided in Rumelhart et al., 1988, “Neurocomputing: Foundations of research,” ch. Learning Representations by Back-propagating Errors, pp. 696-699, Cambridge, MA, USA: MIT Press, which is hereby incorporated by reference. Such a refinement technique is just one of many examples for training the first model each of which is within the scope of the present disclosure.

### EXAMPLES

### Example 1—Introduction

Host-based response assays (HRAs) can often diagnose infectious disease earlier and more precisely than pathogen-based tests. However, the role of RNA alternative splicing (AS) in HRAs remains unexplored, as existing HRAs are restricted to gene expression signatures.

Assays detecting blood transcriptome changes are studied for infectious disease diagnosis. Blood-based RNA alternative splicing (AS) events, which have not been well characterized in pathogen infection, have potential normalization and assay platform stability advantages over gene expression for diagnosis. Accordingly, this set of examples leverages a large prospective cohort of severe acute respiratory syndrome coronavirus 2 (SARS-CoV-2) infection and whole-blood RNA sequencing (RNA-seq) data, to identify a major functional AS program switch upon viral infection.

By using an independent cohort, the accuracy of AS biomarkers for SARS-CoV-2 diagnosis was improved as compared with six reported transcriptome signatures is provided. SARS-CoV-2 is a highly contagious virus that has caused a worldwide pandemic. A large cohort of United States Marine recruits was used in this example set. This example set found that many immune-related genes undergo DAS. Since AS can alter genes and their protein products, these findings complement the existing gene and protein expression-based findings and open new avenues for understanding the molecular mechanisms of human immune responses against SARS-CoV-2.

A subset of the AS-based biomarkers was used to develop microfluidic PCR diagnostic assays. This assay achieves nearly perfect test accuracy (e.g., 61/62=98.4% accuracy) using a naive principal component classifier, which is significantly more accurate than a gene expression PCR assay in the same cohort. Therefore, the RNA splicing framework provided by this set of examples enables a promising avenue for host-response diagnosis of infection.

This example set provided for the identification, optimization, and evaluation of blood AS-based diagnostic assay development for infectious disease. Using SARS-CoV-2 infection as a case study, a robust set of AS signatures superior to the gene expression-based markers derived from the same cohort was found. The robust set of AS signatures was also superior to six previously reported transcriptome signatures. Functional analysis revealed significant enrichment of differential splicing events in immune-specific protein domains and genes. Thus, this example set provided a highly performant set of host-based AS-centered biomarkers for SARS-CoV-2 infection detection and demonstrate a promising avenue for design and implementation of host-based diagnostics by leveraging RNA splicing as robust diagnostic biomarkers.

To identify robust RNA AS events as diagnostic biomarkers, the CHARM cohort, a cohort of healthy subjects and infected subjects, was used. The CHARM cohort is further described in Example 7 below. The CHARM cohort is very homogeneous in age and health status, providing molecular profiling from many individuals with few confounding factors typical of SARS-CoV-2 human studies. Despite the uniqueness of this cohort, this set of examples showed that the AS signatures discovered in this set of examples, found in young adults with mild and asymptomatic infections of the CHARM cohort, generalized exceptionally well to two other cohorts, including older individuals. This demonstrates that the AS signatures discovered in this set of examples are robust, viral-infection induced molecular changes.

Whole-blood specimens were collected and sequenced by RNA-seq as illustrated in FIG. 2A. The AS events from RNA-seq datasets were then quantified by computing the percent or proportion spliced in (PSI) for each AS event using two complementary statistical measurements. This quantification is detailed in Example 5 below. See also, Cieślik et al., 2017, “Cancer transcriptome profiling at the juncture of clinical translation,” Nat. Rev. Genet., 19, pg. 93-109; Zhang et al., 2019, “Deep-learning augmented RNAseq analysis of transcript splicing,” Nat. Methods, 16, pg. 307-310; Trincado et al., 2018, “SUPPA2: fast, accurate, and uncertainty-aware differential splicing analysis across multiple conditions,” Genome Biol., 19, pg. 40, each of which is hereby incorporated by reference in its entirety for all purposes. Because the two measurement approaches relied on in Example 5 related to different empirical information of junction counts and transcript expression levels, respectively, joint statistical modeling of PSIs measured by the two different approaches disclosed in Example 5 enabled the identification of robust, measurement-agnostic AS variations.

With the PSI values determined in accordance with Example 5, a flexible linear mixed model regression framework was leveraged to model PSI variations with respect to disease status while controlling for confounding covariates as illustrated in FIG. 2B and described in Example 6. This allowed for analysis of cross-sectional experimental design as well as more complex experimental designs such as longitudinal studies. The mixed model regression framework of Example 6 was used to investigate AS variations during SARS-CoV-2 infection in a large prospective cohort named COVID-19 Health Response for Marines (CHARM). See Letizia et al., 2020, “SARS-CoV-2 transmission among marine recruits during quarantine,” N. Engl. J. Med., 383, ph. 2407-2416, which is hereby incorporated by reference in its entirety for all purposes. To account for the covariance of multiple RNA-seq data longitudinally sampled from the same subject and to control for confounding variables in the data, the linear mixed models of Example 6 were developed to analyze the dependency between disease progression stages as illustrated in FIG. 2B. This enabled the explicit control for non-disease-related splicing changes (e.g., due to the subject's sex or military training). After estimating the regression coefficients and testing for statistical significance, over 1,000 significant disease-associated AS events were identified.

These AS changes were used to build a host-based AS centered diagnostic assay for SARS-CoV-2 infection. In particular, using the differential AS (DAS) events, a logistic regression classifier was trained that was accurate across cohorts and detected infections undetected by serial PCR tests as illustrated in FIG. 2C. The AS-based classifier outperformed gene expression-based markers derived from the same cohort as well as six previously reported transcriptome signatures, demonstrating the superior consistency and robustness of AS biomarkers for viral infection.

To further optimize a subset of the markers in an AS-based microfluidic biomarker assay, the systems and methods of the present disclosure performed a forward selection to select non-redundant AS signatures as illustrated in FIG. 2D. The microfluidic assay achieved nearly perfect accuracy in an independent cohort (accuracy=98.4%). In some embodiments, the systems and methods of the present disclosure characterized SARS-CoV-2-infection-induced AS variations with functional, network, and biomarker analyses, identifying putative upstream splicing factors and temporal dynamics in splicing regulation (FIG. 2E). Below, the present disclosure provides details regarding the identification and comprehensive evaluations of AS-based diagnostic biomarkers, followed by a functional analysis of DAS events for COVID-19.

### Example 2—Cohort

With the CHARM cohort described in example 1, 1,176 whole-blood RNAseq were performed from n=371 US Marine recruits. Details of this sequencing are provided in Example 8 below. After a 14-day quarantine, whole blood for RNA-seq, immunoglobulin G (IgG) and IgM antibody tests, and PCR testing for SARS-CoV-2 virus were longitudinally collected. Based on the timeline of SARS-CoV-2 PCR test results, samples were divided into control (more than 1 week before first PCR+test) and disease stages of pre (within 2 weeks pre-PCR+), first (first-time PCR+), mid (follow-up PCR+), and post (post-infection; see FIG. 3A).

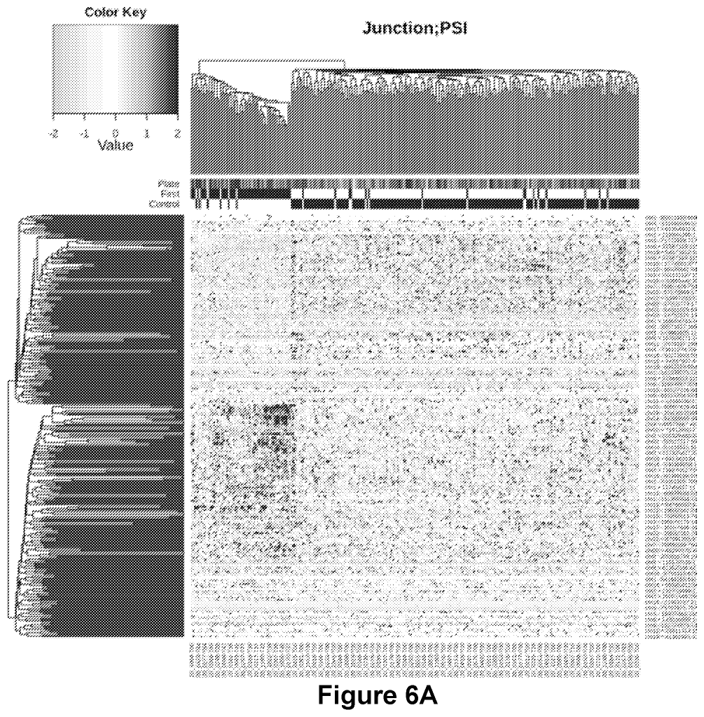

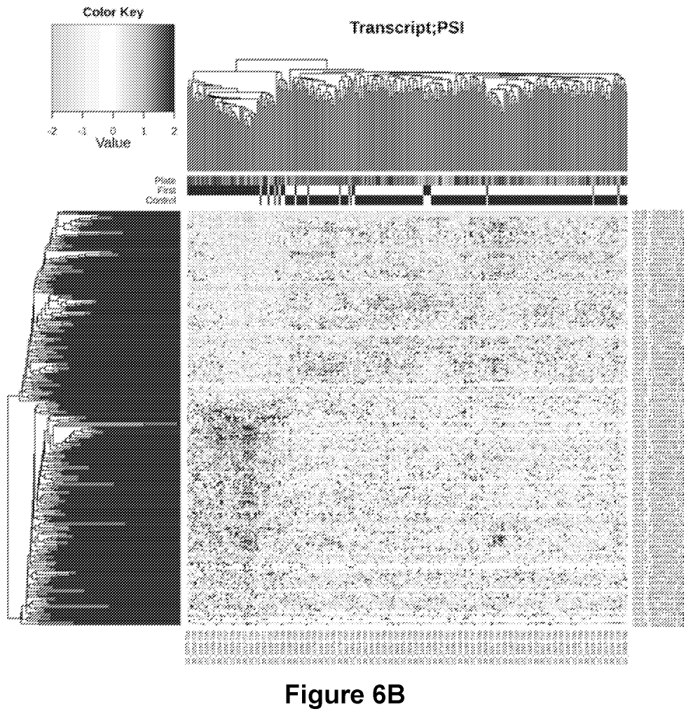

Using the CHARM cohort of longitudinal SARS-CoV-2 infection, four distinct types of AS events were used and a substantial number of DAS events in each of the four types of AS events across the disease stages were found as summarized in FIG. 3B. Most splicing program changes occurred during the PCR+phase of the SARS-CoV-2 infection (first and mid). Qualitatively, it was seen that the DAS events identified at the first versus control comparison can separate infected samples and healthy controls by unsupervised clustering as illustrated in FIGS. 6A and 6B, suggesting an acute, homogeneous immune response mediated by AS regulation to SARS-CoV-2 infection when subjects first turn PCR+ is possible.

Logistic regression models were built using the first PCR+samples from the CHARM cohort to classify active SARS-CoV-2 infection. The PSI values of DAS events were used as features for a given false discovery rate (FDR) cutoff, and the FDR cutoffs were used to generate signature sets of different sizes. Details of these models are provided in Example 9 below.

The models were tested on an independent cohort processed at the Duke Medical Center (herein referred to as the Duke cohort). The Duke cohort provided a challenging test set because it focused on older individuals (average age=46, SD=18.6; Table 1) as opposed to the CHARM cohort of healthy, young, asymptomatic/mildly symptomatic Marine recruits. The Duke cohort is described in Example 7 below.

As a control experiment, the same model training procedure was applied to the gene expression values of top differentially expressed genes (DEGs) ranked by their differential test FDRs. The testing performance peaked at around 300 features for both DAS- and DEG-based classifiers, while including more features afterward induced overfitting for both types of classifiers. Furthermore, when conditioned on the same number of features (n=100-600 features), DAS classifiers always outperformed DEG classifiers (p=0.047, ANOVA; FIG. 4A). Since the set of diagnostic markers needs to be minimized in size while maintaining optimal performance, this suggested the potential of using DAS biomarkers for better diagnostic assay design.

To consolidate observations, six publicly available gene expression signatures were additionally examined for SARS-CoV-2 infection. Details of these publicly available gene expression signatures is found I Thair et al., 2021, “Transcriptomic similarities and differences in host response between SARSCoV-2 and other viral infections,” iScience, 24, pg. 101947; Lee et al., 2020, “Immunophenotyping of COVID-19 and influenza highlights the role of type I interferons in development of severe COVID-19,” Sci. Immunol., 5, eabdl554; Li et al., 2021, “Discovery and validation of a three-gene signature to distinguish COVID-19 and other viral infections in emergency infectious disease presentations: a case-control and observational cohort study,” Lancet. Microbe, 2, e594-e603; McClain et al., 2021, “Dysregulated transcriptional responses to SARS-CoV-2 in the periphery,” Nat. Commun., 12, pg. 1079; Aschenbrenner et al., 2021, “Disease severity-specific neutrophil signatures in blood transcriptomes stratify COVID-19 patients,” Genome Med., 13, pg. 7; Kwan, et al., 2021, “A blood RNA transcriptome signature for COVID-19,” BMC Med. Genom., 14, pg. 155, each of which is hereby incorporated by reference in its entirety for all purposes.

The best-performing signature sets derived from the CHARM cohort were denoted as CHARM DAS and DEG, respectively. All public signature sets were processed identically to CHARM DEG. In order to rigorously compare the quality of signature sets without biases induced by cohort and model differences in previous studies, classifiers were retained on the CHARM cohort using different previously reported gene signature sets as features and the classifiers were tested on the Duke cohort. It was found that the CHARM DAS signature set performed the best among all signature sets, outperforming all DEG-based signatures by a large margin as illustrated in FIG. 4B. The signature set from McClain et al. was excluded from this comparison because the same Duke cohort was used as the discovery set in McClain et al., 2021, Nat. Commun. While this signature set performed best among DEG-based signatures (test area under the receiver operating curve [AUROC]=0.806), it was still subpar to the CHARM DAS signatures. The DAS classifier achieved a testing AUROC of 0.85 on the Duke cohort, demonstrating the superiority of AS signatures compared with previously published gene expression signatures as illustrated in FIG. 4C.

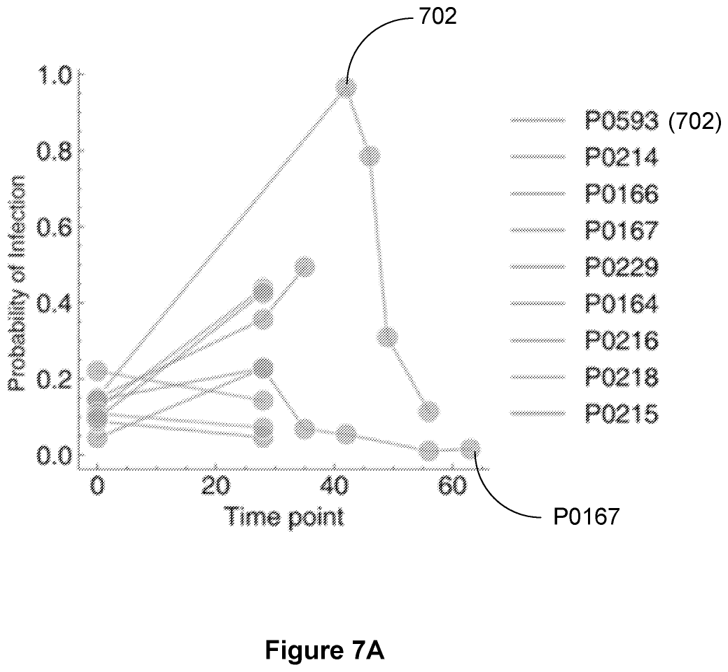

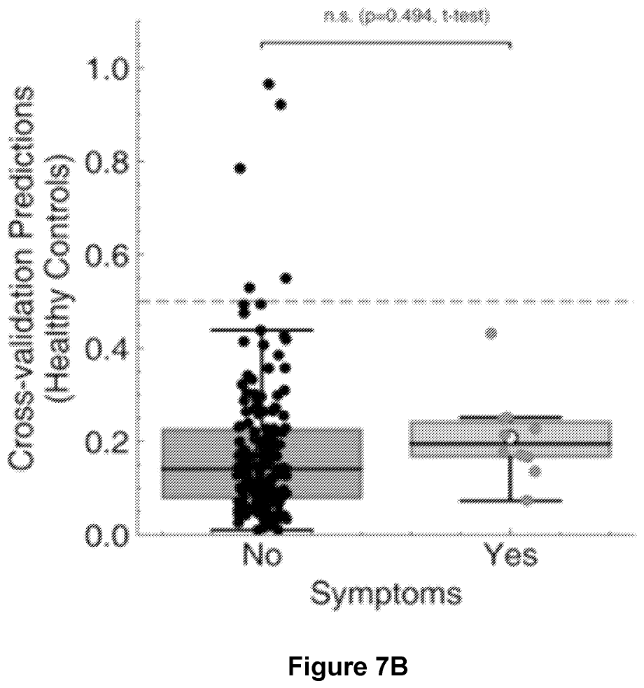

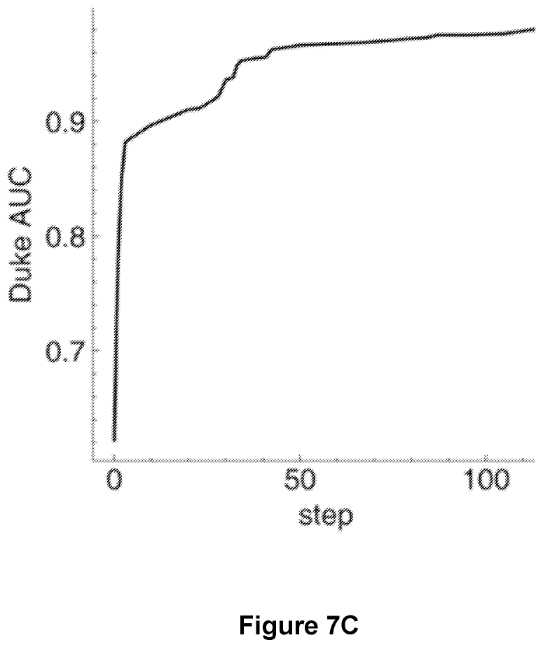

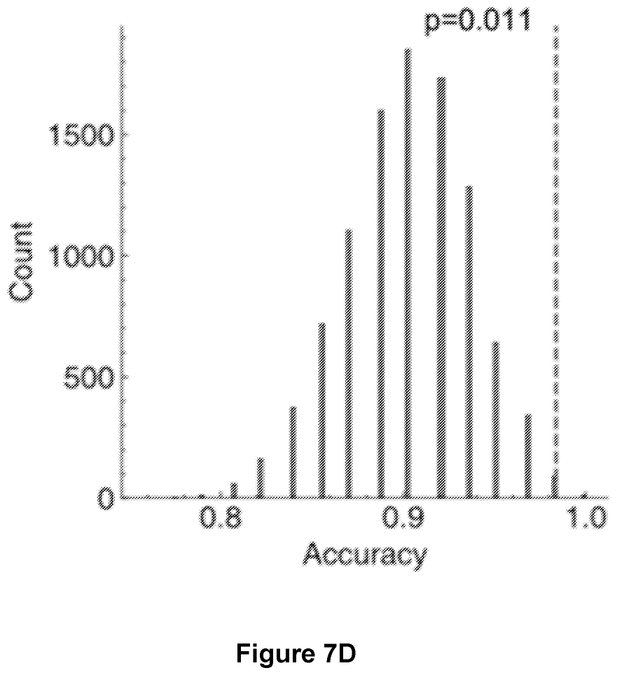

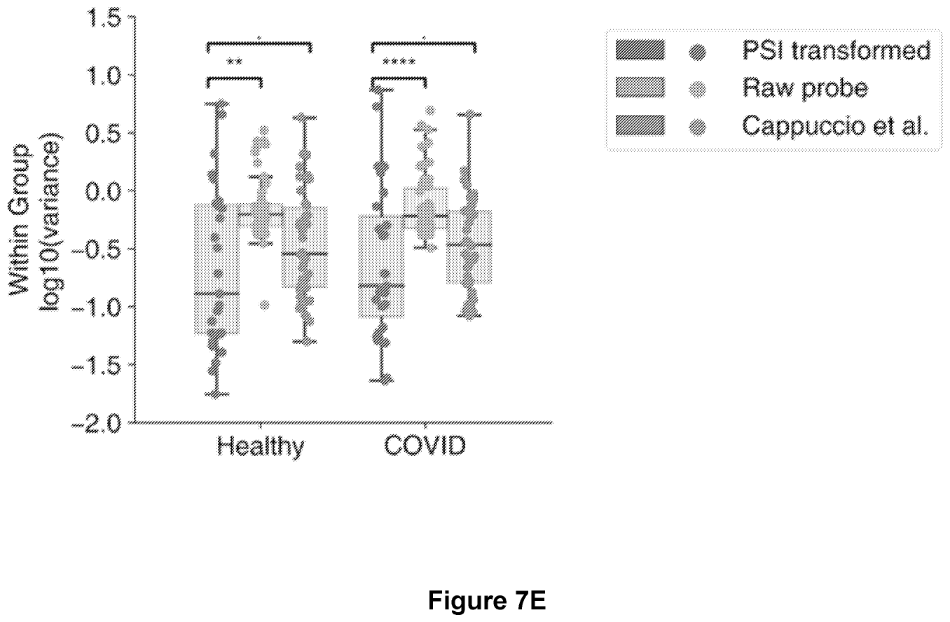

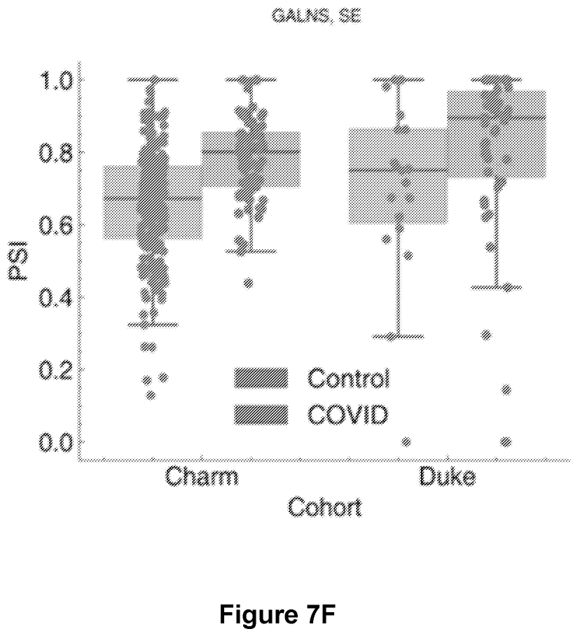

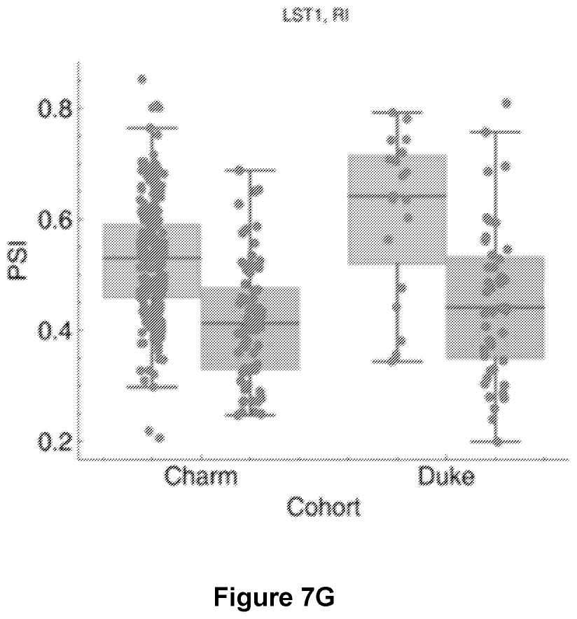

Using the PSI of the CHARM DAS signature set as features, the infection likelihood for all CHARM samples was assessed by a 10-fold cross-validation prediction (AUROC=0.922±0.057) for each individual throughout the time course as summarized in FIG. 7A. As illustrated in FIG. 7A, for one particular subject (P0593) with consistent PCR test results and no symptoms reported, shown in FIG. 4D, a strong peak in predicted probability of infection was observed at day 42, with predicted probability gradually decreasing over time. This subject had negative SARS-CoV-2 antibody tests in early time points (T0 and T42), demonstrating no vaccination (as the cohort was enrolled during May-November 2020) nor pre-exposure to SARS-CoV-2. At day 56, this subject had a positive IgG test (7.01) and IgM test (1.85) specific to SARSCoV-2 viral proteins, suggesting that the PCR tests were false negatives. Together, these results strongly suggest that this subject was an asymptomatic infected subject that consistently tested negative through a series of four PCR tests, and the AS based classifier of this set of examples was accurate enough to capture this case missed by PCR testing and symptom monitoring. Furthermore, the classifier likely generalized the AS patterns in response to SARS-CoV-2 infection beyond just learning symptoms. The classifier predicted a high infection score for the asymptomatic SARS-CoV-2-infected subject (FIG. 3D); conversely, in SARS-CoV-2 PCR−/sero-negative control samples, having symptoms did not significantly increase the predicted infection scores (FIG. 7B).

### Example 3—AS-Based Host-Response Assay Demonstrates Across-Platform Consistency

Encouraged by the superior performance of the CHARM DAS classifier disclosed in Example 2, optimization of a small set of splicing biomarkers to test on microfluidic devices was sought in this example. Compared with Illumina RNA-seq, microfluidic devices are portable as a diagnostic device but are limited in the capacity of measurable probes and present distinct technical biases. A forward selection of biomarkers was performed to select non-redundant biomarkers by optimizing the classifier's performance in both CHARM and Duke cohorts, resulting in a set of 27 AS biomarkers (FIG. 7C). More details on this selection and analysis are disclosed in Example 10 below. The full list of selected biomarkers is set forth in Table 2.

TABLE 2

| Step | AUC | Gene Name | Type | Strnd | Long | Short |
|---|---|---|---|---|---|---|
| 0 | 0.62 | IGLL5 | SE | + | chr22: 22888259- | chr22: 22888259- |
| | | | | | 22893699; | 22895374 |
| | | | | | chr22: 22893818- | |
| | | | | | 22895374 | |
| 1 | 0.78 | LST1 | RI | + | chr6: 31587298- | chr6: 31587318- |
| | | | | | 31587338; | 31588517 |
| | | | | | chr6: 31588497- | |
| | | | | | 31588537 | |
| 2 | 0.85 | GALNS | SE | − | chr16: 88841971- | chr16: 88841971- |
| | | | | | 88842705; | 88856757 |
| | | | | | chr16: 88842829- | |
| | | | | | 88856757 | |
| 3 | 0.88 | EPSTI1 | SE | − | chr13: 42917624- | chr13: 42917624- |
| | | | | | 42919299; | 42926335 |
| | | | | | chr13: 42919332- | |
| | | | | | 42926335 | |
| 10 | 0.9 | LILRB2 | RI | − | chr19: 54276909- | chr19: 54276929- |
| | | | | | 54276949; | 54277549 |
| | | | | | chr19: 54277529- | |
| | | | | | 54277569 | |
| 20 | 0.91 | RIN2 | SE | + | chr20: 19935199- | chr20: 19935199- |
| | | | | | 19935438; | 19956614 |
| | | | | | chr20: 19935607- | |
| | | | | | 19956614 | |
| 23 | 0.91 | PALM2AKAP2 | SE | + | chr9: 110138539- | chr9: 110138539- |
| | | | | | 110156318; | 110168398 |
| | | | | | chr9: 110156497- | |
| | | | | | 110168398 | |
| 28 | 0.92 | HMGN2 | RI | + | chr1: 26473507- | chr1: 26473527- |
| | | | | | 26473547; | 26473702 |
| | | | | | chr1: 26473682- | |
| | | | | | 26473722 | |
| 30 | 0.94 | TUBA8 | 5SS | + | chr22: 18110868- | chr22: 18110839- |
| | | | | | 18121478 | 18121478 |
| 32 | 0.94 | SNHG32 | SE | + | chr6: 31835451- | chr6: 31835451- |
| | | | | | 31836423; | 31837235 |
| | | | | | chr6: 31836517- | |
| | | | | | 31837235 | |
| 33 | 0.95 | KIF22 | A3SS | + | chr16: 29804065- | chr16: 29804065- |
| | | | | | 29804575 | 29804813 |
| 34 | 0.95 | ATP6V0B | 5SS | + | chr1: 43975848- | chr1: 43975107- |
| | | | | | 43976089 | 43976089 |
| 36 | 0.95 | SESN3 | SE | − | chr11: 95185492- | chr11: 95185492- |
| | | | | | 95189778; | 95191403 |
| | | | | | chr11: 95189961- | |
| | | | | | 95191403 | |
| 41 | 0.96 | LST1 | A3SS | + | chr6: 31587733- | chr6: 31587733- |
| | | | | | 31588517 | 31588562 |
| 42 | 0.96 | LRRK1 | A3SS | + | chr15: 101062690- | chr15: 101062690- |
| | | | | | 101065144 | 101065351 |
| 43 | 0.96 | LST1 | 3SS | + | chr6: 31587318- | chr6: 31587318- |
| | | | | | 31588517 | 31588562 |
| 50 | 0.97 | U91328.1 | SE | + | chr6: 25992918- | chr6: 25992918- |
| | | | | | 25993996; | 25997902 |
| | | | | | chr6: 25994142- | |
| | | | | | 25997902 | |
| 57 | 0.97 | LST1 | RI | + | chr6: 31587298- | chr6: 31587318- |
| | | | | | 31587338; | 31588562 |
| | | | | | chr6: 31588542- | |
| | | | | | 31588582 | |
| 67 | 0.97 | IQSEC1 | A3SS | − | chr3: 12901522- | chr3: 12899434- |
| | | | | | 12902772 | 12902772 |
| 75 | 0.97 | RPS3A | SE | + | chr4: 151099714- | chr4: 151099714- |
| | | | | | 151100484; | 151102870 |
| | | | | | chr4: 151100588- | |
| | | | | | 151102870 | |
| 79 | 0.97 | KY | A5SS | − | chr3: 134647497- | chr3: 134647497- |
| | | | | | 134650824 | 134650878 |
| 84 | 0.97 | PHOSPHO1 | 3SS | − | chr17: 49225131- | chr17: 49225004- |
| | | | | | 49226646 | 49226646 |
| 87 | 0.98 | RILP | SE | − | chr17: 1646619- | chr17: 1646619- |
| | | | | | 1646905; | 1647834 |
| | | | | | chr17: 1646989- | |
| | | | | | 1647834 | |
| 96 | 0.98 | MRPS22 | RI | + | chr3: 139350302- | chr3: 139350322- |
| | | | | | 139350342; | 139350976 |
| | | | | | chr3: 139350956- | |
| | | | | | 139350996 | |
| 105 | 0.98 | ZFYVE26 | SE | − | chr14: 67804264- | chr14: 67804264- |
| | | | | | 67805216; | 67805453 |
| | | | | | chr14: 67805305- | |
| | | | | | 67805453 | |
| 112 | 0.98 | PHOSPHO1 | SE | − | chr17: 49225131- | chr17: 49225131- |
| | | | | | 49225655; | 49226646 |
| | | | | | chr17: 49225777- | |
| | | | | | 49226646 | |
| 114 | 0.98 | LILRB2 | A3SS | − | chr19: 54277600- | chr19: 54277597- |
| | | | | | 54277888 | 5427788 |

When tested on a third independent clinical cohort with balanced COVID-19 versus healthy control (n=62), who were not included in any previous analyses, the microfluidic device with the 27 AS biomarkers of Table 2 achieved nearly perfect linear separation (accuracy=98.4%, 95% confidence interval [CI]: 90.2%-99.9%) using a simple principal-component analysis, with a 96.7% (95% CI: 81.5%-99.8%) positive percent agreement (PPA) and 100% (95% CI: 86.3%-100%) negative percent agreement (NPA) as illustrated in FIG. 4E.

The microfluidic PCR data was re-analyzed based on gene expression markers from a recent report that investigated the same CHARM cohort. This report is Cappuccio et al., 2022, “Earlier detection of SARSCoV-2 infection by blood RNA signature microfluidics assay,” Clin. Transl. Discov. 2, e47. which is hereby incorporated by reference. Based on a signature set discovered by whole-blood RNA-seq DEG biomarkers (n=41), Cappuccio profiled a subset of n=275 subjects from the CHARM cohort on the microfluidic PCR platform. Applying the same principal components linear classifier to the DEG principal-component analysis (PCA), the model accuracy was 90.2%, with PPA=85.7% and NPA=93.6% (FIG. 4F). To account for the sample size differences between the two microfluidic assays, downsampling from the gene expression assay to match the sample size of the AS assay was performed and the accuracy was computed over 10,000 downsamples. When sample sizes were matched, the AS biomarker assay was still significantly more accurate than the gene expression assay (p=0.011; FIG. 7D). It was further demonstrated that the within-group, between-individual variability of our AS biomarker assay was statistically smaller than both non-PSI-transformed raw probe values and gene expression probes by Cappuccio et al., (FIG. 7E). Since this effect is specific to PSI-transformed probe values, it suggests the AS biomarker assay had a smaller technical variance, likely due to its internal normalization property. See Example 12.

Thus, this example identifies a highly performant set of host-based AS-centered biomarkers for SARS-CoV-2 infection detection and demonstrates a promising avenue for design and implementation of host-based diagnostic by leveraging RNA splicing as robust diagnostic biomarkers.

### Example 4—Interpreting Biological Important of AS Biomarkers

Interrogation of the biological basis of the superior performance of the AS-based diagnostic markers described in Example 3 was sought in this example. To understand the functional characteristics of genes undergoing DAS in response to SARS-CoV-2 infection, blood-specific functional networks (hb.flatironinstitute.org/) were employed. See Wong et al., 2018, “Giant 2.0: genome-scale integrated analysis of gene networks in tissues,” Nucleic Acids Res., 46, W65-W70, which is hereby incorporated by reference in its entirety for all purposes. Tissue-specific functional networks capture tissue-specific protein function, interactions, and pathway activities. See Greene et al., 2015, “Understanding multicellular function and disease with human tissue-specific networks,” Nat. Genet., 47, pg. 569-576, which is hereby incorporated by reference in its entirety for all purposes.

Functional networks in HumanBase were used to analyze the blood tissue-specific network modules and functional annotation enrichment for differentially spliced genes across the disease stages. See Wong et al., 2018. Genes with any significant DAS events were included as alternatively spliced genes in the functional network analysis. Detected modules for the whole blood-specific network were analyzed for the enriched Gene Ontology (GO) terms. To reduce and cluster similar GO terms, the ReviGO web server (revigo.irb.hr) was used to summarize GO terms for analysis of the temporal changes. See supek et al., 2011,). REVIGO summarizes and visualizes long lists of gene ontology terms,” PLoS One, 6, e21800, which is hereby incorporated by reference in its entirety.

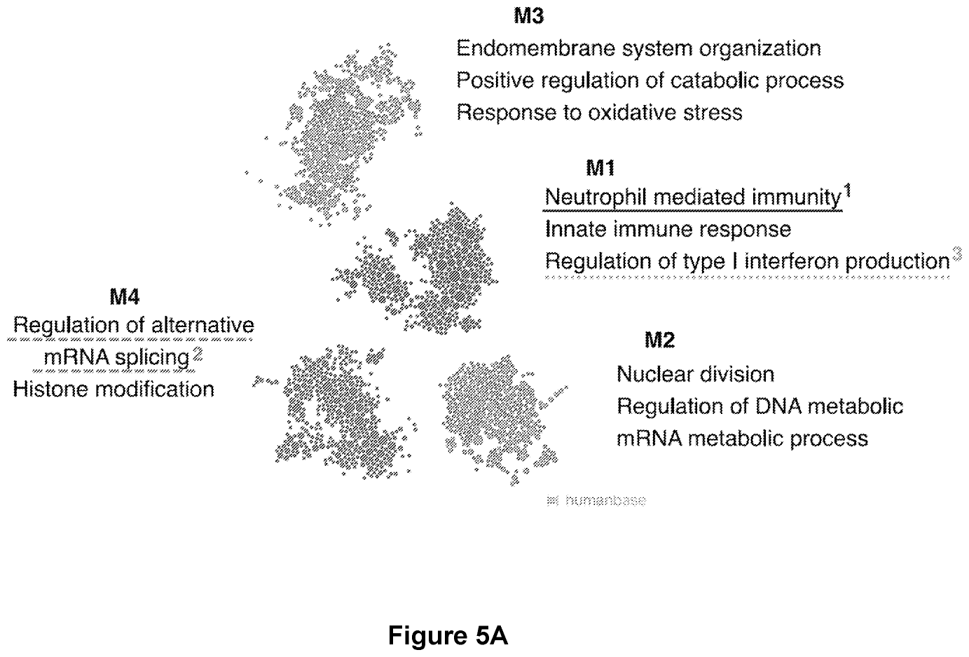

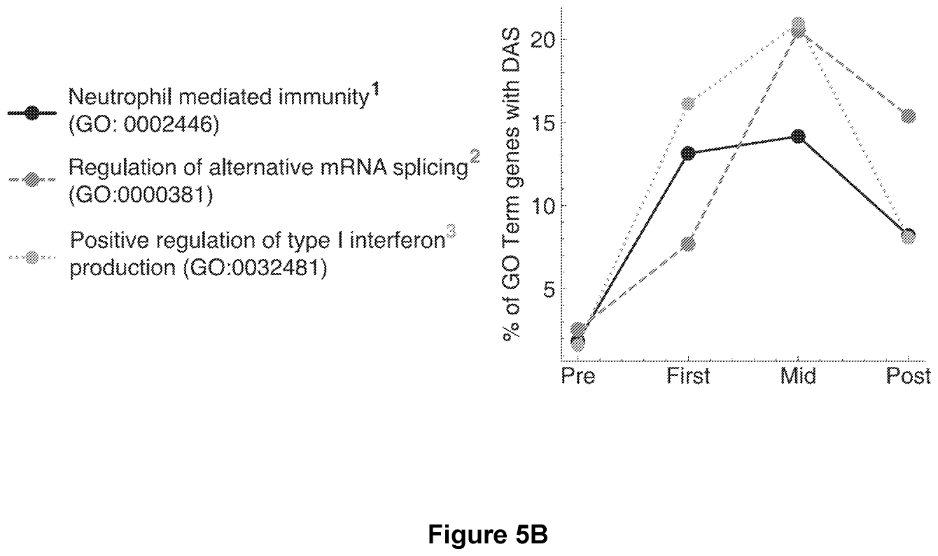

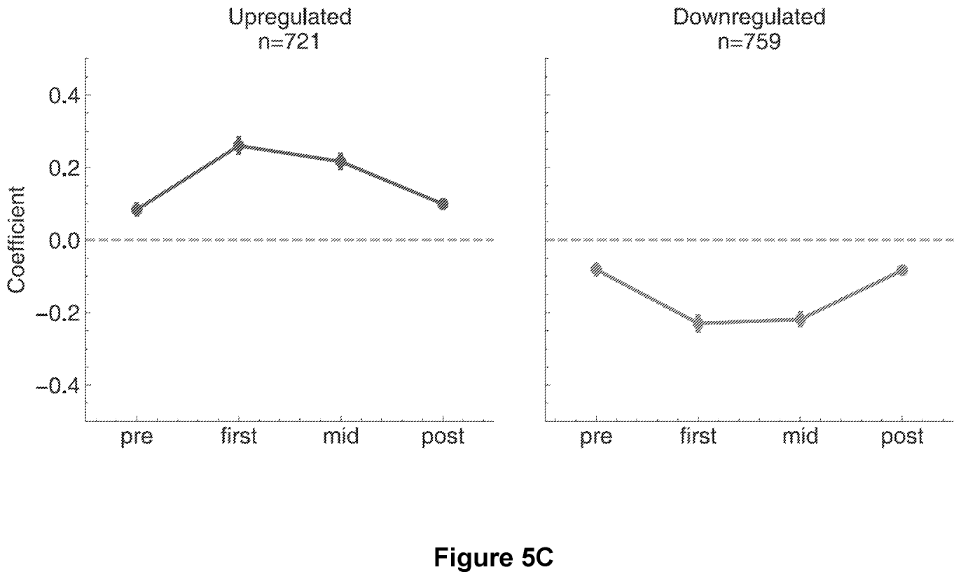

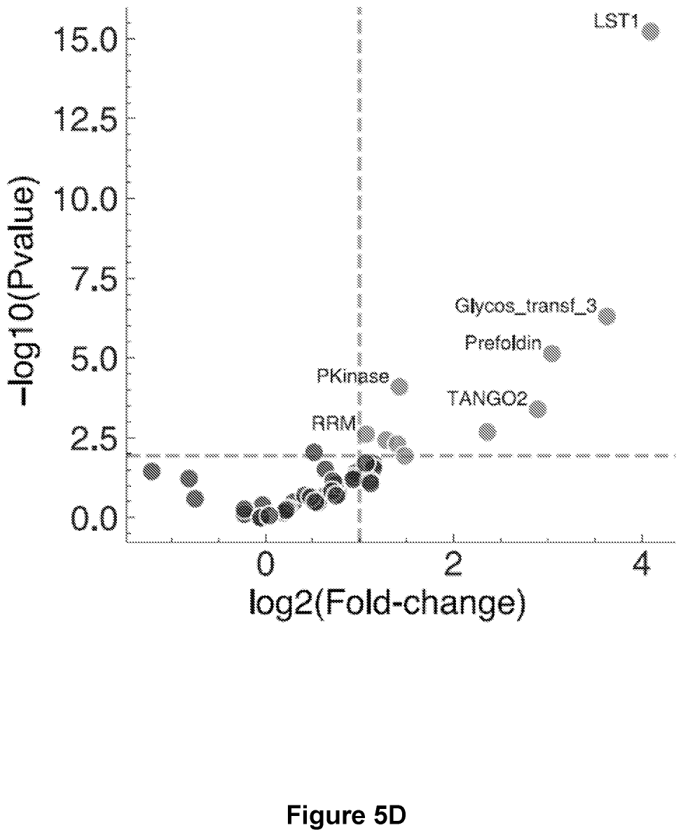

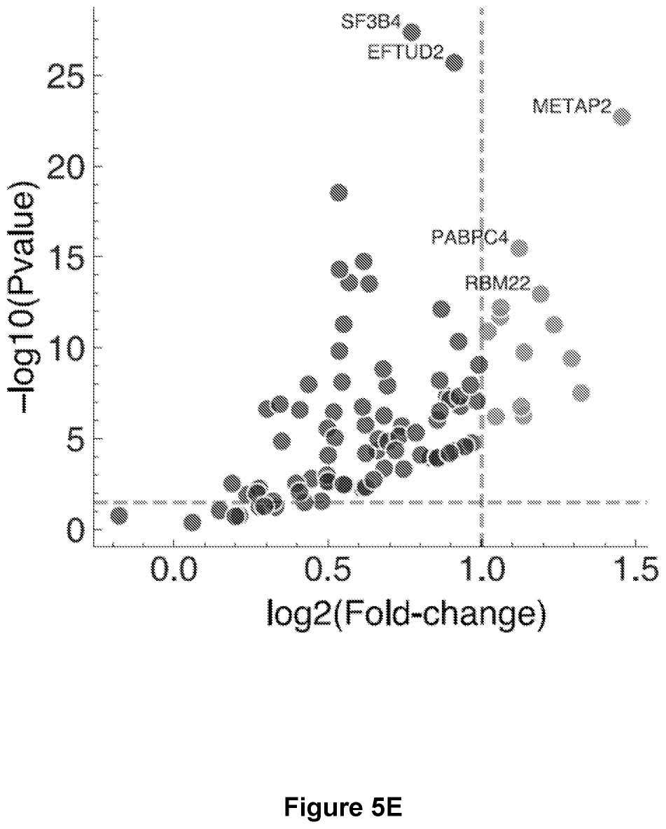

Louvain community clustering on the whole blood tissue-specific network of the genes undergoing DAS in the data reveals four modules with statistically significant top Gene Ontology (GO) terms (FDR<0.01; FIG. 5A). FIG. 5A illustrates whole blood tissue-specific gene network enrichment of genes undergoing DAS. These included a module focused on innate immune activation and chemokine signaling (M1) and a module on cell division to potentially facilitate proliferation of T cells as part of the adaptive immunity (M2), as well as a module representing phagocytosis and oxidative stress (M3). A module (M4) of genes involved in splicing regulation appeared to themselves be significantly alternatively spliced. Representative Gene Ontology terms for each module (M1, M2, M3, and M4) are shown in FIG. 5A.

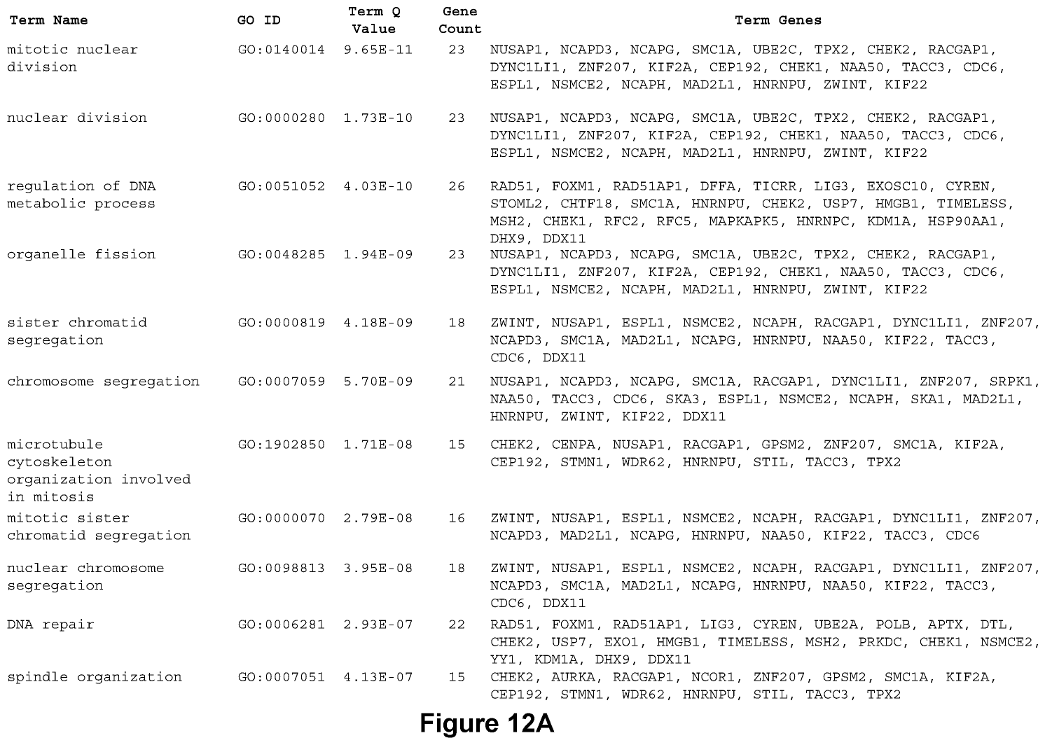

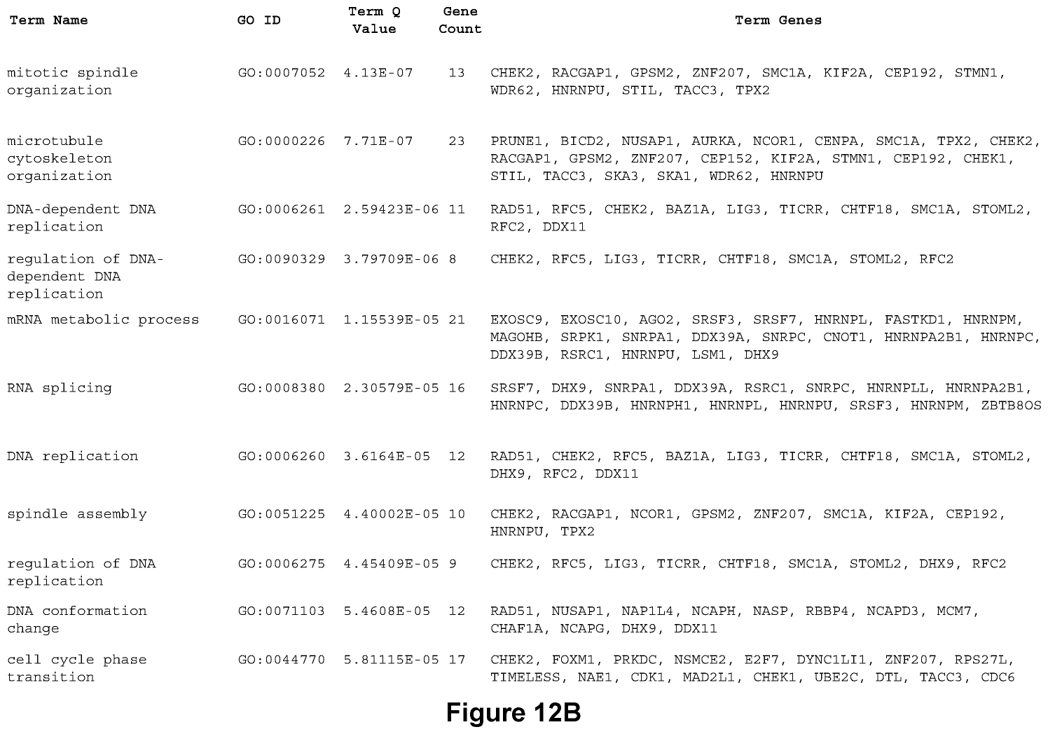

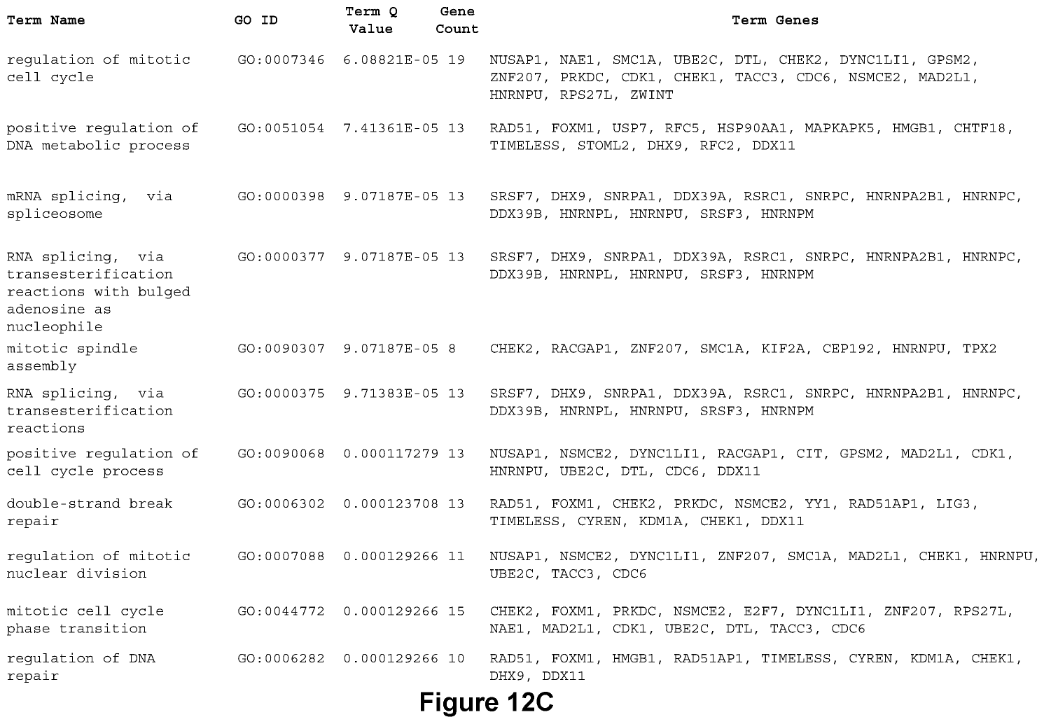

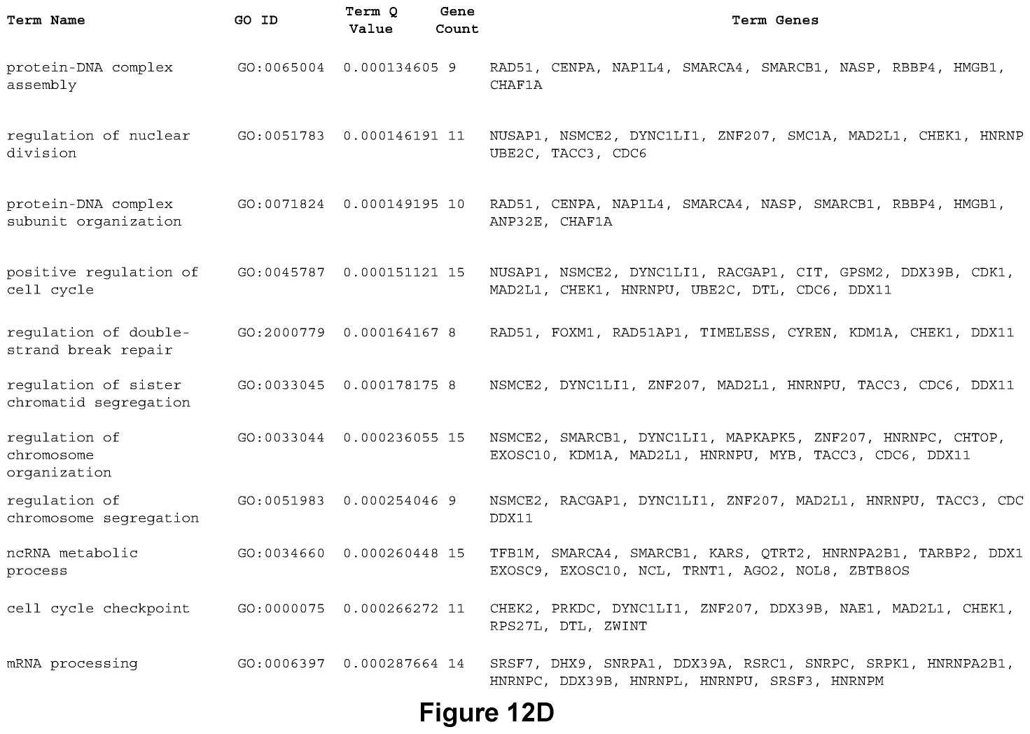

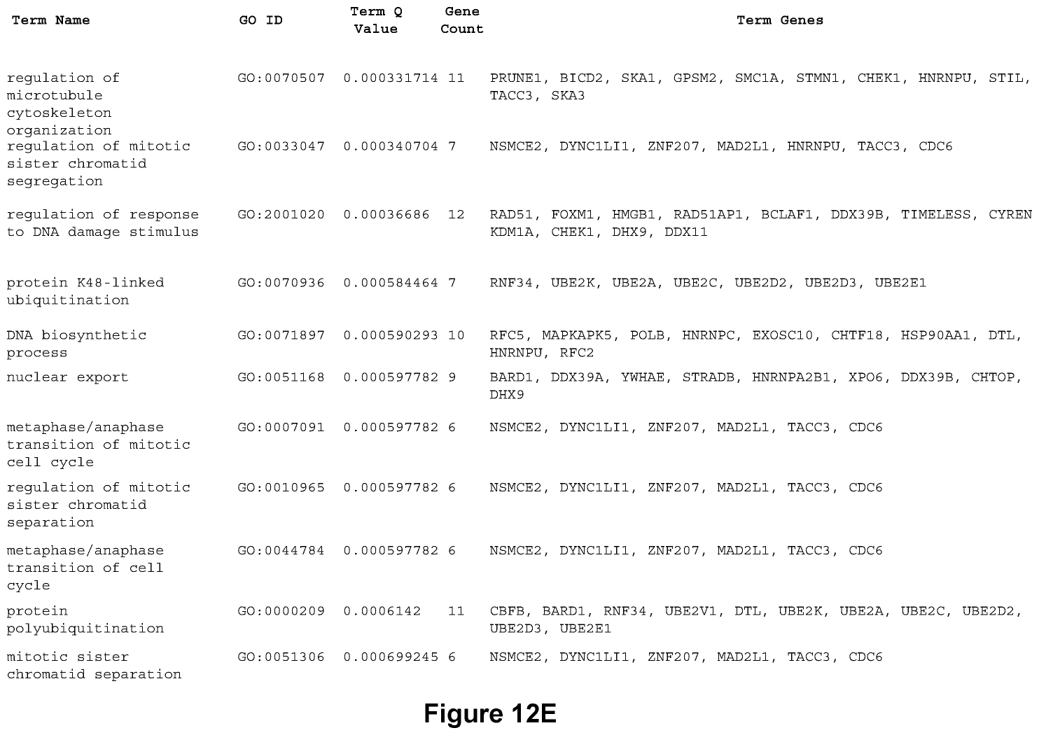

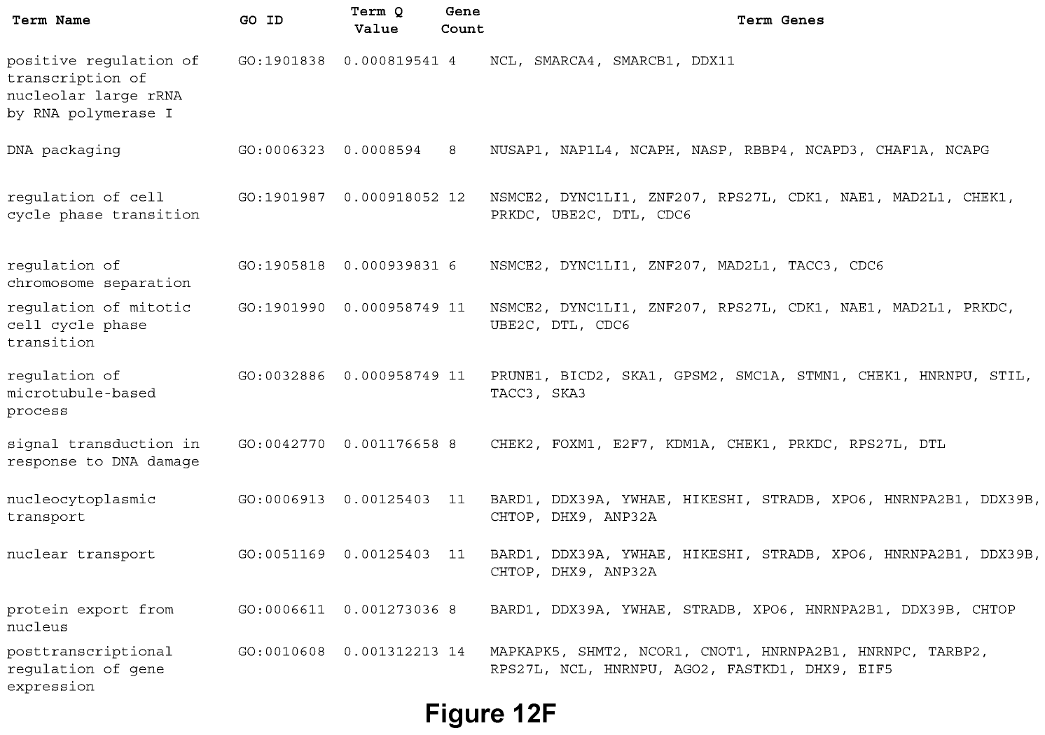

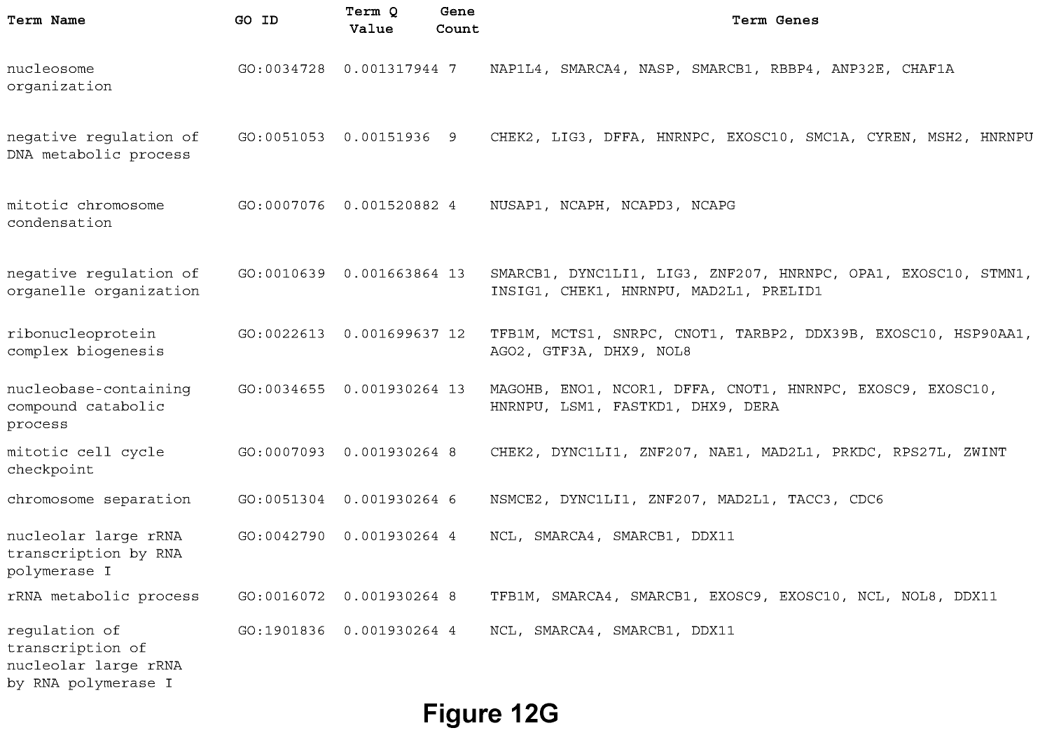

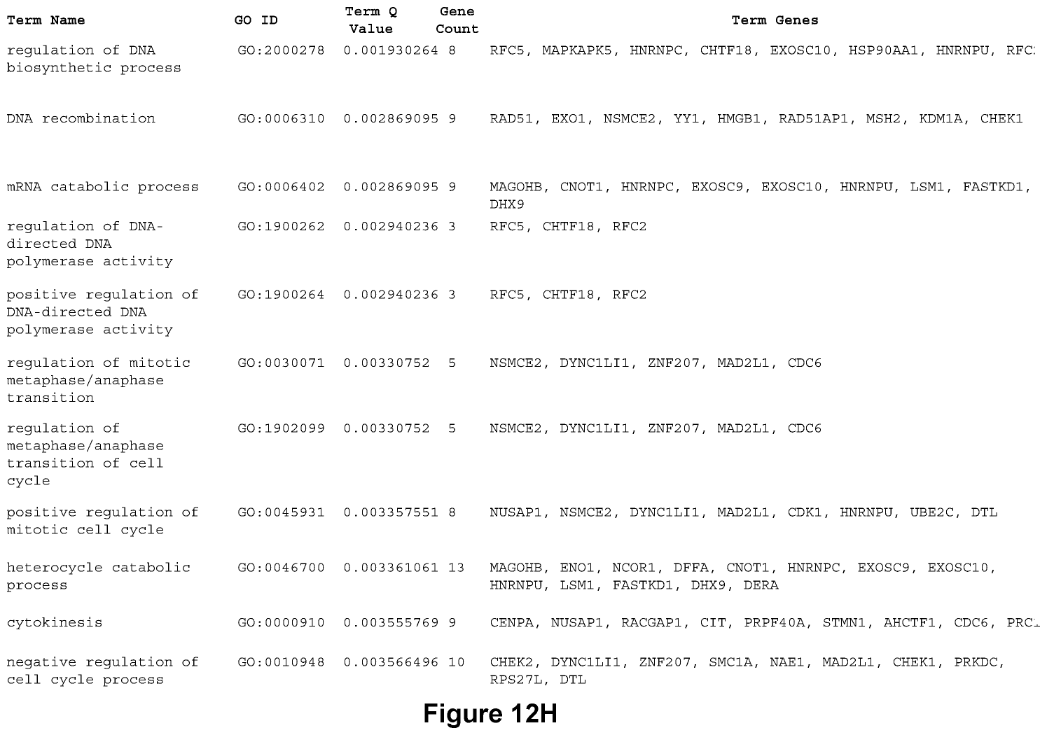

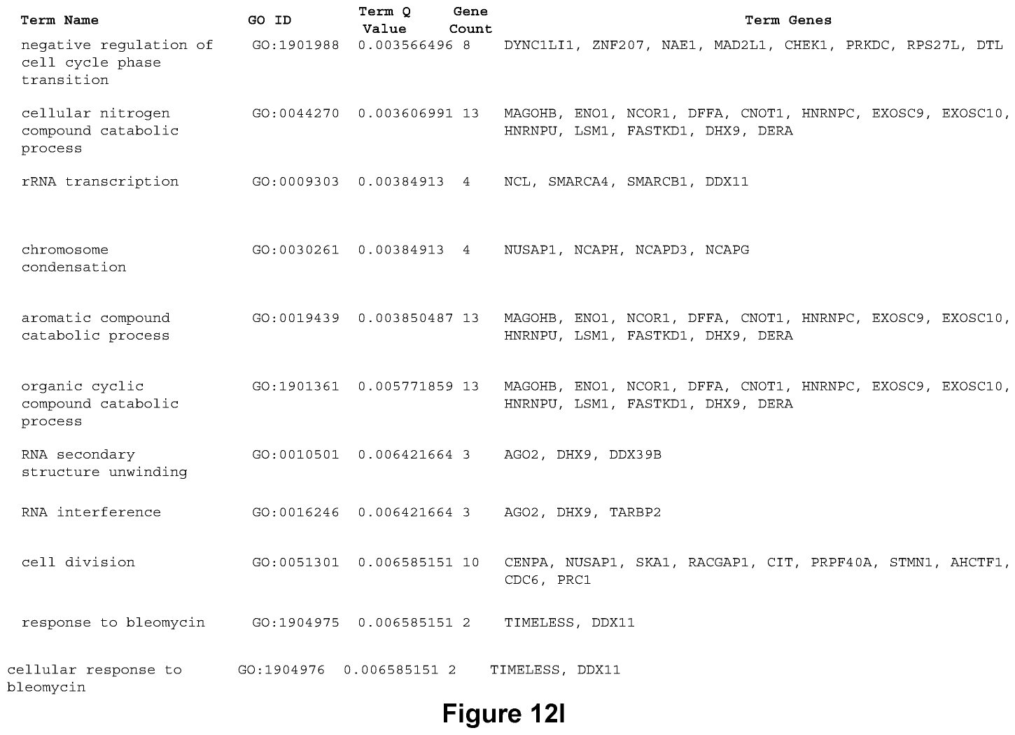

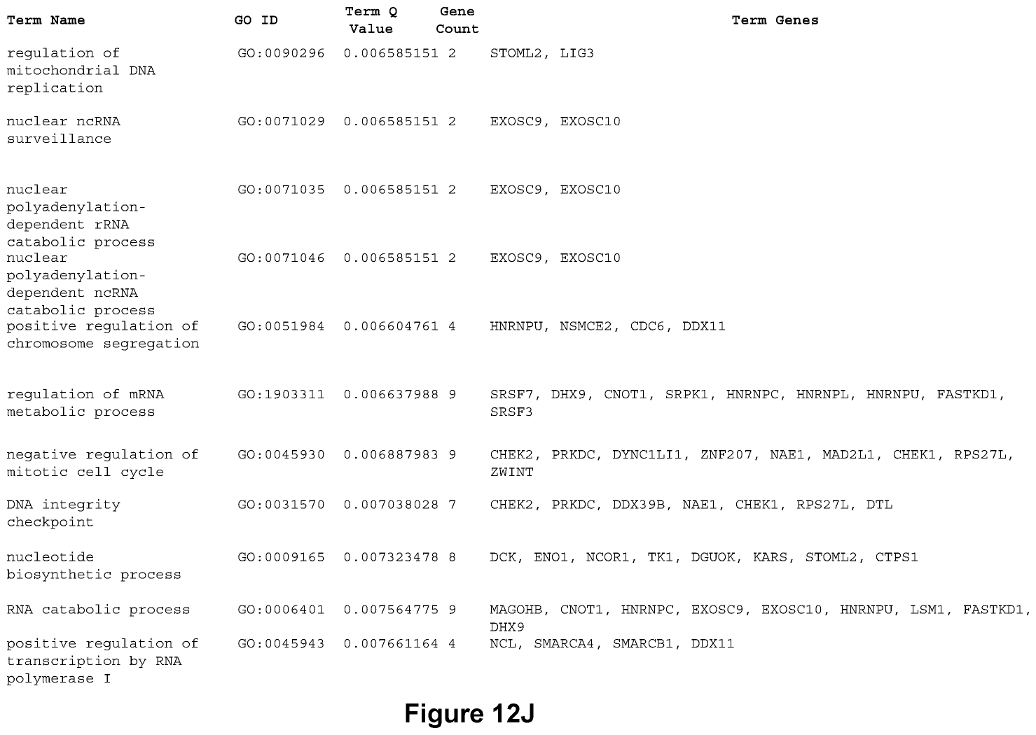

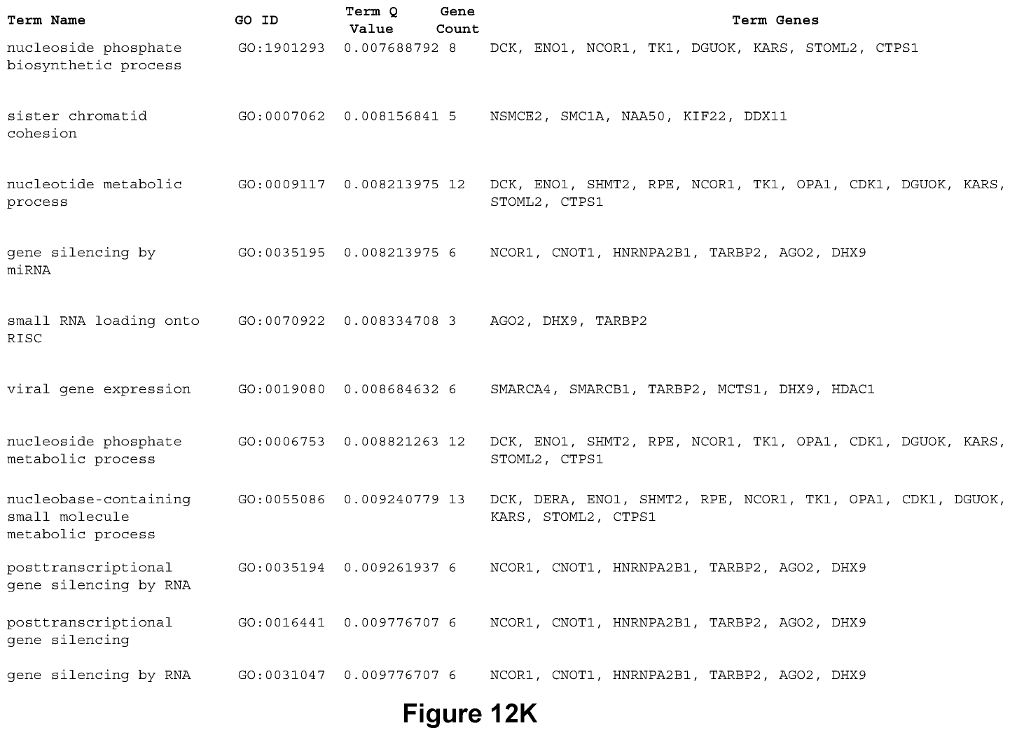

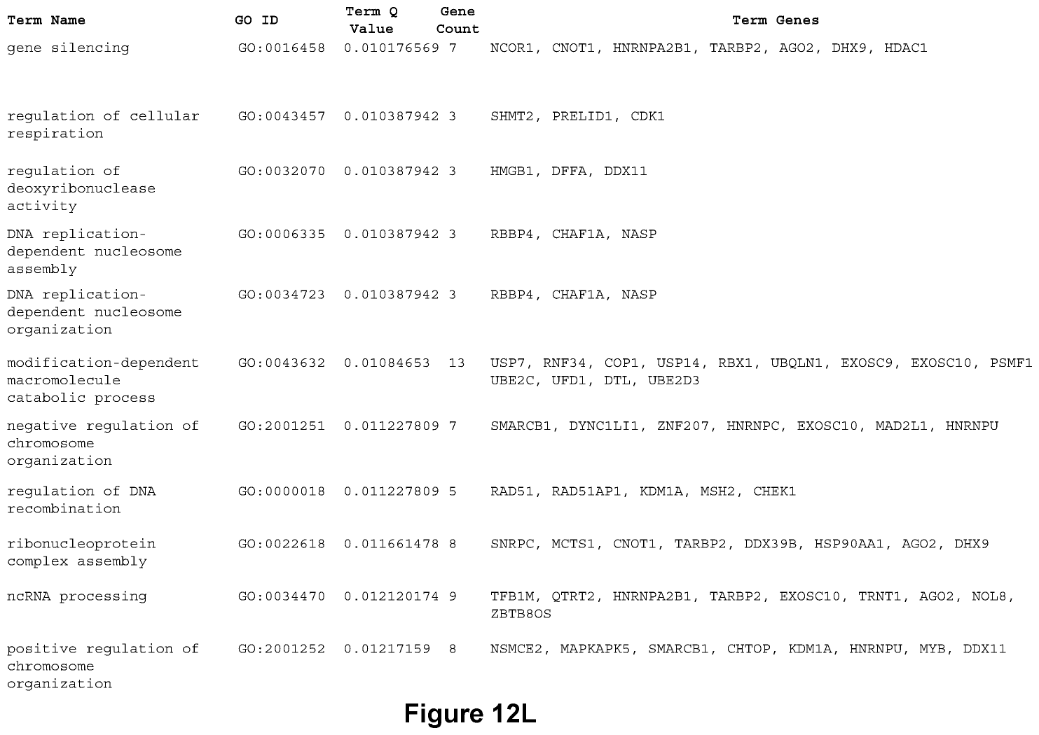

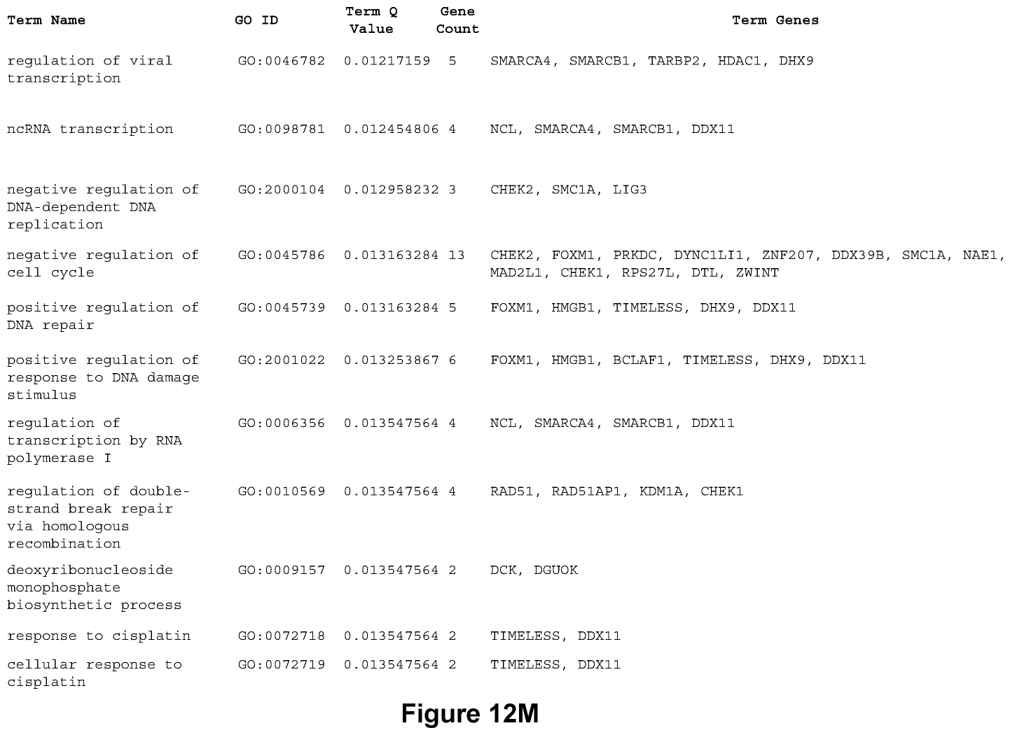

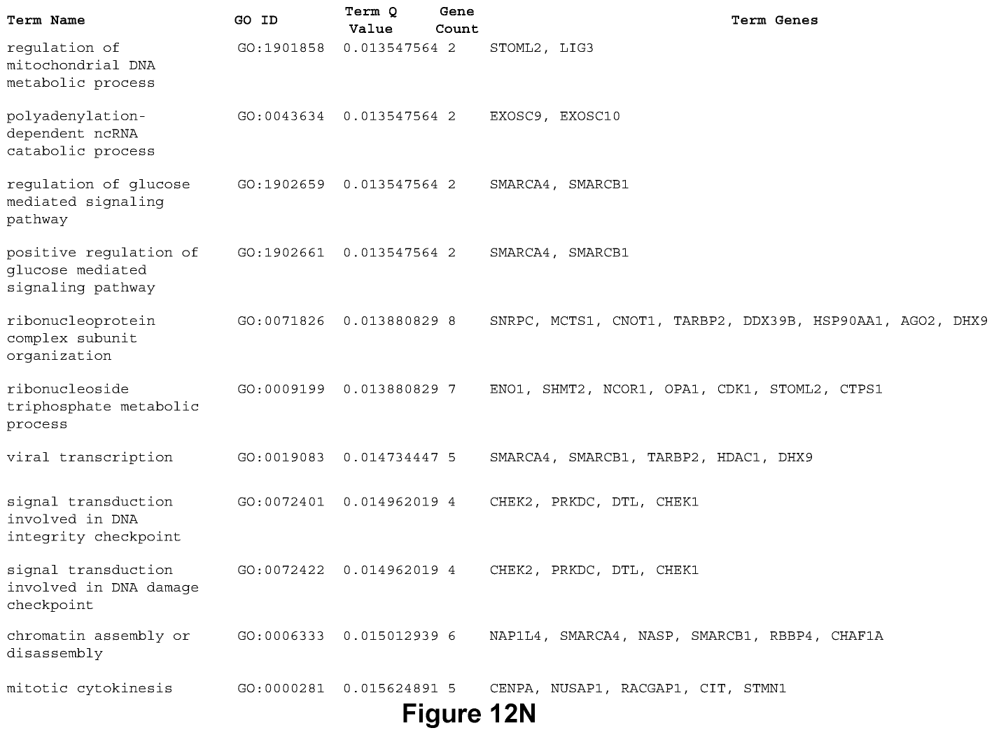

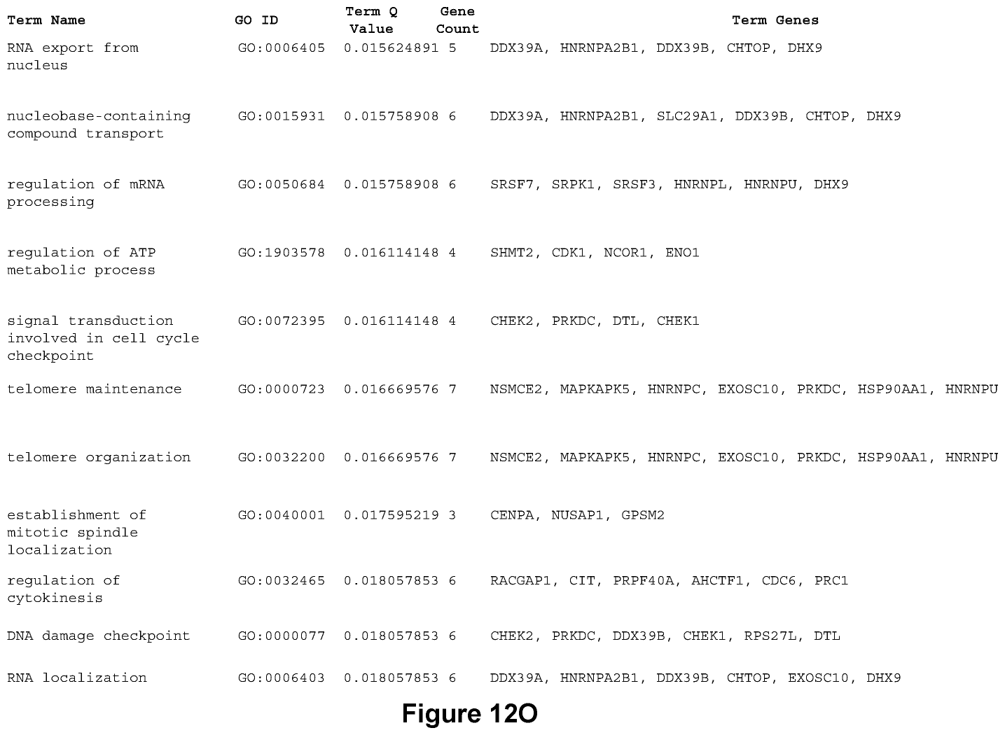

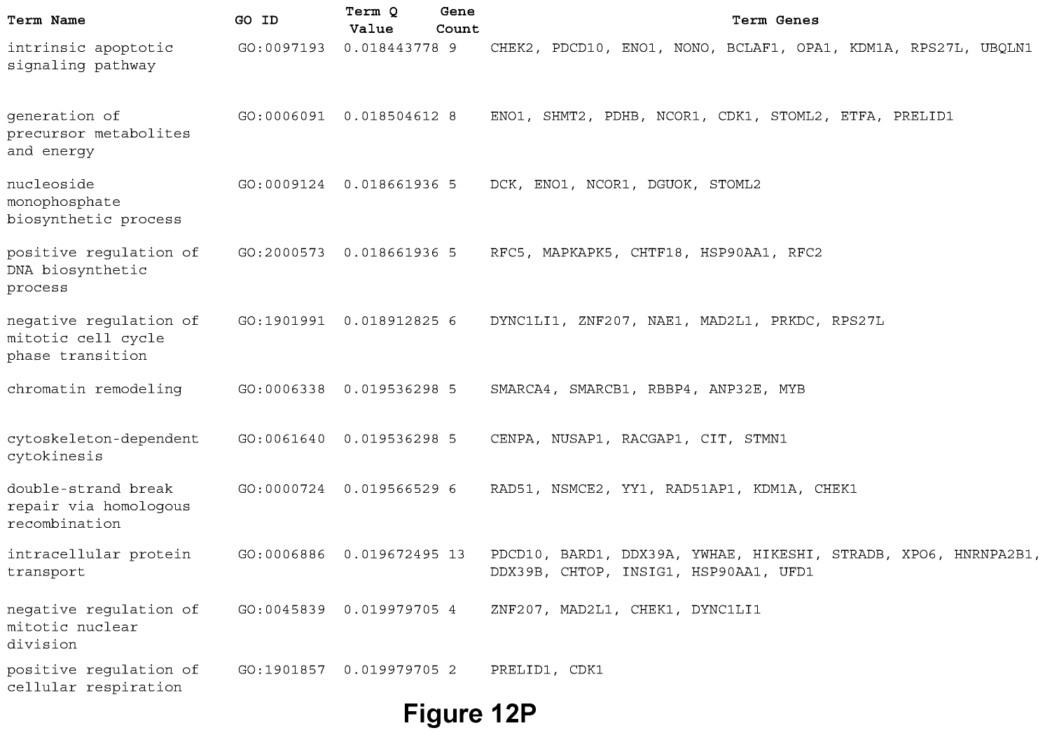

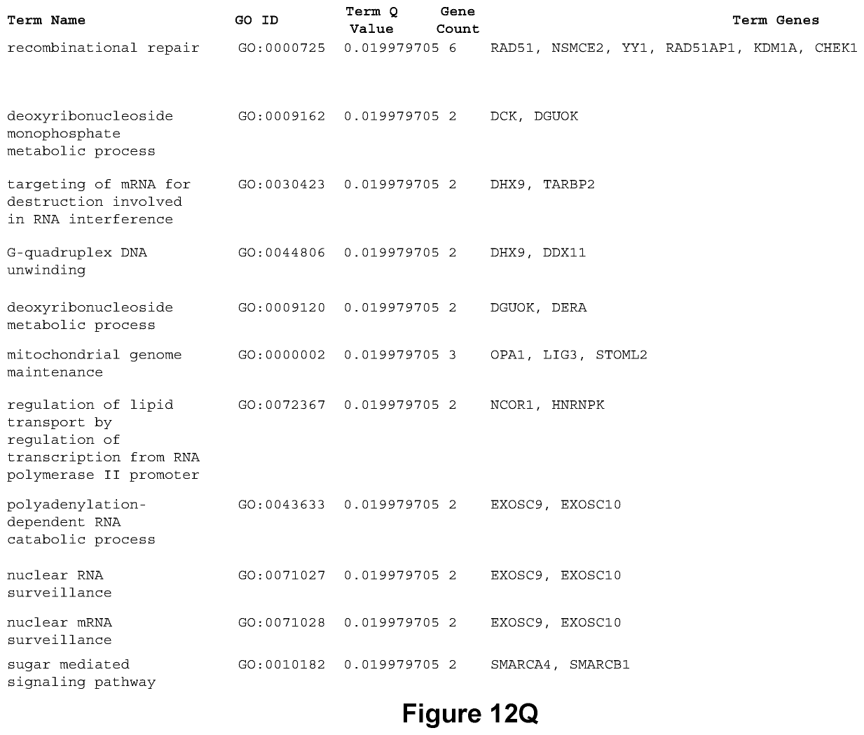

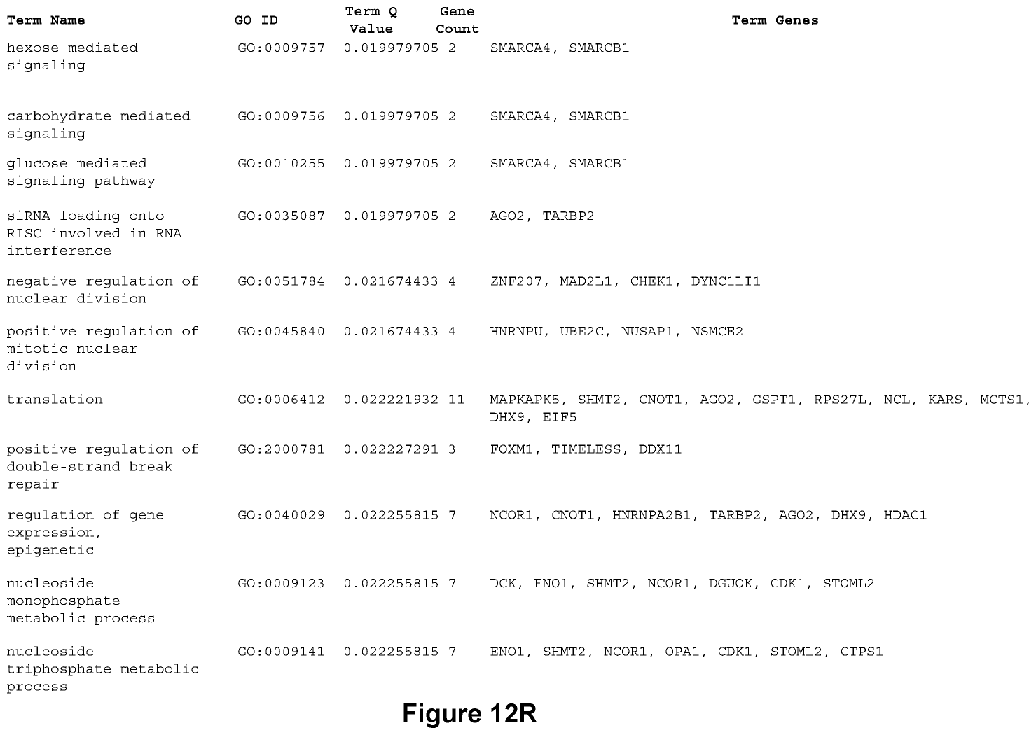

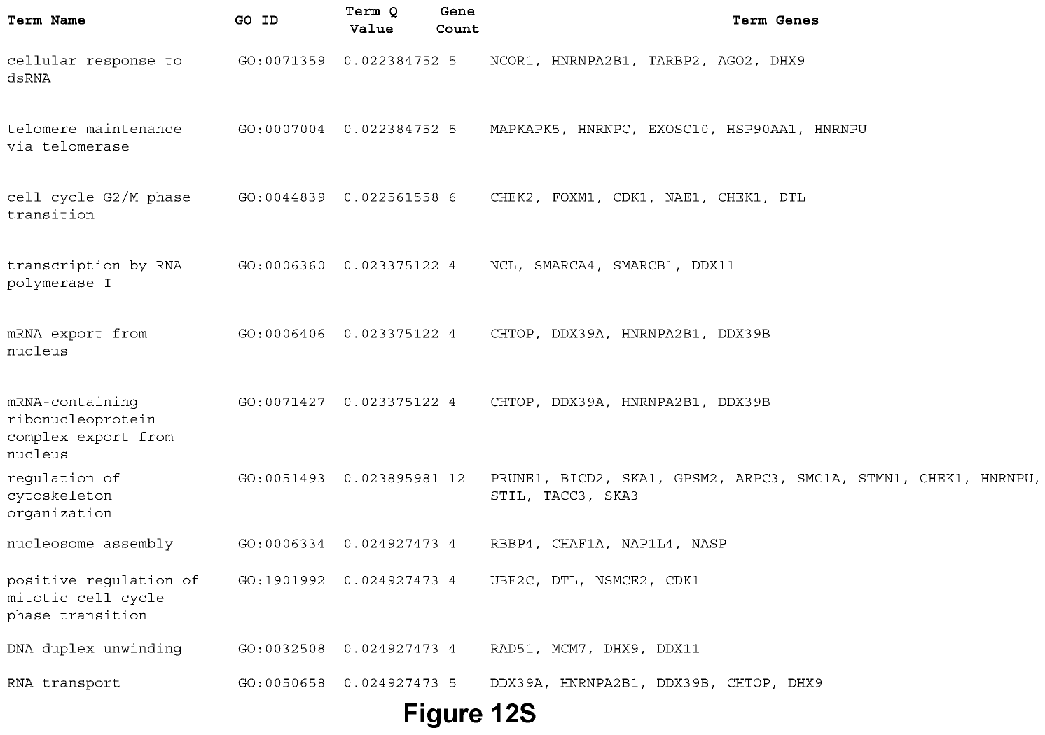

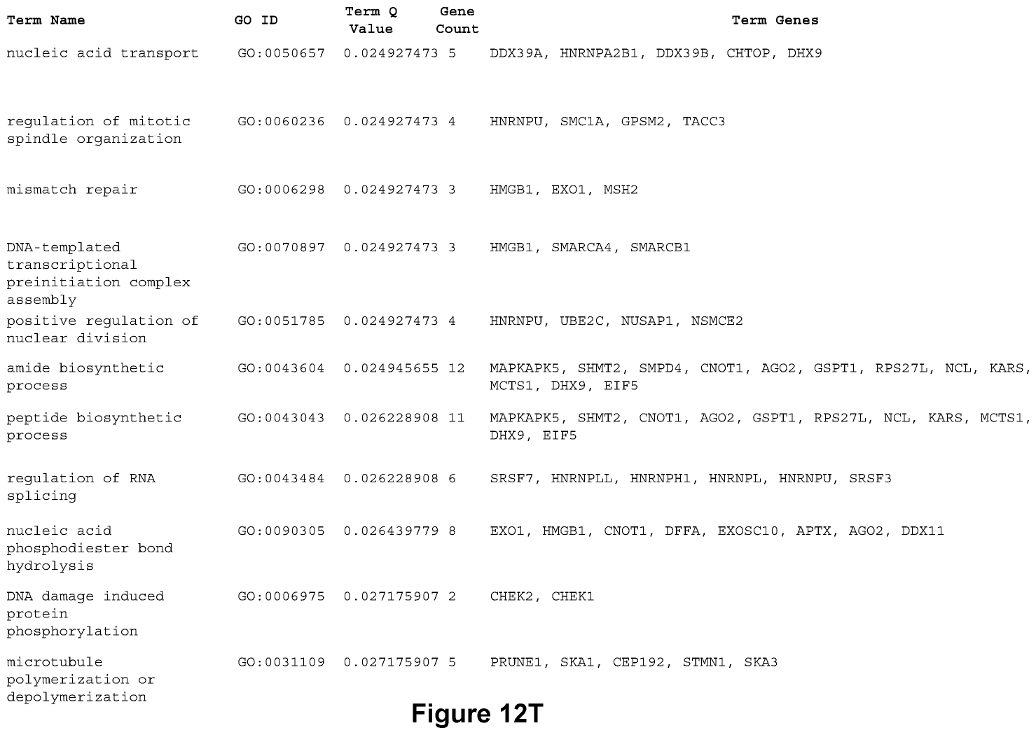

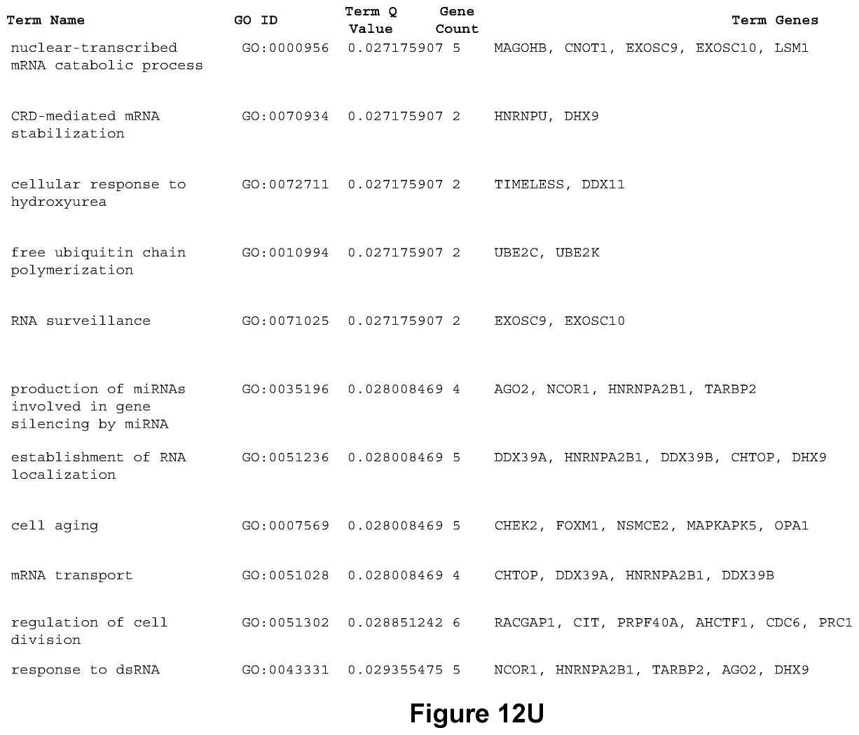

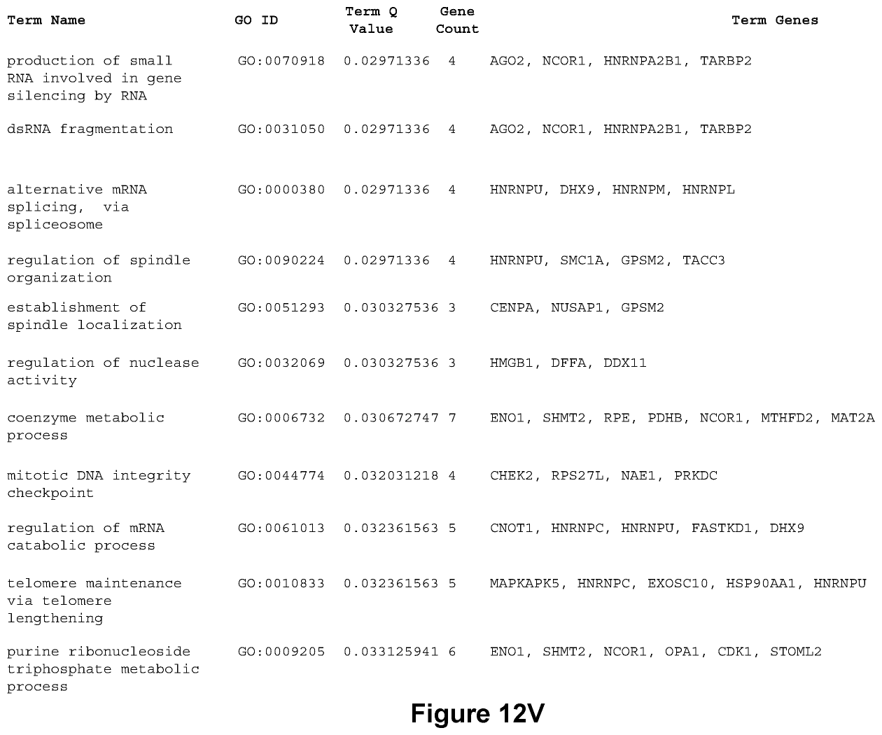

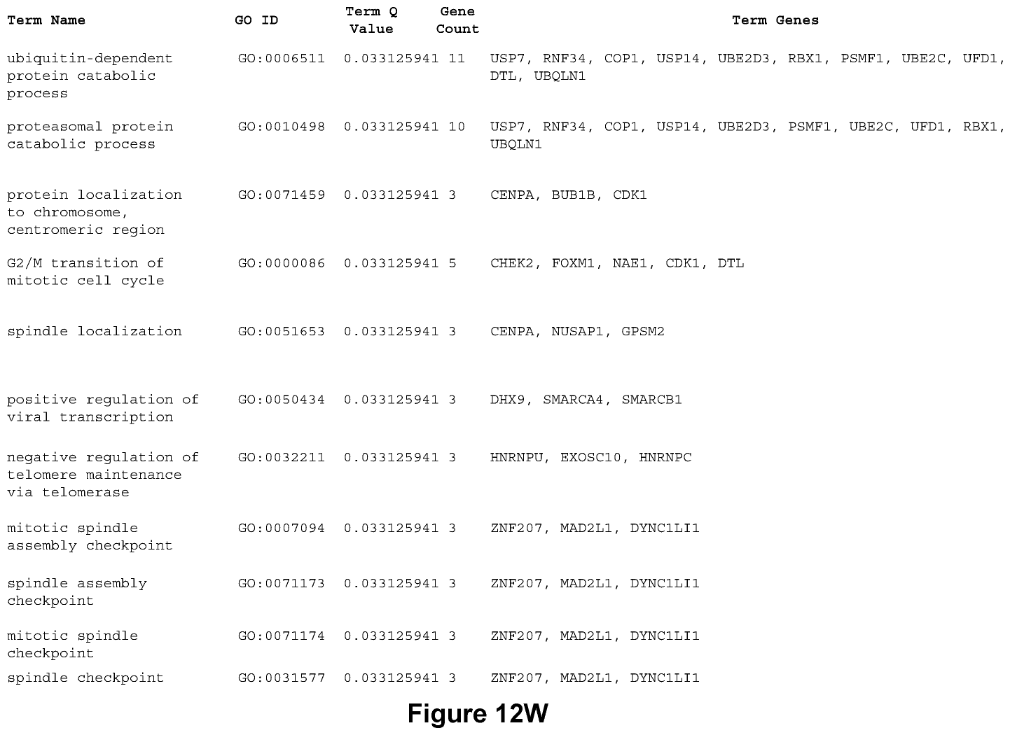

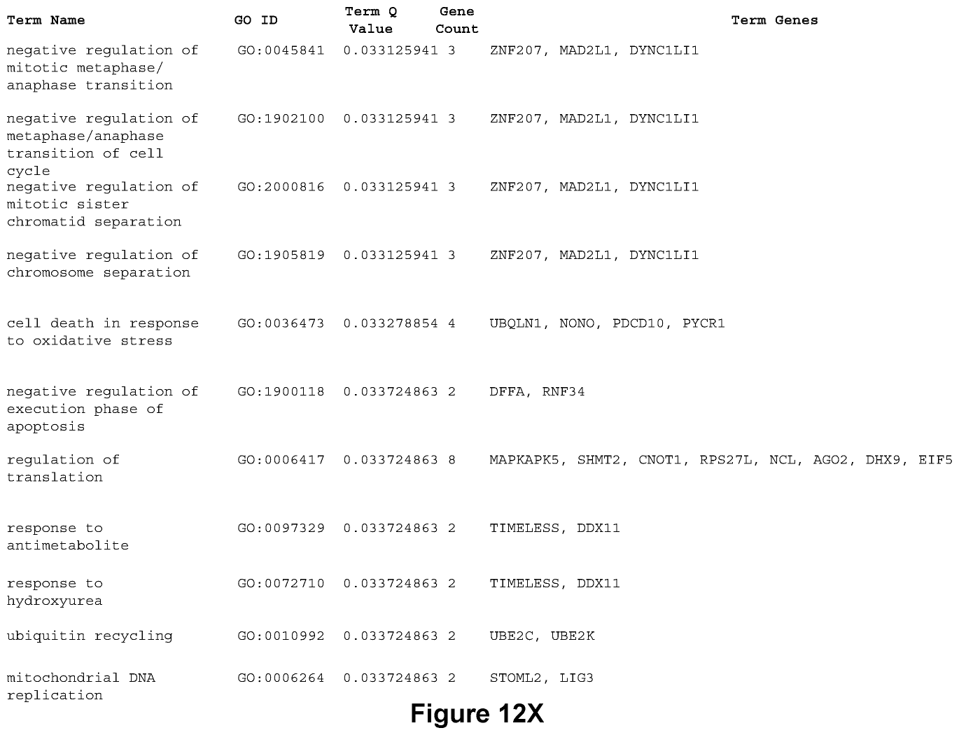

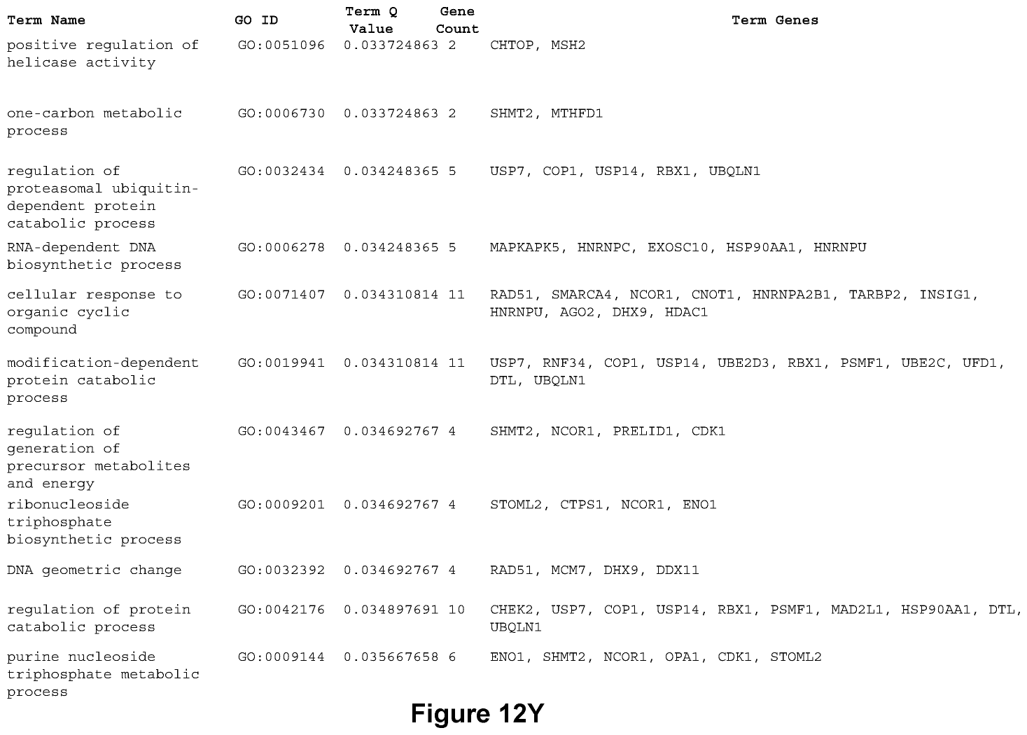

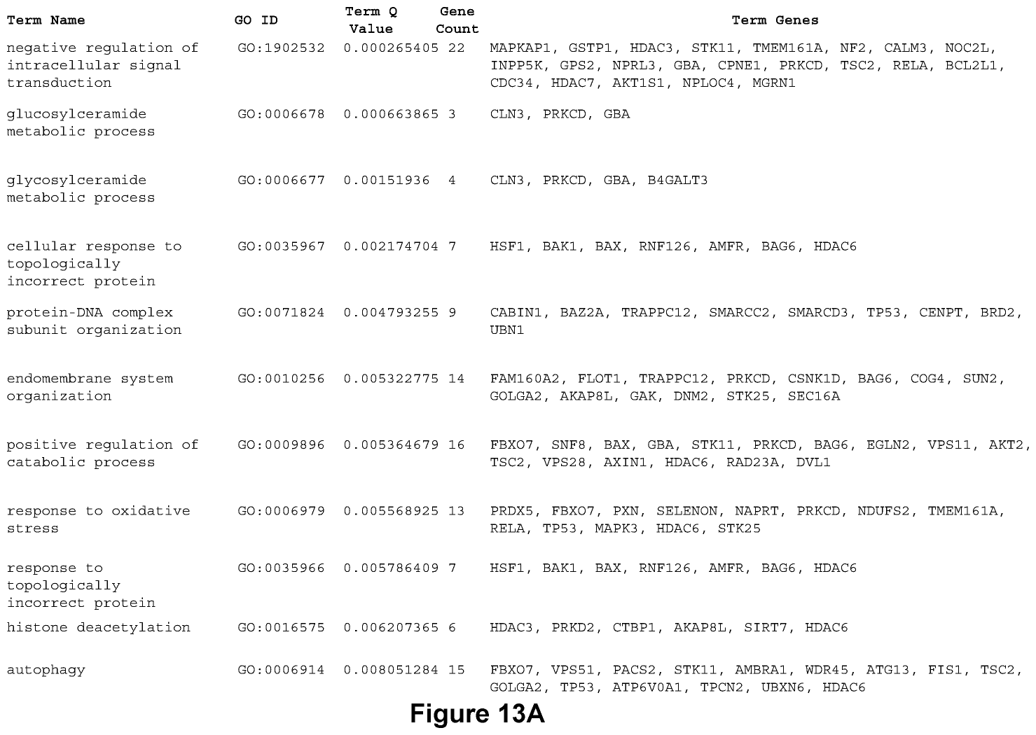

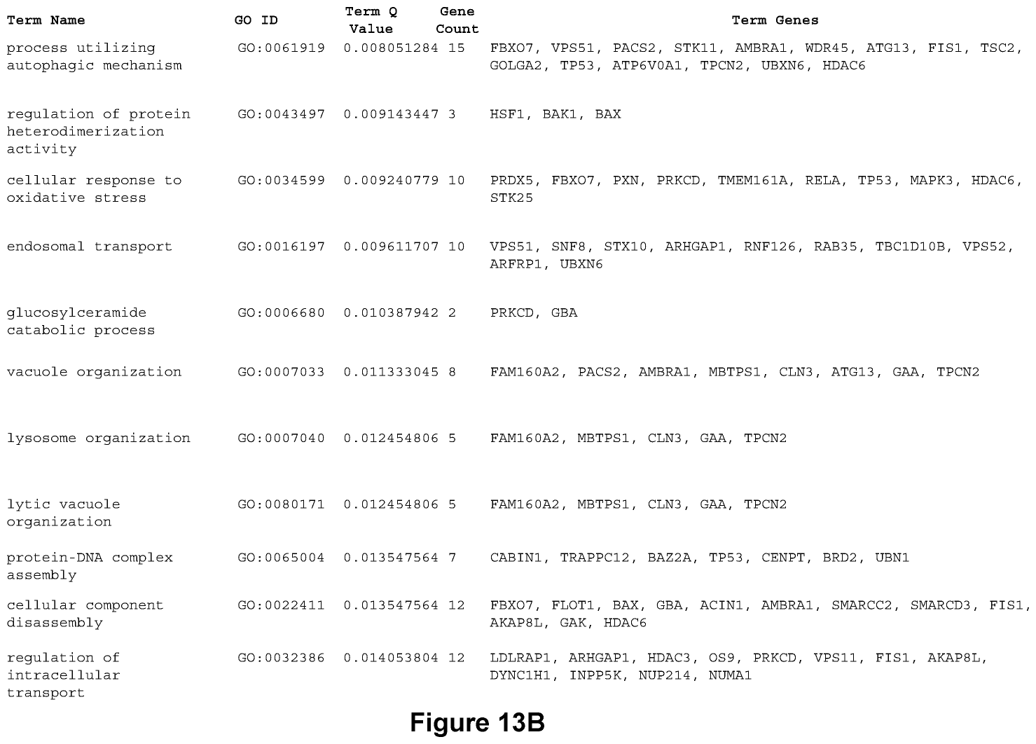

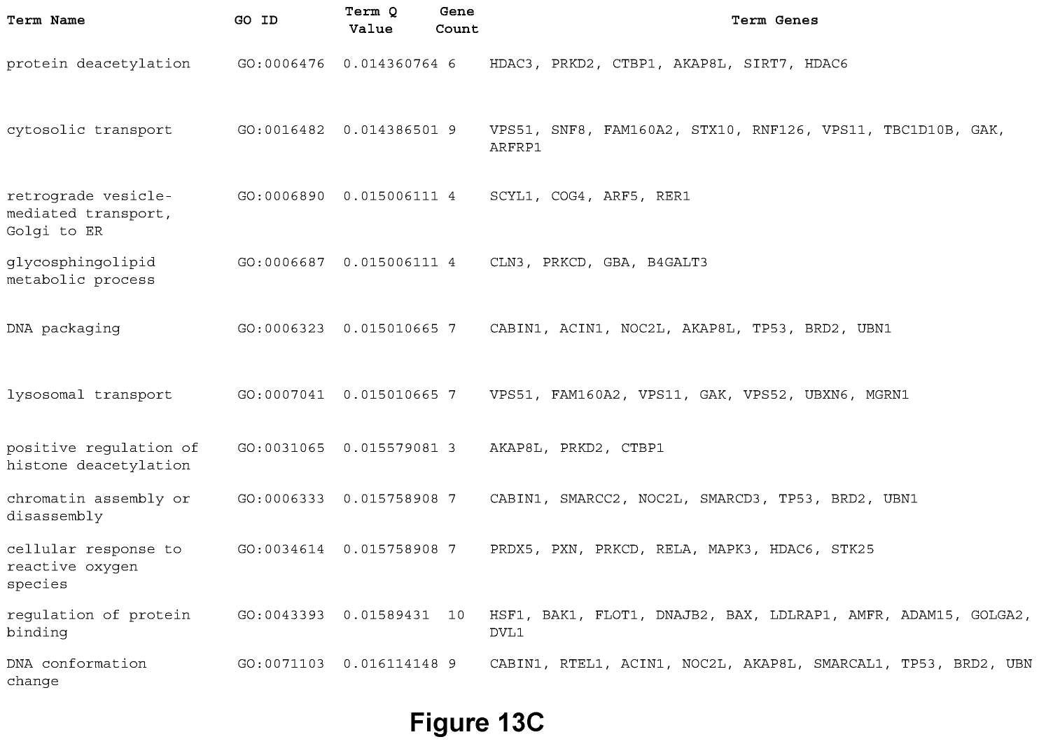

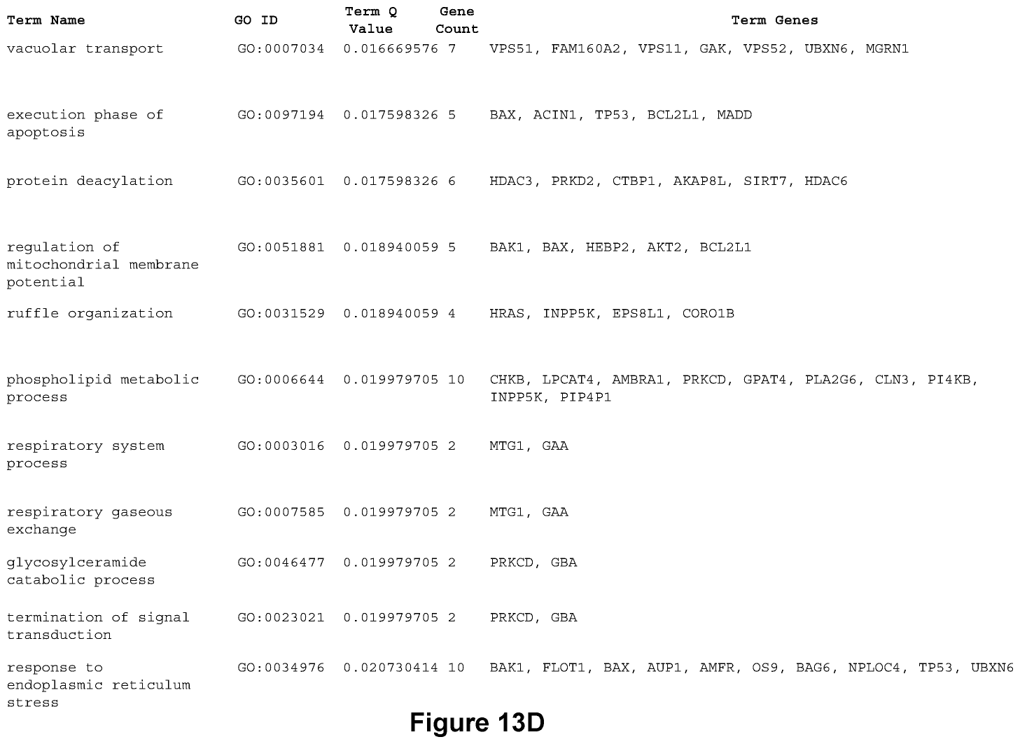

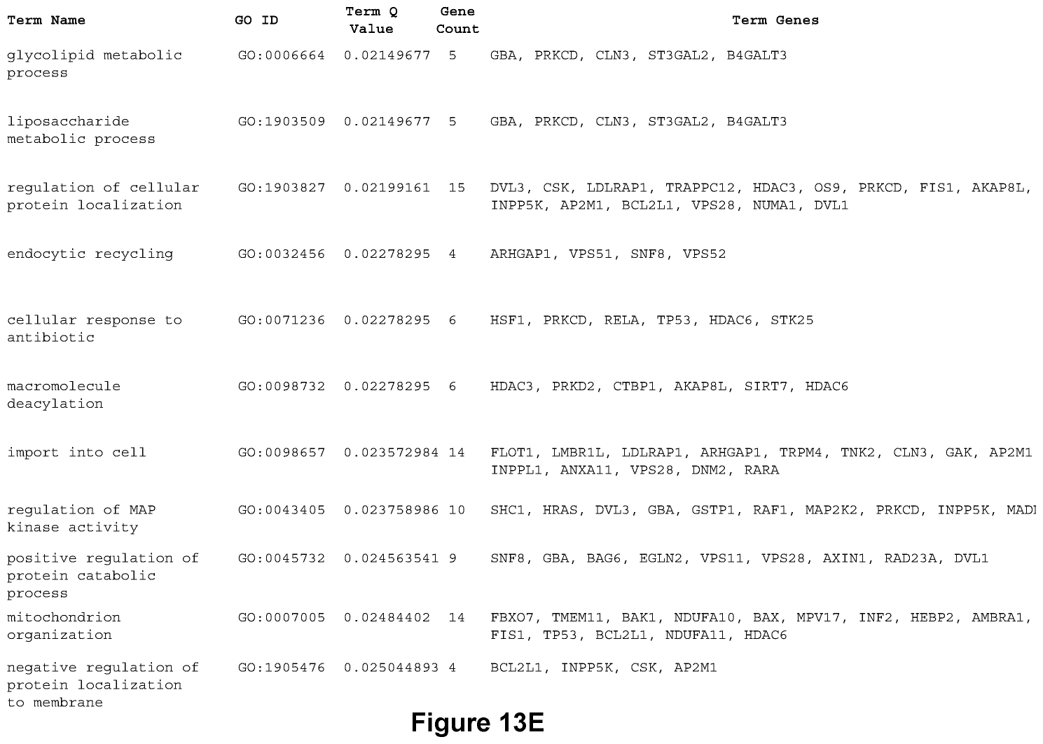

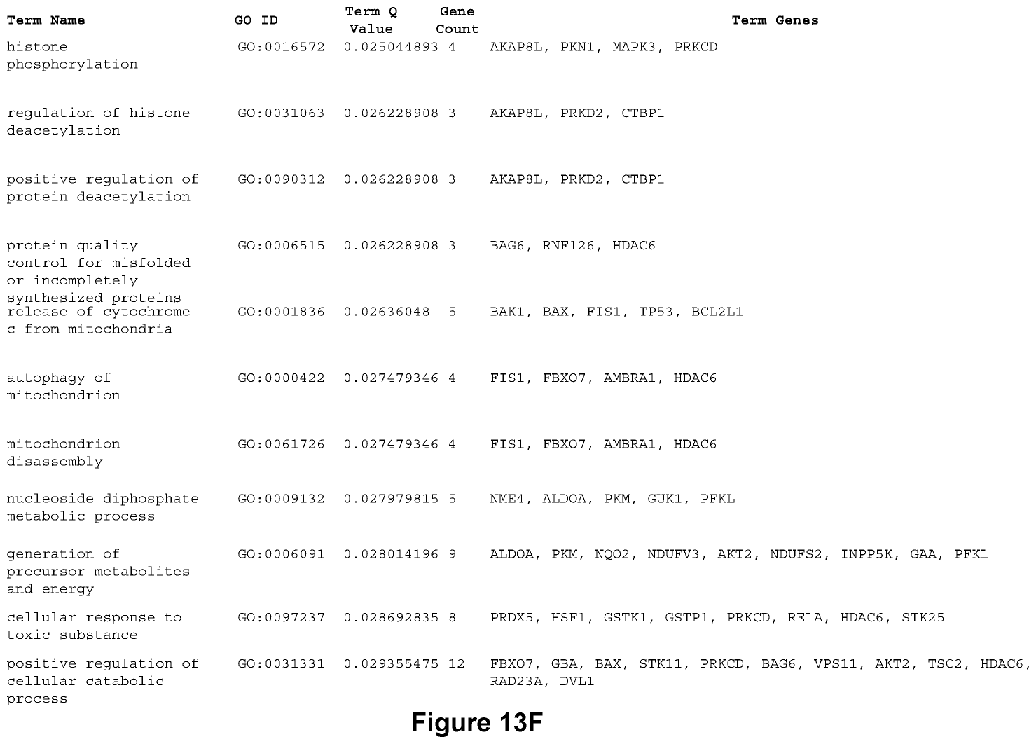

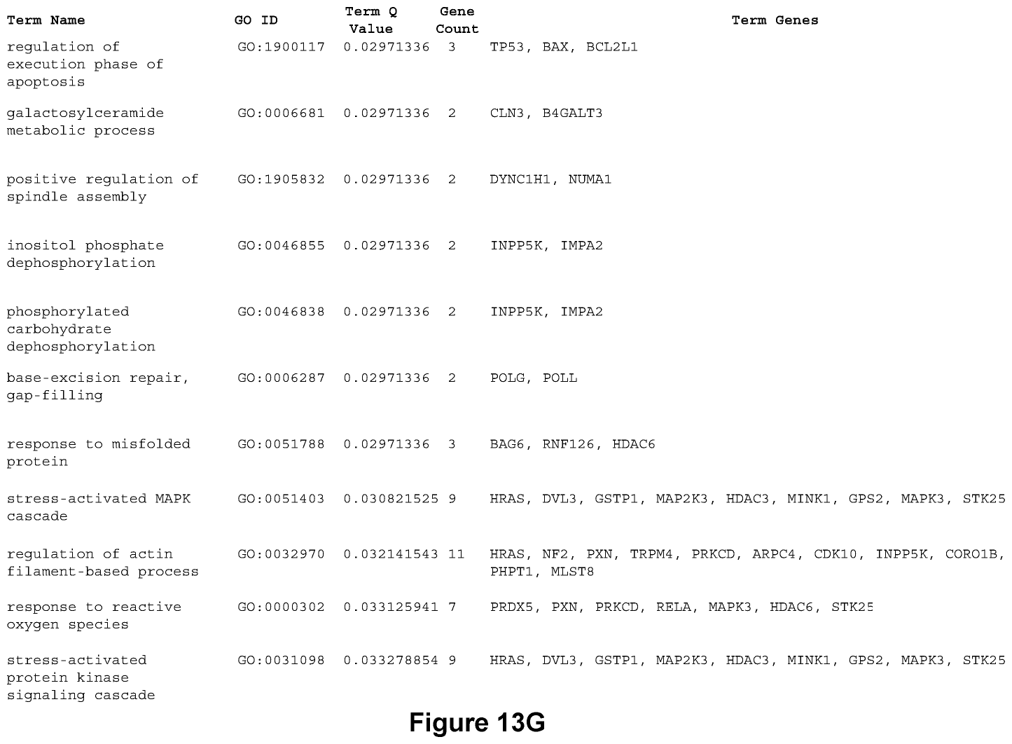

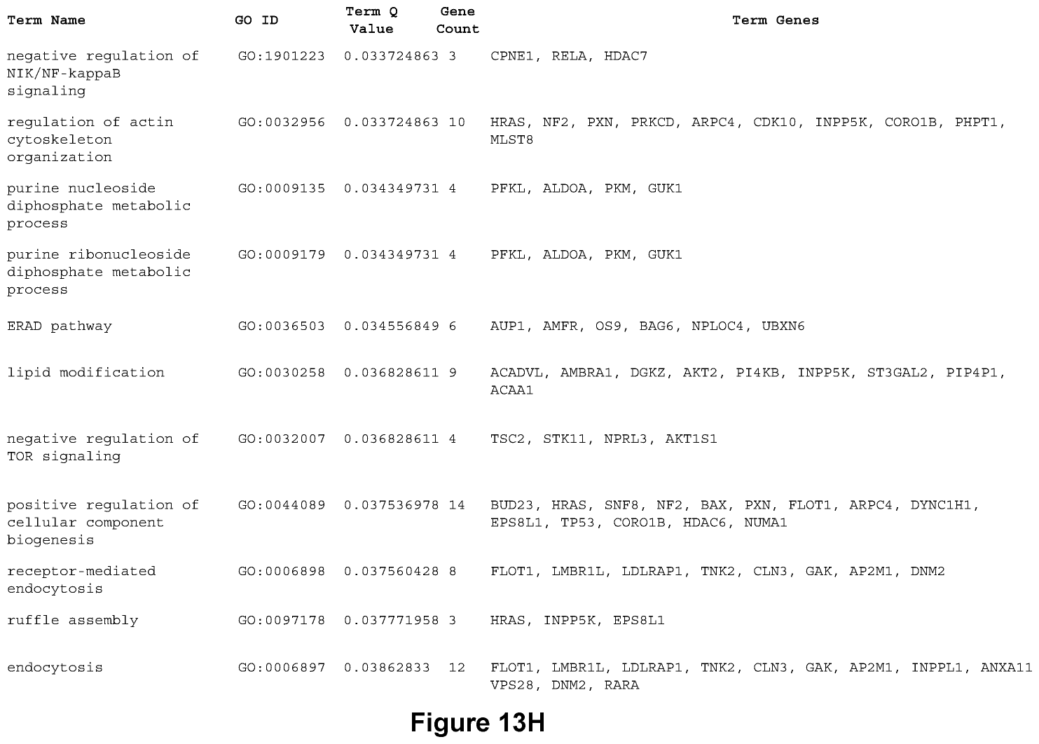

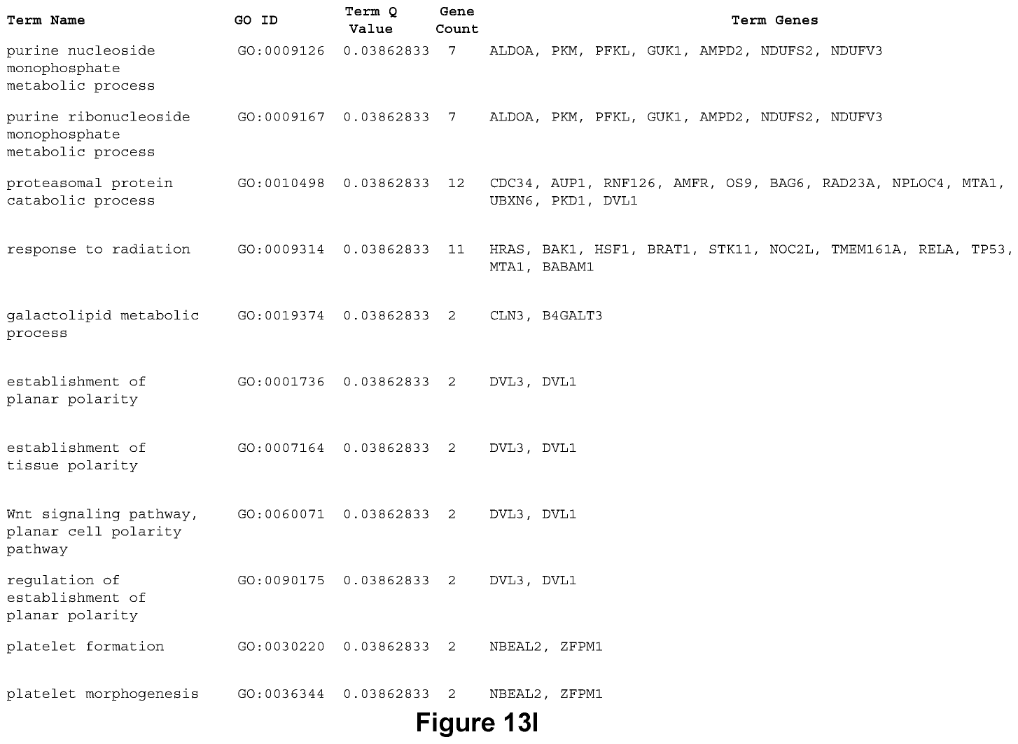

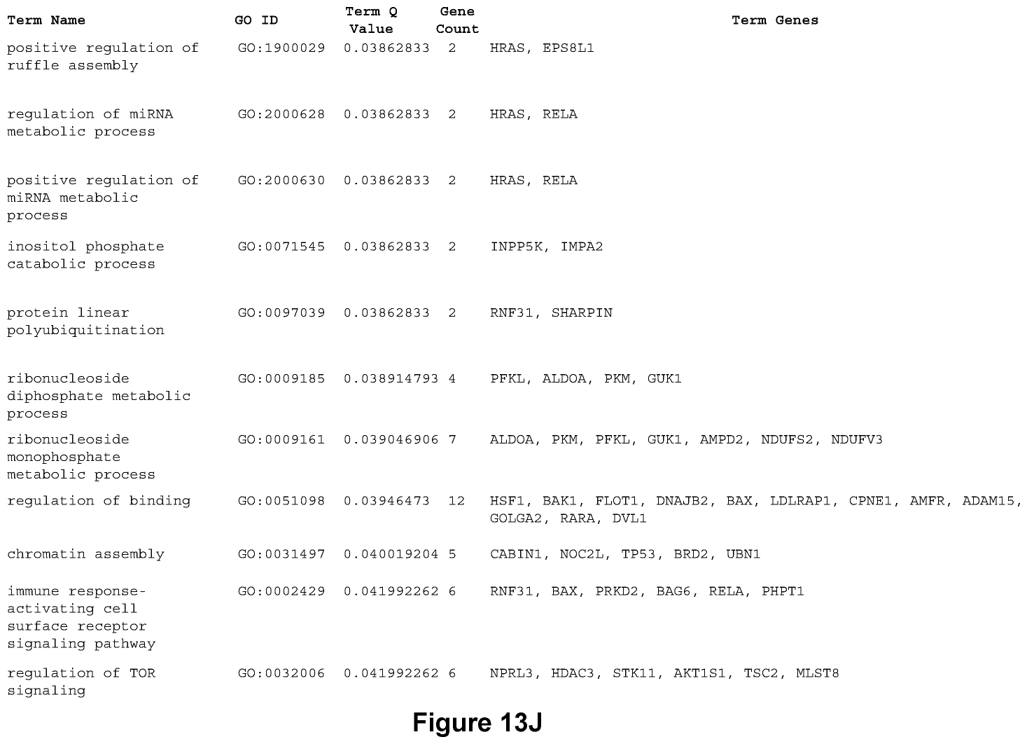

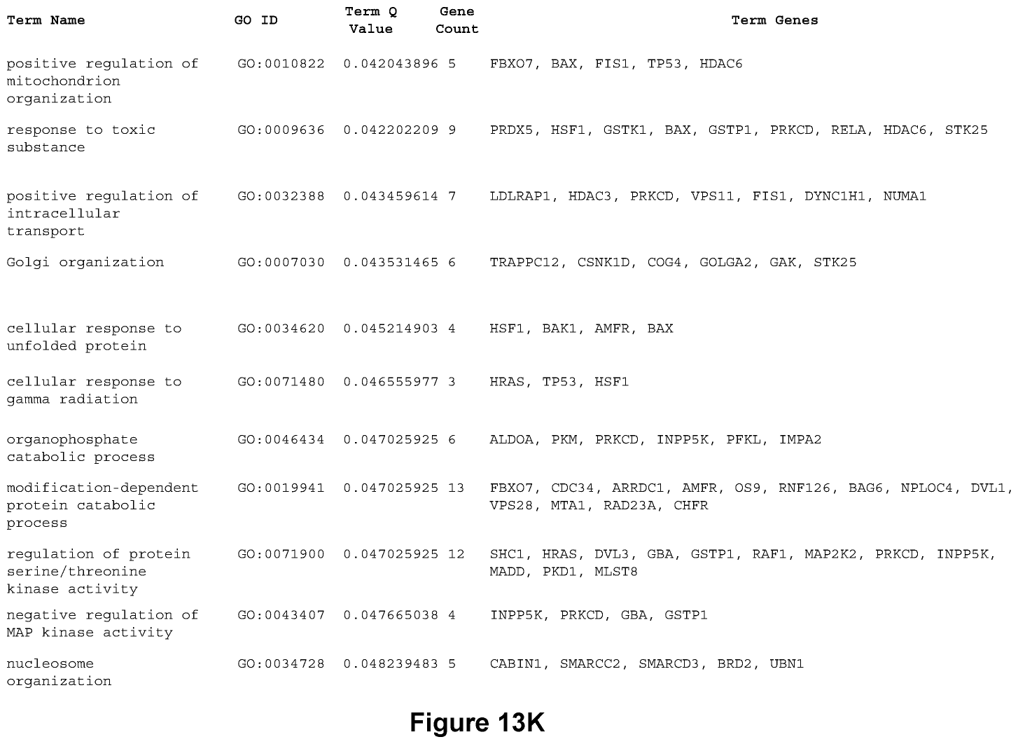

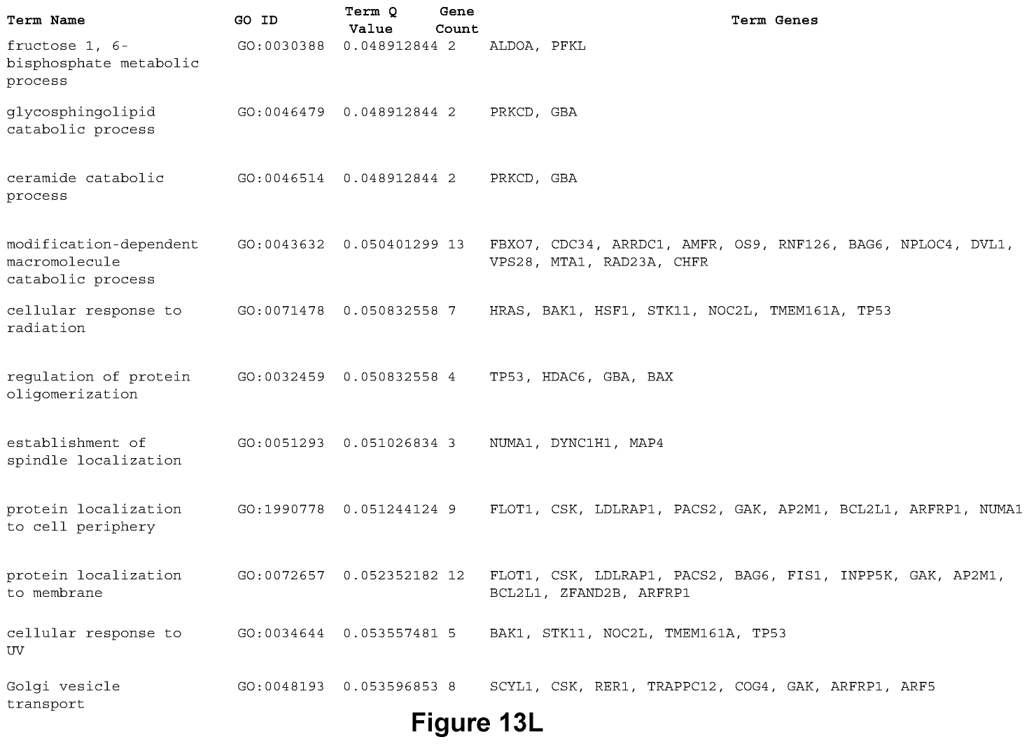

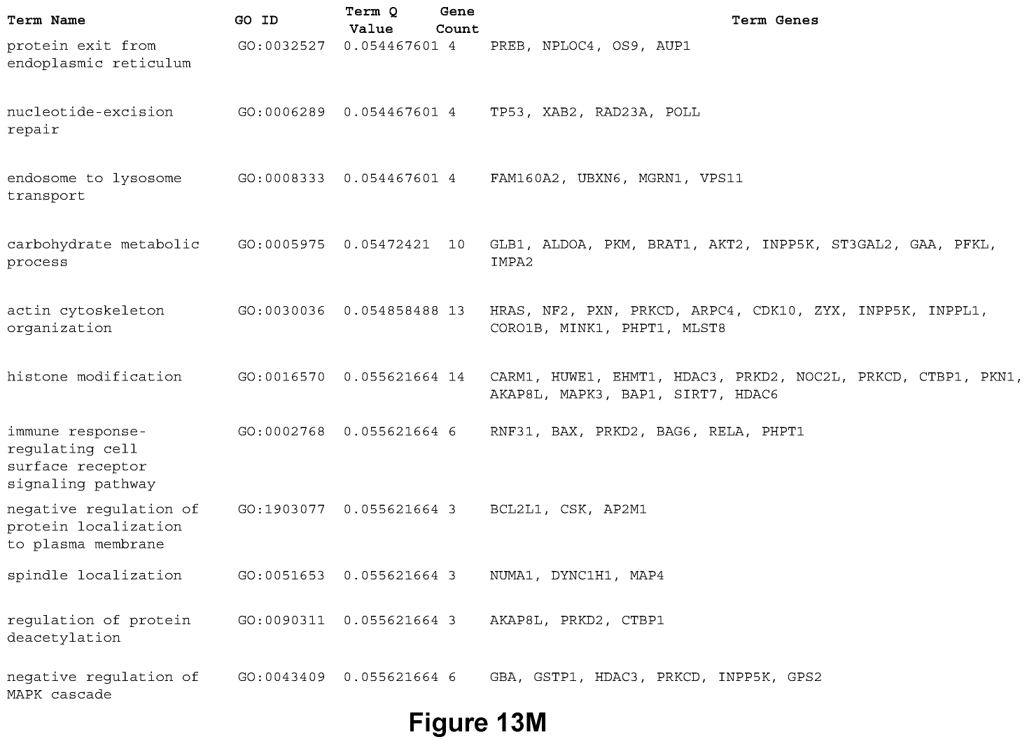

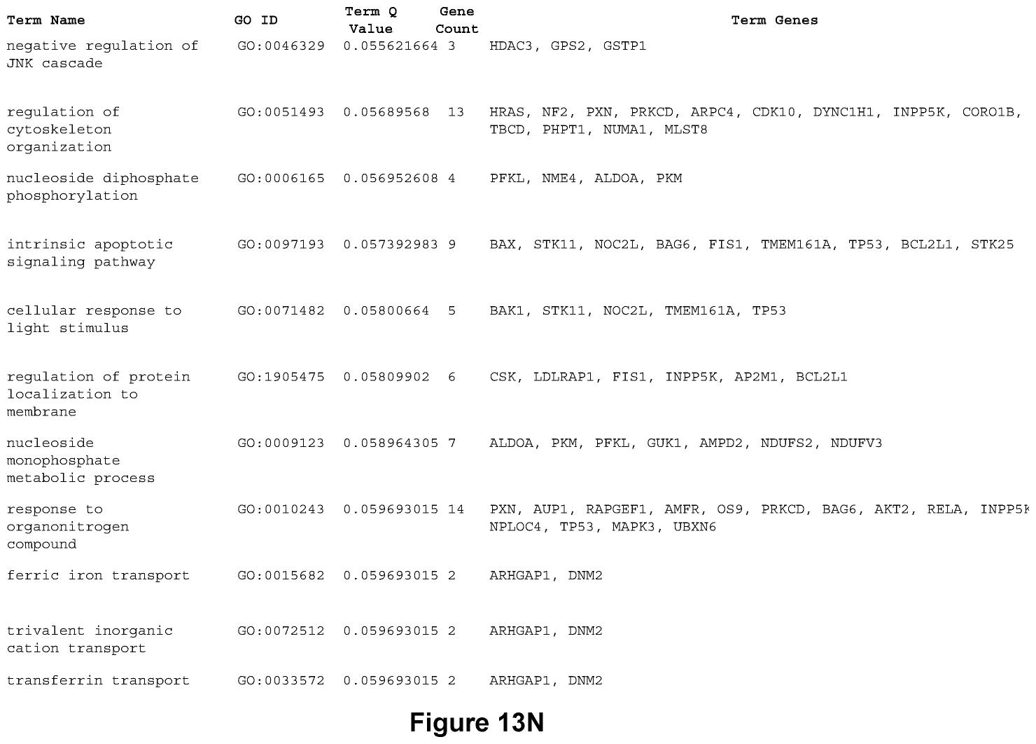

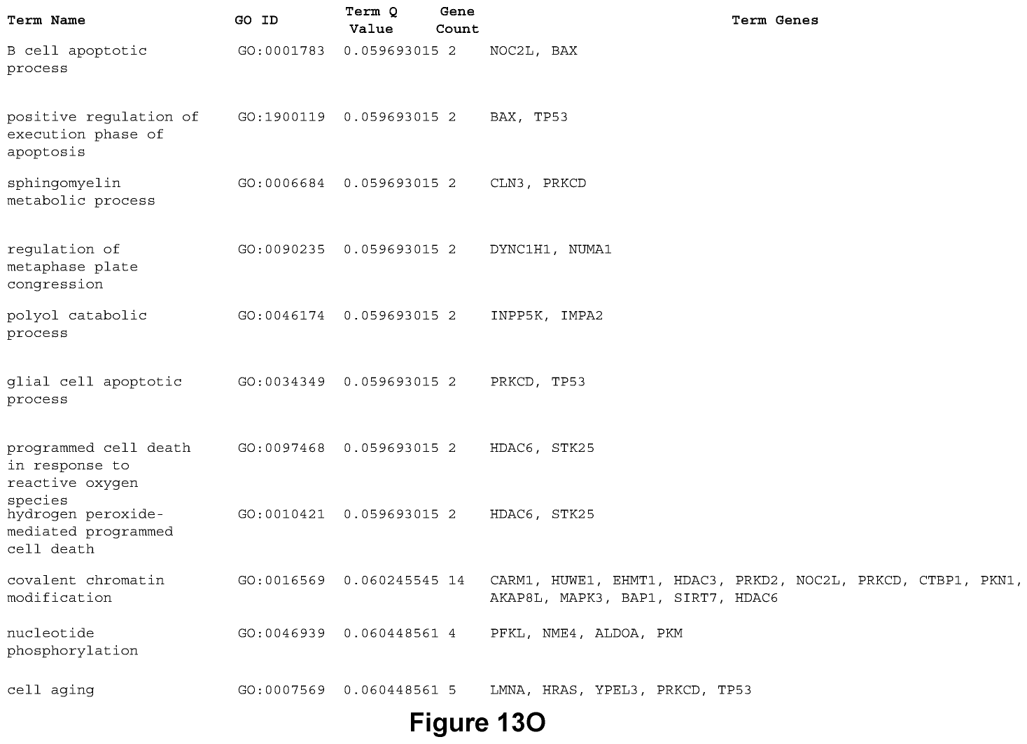

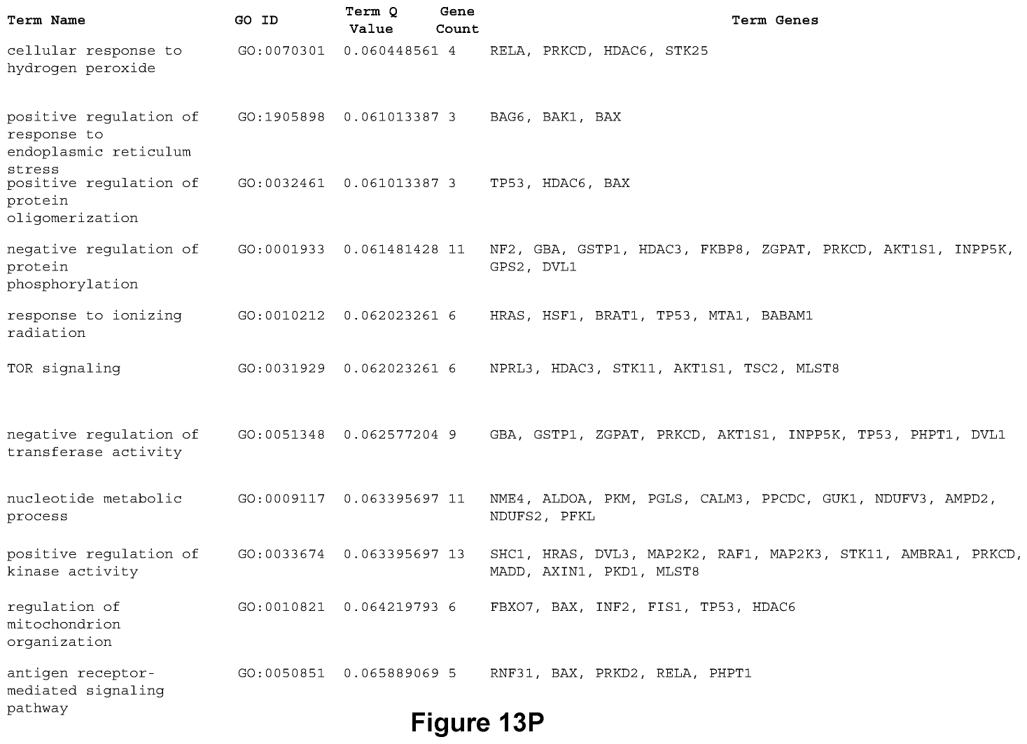

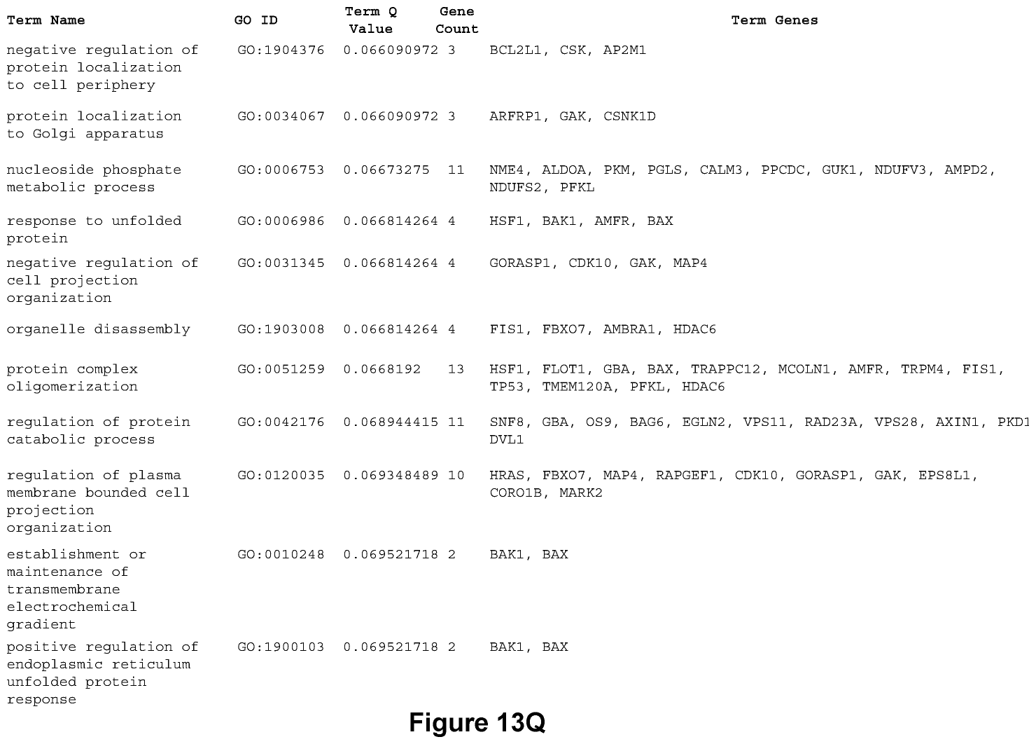

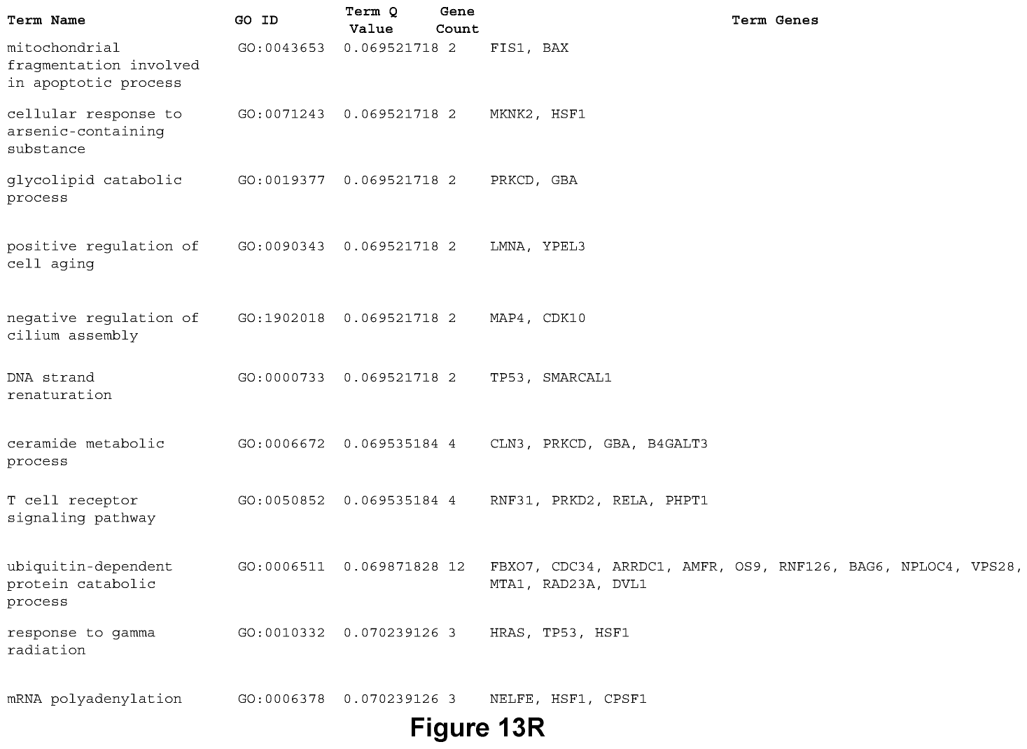

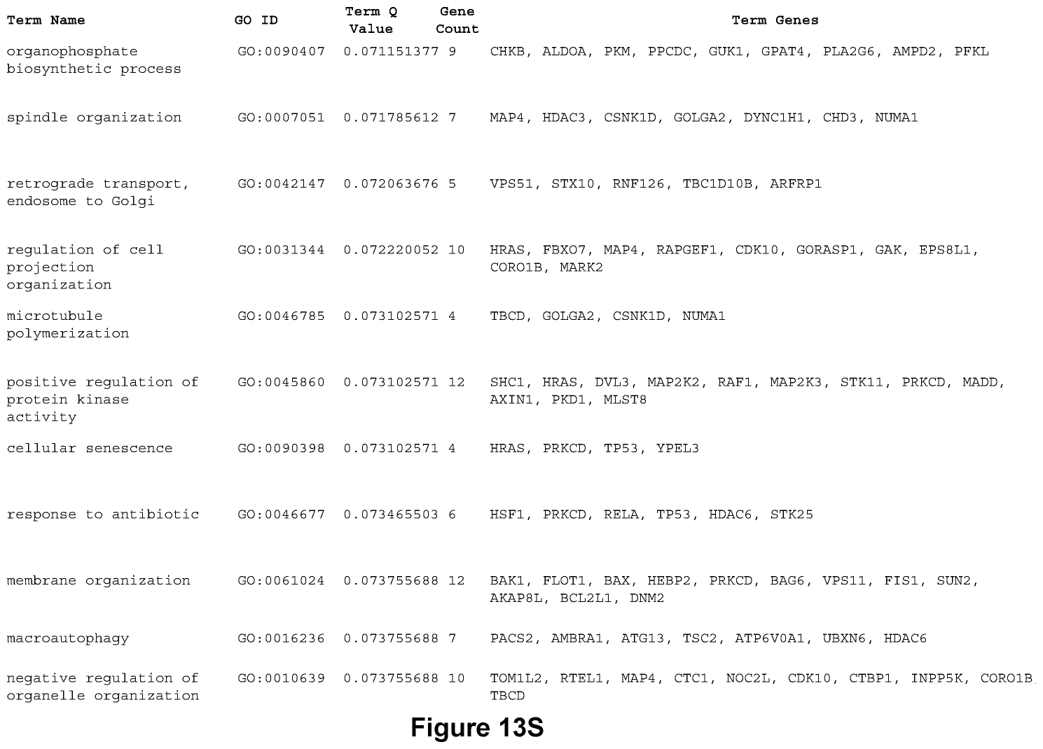

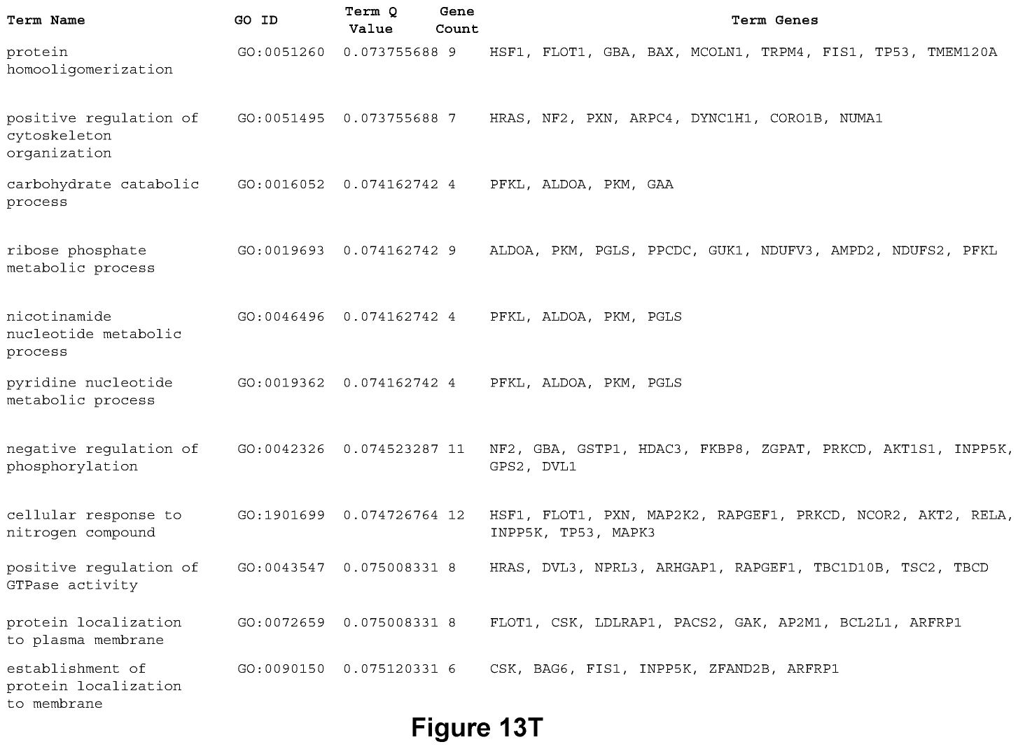

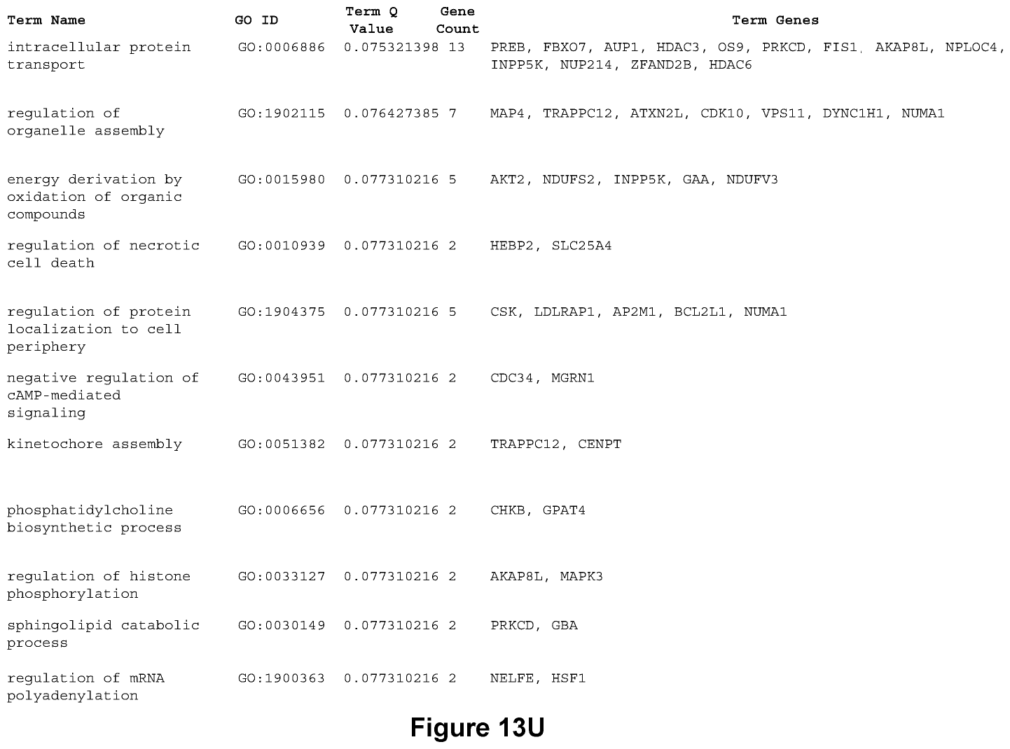

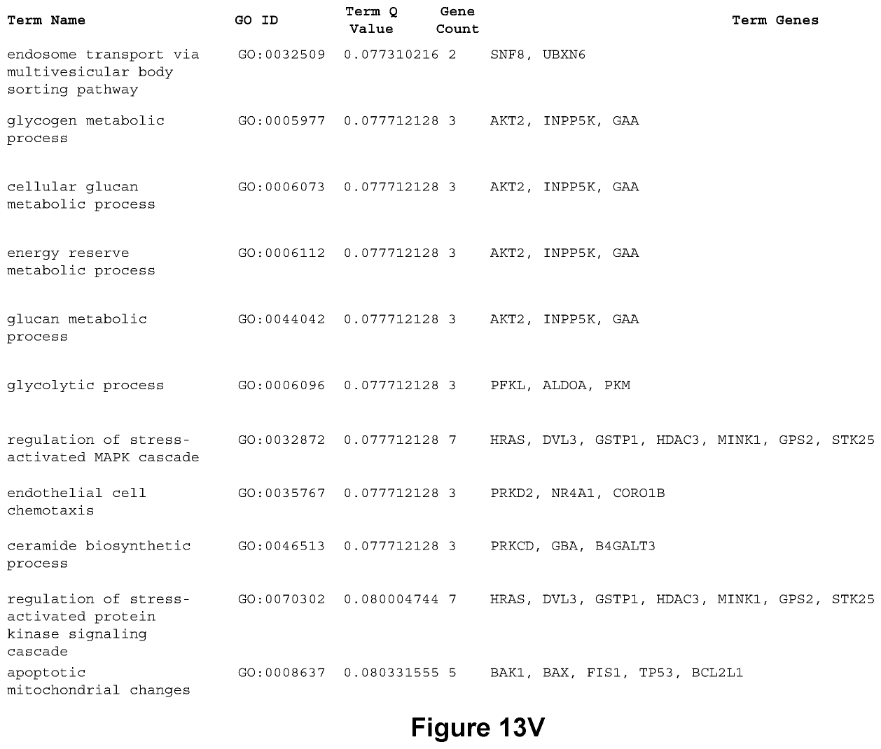

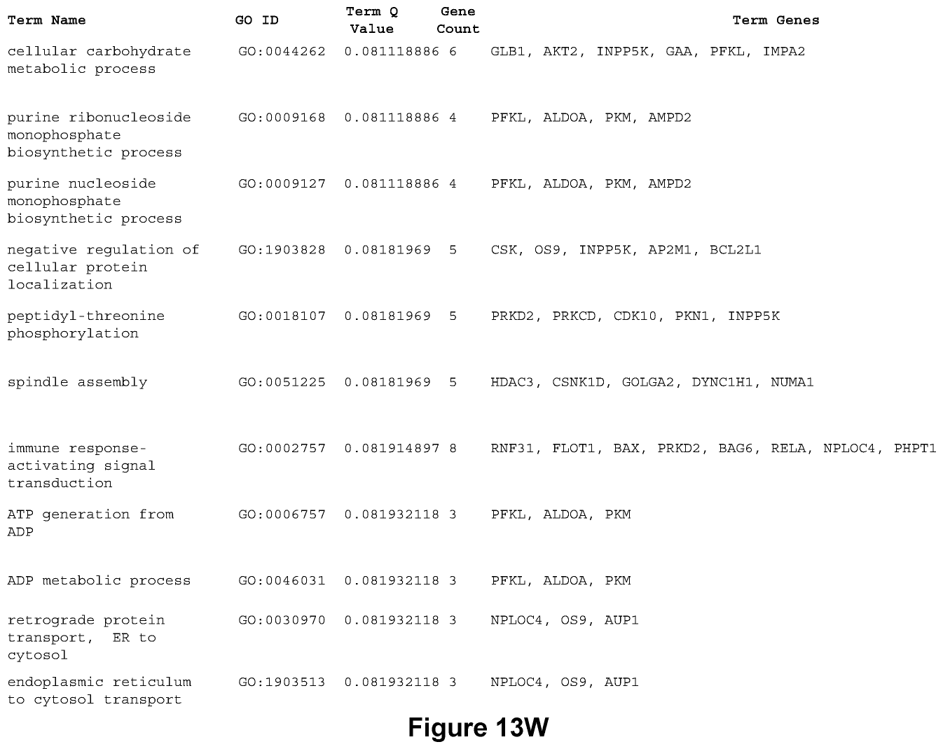

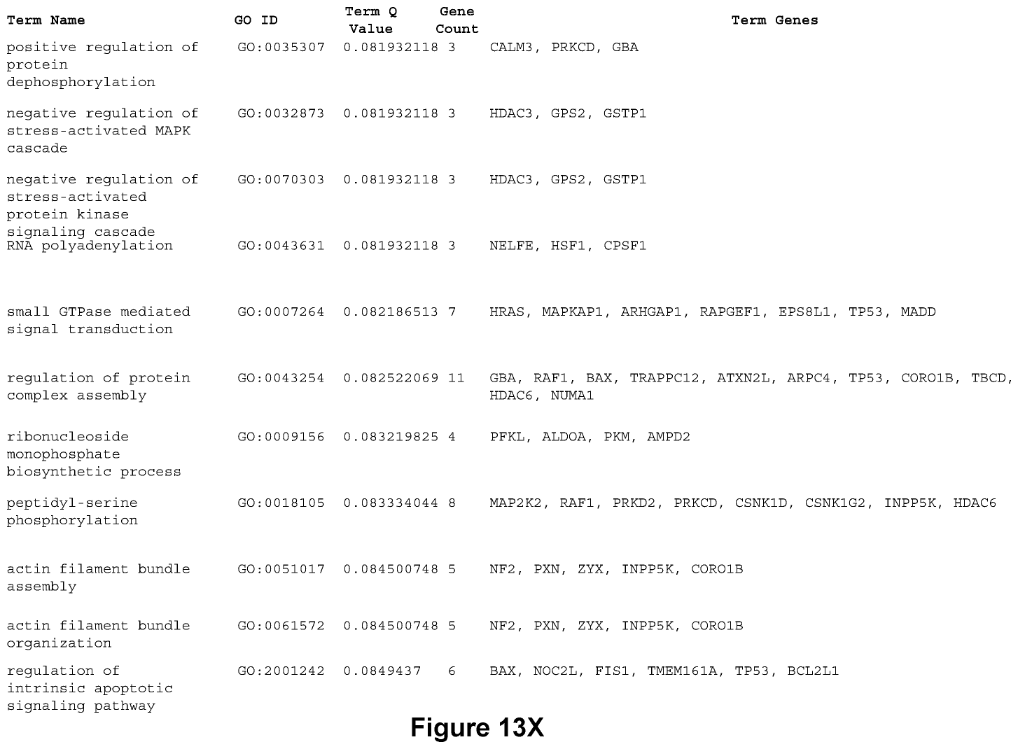

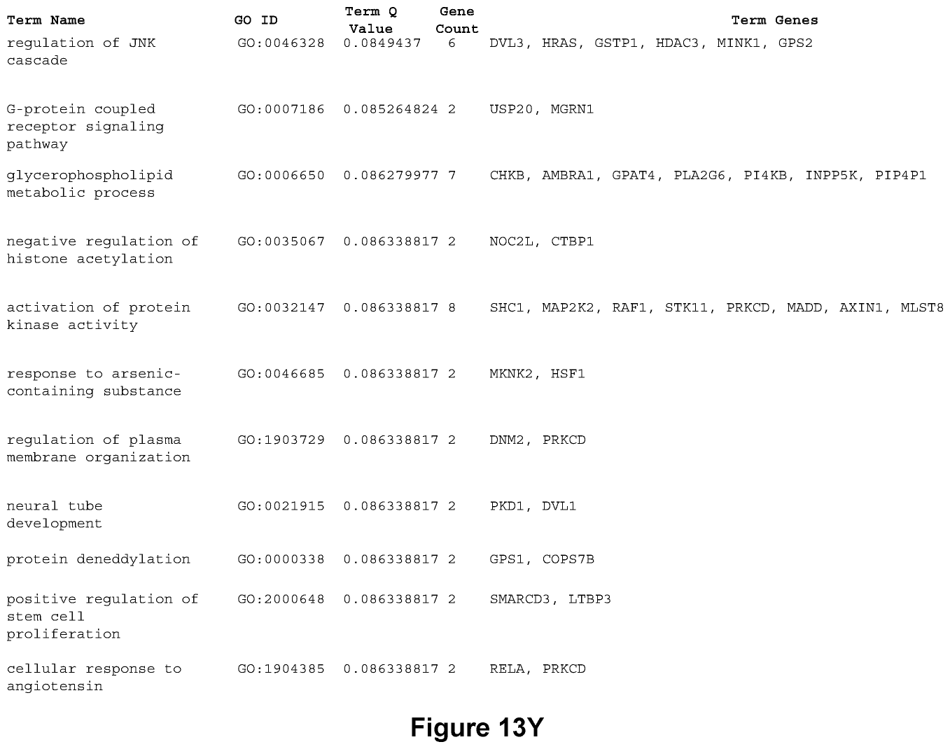

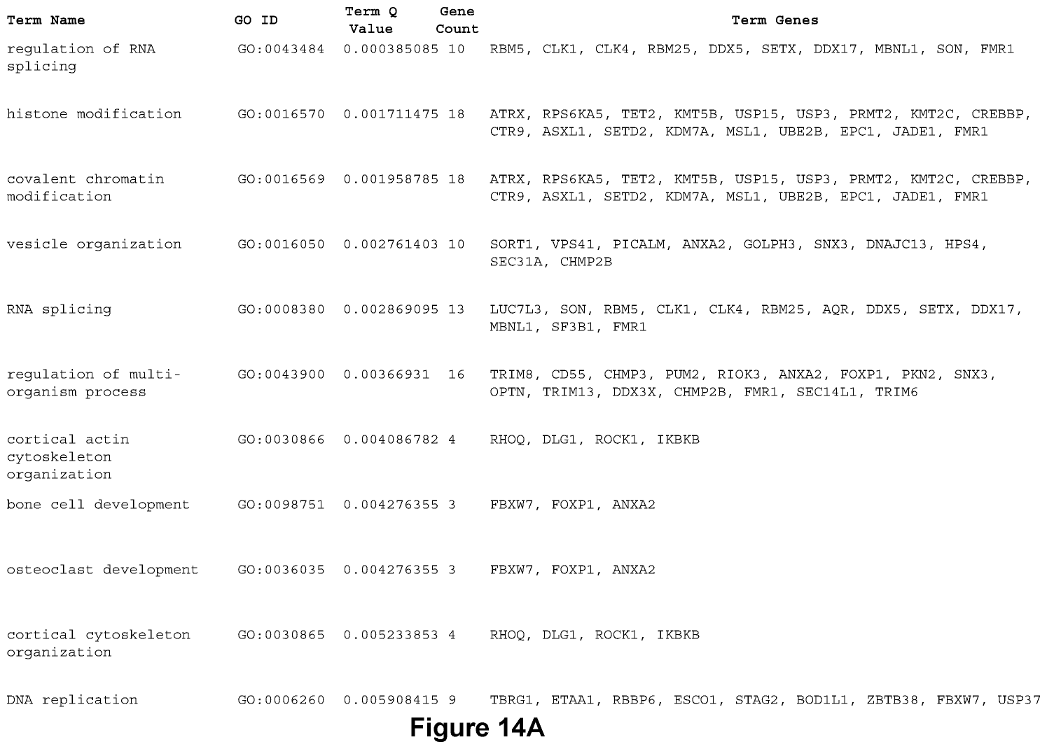

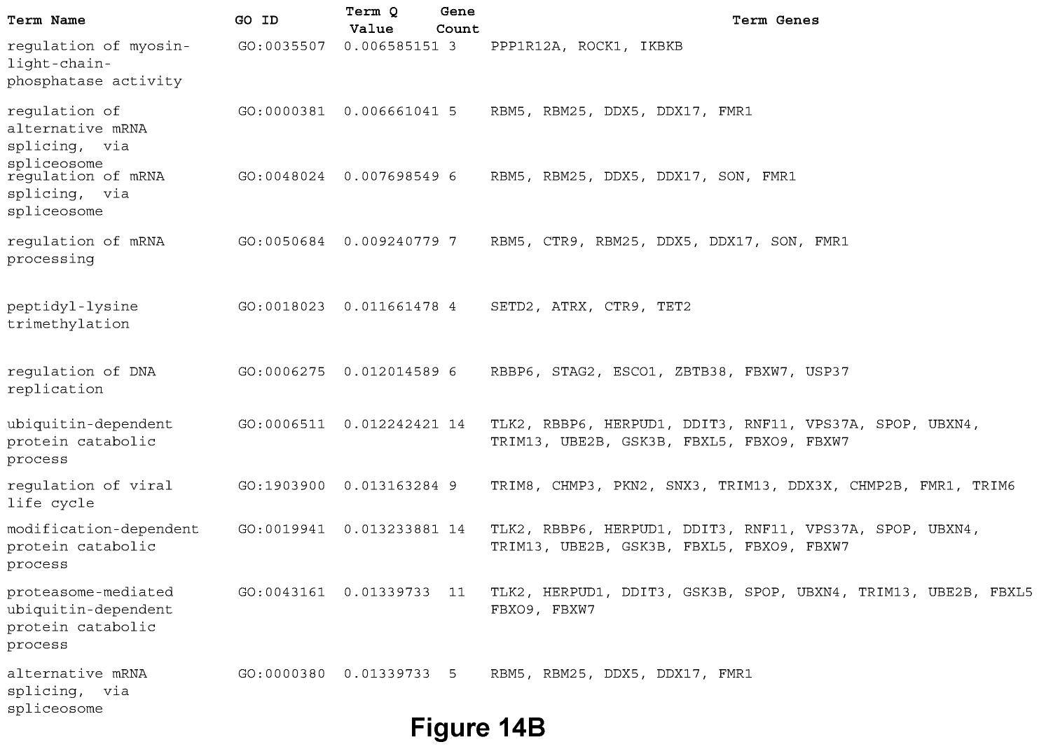

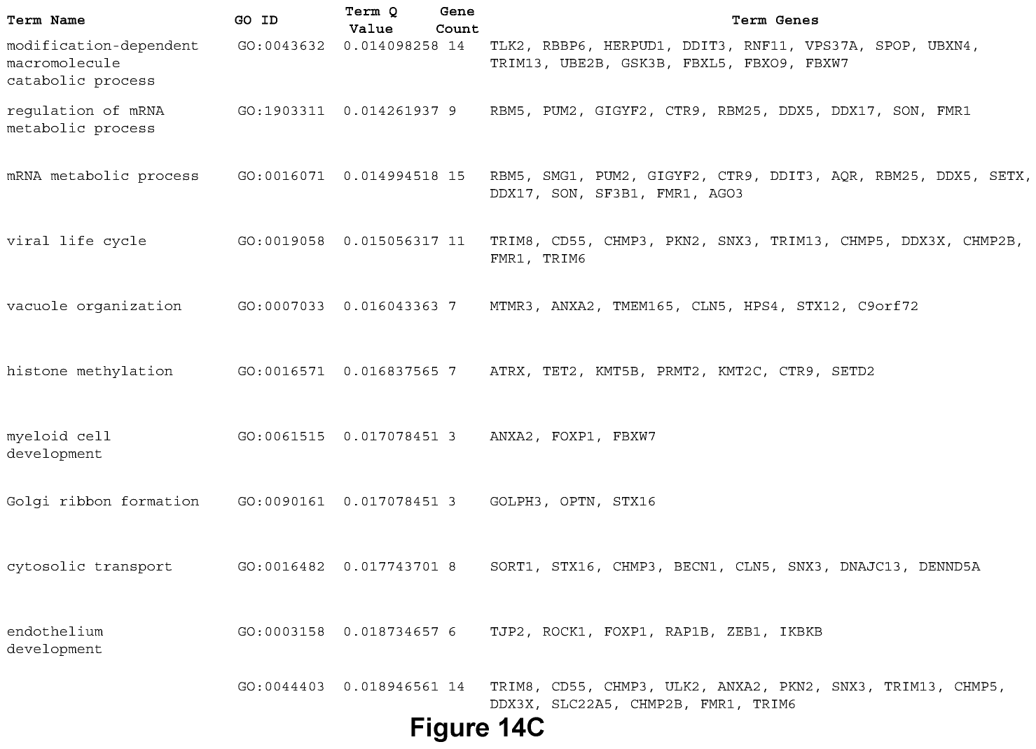

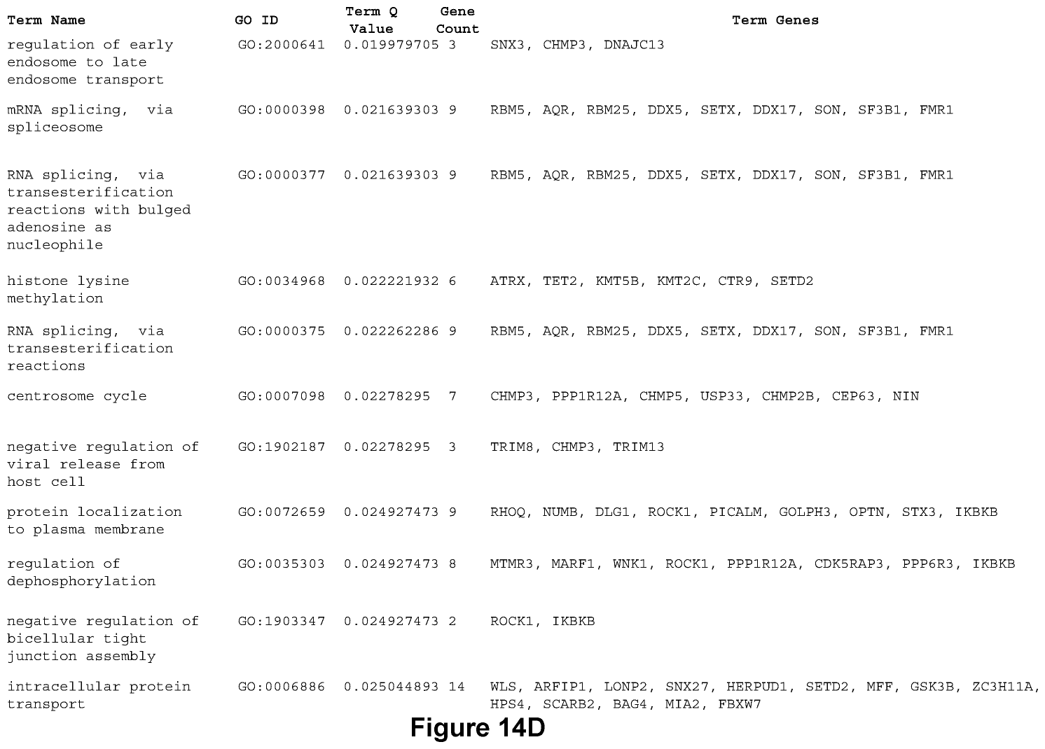

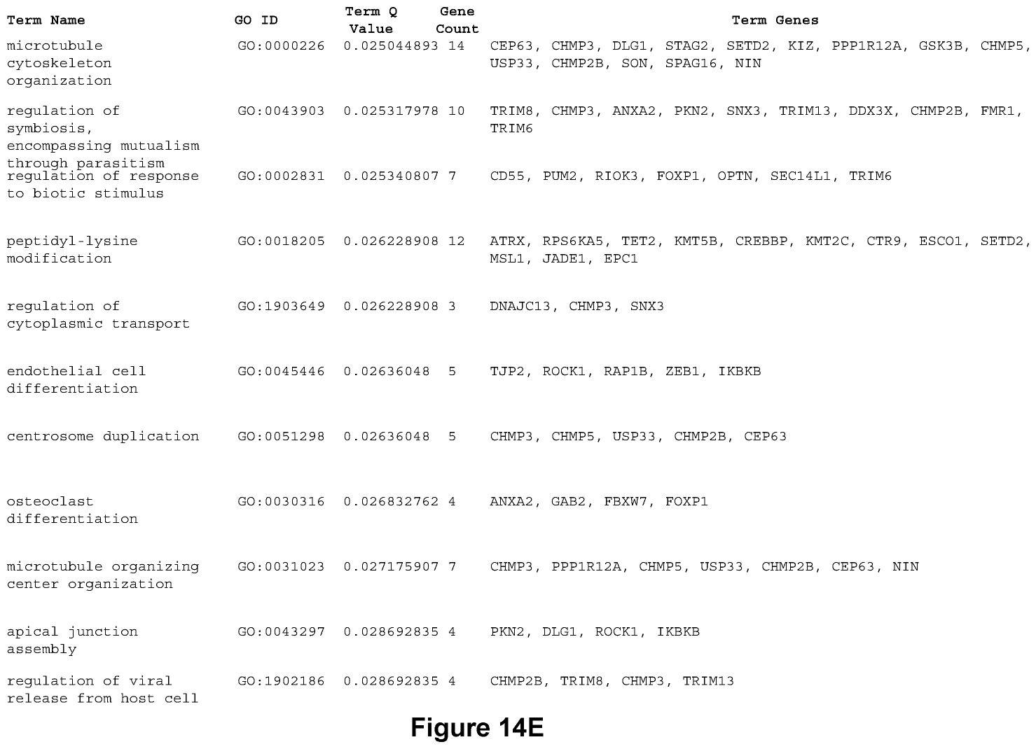

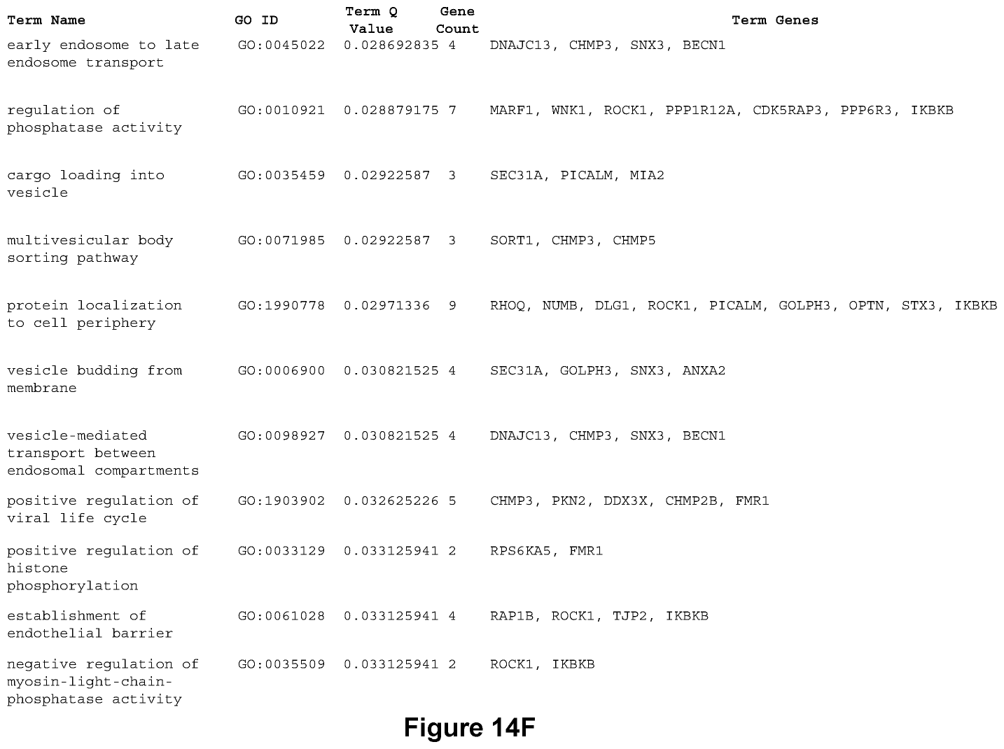

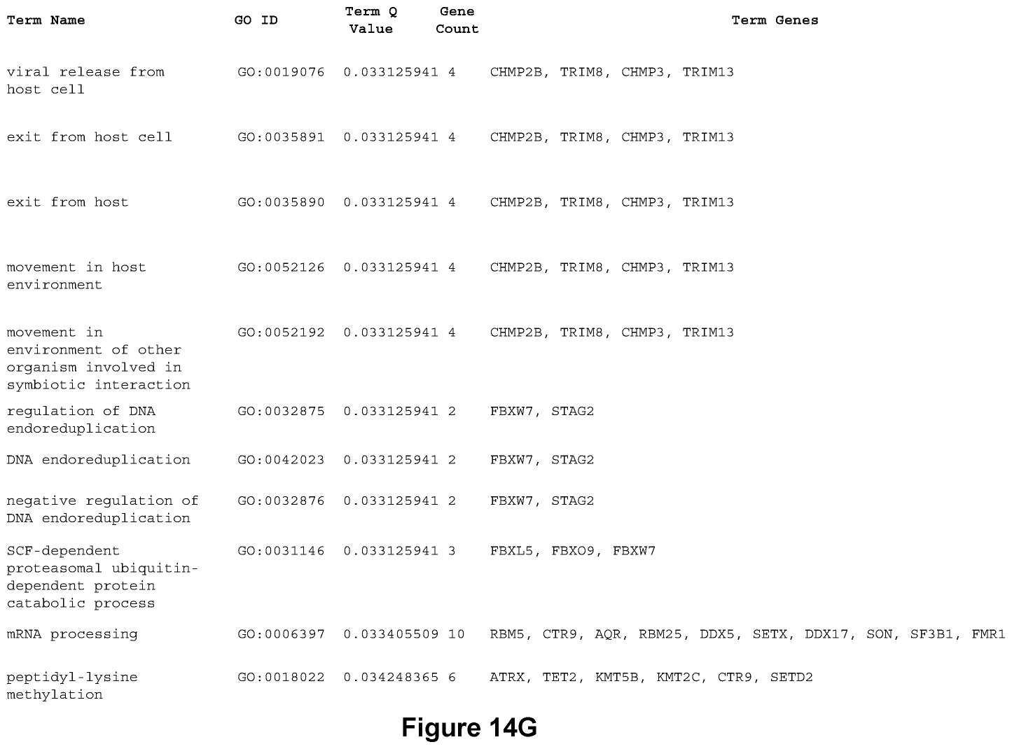

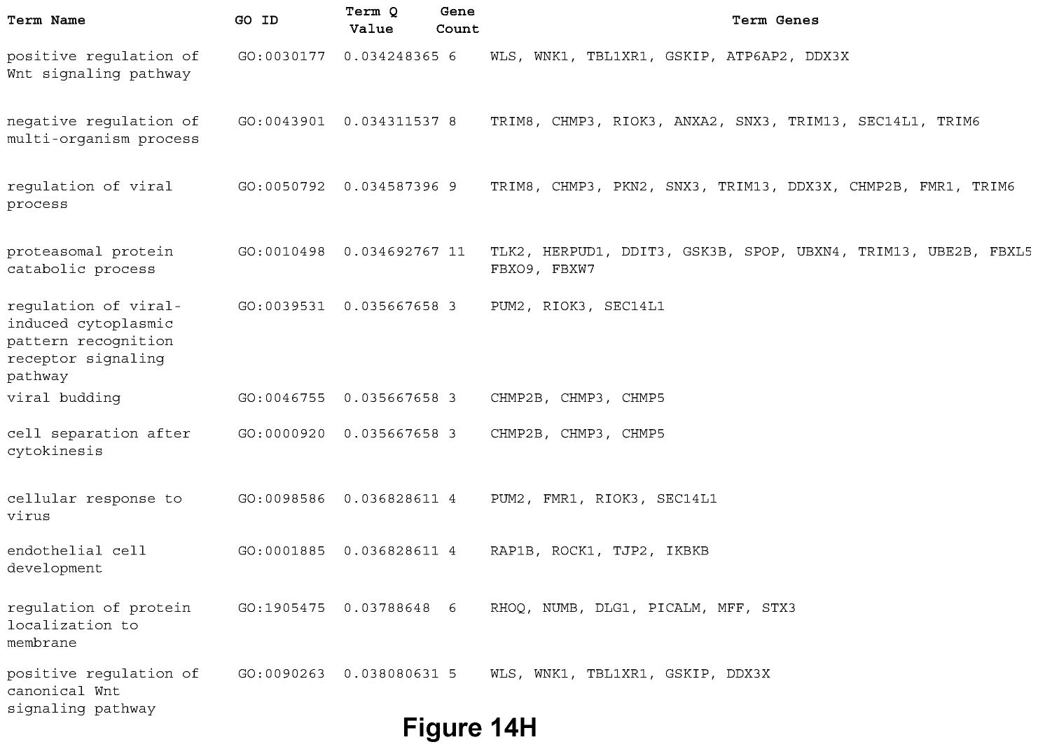

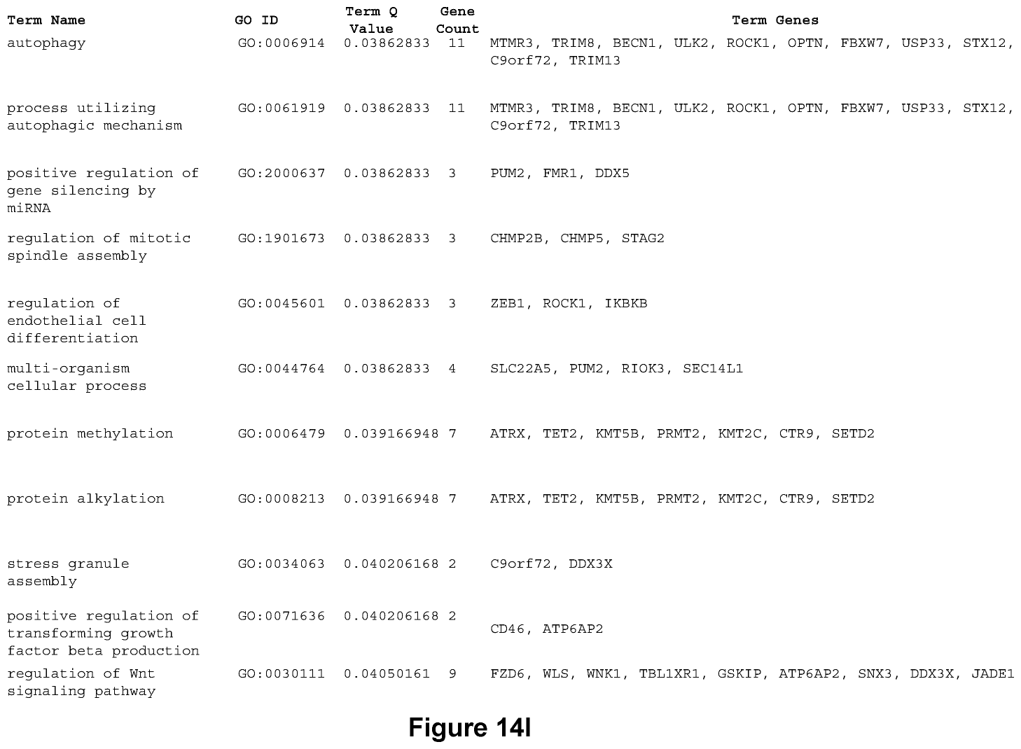

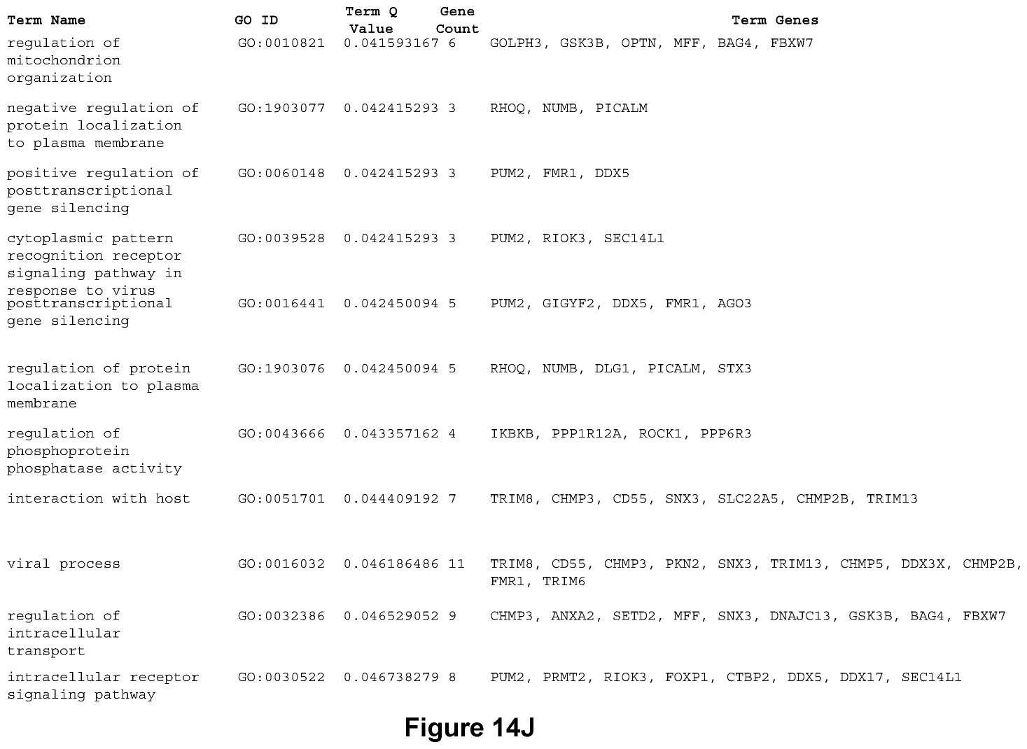

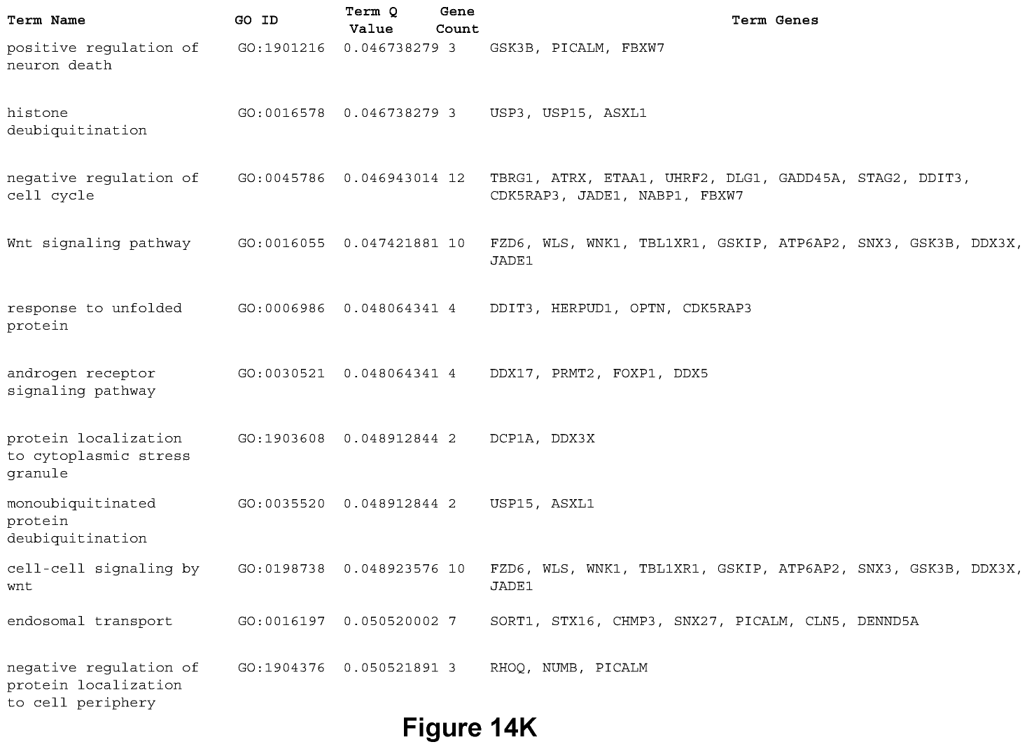

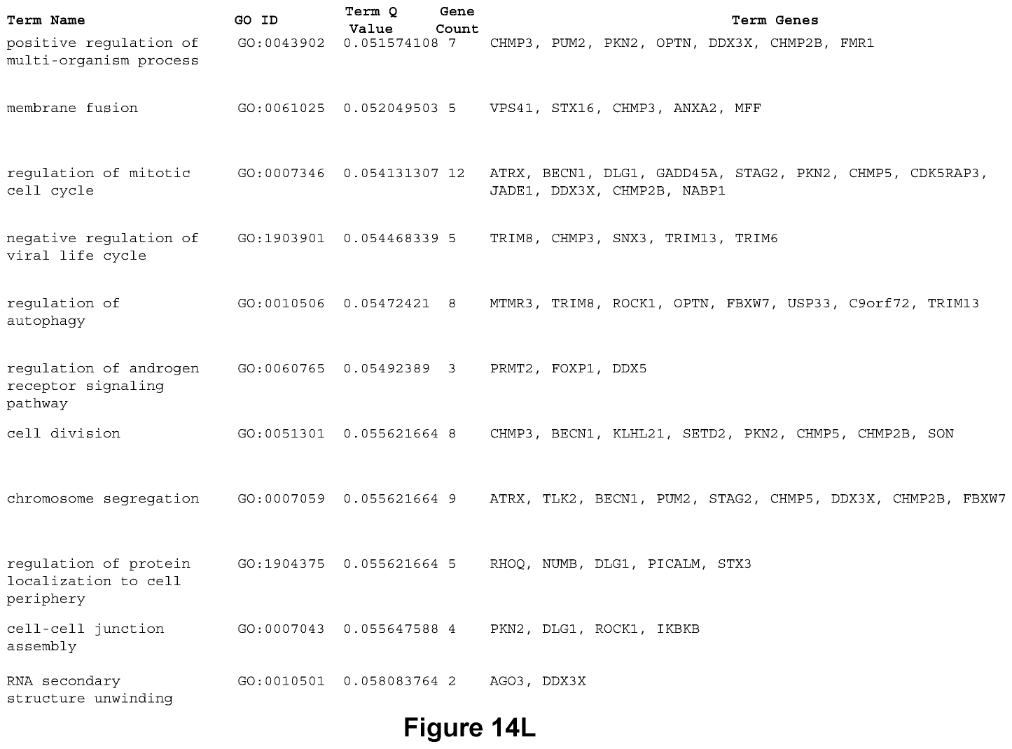

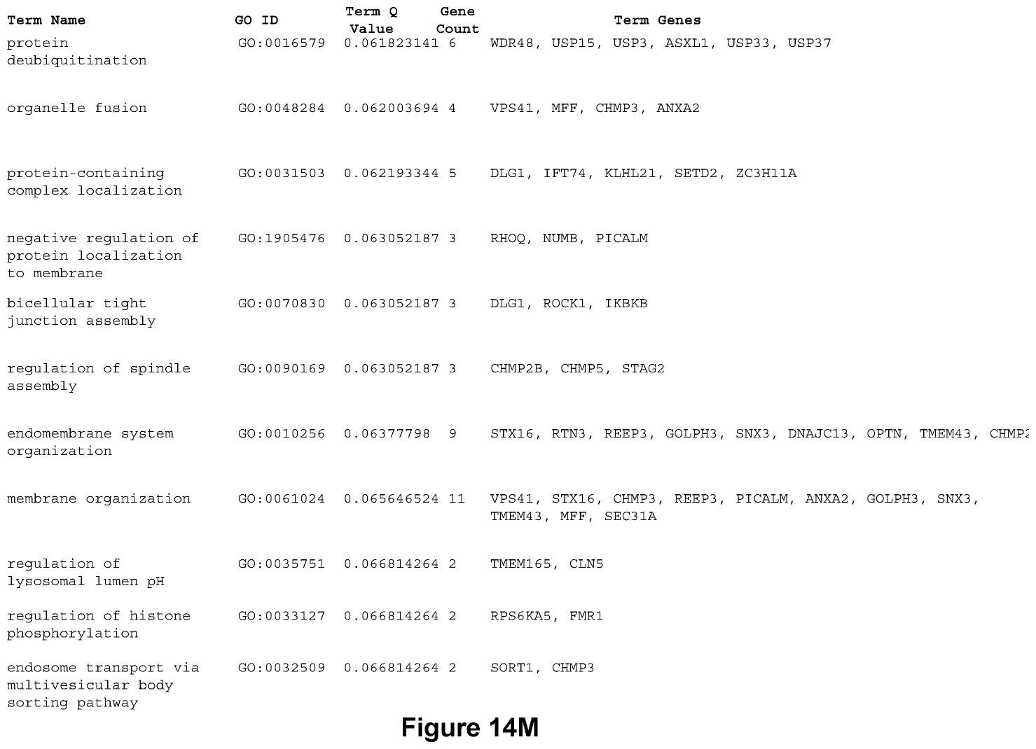

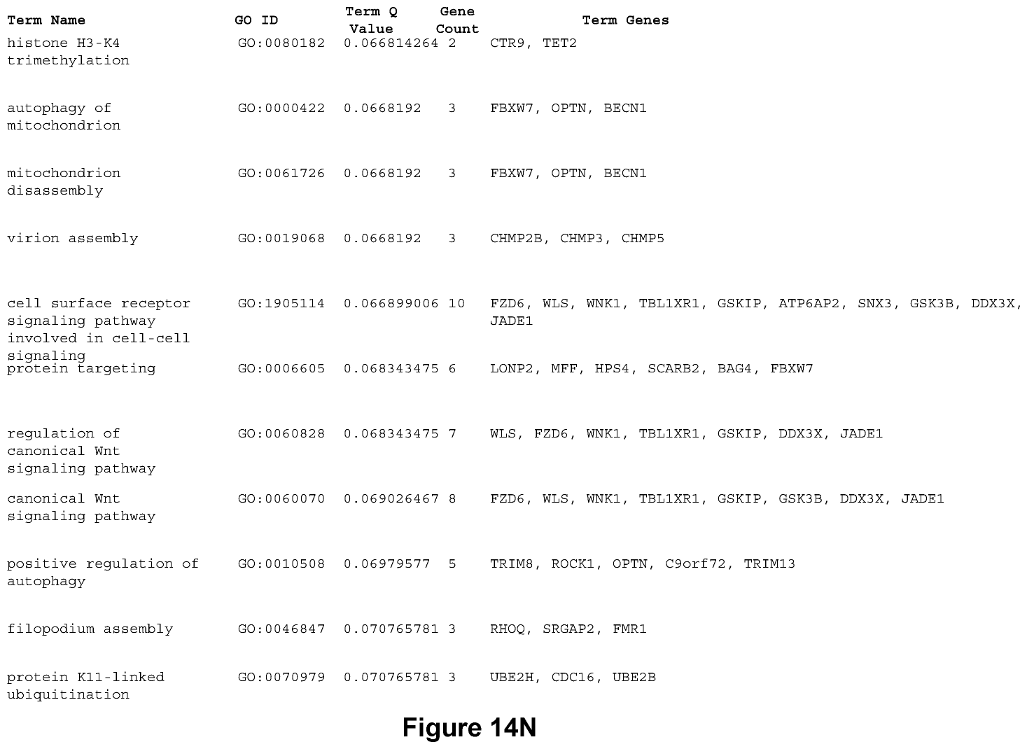

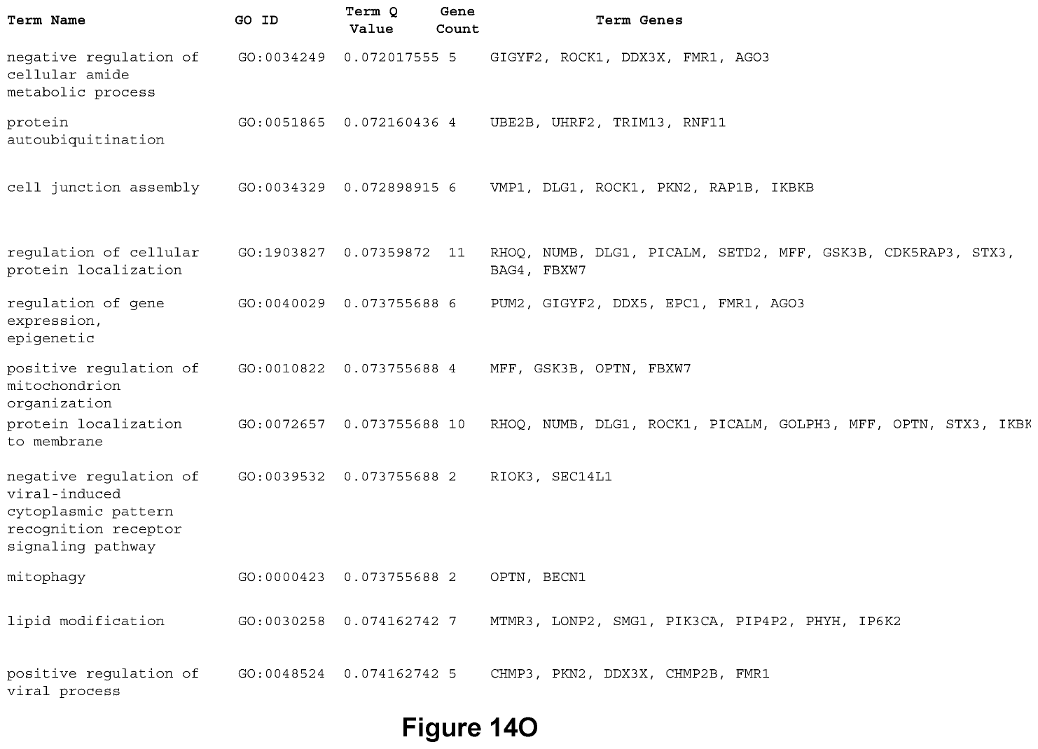

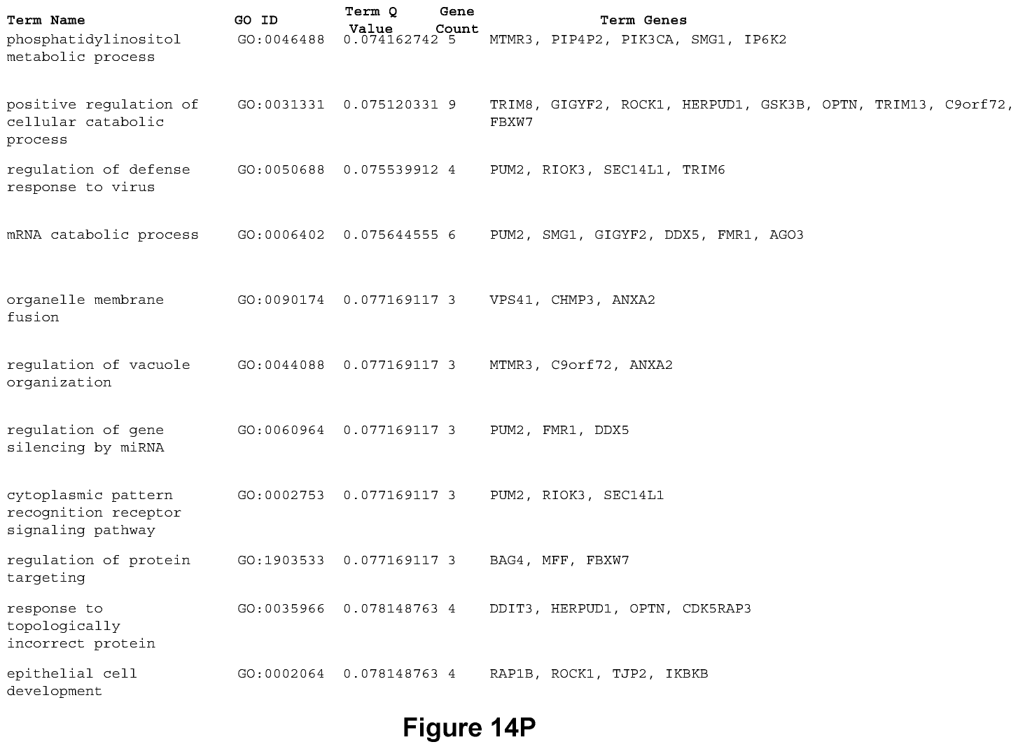

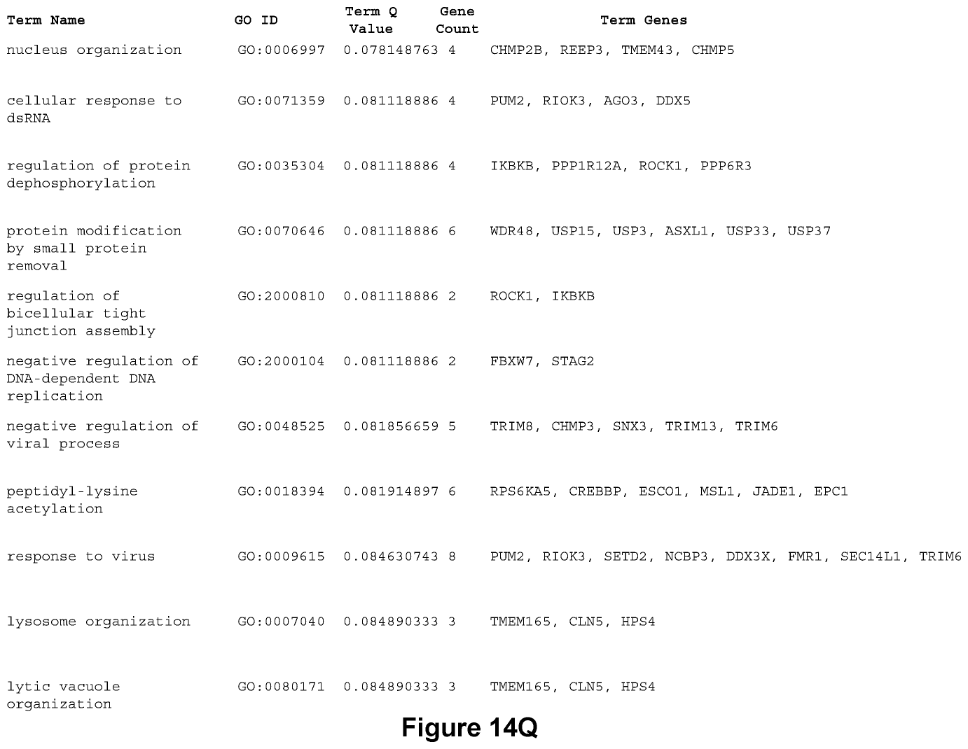

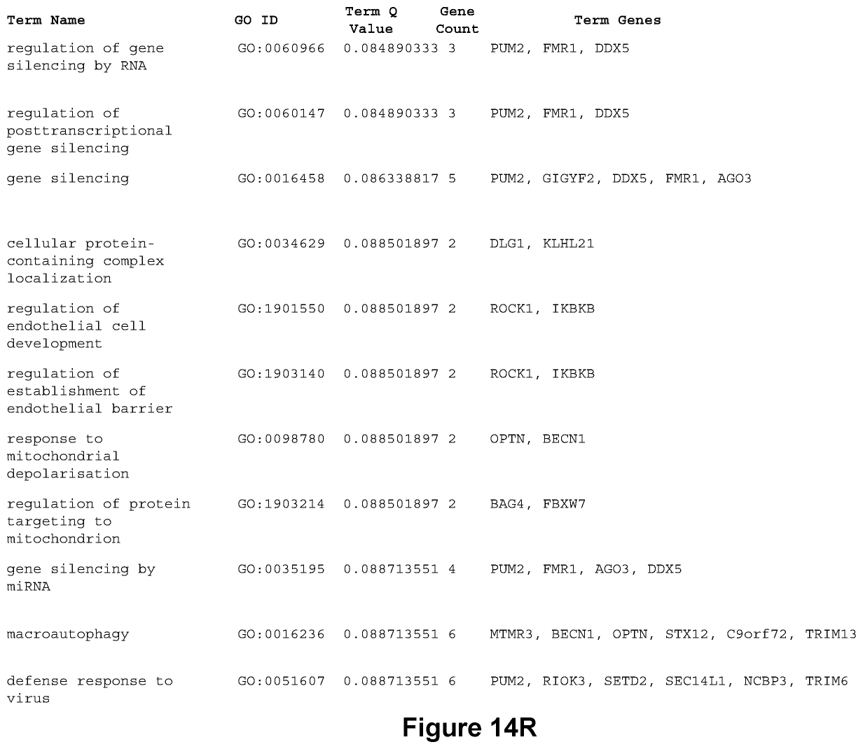

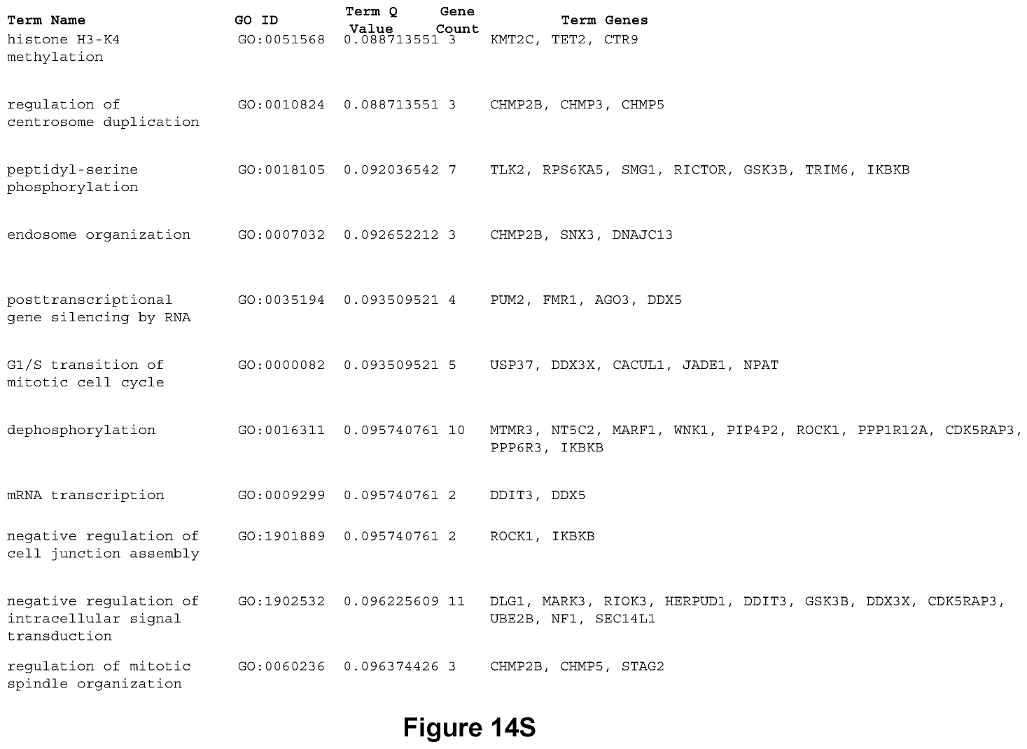

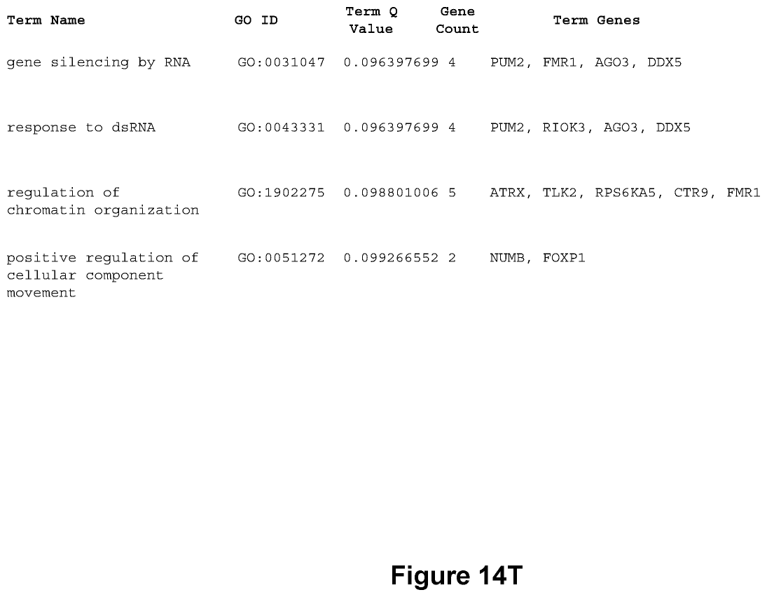

FIG. 11 shows a more comprehensive list of terms for M1. FIG. 12 shows a more comprehensive list of terms for M2. FIG. 13 shows a more comprehensive list of gene ontology terms for M3. FIG. 14 shows a more comprehensive list of gene ontology terms for M4. The full list of gene ontology terms for M1, M2, M3, and M4 is found in Table S5 of Zhang et al., 2023, “Blood RNA alternative splicing events as diagnostic biomarkers for infectious disease,” Cell Reports Methods 3, 100395, which is hereby incorporated by reference.

The genes in cluster M1 are MABCA7, ABCG1, ABI3, ADA, ADA2, ADAM8, ADAR, ADGRE2, ADGRE3, ADGRE5, ANPEP, APAF1, APOL1, APOL2, APOL3, APOL4, ARAP1, ARHGAP25, ARHGAP30, ARHGAP4, ARHGAP9, ARHGEF3, ARHGEF40, ARRB1, ARRB2, ATG16L2, BEST1, BIN2, BMP2K, BTN2A1, BTN3A2, BTN3A3, Clorfl62, C1QC, ClR, C2, CA13, CALHM6, CARD16, CARD19, CARD8, CASP1, CASP10, CASP4, CASP8, CCDC88B, CD163, CD19, CD247, CD300A, CD300LF, CD302, CD33, CD37, CD38, CD3D, CD3G, CD47, CD68, CD74, CEACAM3, CEACAM4, CELF2, CFB, CFD, CFLAR, CFP, CHI3L1, CLEC2D, CLEC4E, CLEC7A, CMKLR1, CNN2, CNP, CORO1A, CPPED1, CRIP1, CSF2RA, CSF2RB, CTSB, CTSS, CXCL3, CYBC1, CYFIP2, CYLD, CYTH4, DAPK2, DDB2, DDX58, DDX60L, DGKA, DNAJC4, DOCK5, DOCK8, DOK1, DPEP2, DYSF, EIF2AK2, EOMES, EPB41L3, EPHB2, EPSTI1, EVL, FAS, FCER1G, FCGR1B, FCGR2A, FCGR2B, FCGR3A, FCGR3B, FCGRT, FCHO2, FES, FGD2, FGD3, FGFR1, FHL3, FKBP11, FLVCR2, FMNL1, FNBP1, FOSL2, FXYD5, FXYD6, GALM, GAPT, GBP3, GCA, GCH1, GCSAM, GIMAP4, GK, GLRX, GLT1D1, GLUL, GMFG, GMIP, GNLY, GPR18, GPSM3, GRAMDIB, GRK2, GRK3, GSDMD, GSN, GUCY1B1, GUSB, HCK, HERC5, HFE, HLA-A, HLA-C, HLA-DMA, HLA-DMB, HLA-F, HPS1, HSH2D, ICAM3, IFI16, IFI27, IFI44, IFI6, IFITM2, IFNAR1, IGFLR1, IKZF1, IL16, IL1RN, IL32, IL4R, INPP4A, IRF7, ISG20, ITGAL, ITGAX, ITGB7, ITK, ITPK1, IVD, JAML, KCNAB2, KCNJ15, KLRK1, KMO, LAIR1, LAT2, LCK, LEF1, LGALS3, LGALS3BP, LGALS9, LILRA1, LILRA2, LILRA5, LILRA6, LILRB2, LILRB3, LILRB4, LILRB5, LIMD2, LRMP, LRRK1, LRRK2, LSP1, LST1, LY6E, LY75, LYL1, LYZ, MANBA, MAP3K1, MARCO, MATK, MBP, MDK, MDM2, MEI1, MFNG, MGAM, MICAL1, MLKL, MME, MOV10, MPP1, MR1, MS4A6A, MSL3, MTSS1, MVP, MX1, MX2, MXD1, MYO1F, MYO1G, MYOSA, MZB1, NAALADL1, NAGK, NAV1, NCF1, NCF4, NEXN, NFATC 1, NFKBIZ, NLRP1, NMI, NPL, OAS2, OAS3, P2RX5, PADI2, PARP14, PARP9, PCNX1, PCSK7, PDLIM1, PDXK, PDZKIIPI, PGGHG, PIK3R5, PIP5KIB, PLCB2, PLEKHG2, PLSCR1, PLXNC1, PML, PPP1R18, PPT1, PROK2, PSMB10, PSMB8, PSME1, PSME2, PSTPIP1, PTGS1, PTK2B, PTPN6, PTPRC, PTPRE, PTPRO, PYCARD, RAB31, RAB37, RASAL3, RCAN3, RCN3, RCSD1, RELB, RELT, RENBP, RGS19, RGS2, RGS3, RHBDF2, RIN2, RIPK3, RIPOR2, RNASE1, RNF19B, RNF213, RUBCNL, SAMHD1, SEL1L3, SELP, SERPINA1, SERPINB1, SERPINF1, SH3BP2, SH3TC1, SHISA5, SIGLEC1, SIGLEC10, SIPA1, SIRPA, SIRPB1, SLC11A1, SLC12A6, SLC15A3, SLC15A4, SLC24A4, SLC25A37, SLC36A1, SLC43A2, SLC6A6, SLC7A7, SNCA, SOD2, SP110, SP140, ST8SIA4, STAP1, STAT1, STEAP4, STXBP2, TAL1, TAP1, TAPBP, TBXAS1, TCF7, TCIRG1, TCN2, TEP1, TESC, TFEB, THEMIS2, TIMP2, TLR1, TLR4, TMC6, TMC8, TMEM71, TNFAIP2, TNFAIP8, TNFRSF14, TNFRSF17, TNIP1, TRAF3, TRAF3IP3, TRIM14, TRIM69, TSPAN32, TSPAN4, TYMP, TYROBP, UPP1, VAMP8, VCAN, WDFY4, WIPF1, XAF1, XRN1, ZBP1, ZC3HAV1, ZFYVE26, and ZNF438.

The genes in the M2 cluster are ACP1, AGO2, AHCTF1, ALG8, ANP32A, ANP32E, APTX, ARNTL2, ARPC3, ATIC, ATP 11C, ATP2A2, AURKA, B3GALNT1, BARD1, BAZ1A, BCLAF1, BICD2, BID, BIRC5, BRAP, BUB1B, C17orf80, Clorfl74, CAAP1, CAMTA1, CANX, CBFB, CBX3, CCDC90B, CCNB2, CDC45, CDC6, CDK1, CDK2AP1, CDK8, CDV3, CENPA, CENPN, CENPU, CEP128, CEP152, CEP192, CEP295, CHAF1A, CHEK1, CHEK2, CHTF18, CHTOP, CIT, CKLF, CMAS, CMC2, CNBP, CNOT1, COMMD3, COMMD4, COP1, COPS8, COX20, CRYZ, CSTF1, CTNNAL1, CTPS1, CUTC, CYREN, DAZAP1, DCAF13, DCK, DDX11, DDX23, DDX39A, DDX39B, DDX52, DECR1, DEPDC1, DEPDCIB, DERA, DFFA, DGUOK, DHX9, DNAJA3, DTL, DUT, DYNC1LI1, E2F7, EFTUD2, EIF4H, EIF5, EMC1, ENO1, ERGIC2, ESPL1, ETFA, EWSR1, EXO1, EXOSC10, EXOSC8, EXOSC9, FAM120A, FASTKD1, FECH, FNTA, FOXM1, FUS, GATAD2A, GNL2, GPSM2, GSPT1, GTF2I, GTF3A, HDAC1, HDGF, HIKESHI, HMBS, HMGB1, HMGN2, HMMR, HNRNPA2B1, HNRNPA3, HNRNPC, HNRNPH1, HNRNPK, HNRNPL, HNRNPLL, HNRNPM, HNRNPU, HSP90AA1, IDH3A, IDI1, INSIG1, ITGB3BP, KARS, KDM1A, KIAA1586, KIF22, KIF2A, KNOP1, LIG3, LMBR1, LMNB1, LRRC42, LSM1, LSM8, LYRM1, MAD2L1, MAGOHB, MAPKAPK5, MAT2A, MCM7, MCTS1, MEAF6, MELK, METTL2A, METTL9, MMD, MRPL1, MRPL20, MRPL22, MRPL33, MRPS18B, MRPS18C, MRPS22, MRPS25, MSH2, MTHFD1, MTHFD2, MXD3, MYB, NAA25, NAA50, NAE1, NANS, NAP1L4, NARS, NASP, NCAPD3, NCAPG, NCAPH, NCAPH2, NCL, NCOR1, NGLY1, NIPA2, NOL8, NONO, NOP56, NSMAF, NSMCE2, NUDT6, NUF2, NUP54, NUSAP1, OPA1, ORC1, OXCT1, PCCB, PDCD10, PDCD2, PDHB, PITPNB, PKMYT1, PLS1, POC5, POLB, PPHLN1, PRC1, PRELID1, PRKDC, PRPF40A, PRUNE1, PSMA3, PSMA4, PSMC1, PSME4, PSMF1, PUS7L, PWP2, PYCR1, QTRT2, RACGAP1, RAD51, RAD51AP1, RANBP1, RBBP4, RBX1, RFC2, RFC5, RNF34, RPE, RPP38, RPS27L, RRP7A, RSRC1, SCO1, SEC11C, SEC61G, SERBP1, SFXN2, SHMT2, SKA1, SKA3, SLC19A1, SLC25A40, SLC27A2, SLC29A1, SLC38A5, SLC39A10, SMARCA4, SMARCB1, SMC1A, SMPD4, SNRPA1, SNRPC, SPATS2, SPC24, SRP19, SRPK1, SRRM1, SRSF2, SRSF3, SRSF7, SSR1, STIL, STMN1, STOML2, STRADB, TACC3, TARBP2, TFB1M, THADA, THAP12, TICRR, TIMELESS, TK1, TMEM126B, TMEM183A, TPGS2, TPX2, TRAIP, TRNT1, TROAP, TRRAP, UBA2, UBAP2, UBE2A, UBE2C, UBE2D2, UBE2D3, UBE2E1, UBE2K, UBE2V1, UBL5, UBQLN1, UFD1, USP14, USP39, USP7, VRK2, VTI1B, WDR62, XPO6, YWHAE, YY1, ZBTB8OS, ZNF138, ZNF207, ZNF317, ZNF616, and ZWINT.

The genes in the M3 cluster are ABHD14B, ABHD4, ABTB1, ACAA1, ACADVL, ACIN1, ACSF2, ACY1, ADAM15, ADGRL1, AGTRAP, AKAP8L, AKT1S1, AKT2, ALDH16A1, ALDOA, AMBRA1, AMFR, AMPD2, ANGEL1, ANO10, ANXA11, AP1M1, AP2B1, AP2M1, APLP2, ARF5, ARFGAP2, ARFRP1, ARHGAP1, ARHGAP17, ARID3A, ARL8A, ARMCX6, ARPC4, ARRDC1, ARSA, ASCC2, ATG13, ATP13A1, ATP6AP1, ATP6VOA1, ATP6V0B, ATRIP, ATXN2L, AUP1, AXIN1, B4GALT3, BABAM1, BAG1, BAG6, BAK1, BAP1, BAX, BAZ2A, BCAS3, BCL2L1, BCL7B, BLVRB, BRAT1, BRD2, BRD3, BUD23, C11orf49, CABIN1, CALCOCO1, CALM3, CANT1, CAPZB, CARM1, CCDC12, CCDC130, CCM2, CDC34, CDK10, CDK11A, CDS2, CELSR2, CENPT, CERS2, CHD3, CHD4, CHFR, CHKB, CIC, CIZ1, CLASRP, CLK3, CLN3, CLPP, CMTR1, COG4, COMMD7, COPS7B, COQ6, COQ7, CORO1B, CPNE1, CPSF1, CRELD1, CSK, CSNK1D, CSNK1G2, CSRP1, CTBP1, CTC1, CUL9, CYB5R1, DBNL, DDX56, DGKZ, DHX34, DNAJB2, DNM2, DRAP1, DVL1, DVL3, DYNC1H1, EEF2KMT, EGLN2, EHBP1L1, EHMT1, ELOB, ELOF1, EML3, ENTPD6, EPS8L1, EXTL3, FAM160A2, FAM214B, FASTK, FBRS, FBXO7, FDXR, FIS1, FKBP8, FLII, FLOT1, GAA, GABARAP, GAK, GALNS, GATD1, GBA, GGA3, GLB1, GLG1, GLMP, GNB2, GOLGA2, GORASP1, GPAT4, GPS1, GPS2, GRAMD1A, GRINA, GRK6, GSTK1, GSTP1, GTPBP1, GTPBP2, GUK1, HDAC3, HDAC6, HDAC7, HDLBP, HEBP2, HEMK1, HIF1AN, HNRNPUL1, HRAS, HSF1, HUWE1, IDH3B, IMPA2, IMPDH1, INF2, INPP5K, INPPL1, INTS11, IQSEC1, KLHDC4, KRI1, KXD1, L3MBTL2, LDB1, LDLRAP1, LIMS2, LMBR1L, LMNA, LPAR2, LPCAT3, LPCAT4, LTBP3, MADD, MAGED1, MAGED4B, MAP2K2, MAP2K3, MAP4, MAP7D1, MAPK3, MAPK8IP3, MAPKAP1, MARK2, MBD1, MBTPS1, MCOLN1, MCRIP2, MED24, MED9, MEF2D, METTL17, METTL22, METTL26, MFSD10, MGAT1, MGAT4B, MGRN1, MIB2, MINDY1, MINK1, MKNK2, MLF2, MLST8, MLX, MPST, MPV17, MRPL21, MSRA, MTA1, MTG1, MTX1, NADSYN1, NAPRT, NARF, NBEAL2, NCOR2, NDRG2, NDUFA10, NDUFA11, NDUFS2, NDUFV3, NELFE, NF2, NFYC, NME4, NOC2L, NOSIP, NPLOC4, NPRL3, NQO2, NR4A1, NSFL1C, NUMA1, NUP214, OAZ2, OGFOD3, ORAI2, OS9, PACS2, PBXIP1, PDAP1, PDLIM2, PDLIM7, PET100, PFKL, PGLS, PGS1, PHC2, PHPT1, PI4 KB, PIGQ, PIGT, PIP4P1, PISD, PKD1, PKM, PKN1, PLA2G6, PLD3, PLPPR2, POLG, POLL, POLR2E, PPCDC, PPDPF, PPP1R35, PPP2R2D, PRCC, PRDX5, PREB, PRKAG1, PRKCD, PRKD2, PRR13, PRR5, PSMD13, PSMD4, PTDSS2, PXN, R3HDM4, RAB24, RAB35, RAD23A, RAF1, RALY, RANBP10, RAPGEF1, RARA, RARG, RBM23, RELA, RER1, RETREG2, REX1BD, RGL2, RNF10, RNF123, RNF126, RNF167, RNF31, RNH1, RNPEPL1, RPN2, RPS6KB2, RTEL1, SCAP, SCNM1, SCYL1, SEC16A, SELENBP1, SELENON, SH2D3A, SH3GLB2, SHARPIN, SHC1, SHKBP1, SIGIRR, SIRT7, SKIV2L, SLC25A39, SLC25A4, SLC44A2, SMARCAL1, SMARCC2, SMARCD3, SMIM29, SNF8, SNRNP70, SPATA2L, ST3GAL1, ST3GAL2, ST6GALNAC4, STARD10, STAT6, STK11, STK16, STK25, STK40, STRN4, STX10, STYXL1, SUN2, TANGO2, TARS2, TBC1D10B, TBCD, TCF25, TEX261, TLN1, TMC4, TMED1, TMEM11, TMEM120A, TMEM134, TMEM161A, TMEM208, TMEM259, TMUB2, TMX2, TNK2, TOLLIP, TOM1L2, TP53, TPCN2, TRAPPC12, TRIM26, TRIP6, TRPM4, TSC2, TSPO, TTLL3, TTYH3, TUBA4A, TUSC2, TXNDC11, U2AF1L4, UBALD1, UBL7, UBN1, UBXN11, UBXN6, UNC45A, UROD, USF2, USP20, VAMP2, VPS11, VPS26B, VPS28, VPS51, VPS52, VRK3, WBP1, WDR45, WDTC1, XAB2, YIF1B, YPEL3, ZFAND2B, ZFPL1, ZFPM1, ZGPAT, ZMIZ2, ZNF384, ZNF76, and ZYX.

The genes in the M4 cluster are ABCD4, ABHD18, ACAD8, ACO1, ACSL1, ACSL4, ADD3, AFF1, AFTPH, AGO3, AKAP10, AKAP11, ALCAM, ALDH3A2, ANKRD10, ANKZF1, ANXA2, ANXA4, AQR, ARFIP1, ARGLU1, ARHGEF9, ARID4A, ARID4B, ASAH1, ASAP1, ASTE1, ASXL1, ATAD2B, ATG12, ATP11B, ATP6AP2, ATP6V1C1, ATRX, BAG4, BBIP1, BCL2L13, BDP1, BECN1, BNIP2, BOD1L1, BPTF, BRWD3, C5orfl5, C9orf72, CACUL1, CASC4, CCDC66, CCNDBP1, CCPG1, CD46, CD55, CDC16, CDC42BPA, CDK19, CDK5RAP3, CEP63, CHMP2B, CHMP3, CHMP5, CLDN12, CLDND1, CLINT1, CLK1, CLK4, CLMN, CLN5, CPEB4, CREB5, CREBBP, CREBRF, CTBP2, CTR9, CUL2, DCAF10, DCAF6, DCP1A, DCP1B, DDIT3, DDX17, DDX3X, DDX5, DDX59, DENND5A, DLG1, DNAJB14, DNAJB4, DNAJC13, DSTN, EFCAB14, ELOVL7, EMC3, EPC1, ESCO1, ETAA1, FAM13B, FAM199X, FAM200B, FAM76B, FAN1, FAXDC2, FBXL5, FBXO9, FBXW7, FGFR10P2, FKBP7, FMO5, FMR1, FOXP1, FUBP3, FZD6, GAB2, GABARAPL1, GADD45A, GALC, GBE1, GIGYF2, GNG2, GOLGA8A, GOLGA8B, GOLPH3, GSK3B, GSKIP, HBP1, HDHD2, HECTD1, HECTD4, HELZ, HERPUD1, HIPK1, HIVEP2, HNRNPH3, HP1BP3, HPS4, HPS5, IDS, IFT74, IGF2R, IKBKB, IP6K2, ISCU, ITSN2, JADE1, JAK1, JAZF1, KCTD20, KDELR2, KDM6A, KDM7A, KIZ, KLHL21, KMT2C, KMT5B, LARP7, LCORL, LEMD3, LEPROTL1, LIMK2, LONP2, LRRFIP1, LUC7L, LUC7L3, MAP2K4, MAP4K4, MAP4K5, MARF1, MARK3, MAX, MBNL1, MED13L, MED23, MFF, MFSD1, MIA2, MIB1, MICU2, MKLN1, MKRN1, MLH3, MOB1B, MON2, MPPE1, MPZL1, MSL1, MTMR3, MXI1, MYL12A, NABP1, NBPF14, NCBP3, NCOA1, NCOA2, NECAP1, NEDD9, NF1, NFAT5, NFATC3, NIN, NPAT, NPEPPS, NPTN, NT5C2, NUMB, OGA, OPTN, PAM, PARD3, PHF21A, PHF3, PHIP, PHYH, PICALM, PIK3CA, PIP4P2, PKIG, PKN2, PLAGL1, PNISR, PPP1R12A, PPP1R3E, PPP6R3, PRDM2, PRMT2, PRRC2C, PTP4A2, PTPN4, PUM2, QKI, RAB2B, RAB40B, RABEP1, RABGAP1L, RAP1B, RBBP6, RBM25, RBM39, RBM5, RBM6, REEP3, REPS1, REXO2, RHOQ, RICTOR, RIOK3, RIT1, RNF11, RNF111, RNF13, RNF170, RNF44, RNPC3, ROCK1, RPGR, RPS6KA3, RPS6KA5, RTN3, SAMD8, SARAF, SCARB2, SEC14L1, SEC31A, SECISBP2L, SELENOF, SERINC1, SERP1, SETD2, SETD5, SETX, SF1, SF3B1, SGCE, SH3YL1, SHPRH, SIK3, SLC18B1, SLC22A5, SLC35A5, SLC35F5, SLTM, SMG1, SNRK, SNX27, SNX3, SON, SORT1, SPAG16, SPATS2L, SPG21, SPOP, SREK1, SRGAP2, SRSF11, SRSF5, SSR3, STAG2, STK38L, STX12, STX16, STX3, STXBP5, SUGP2, SUPT20H, SYNRG, TAB3, TARBP1, TBCEL, TBL1XR1, TBRG1, TCEAL8, TCP11L2, TET2, TFPI, TIAL1, TJP2, TLE4, TLK2, TMCO3, TMEM123, TMEM135, TMEM165, TMEM248, TMEM260, TMEM39A, TMEM43, TMEM9B, TPM1, TPP2, TRAM1, TRAPPC6B, TRIM13, TRIM6, TRIM8, TTC31, TTC32, TUBA1A, TUT7, UBE2B, UBE2H, UBXN4, UHRF2, ULK2, USF3, USP15, USP3, USP32, USP33, USP37, USP48, VMP1, VPS13B, VPS37A, VPS41, WDR11, WDR26, WDR44, WDR48, WDSUB1, WLS, WNK1, YIPF1, YIPF4, YIPF6, ZBTB38, ZBTB44, ZC3H11A, ZC3H13, ZC3H4, ZEB1, ZFAND1, ZFAND6, ZNF12, ZNF177, ZNF185, ZNF195, ZNF267, ZNF302, ZNF347, ZNF562, and ZNF638.

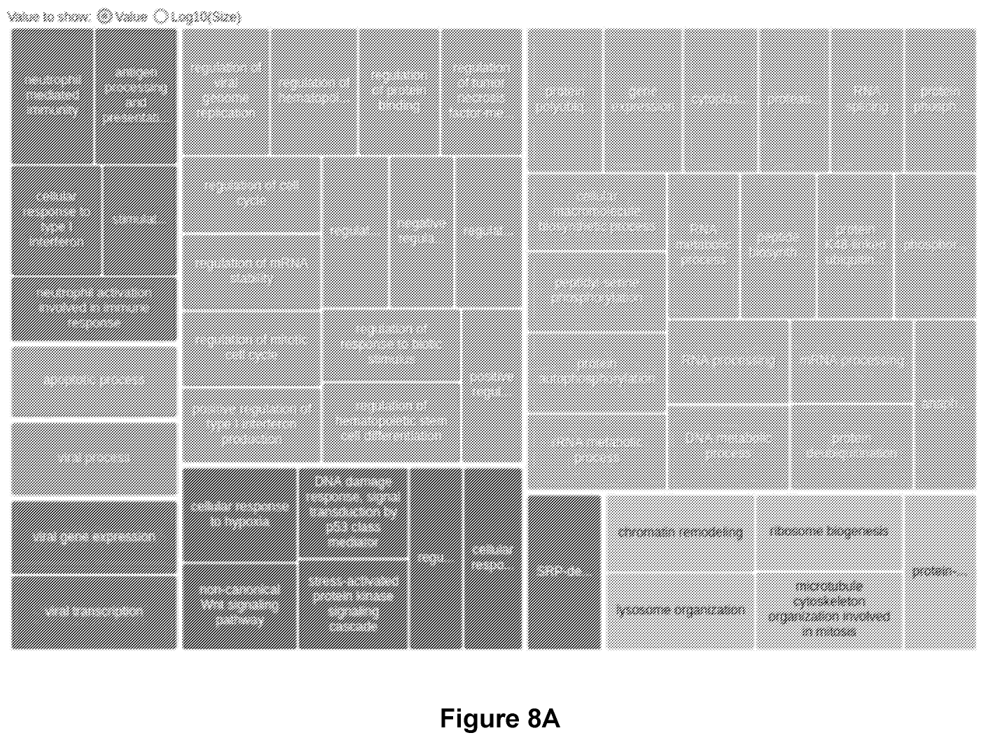

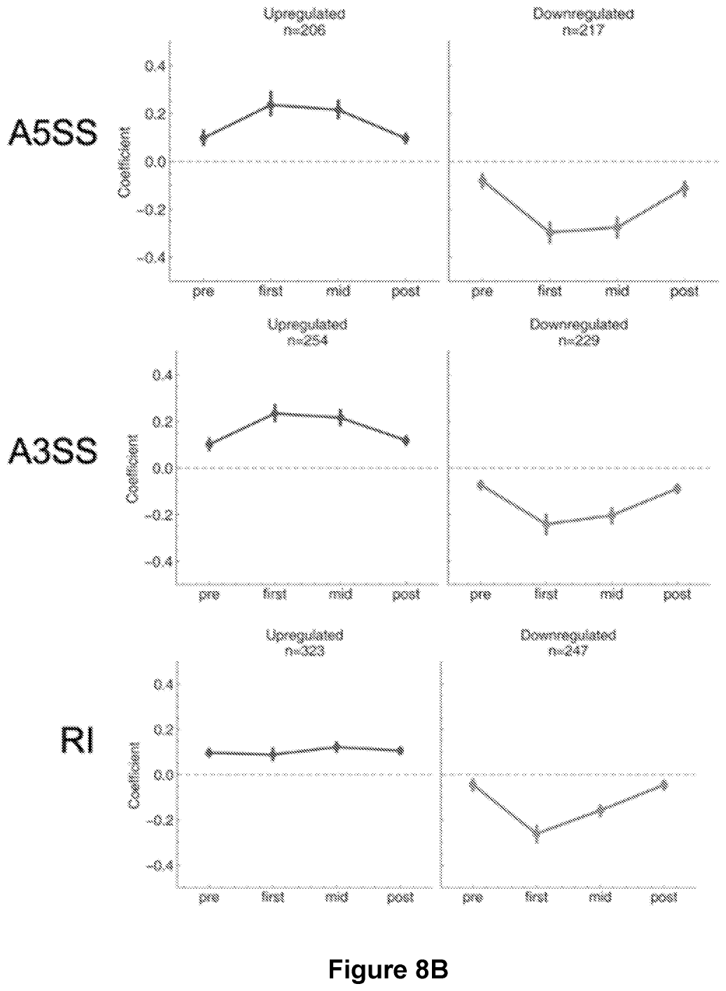

The temporal dynamics of biological processes affected by DAS were examined (FIG. 5A). Specifically, the significantly enriched Biological Process GO terms were summarized into clusters (FIG. 8A) and the largest three GO clusters for temporal changes in numbers of DAS genes at each stage through pre-infection, infection (first and mid), and post-infection were analyzed. Genes related to neutrophil-mediated immunity were differentially spliced at a comparable ratio between first and mid; by contrast, regulation of type I interferon production was substantially more differentially spliced at mid compared with first. Interestingly, the regulation of AS also came up as strongly enriched in DAS genes, forming an auto-regulatory loop of splicing factors and target AS exons (Table 3).

TABLE 3

| | Event | | |
|---|---|---|---|
| Event ID | Type | Gene | Condition |
| chr14:+:73099666:73099750:73103191:73103478 | RI | RBM25 | final@First |
| chr3:+:152445539:152446703:152446757:152447619 | SE | MBNL1 | final@First |
| chr19:−:38840559:38841612:38841682:38843841 | SE | HNRNPL | final@First |
| chr14:+:73111155:73111308:73111382:73111527 | SE | RBM25 | final@First |
| chr19:−:38840559:38841612:38841682:38843841 | SE | HNRNPL | final@Mid |
| chr22:−:38487878:38488115:38492055:38492115 | RI | DDX17 | final@Mid |
| chr14:+:73077536:73083493:73083551:73088000 | SE | RBM25 | final@Mid |
| chr14:+:73111155:73111308:73111382:73111527 | SE | RBM25 | final@Mid |
| chr3:+:152445539:152446703:152446757:152447619 | SE | MBNL1 | final@Mid |
| chrX:−:132406392:132439434:132439572:132489813 | SE | MBNL3 | final@Mid |
| chr12:+:54282874:54283078:54283234:54283811 | SE | HNRNPA1 | final@Mid |
| chr1:−:244846706:244847001:244847053:244848787 | SE | HNRNPU | final@Mid |
| chrX:+:147937600:147938098:147938161:147940575 | SE | FMR1 | final@Mid |
| chr12:+:54284317:54284548:54284317:54286661 | A3SS | HNRNPA1 | final@Post |
| chr12:+:54282874:54283078:54283234:54283811 | SE | HNRNPA1 | final@Post |
| chr14:+:73110830:73111155:73111527:73111802 | RI | RBM25 | final@Post |
| chr14:+:73099666:73099750:73103191:73103478 | RI | RBM25 | final@Post |
| chr1:−:244860334:244860474:244862460:244862534 | RI | HNRNPU | final@Post |
| chr1:−:244862730:244863616:244862730:244863673 | A5SS | HNRNPU | final@Post |
| chr1:−:244846706:244847001:244847053:244848787 | SE | HNRNPU | final@Post |
| chr3:+:152445539:152446703:152446757:152447619 | SE | MBNL1 | final@Post |

The temporal dynamics of the DAS events on the exon level were analyzed (FIG. 5C). By meta-analyzing the estimated coefficients from the linear mixed models, the temporal changes of AS differences where the molecular level response was the most dramatic at first, followed by mid, were observed. A set of DAS events with consistent cross-cohort patterns was also observed (Table 4; two representative signatures in FIGS. 7F and 7G). GALNS is a lysosomal exohydrolase involved in innate immune response. See Rivera-Colón et al., 2012, “The structure of human GALNS reveals the molecular basis for mucopolysaccharidosis IV,” A. J. Mol. Biol., 423, pg. 736-751, which is hereby incorporated by reference in its entirety for all purposes.

TABLE 4
DAS events with consistent cross-cohort patterns.

| | | | | Charm | Charm | Duke | Duke | Charm | Duke |
|---|---|---|---|---|---|---|---|---|---|
| | Event | | | control | covid | control | covid | delta | Delta |
| Event ID | Type | Gene name | Importance | PSI | PSI | PSI | PSI | PSI | PSI |
| chr22: +: 22888259: | SE | IGLL5 | 1.28 | 0.7442 | 0.8101 | 0.8705 | 0.9077 | 0.0658 | 0.0372 |
| 22893699: | | | | | | | | | |
| 22893818: | | | | | | | | | |
| 22895374 | | | | | | | | | |
| chr6: +: 31587199: | RI | LST1 | −1.17 | 0.5265 | 0.48 | 0.612 | 0.451 | −.0465 | −0.161 |
| 31587318: | | | | | | | | | |
| 31588517: | | | | | | | | | |
| 31588909 | | | | | | | | | |
| chr16: −: 88841971: | SE | GALNS | 1.17 | 0.6541 | 0.6976 | 0.6995 | 0.7975 | 0.0435 | 0.098 |
| 88842705: | | | | | | | | | |
| 88842829: | | | | | | | | | |
| 88856757 | | | | | | | | | |
| chr13: −: 42917624: | SE | EPSTI1 | 1.12 | 0.4101 | 0.4753 | 0.5151 | 0.5987 | 0.0653 | 0.0835 |
| 42919299: | | | | | | | | | |
| 42919332: | | | | | | | | | |
| 42926335 | | | | | | | | | |
| chr16: −: 16219947: | SE | ABCC6 | −0.95 | 0.7329 | 0.6599 | 0.6641 | 0.5349 | −0.073 | −.1293 |
| 16220272: | | | | | | | | | |
| 16220388: | | | | | | | | | |
| 16221648 | | | | | | | | | |
| chr11: −: 60208694: | SE | MS4A4E | −0.92 | 0.9198 | 0.9007 | 0.9393 | 0.9224 | −.0192 | −.0169 |
| 60212973: | | | | | | | | | |
| 60213132: | | | | | | | | | |
| 60214570 | | | | | | | | | |
| chr22: −: 50531773: | A3SS | ODF3B | −0.91 | 0.5211 | 0.5042 | 0.5619 | 0.5186 | −.0169 | −.0433 |
| 50531968: | | | | | | | | | |
| 50531702: | | | | | | | | | |
| 50531968 | | | | | | | | | |
| chr19: −: 19146022: | SE | MEF2B | −0.82 | 0.5587 | 0.5452 | 0.409 | 0.3439 | −.0135 | −.0651 |
| 19146272: | | | | | | | | | |
| 19146405: | | | | | | | | | |
| 19146554 | | | | | | | | | |
| chr20: +: 5127149: | SE | CDS2 | −0.75 | 0.8261 | 0.7989 | 0.8219 | 0.7677 | −.0271 | −.0542 |
| 5173522: | | | | | | | | | |
| 5173659: | | | | | | | | | |
| 5175182 | | | | | | | | | |
| chr6: −: 30679389: | A5SS | PPP1R18 | 0.7 | 0.3562 | 0.4121 | 0.4259 | 0.597 | 0.0559 | 0.1711 |
| 30684407: | | | | | | | | | |
| 30679389: | | | | | | | | | |
| 30687113 | | | | | | | | | |
| chr6: +: 44229906: | RI | SLC29A1 | −0.7 | 0.2293 | 0.2186 | 0.1899 | 0.1218 | −.0108 | −.0681 |
| 44230046: | | | | | | | | | |
| 44230346: | | | | | | | | | |
| 44230481 | | | | | | | | | |
| chr1: +: 161673959: | RI | FCGR2B | −0.68 | 0.7107 | 0.6993 | 0.6898 | 0.6065 | −.0114 | −.0833 |
| 161674073: | | | | | | | | | |
| 161677327: | | | | | | | | | |
| 161677365 | | | | | | | | | |
| chr1: +: 26473482: | RI | HMGN2 | −0.66 | 0.2918 | 0.2706 | 0.1481 | 0.0897 | −.0212 | −.0584 |
| 26473527: | | | | | | | | | |
| 26473702: | | | | | | | | | |
| 26474135 | | | | | | | | | |
| chr22: +: 18110868: | A5SS | TUBA8 | 0.64 | 0.0776 | 0.0918 | 0.1072 | 0.1326 | 0.0143 | 0.0254 |
| 18121478: | | | | | | | | | |
| 18110839: | | | | | | | | | |
| 18121478 | | | | | | | | | |
| chr6: +: 31835451: | SE | SNHG32 | 0.62 | 0.4274 | 0.4489 | 0.4774 | 0.5805 | 0.0216 | 0.103 |
| 31836423: | | | | | | | | | |
| 31836517: | | | | | | | | | |
| 31837235 | | | | | | | | | |
| chr16: +: 29804065: | A3SS | KIF22 | −0.62 | 0.3204 | 0.2715 | 0.1281 | 0.0669 | −.0489 | −.0612 |
| 29804575: | | | | | | | | | |
| 29804065: | | | | | | | | | |
| 29804813 | | | | | | | | | |
| chr5: −: 139388131: | SE | MZB1 | 0.59 | 0.8638 | 0.9194 | 0.9538 | 0.9849 | 0.0556 | 0.0311 |
| 139388460: | | | | | | | | | |
| 139388585: | | | | | | | | | |
| 139389679 | | | | | | | | | |
| chr11: −: 95185492: | SE | SESN3 | 0.57 | 0.6755 | 0.7428 | 0.7165 | 0.8195 | 0.0673 | 0.103 |
| 95189778: | | | | | | | | | |
| 95189961: | | | | | | | | | |
| 95191403 | | | | | | | | | |
| chr17: −: 78973343: | A3SS | LGALS3BP | −0.57 | 0.2208 | 0.2012 | 0.16 | 0.1383 | −.0196 | −.0217 |
| 78974687: | | | | | | | | | |
| 78973222: | | | | | | | | | |
| 78974687 | | | | | | | | | |
| chr6: +: 31587733: | A3SS | LST1 | 0.54 | 0.4672 | 0.5082 | 0.5322 | 0.6179 | 0.041 | 0.0857 |
| 31588517: | | | | | | | | | |
| 31587733: | | | | | | | | | |
| 31588562 | | | | | | | | | |
| chr6: +: 31587318: | A3SS | LST1 | 0.53 | 0.6194 | 0.6437 | 0.6455 | 0.6855 | 0.0242 | 0.0401 |
| 31588517: | | | | | | | | | |
| 31587318: | | | | | | | | | |
| 31588562 | | | | | | | | | |
| chr1: +: 26473482: | RI | HMGN2 | −0.53 | 0.2781 | 0.2452 | 0.2184 | 0.141 | −.0329 | −.0774 |
| 26473732: | | | | | | | | | |
| 26474084: | | | | | | | | | |
| 26474135 | | | | | | | | | |
| chr9: −: 121467931: | SE | GGTA1P | 0.52 | 0.0925 | 0.1103 | 0.0983 | 0.1504 | 0.0177 | 0.0521 |
| 121479065: | | | | | | | | | |
| 121479334: | | | | | | | | | |
| 121499649 | | | | | | | | | |
| chr6: +: 25992918: | SE | U91328.1 | 0.51 | 0.0837 | 0.1151 | 0.0528 | 0.0872 | 0.0314 | 0.0344 |
| 25993996: | | | | | | | | | |
| 25994142: | | | | | | | | | |
| 25997902 | | | | | | | | | |
| chr2: −: 200859746: | SE | CLK1 | 0.49 | 0.4286 | 0.4467 | 0.7094 | 0.7604 | 0.018 | 0.051 |
| 200860124: | | | | | | | | | |
| 200860215: | | | | | | | | | |
| 200861237 | | | | | | | | | |
| chr6: +: 31587733: | SE | LST1 | −0.45 | 0.8199 | 0.7775 | 0.8448 | 0.7813 | −.0425 | −.0636 |
| 31587943: | | | | | | | | | |
| 31587966: | | | | | | | | | |
| 31588517 | | | | | | | | | |
| chr6: +: 31587199: | RI | LST1 | −0.44 | 0.4599 | 0.4442 | 0.5323 | 0.4881 | −.0157 | −.0442 |
| 31587318: | | | | | | | | | |
| 31588562: | | | | | | | | | |
| 31588909 | | | | | | | | | |
| chr22: −: 38740266: | RI | SUN2 | 0.43 | 0.1393 | 0.1573 | 0.1165 | 0.1307 | 0.018 | 0.0142 |
| 38740432: | | | | | | | | | |
| 38741006: | | | | | | | | | |
| 38741050 | | | | | | | | | |
| chr8: +: 103021225: | SE | ATP6V1C1 | −0.43 | 0.8147 | 0.7969 | 0.7306 | 0.7224 | −.0178 | −.0082 |
| 103040797: | | | | | | | | | |
| 103040968: | | | | | | | | | |
| 103042339 | | | | | | | | | |
| chr12: −: 57517268: | RI | DDIT3 | −0.42 | 0.196 | 0.1835 | 0.2974 | 0.2477 | −.0125 | −.0497 |
| 57517438: | | | | | | | | | |
| 57517705: | | | | | | | | | |
| 57517753 | | | | | | | | | |
| chr10: −: 45461411: | SE | MARCHF8 | 0.42 | 0.1823 | 0.1953 | 0.1547 | 0.1912 | 0.0131 | 0.0365 |
| 45463150: | | | | | | | | | |
| 45463996: | | | | | | | | | |
| 45464238 | | | | | | | | | |
| chr3: −: 12901522: | A3SS | IQSEC1 | 0.42 | 0.7796 | 0.8297 | 0.8193 | 0.8357 | 0.0501 | 0.0163 |
| 12902772: | | | | | | | | | |
| 12899434: | | | | | | | | | |
| 12902772 | | | | | | | | | |
| chr11: +: 72822477: | RI | ATG16L2 | 0.41 | 0.4832 | 0.4995 | 0.5333 | 0.573 | 0.0163 | 0.0397 |
| 72822543: | | | | | | | | | |
| 72822847: | | | | | | | | | |
| 72824842 | | | | | | | | | |
| chr7: −: 27895416: | SE | JAZF1 | −0.4 | 0.3479 | 0.3164 | 0.2597 | 0.2449 | −.0315 | −.0148 |
| 27913391: | | | | | | | | | |
| 27913453: | | | | | | | | | |
| 27991908 | | | | | | | | | |
| chr17: +: 34998603: | RI | LIG3 | −0.39 | 0.2304 | 0.2113 | 0.2048 | 0.1886 | −.0191 | −.0162 |
| 34998727: | | | | | | | | | |
| 34999306: | | | | | | | | | |
| 34999449 | | | | | | | | | |
| chr15: +: 101066741: | SE | LRRK1 | −0.38 | 0.5705 | 0.5392 | 0.5774 | 0.4627 | −.0314 | −.1148 |
| 101067239: | | | | | | | | | |
| 101067387: | | | | | | | | | |
| 101068670 | | | | | | | | | |
| chr4: +: 151099714: | SE | RPS3A | 0.38 | 0.0839 | 0.1032 | 0.0822 | 0.5107 | 0.0194 | 0.4285 |
| 151100484: | | | | | | | | | |
| 151100588: | | | | | | | | | |
| 151102870 | | | | | | | | | |
| chr3: −: 134647497: | A5SS | KY | 0.37 | 0.3474 | 0.3673 | 0.3568 | 0.4304 | 0.0199 | 0.0737 |
| 134650824: | | | | | | | | | |
| 134647497: | | | | | | | | | |
| 134650878 | | | | | | | | | |
| chr1: +: 112675049: | SE | MOV10 | −0.36 | 0.1631 | 0.1396 | 0.1336 | 0.0746 | −.0235 | −0.059 |
| 112685034: | | | | | | | | | |
| 112685162: | | | | | | | | | |
| 112688934 | | | | | | | | | |
| chr3: −: 71772828: | SE | PROK2 | −0.35 | 0.4667 | 0.4471 | 0.5519 | 0.525 | −.0196 | −.0269 |
| 71774444: | | | | | | | | | |
| 71774507: | | | | | | | | | |
| 71781466 | | | | | | | | | |
| chr17: −: 49225131: | A3SS | PHOSPHO1 | 0.35 | 0.5928 | 0.6187 | 0.5794 | 0.6472 | 0.0259 | 0.0678 |
| 49226646: | | | | | | | | | |
| 49225004: | | | | | | | | | |
| 49226646 | | | | | | | | | |
| chr17: −: 1646619: | SE | RILP | −0.34 | 0.8591 | 0.8266 | 0.8928 | 0.8779 | −.0326 | −.0149 |
| 1646905: | | | | | | | | | |
| 1646989: | | | | | | | | | |
| 1647834 | | | | | | | | | |
| chr6: −: 24840775: | A3SS | RIPOR2 | −0.32 | 0.8525 | 0.8336 | 0.7731 | 0.6966 | −.0189 | −.0765 |
| 24842861: | | | | | | | | | |
| 24839599: | | | | | | | | | |
| 24842861 | | | | | | | | | |
| chr12: −: 10701959: | RI | YBX3 | 0.32 | 0.0292 | 0.0469 | 0.0382 | 0.0457 | 0.0177 | 0.0075 |
| 10702134: | | | | | | | | | |
| 10704059: | | | | | | | | | |
| 10704148 | | | | | | | | | |
| chr1: −: 247310456: | SE | ZNF496 | −0.31 | 0.5574 | 0.527 | 0.5957 | 0.475 | −.0303 | −.1208 |
| 247322697: | | | | | | | | | |
| 247322805: | | | | | | | | | |
| 247323153 | | | | | | | | | |
| chr11: +: 65583765: | A5SS | EHBP1L1 | −0.31 | 0.9251 | 0.9072 | 0.9365 | 0.9191 | −.0179 | −.0174 |
| 65584240: | | | | | | | | | |
| 65582016: | | | | | | | | | |
| 65584240 | | | | | | | | | |
| chr6: +: 31587318: | SE | LST1 | −0.28 | 0.1705 | 0.1533 | 0.1592 | 0.158 | −.0172 | −.0013 |
| 31587943: | | | | | | | | | |
| 31587966: | | | | | | | | | |
| 31588562 | | | | | | | | | |
| chr3: −: 134610383: | SE | KY | −0.28 | 0.6526 | 0.6327 | 0.6432 | 0.5695 | −.0199 | −.0737 |
| 134610521: | | | | | | | | | |
| 134610707: | | | | | | | | | |
| 134619147 | | | | | | | | | |
| chr3: −: 134627818: | A5SS | KY | −0.27 | 0.6526 | 0.6327 | 0.6432 | 0.5695 | −.0199 | −.0737 |
| 134629403: | | | | | | | | | |
| 134627818: | | | | | | | | | |
| 134629620 | | | | | | | | | |
| chr10: +: 78037438: | RI | RPS24 | 0.27 | 0.1049 | 0.1239 | 0.1748 | 0.6257 | 0.019 | 0.4509 |
| 78037441: | | | | | | | | | |
| 78040203: | | | | | | | | | |
| 78040225 | | | | | | | | | |
| chr1: +: 40769415: | A3SS | NFYC | −0.27 | 0.3081 | 0.2924 | 0.25 | 0.2304 | −.0157 | −.0195 |
| 40770396: | | | | | | | | | |
| 40769415: | | | | | | | | | |
| 40770708 | | | | | | | | | |
| chr20: +: 58670603: | SE | STX16 | 0.26 | 0.906 | 0.9175 | 0.8892 | 0.9355 | 0.0115 | 0.0463 |
| 58671153: | | | | | | | | | |
| 58671297: | | | | | | | | | |
| 58673630 | | | | | | | | | |
| chr2: −: 217821938: | SE | TNS1 | 0.26 | 0.0726 | 0.0922 | 0.1223 | 0.1663 | 0.0196 | 0.044 |
| 217829843: | | | | | | | | | |
| 217829882: | | | | | | | | | |
| 217831454 | | | | | | | | | |
| chr14: −: 94388660: | SE | SERPINA1 | 0.25 | 0.1765 | 0.2095 | 0.0896 | 0.1329 | 0.033 | 0.0433 |
| 94388818: | | | | | | | | | |
| 94389010: | | | | | | | | | |
| 94390456 | | | | | | | | | |
| chr14: +: 105486972: | A3SS | CRIP1 | −0.25 | 0.8332 | 0.8452 | 0.8866 | 0.9328 | 0.012 | 0.0462 |
| 105487198: | | | | | | | | | |
| 105486972: | | | | | | | | | |
| 105487212 | | | | | | | | | |
| chr15: +: 88639594: | A3SS | ISG20 | −0.24 | 0.7235 | 0.7092 | 0.7083 | 0.7014 | −.0143 | −.0069 |
| 88652109: | | | | | | | | | |
| 88639594: | | | | | | | | | |
| 88652169 | | | | | | | | | |
| chr10: +: 49988938: | SE | AGAP6 | −0.23 | 0.4161 | 0.4533 | 0.4433 | 0.5283 | 0.0371 | 0.085 |
| 49989307: | | | | | | | | | |
| 49989376: | | | | | | | | | |
| 49991675 | | | | | | | | | |
| chr17: −: 5532984: | A5SS | NLRP1 | 0.23 | 0.3592 | 0.3818 | 0.3784 | 0.4216 | 0.0226 | 0.0432 |
| 5533291: | | | | | | | | | |
| 5532984: | | | | | | | | | |
| 5533303 | | | | | | | | | |
| chr1: +: 28887210: | A3SS | EPB41 | 0.22 | 0.2291 | 0.1992 | 0.2305 | 0.1649 | −.0299 | −.0656 |
| 28987430: | | | | | | | | | |
| 28887210: | | | | | | | | | |
| 28987447 | | | | | | | | | |
| chr2: −: 69996888: | SE | PCBP1-AS1 | −0.22 | 0.2753 | 0.2969 | 0.2703 | 0.3337 | 0.0216 | 0.0634 |
| 70032132: | | | | | | | | | |
| 70032255: | | | | | | | | | |
| 70051202 | | | | | | | | | |
| chr6: +: 37014100: | A3SS | FGD2 | 0.22 | 0.5724 | 0.5997 | 0.6588 | 0.6664 | 0.0273 | 0.0076 |
| 37014390: | | | | | | | | | |
| 37014100: | | | | | | | | | |
| 37014580 | | | | | | | | | |
| chr14: +: 55129300: | SE | LGALS3 | 0.21 | 0.7737 | 0.7949 | 0.8936 | 0.8951 | 0.0212 | 0.0015 |
| 55137369: | | | | | | | | | |
| 55137391: | | | | | | | | | |
| 55138044 | | | | | | | | | |
| chrX: −: 119620051: | SE | SEPTIN6 | 0.21 | 0.3645 | 0.3766 | 0.4985 | 0.5852 | 0.012 | 0.0867 |
| 119625334: | | | | | | | | | |
| 119625379: | | | | | | | | | |
| 119629317 | | | | | | | | | |
| chr8: +: 143018586: | A3SS | LY6E | 0.21 | 0.7599 | 0.774 | 0.757 | 0.7873 | 0.0141 | 0.0303 |
| 143020882: | | | | | | | | | |
| 143018586: | | | | | | | | | |
| 143020887 | | | | | | | | | |
| chr1: +: 29053312: | SE | EPB41 | 0.21 | 0.229 | 0.196 | 0.2225 | 0.1606 | −0.033 | −0.062 |
| 29060421: | | | | | | | | | |
| 29060484: | | | | | | | | | |
| 29064981 | | | | | | | | | |
| chr16: −: 74472411: | SE | GLG1 | 0.21 | 0.1236 | 0.1818 | 0.1943 | 0.2042 | 0.0582 | 0.0099 |
| 74472525: | | | | | | | | | |
| 74472634: | | | | | | | | | |
| 74474545 | | | | | | | | | |
| chr2: −: 37122664: | SE | EIF2AK2 | −0.21 | 0.9685 | 0.9339 | 0.7905 | 0.7418 | −.0345 | −.0488 |
| 37126288: | | | | | | | | | |
| 37126411: | | | | | | | | | |
| 37135483 | | | | | | | | | |
| chr2: +: 201272776: | SE | CASP8 | −0.21 | 0.8015 | 0.7915 | 0.8388 | 0.7992 | −0.01 | −.0397 |
| 201272897: | | | | | | | | | |
| 201272942: | | | | | | | | | |
| 201274888 | | | | | | | | | |
| chr11: +: 120407823: | SE | ARHGEF12 | −0.21 | 0.7516 | 0.7788 | 0.7609 | 0.7901 | 0.0272 | 0.0291 |
| 120409393: | | | | | | | | | |
| 120409450: | | | | | | | | | |
| 120420752 | | | | | | | | | |
| chr3: −: 98521442: | SE | CLDND1 | −0.2 | 0.1324 | 0.1168 | 0.1155 | 0.1081 | −.0156 | −.0074 |
| 98521652: | | | | | | | | | |
| 98521718: | | | | | | | | | |
| 98522848 | | | | | | | | | |
| chr6: +: 138406151: | A5SS | HEBP2 | 0.2 | 0.1343 | 0.151 | 0.1598 | 0.1728 | 0.0168 | 0.013 |
| 138412879: | | | | | | | | | |
| 138406033: | | | | | | | | | |
| 138412879 | | | | | | | | | |
| chr16: −: 30095434: | A3SS | YPEL3 | 0.18 | 0.818 | 0.8286 | 0.8352 | 0.8512 | 0.0106 | 0.016 |
| 30096096: | | | | | | | | | |
| 30095335: | | | | | | | | | |
| 30096096 | | | | | | | | | |
| chr22: +: 24171995: | SE | CABIN1 | 0.18 | 0.1178 | 0.1302 | 0.1409 | 0.1591 | 0.0124 | 0.0183 |
| 24176110: | | | | | | | | | |
| 24176275: | | | | | | | | | |
| 24177503 | | | | | | | | | |
| chr14: +: 21087000: | RI | ARHGEF40 | 0.18 | 0.5987 | 0.5877 | 0.5182 | 0.4781 | −0.011 | −.0402 |
| 21087105: | | | | | | | | | |
| 21087319: | | | | | | | | | |
| 21087463 | | | | | | | | | |
| chr6: −: 7301572: | A3SS | SSR1 | −0.18 | 0.9619 | 0.9488 | 0.9178 | 0.8955 | −0.013 | −.0224 |
| 7303549: | | | | | | | | | |
| 7301368: | | | | | | | | | |
| 7303549 | | | | | | | | | |
| chr19: −: 49873302: | A5SS | AKT1S1 | 0.17 | 0.1253 | 0.154 | 0.1859 | 0.1914 | 0.0287 | 0.0055 |
| 49876602: | | | | | | | | | |
| 49873302: | | | | | | | | | |
| 49877236 | | | | | | | | | |
| chr3: −: 48501293: | A5SS | SHISA5 | 0.16 | 0.8309 | 0.8061 | 0.8089 | 0.7868 | −.0248 | −.0221 |
| 48504018: | | | | | | | | | |
| 48501293: | | | | | | | | | |
| 48504422 | | | | | | | | | |
| chr17: +: 75704814: | A3SS | SAP30BP | −0.16 | 0.2544 | 0.2415 | 0.2048 | 0.1536 | −.0128 | −.0512 |
| 75705610: | | | | | | | | | |
| 75704814: | | | | | | | | | |
| 75706007 | | | | | | | | | |
| chr1: −: 151366904: | SE | SELENBP1 | 0.14 | 0.0176 | 0.0294 | 0.0107 | 0.0162 | 0.0118 | 0.0055 |
| 151367015: | | | | | | | | | |
| 151367175: | | | | | | | | | |
| 151368198 | | | | | | | | | |
| chr19: +: 965080: | SE | ARID3A | −0.12 | 0.9481 | 0.9301 | 0.7895 | 0.7641 | −0.018 | −.0254 |
| 966571: | | | | | | | | | |
| 966868: | | | | | | | | | |
| 968404 | | | | | | | | | |
| chr1: −: 154601040: | RI | ADAR | 0.11 | 0.9419 | 0.928 | 0.8602 | 0.8367 | −.0139 | −.0235 |
| 154602248: | | | | | | | | | |
| 154602384: | | | | | | | | | |
| 154602626 | | | | | | | | | |
| chr10: +: 71712813: | A3SS | CDH23 | −0.11 | 0.7705 | 0.8102 | 0.6616 | 0.7128 | 0.0397 | 0.0512 |
| 71713051: | | | | | | | | | |
| 71712813: | | | | | | | | | |
| 71715201 | | | | | | | | | |
| chrX: +: 48786645: | SE | GATA1 | 0.11 | 0.6343 | 0.654 | 0.5727 | 0.6464 | 0.0197 | 0.0737 |
| 48791090: | | | | | | | | | |
| 48791329: | | | | | | | | | |
| 48791843 | | | | | | | | | |
| chr1: +: 24662804: | SE | SRRM1 | −0.11 | 0.4036 | 0.4212 | 0.4268 | 0.4901 | 0.0175 | 0.0633 |
| 24663182: | | | | | | | | | |
| 24663224: | | | | | | | | | |
| 24666814 | | | | | | | | | |
| chr11: −: 85983973: | SE | PICALM | 0.09 | 0.1729 | 0.1863 | 0.1984 | 0.1989 | 0.0134 | 0.0005 |
| 85990249: | | | | | | | | | |
| 85990399: | | | | | | | | | |
| 85996825 | | | | | | | | | |
| chr3: +: 127673081: | A3SS | ABTB1 | −0.08 | 0.1037 | 0.0917 | 0.0877 | 0.0762 | −0.012 | −.0114 |
| 127674344: | | | | | | | | | |
| 127673081: | | | | | | | | | |
| 127674390 | | | | | | | | | |
| chr7: +: 28818179: | SE | CREB5 | −0.08 | 0.1377 | 0.1265 | 0.2161 | 0.1212 | −.0112 | −.0949 |
| 28818760: | | | | | | | | | |
| 28818789: | | | | | | | | | |
| 28819115 | | | | | | | | | |
| chr17: +: 40129627: | SE | MSL1 | −0.07 | 0.951 | 0.9398 | 0.9755 | 0.9499 | −.0113 | −.0256 |
| 40131536: | | | | | | | | | |
| 40131584: | | | | | | | | | |
| 40132033 | | | | | | | | | |
| chr7: +: 102433661: | A3SS | ORAI2 | −0.06 | 0.036 | 0.0498 | 0.0239 | 0.0344 | 0.0138 | 0.0105 |
| 102436201: | | | | | | | | | |
| 102433661: | | | | | | | | | |
| 102436229 | | | | | | | | | |
| chr10: +: 71617393: | SE | CDH23 | 0.06 | 0.1908 | 0.1546 | 0.2403 | 0.2235 | −.0363 | −.0167 |
| 71635237: | | | | | | | | | |
| 71635297: | | | | | | | | | |
| 71643860 | | | | | | | | | |
| chrX: −: 80682628: | A5SS | BRWD3 | 0.05 | 0.6359 | 0.6526 | 0.6814 | 0.7343 | 0.0167 | 0.0528 |
| 80684009: | | | | | | | | | |
| 80682628: | | | | | | | | | |
| 80684089 | | | | | | | | | |
| chr19: −: 39433664: | RI | RPS16 | −0.04 | 0.1941 | 0.214 | 0.1983 | 0.3352 | 0.0199 | 0.137 |
| 39433761: | | | | | | | | | |
| 39435657: | | | | | | | | | |
| 39435708 | | | | | | | | | |
| chr2: +: 127634175: | RI | MYO7B | 0.03 | 0.7079 | 0.6753 | 0.5164 | 0.4879 | −.0326 | −.0286 |
| 127634289: | | | | | | | | | |
| 127634595: | | | | | | | | | |
| 127634683 | | | | | | | | | |
| chr17: +: 74466743: | SE | CD300A | 0.02 | 0.7691 | 0.7846 | 0.7906 | 0.8458 | 0.0155 | 0.0552 |
| 74473535: | | | | | | | | | |
| 74473874: | | | | | | | | | |
| 74474531 | | | | | | | | | |
| chr20: +: 32052486: | SE | HCK | −0.02 | 0.1367 | 0.126 | 0.0939 | 0.0736 | −.0108 | −.0203 |
| 32054215: | | | | | | | | | |
| 32054281: | | | | | | | | | |
| 32071661 | | | | | | | | | |
| chr14: −: 94388663: | SE | SERPINA1 | −0.02 | 0.4897 | 0.5066 | 0.4527 | 0.4913 | 0.0169 | 0.0387 |
| 94388800: | | | | | | | | | |
| 94389010: | | | | | | | | | |
| 94390456 | | | | | | | | | |
| chr16: −: 31201841: | SE | PYCARD | −0.01 | 0.9074 | 0.8925 | 0.8997 | 0.8896 | −.0149 | −.0101 |
| 31202146: | | | | | | | | | |
| 31202203: | | | | | | | | | |
| 31202416 | | | | | | | | | |
| chr9: +: 93110567: | RI | CARD19 | −0.01 | 0.5701 | 0.5567 | 0.5109 | 0.4535 | −.0134 | −.0575 |
| 93110721: | | | | | | | | | |
| 93112217: | | | | | | | | | |
| 93112289 | | | | | | | | | |
| chr19: +: 13075671: | SE | NFIX | 0.01 | 0.8095 | 0.7525 | 0.7531 | 0.6966 | −.057 | −.0565 |
| 13078612: | | | | | | | | | |
| 13078735: | | | | | | | | | |
| 13081679 | | | | | | | | | |
| chr21: +: 41426263: | SE | MX1 | 0.01 | 0.8691 | 0.8829 | 0.8386 | 0.892 | 0.0138 | 0.0533 |
| 41427205: | | | | | | | | | |
| 41427304: | | | | | | | | | |
| 41427754 | | | | | | | | | |

For Table 4, the alternative splicing event “SE” has first element: chromosome, second element: strand, third element: upstream exon end, fourth element; cassette exon start, fifth element: cassette exon end, and sixth element: downstream exon end.

For Table 4, the alternative splicing event “A3SS” has first element: chromosome, second element: strand, third element: long isoform upstream exon end, fourth element: long isoform downstream exon start, fifth element: short isoform upstream exon end, and sixth element: short isoform downstream exon start.

For Table 4, the alternative splicing event “A5SS” has first element: chromosome, second element: strand, third element: long isoform upstream exon end, fourth element: long isoform downstream exon start, fifth element: short isoform upstream exon end, and sixth element: short isoform downstream exon start.

For Table 4, the alternative splicing event “RI” has first element: chromosome, second element: strand, third element: upstream exon start, fourth element: upstream exon end, fifth element: downstream exon start, and sixth element: downstream exon end.

Increased inclusion of exon 2, which belongs to the sulfatase and phosphodiest protein domain families, suggests enhanced enzymatic activity to facilitate the activation of innate immune pathways. The second event is a retained intron (RI) event in LST1, which is thought to be involved in modulating immune responses. See Rollinger-Holzinger et al., 2000, LST1: A gene with extensive alternative splicing and immunomodulatory function,” J. Immunol., 164, pg. 3169-3176, which is hereby incorporated by reference in its entirety for all purposes. Inclusion of the target intron will disrupt the LST1 protein domain and introduce multiple premature stop codons; the systems and methods of the present disclosure analysis shows that this disruptive AS event is significantly reduced during viral infection. This demonstrates that AS can modulate gene functions independently of the cellular transcriptional control.

For functional characterizations of the DAS events, a focus on the protein domains (Pfam database) and experimentally annotated RNA-binding protein (RBP) binding sites (CLIPdb and ENCODE databases) retained in the DAS exons was made. More details on this analysis is disclosed in Example 11. See Mistry et al., 2021, “Pfam: the protein families database in 2021,” Nucleic Acids Res., 49, D412-D419; Yang et al., 2015, “CLIPdb: a CLIP-seq database for protein-RNA interactions,” BMC Genom., 16, pg. 51; Van Nostrand et al., 2020, “A large-scale binding and functional map of human RNA-binding proteins,” Nature, 583, pg. 711-719, each of which is hereby incorporated by reference in its entirety for all purposes. For protein domain analysis, LST1 is the most enriched protein family (FIG. 4D). Other enriched domains include those that represent post-translational regulation and signaling cascades (glycosyl transfer family a/b and PKinase) and cell migration and cytokine secretion (prefoldin, TANGO2), as well as post-transcriptional regulation (RRM) in response to the viral infection.

A number of RBPs whose binding sites are significantly enriched in the DAS exons compared with all exons were identified, including RBM22, METAP2, and PABPC4 (FIG. 4E). PABPC4 is known to be upregulated in activated T cells and might be necessary for the regulation of stability of labile mRNA species in activated T cells. See Yang et al., 1995, “iPABP, an inducible poly(A)-binding protein detected in activated human T cells,” Mol. Cell Biol., 15, pg. 6770-6776; Turner et al., 2018, “RNA-binding proteins control gene expression and cell fate in the immune system,” Nat. Immunol., 19, pg. 120-129, each of which is hereby incorporated by reference in its entirety for all purposes.

Thus, in this example, the biological basis for the DAS biomarkers during SARS-CoV-2 infection was systematically characterized, identifying significant splicing changes involved in immune response both through RBP regulation and protein domain functional modulation.

The large and homogeneous discovery CHARM cohort used in this set of examples may have contributed to the superiority of our SARS-CoV-2 signatures. Compared with previously published signature sets, both CHARM DEG and DAS signatures demonstrated better performance (FIG. 4B). Statistically, the uncertainty of identifying the optimal signature set can stem from either limited sample size (data uncertainty) or inexact model estimation (model uncertainty), which both could lead to an underperforming, less-generalizable signature set.

In clinical settings, gene and protein expression is usually the primary analysis of interest, partially because of the straightforward functional interpretations. While splicing analysis appears to be underrepresented in such settings, this set of examples demonstrates that the analysis of AS is also a powerful tool and can shed new light on the molecular mechanisms that otherwise could be missed by analyses based solely on gene and protein expression.

A host-based diagnostic tool can potentially reduce the spread of infectious diseases by detecting pathogen infections prior to symptom manifest, complementing the conventional pathogen-based diagnosis largely restricted by symptom onset. Leveraging the large longitudinal cohort, this set of examples developed a host-based, AS-centered predictive model that can accurately identify SARS-CoV-2 infected samples from serial PCR false negative tests.

One limitation of the present set of examples is that the CHARM cohort is homogeneous in age and health status with few confounding factors, unlike most typical human studies of SARS-CoV-2. To provide as fair a comparison as possible of the gene set signatures rather than the generalizability of the models, the same modeling methodology was applied using the same training data to evaluate all signatures. Due to the cohort differences, the DEG performances trained in the CHARM cohort may be subpar to the model trained using the previous published cohorts. Furthermore, the relative performance of signatures could be attributed to many factors, including but not limited to training data quality and sample size, methodology for signature selection, and ensuring biological relevance of the signature cohort similarity. While the results for any particular signature may not result from the difference between DAS and DEG approaches, the failure of any DEG signature to outperform DAS supports the value of the DAS methodology.

Raw sequencing data and subject metadata used in this set of examples is deposited to NCBI GEO with accession number GSE198449. Code implementation, processed results, and reproducible analyses used in this set of examples are publicly available at the github.com/zj-zhang/CHARM-AlternativeSplicing, which is hereby incorporated by reference in its entirety for all purposes.

### Example 5—Quantification of Alternative Splicing Events

A uniform pipeline was employed to process all RNA-seq fastq files. In particular, STAR (v2.7.4) was used to align the reads to hg38 genome build with Gencode v34 index. See Dobin et al., 2013, “STAR: ultrafast universal RNA-seq aligner,” Bioinformatics, 29, pg. 15-21; Harrow et al., 2006, “GENCODE: producing a reference annotation for ENCODE,” Genome Biol., 7(1), pg. S4.1-54.9, each of which is hereby incorporated by reference in its entirety for all purposes. To quantify gene expression levels, kallisto (v0.46.0) was used to pseudo-align RNA-seq reads to Gencode v34 transcripts. See Bray et al., 2016, “Near-optimal probabilistic RNA-seq quantification,” Nat. Biotechnol., 34, pg. 525-527, which is hereby incorporated by reference in its entirety for all purposes. In some embodiments, Gencode v34 genome annotation was used as the reference gene annotations wherever applicable.

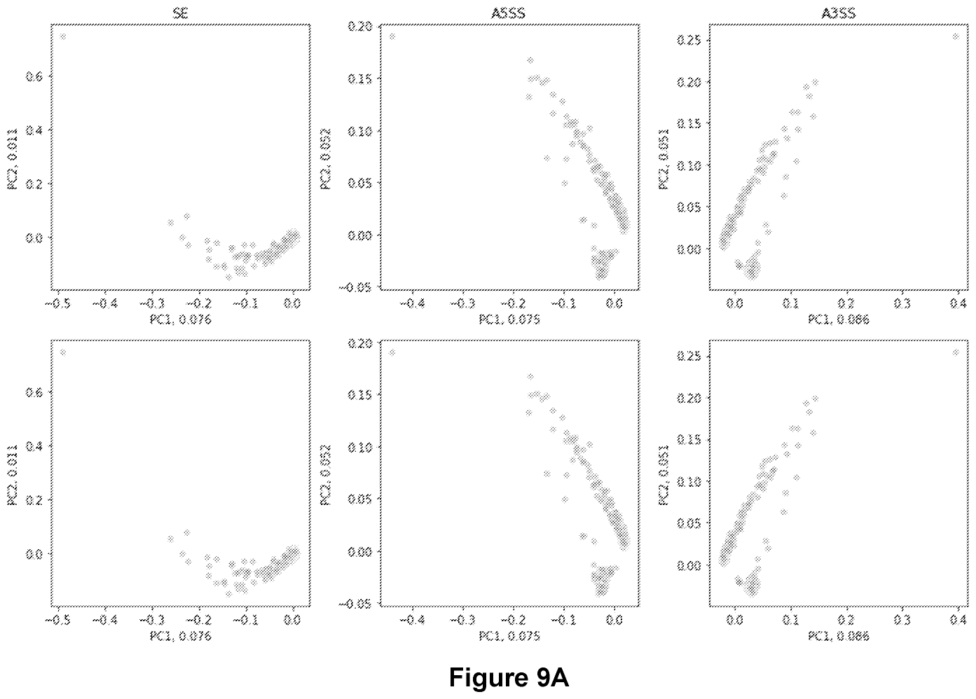

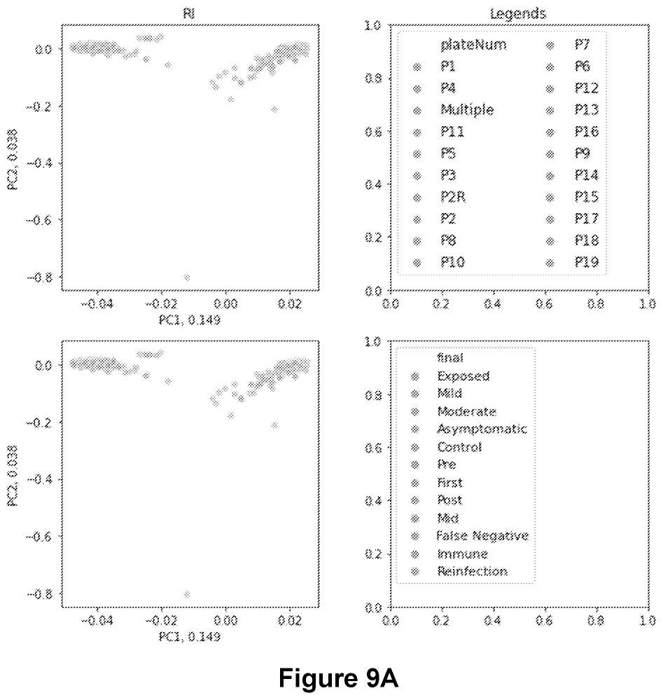

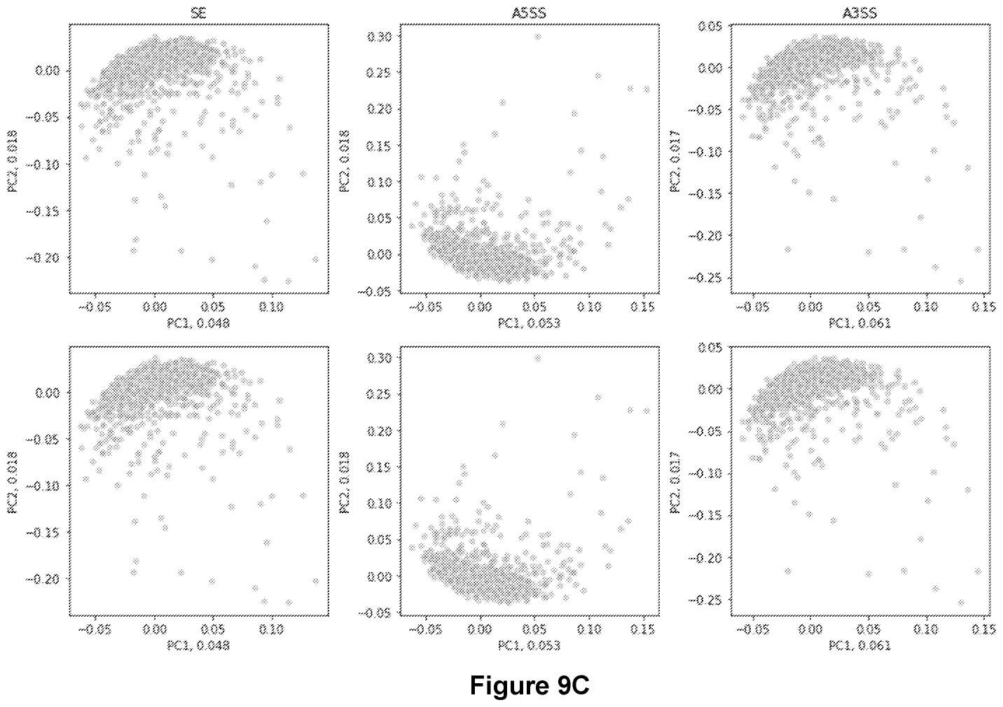

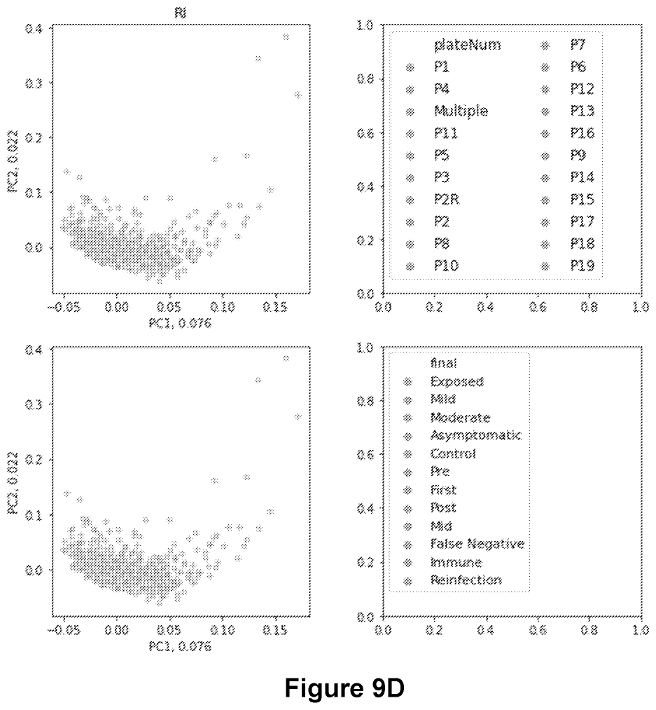

To reduce potential counting bias, two distinct approaches leveraging different aspects of RNA-seq reads to quantify Percent or Proportion Spliced In (PSI) were employed and combined. Using genome read alignment generated by STAR as input, the junction read counts for alternative splicing events were counted by DARTS/rMATS-turbo. See Zhang et al, 2019; Shen et al., 2014, “rMATS: robust and flexible detection of differential alternative splicing from replicate RNA-Seq data,” Proc. Natl. Acad. Sci. USA, 111, E5593-E5601, each of which is hereby incorporated by reference in its entirety for all purposes. Using the transcript quantifications generated by kallisto as input, the ratio between longer and shorter isoforms were computed by SUPPA2. See Trincado et al., 2018. Four basic types of alternative splicing events were analyzed: skipped exons, alternative 5′ splice sites, alternative 3′ splice sites, and retained introns. This approach allowed for the identification of measurement-agnostic differential splicing events for applicable event types and overcome the technical bias due to relatively shallow sequencing coverage per RNA-seq dataset. See FIGS. 9A through 9D.

### Example 6—Mixed Model Analysis of Longitudinal Splicing Changes

To identify the alternatively spliced exons upon SARS-CoV-2 infection, a linear mixed model regression framework was employed to study the dependency of exon usage on disease stage, sex, and potential confounding factors:

$$logit ( {ψ}_{i j l} ) = {μ}_{I} + α {Sex}_{j} + β Diseas {e}_{j} + {P}_{i j} + {δ}_{i} 1 ( {ψ}_{i j l} ∈ {Ψ}_{JCT} ) + {\sum }_{k} {γ}_{k} P {C}_{k j} + {ε}_{i j}$$

where ψᵢⱼₗ is an inclusion level for alternative splicing event i in the RNA-seq sample j measured by approach l, l is the first abundance metric or the second abundance metric, wherein the first abundance metric comprises exon-exon splice junction counts and the second abundance metric comprises isoform ratios, μ_{I} is a baseline inclusion level for alternative splicing event i, Sexⱼ is an annotated sex for sample j with regression coefficient α, Diseaseⱼ is an annotated disease stage for sample j with regression coefficient β, Pᵢⱼ is a random effect for sample j to account for covariance among multiple RNA sequencing samples derived from the same subject, δᵢ quantifies a difference between measurement approaches for alternative splicing event i if ψᵢⱼₗ is measured by counting exon-exon splice junctions ψᵢⱼₗ∈ψ_{JCT} as compared to isoform ratios, 1(⋅) is an indicator function, and γₖ is a coefficient for each of k principal components for sample j PCₖⱼ.

To control for potential batch effects, principal component analysis using all PSI levels was performed. Among the first 10 principal components (PC), PCs with no correlation with the biological variables of sex or disease stage (Pearson correlation p>0.01) were considered potential confounders. These were included in the regression model to estimate their coefficients γₖ and control for their potential confounding effects.

The regression fixed-effect coefficients and random-effect variance components were estimated by python implementation statsmodels (v0.11.1). Statistical significance was determined by Wald tests and P-values were multiple-testing corrected by Benjamini-Hochberg False Discovery Rate (FDR). Alternative splicing events with FDR<0.05 for β were considered as disease-stage dependent. Detailed code implementation and reproducible analyses are publicly available at the URL github.com/zj-zhang/CHARM-AlternativeSplicing, which is hereby incorporated by reference in its entirety for all purposes.

To separate true infection-induced AS changes from those induced by military training, a modified version of the above model was run and training-specific AS events were identified that were excluded from further analysis. To this end, a modified version of the disease-specific model was run on all control samples including time since enrollment as a variable:

$$logit ( {ψ}_{i j l} ) = {μ}_{I} + α {Sex}_{j} + {β}_{1} T i m e P o i n {t}_{j} + {β}_{2} {Sex}_{j} + T i m e P o i n {t}_{j} + {P}_{i j} + {δ}_{i} 1 ( {ψ}_{i j l} ∈ {Ψ}_{J C T} ) + {\sum }_{k} {γ}_{k} P {C}_{k j} + {ε}_{i j}$$

where TimePointⱼ is the collection time of sample j with respect to the initial enrollment of the subject. Alternative splicing events with FDR<0.05 for either the main time effect β₁ or its interaction term with sex β₂ were deemed as subject to military training effects and as such excluded from the downstream analyses.

### Example 7—Cohort Definition and Experimental Design

In the COVID-19 Health Action Response (CHARM) study, whole-blood specimens were collected from n=371 US Marine recruits in a longitudinal cohort recruited by the study entering basic training at Parris Island, South Carolina from May to November, 2020.17 301 out of 371 subjects (81.1%) had repeated measures. Biospecimens were sequenced by Illumina high-throughput sequencing with paired-end reads of 101 bp at an average depth of 25 million. All study participants were tested for SARS-CoV-2 by PCR, had serum drawn to assess antibody status, and were administered a symptom questionnaire as well as demographic information at enrollment, and approximately 7, 14, 28, 42, and 56 days afterward. SARS-CoV-2 qPCR testing was performed in mid-turbinate nares swabs and were performed within 48 h of sample collection at high complexity Clinical Laboratory Improvement Amendments-certified laboratories using the US Food and Drug Administration-authorized Thermo Fisher TaqPath COVID-19 Combo Kit (Thermo Fisher Scientific, Waltham, MA, USA). Lab24Inc (Boca Raton, FL, USA) performed PCR testing from study initiation (May 11, 2020) until Aug. 24, 2020, and the Naval Medical Research Center (Silver Spring, MD, USA) from Aug. 24, 2020, until the conclusion of the study (Nov. 2, 2020).

Depending on the infection status determined by PCR and antibody tests, RNA-seq samples were annotated to five disease stages. RNA-seq samples from initial enrollment with negative PCR tests (PCR−) and negative antibody calls were annotated as healthy controls. The RNA-seq samples collected at the last PCR− test before the subject turned PCR positive (PCR+) were annotated as pre-infection, or Pre. The RNA-seq samples at the first time a subject turned PCR+ were annotated as first-time infection, or First. Following the initial PCR+, all other RNA-seq samples while the subject remained PCR+ were annotated as mid-infection, or Mid. Finally, the RNA-seq samples from the subjects that turned PCR− after the previous infection were annotated as post-infection, or Post. None of the SARS-CoV-2 infected subjects in the CHARM cohort required hospitalization nor any treatment.

An independent test set from a cohort of 47 COVID-19 patients and 19 healthy controls from Duke University Hospital (herein referred to as Duke cohort) was profiled by whole-blood RNA-seq and processed using the same software pipeline as the CHARM study. The Duke cohort consisted COVID-19 patients with distinct characteristics from the CHARM training set (see Table 1). The Duke cohort remained unexposed to the classifier during training and validation. Prediction accuracy was measured using AUROC for the binary classification of infected subjects vs healthy controls in the held-out Duke dataset as an independent evaluation.

TABLE 1
Description of the Duke Cohort

| Subject | Age at | | | Days since | |
|---|---|---|---|---|---|
| ID | enrollment | Gender | Age | onset | Pathogen |
| 1 | 60.2 | MALE | White | 8 | COVID-19 |
| 2 | 33.5 | MALE | White | 6 | COVID-19 |
| 3 | 28.8 | MALE | White | 5 | COVID-19 |
| 4 | 27.9 | FEMALE | White | 9 | COVID-19 |
| 5 | 31.5 | FEMALE | White | 9 | COVID-19 |
| 6 | 30.7 | MALE | White | 9 | COVID-19 |
| 7 | 32.2 | MALE | White | 12 | COVID-19 |
| 8 | 30 | FEMALE | Asian | 14 | COVID-19 |
| 9 | 28.9 | FEMALE | White | 13 | COVID-19 |
| 10 | 32.6 | FEMALE | Black- | 14 | COVID-19 |
| | | | African | | |
| | | | American | | |
| 11 | 31.8 | FEMALE | White | 14 | COVID-19 |
| 12 | 28.6 | FEMALE | White | 17 | COVID-19 |
| 13 | 29.9 | FEMALE | White | 16 | COVID-19 |
| 14 | 28.1 | MALE | White | 15 | COVID-19 |
| 15 | 26.6 | FEMALE | Asian | 15 | COVID-19 |
| 16 | 50.1 | MALE | White | 21 | COVID-19 |
| 17 | 33.1 | MALE | White | 16 | COVID-19 |
| 18 | 29.5 | MALE | Asian | 16 | COVID-19 |
| 19 | 29.9 | FEMALE | White | 9 | COVID-19 |
| 20 | 29.7 | MALE | White | 11 | COVID-19 |
| 21 | 46.3 | MALE | White | 19 | COVID-19 |
| 22 | 56.1 | FEMALE | Asian | 20 | COVID-19 |
| 23 | 54.4 | FEMALE | White | 26 | COVID-19 |
| 24 | 51.8 | MALE | White | 19 | COVID-19 |
| 25 | 50.3 | FEMALE | White | 11 | COVID-19 |
| 26 | 20.1 | MALE | White | 8 | COVID-19 |
| 27 | 61.4 | MALE | Native | 17 | COVID-19 |
| | | | Hawaiian- | | |
| | | | Pacific | | |
| | | | Islander | | |
| 28 | 63.6 | FEMALE | White | 17 | COVID-19 |
| 29 | 52.1 | FEMALE | White | 34 | COVID-19 |
| 30 | 59 | MALE | Black- | 16 | COVID-19 |
| | | | African | | |
| | | | American | | |
| 31 | 60.4 | MALE | Black- | 13 | COVID-19 |
| | | | African | | |
| | | | American | | |
| 32 | 29.3 | MALE | Asian | 28 | COVID-19 |
| 33 | 30.6 | MALE | White | 29 | COVID-19 |
| 34 | 62.6 | FEMALE | White | 30 | COVID-19 |
| 35 | 71.7 | MALE | Unknown- | 1 | COVID-19 |
| | | | Not reported | | |
| 36 | 43.3 | FEMALE | White | 33 | COVID-19 |
| 37 | 35.9 | FEMALE | Asian | 10 | COVID-19 |
| 38 | 91.5 | FEMALE | Black- | 7 | COVID-19 |
| | | | African | | |
| | | | American | | |
| 39 | 31.3 | FEMALE | Black- | 18 | COVID-19 |
| | | | African | | |
| | | | American | | |
| 40 | 67.7 | MALE | Black- | 11 | COVID-19 |
| | | | African | | |
| | | | American | | |
| 41 | 69.1 | FEMALE | Black- | 9 | COVID-19 |
| | | | African | | |
| | | | American | | |
| 42 | 64.5 | MALE | White | 14 | COVID-19 |
| 43 | 76.4 | MALE | White | 3 | COVID-19 |
| 44 | 70 | MALE | Black- | 2 | COVID-19 |
| | | | African | | |
| | | | American | | |
| 45 | 33.4 | MALE | White | 9 | COVID-19 |
| 46 | 88.8 | FEMALE | Black- | 13 | COVID-19 |
| | | | African | | |
| | | | American | | |
| 47 | 69.9 | FEMALE | Black- | 4 | COVID-19 |
| | | | African | | |
| | | | American | | |
| 48 | 18 | MALE | White | | healthy |
| 49 | 19 | FEMALE | Asian | | healthy |
| 50 | 18 | MALE | White | | healthy |
| 51 | 18 | FEMALE | Asian | | healthy |
| 52 | 18 | FEMALE | White | | healthy |
| 53 | 18 | FEMALE | White | | healthy |
| 54 | 18 | MALE | Asian | | healthy |
| 55 | 20 | MALE | Asian | | healthy |
| 56 | 19 | MALE | White | | healthy |
| 57 | 18 | FEMALE | White | | healthy |
| 58 | 19 | MALE | White | | healthy |
| 59 | 18 | MALE | White | | healthy |
| 60 | 19 | MALE | Asian | | healthy |
| 1 | 18 | FEMALE | Asian | | healthy |
| 62 | 18 | MALE | Other/More | | healthy |
| | | | than one race | | |
| 63 | 18 | FEMALE | White | | healthy |
| 64 | 18 | FEMALE | Asian | | healthy |
| 65 | 18 | MALE | White | | healthy |
| 66 | 19 | MALE | White | | healthy |

### Example 8—RNA Library Preparation and Sequencing

Total RNA from PAXgene preserved blood was extracted using the Agencourt RNAdvance Blood Kit (Beckman Coulter) on a BioMek FXP Laboratory Automation Workstation (Beckman Coulter). Concentration and integrity (RIN) of isolated RNA were determined using Quant-iT RiboGreen RNA Assay Kit (Thermo Fisher) and an RNA Standard Sensitivity Kit (DNF-471, Agilent Technologies, Santa Clara, CA, USA) on a Fragment Analyzer Automated CE system (Agilent Technologies), respectively. Subsequently, cDNA libraries were constructed from total RNA using the Universal Plus mRNA-Seq kit (Tecan Genomics, San Carlos, CA, United States) in a Biomek i7 Automated Workstation (Beckman Coulter). Briefly, mRNA was isolated from purified 300 ng total RNA using oligo-dT beads and used to synthesize cDNA following the manufacturer's instructions. The transcripts for rRNA and globin were further depleted using the AnyDeplete kit (Tecan Genomics) prior to the amplification of libraries. Library concentration was assessed fluorometrically using the Qubit dsDNA HS Kit (Thermo Fisher), and quality was assessed with the Genomic DNA 50 Kb Analysis Kit (DNF-467, Agilent Technologies). Following library preparation, samples were pooled, and preliminary sequencing of cDNA libraries (average read depth of 90,000 reads) was performed using a MiSeq system (Illumina) to confirm library quality and concentration. Deep sequencing was subsequently performed using an S4 flow cell in a NovaSeq sequencing system (Illumina) (average read depth 30 million pairs of 2×100 bp reads) at New York Genome Center.

### Example 9—Machine Learning Predictor Training and Evaluation

Logistic regression was employed as the classifier to distinguish infected subjects from the healthy controls. To train the classifier, samples with first-time PCR+(First) and healthy controls without a pre-infection (Pre) sample were considered, while subjects with both control and pre-infection samples were also held-out to identify potential early immune responses markers. To address the class imbalance, positive samples were up-weighted by their ratios in the training set, while negative samples were kept a default weight of 1. Parameters were trained with 10-fold cross validation on the CHARM samples.

Six publicly available gene expression signatures for SARS-CoV-2 infection were also examined. See Thair et al., 2021; Lee et al., 2020; Li et al., 2021; McClain et al., 2021, Nat. Commun.; Aschenbrenner et al., 2021; Kwan, et al., 2021. The best performing signature sets derived from the CHARM cohort were denoted as CHARM DAS and DEG, respectively. To rigorously compare the quality of signature sets and remove other confounding effects (such as differences in discovery cohorts and machine learning algorithms used in previous studies), the classifiers were re-trained using the same logistic regression classifier on the same CHARM cohort, tested using the same held-out Duke cohort, while only varying the gene signature sets. All public signature sets were processed identically to CHARM DEG, that is, getting the gene expression matrix based on the signature set as features, training a logistic regression using these features on the CHARM cohort, then testing on Duke cohort. Classification accuracies were measured by area under the receiver-operating curves (AUROC). These results are illustrated in FIG. 4B, as further discussed in Examples 2 and 4.

### Example 10—Microfluidic Marker Selection and Analysis

To select a smaller set of biomarkers for testing on microfluidic devices, AS event was ranked by its absolute coefficient values in the classifier trained in the CHARM cohort, and a forward selection was performed to select non-redundant signatures. This went from the top of the ranked AS event list, and only added one event to the feature set, if adding it to the current feature set improved the discriminative power in the Duke cohort. A set of n=27 AS biomarkers were selected by this process and transferred to Fluidigm Corp for independent validation on a clinical cohort of n=31 SARS-CoV-2 infected samples vs n=31 healthy controls. The set of n=27 AS biomarkers is disclosed in Table 2 of Example 3 above.

Previously published microfluidic PCR data was reanalyzed based on gene expression markers by following the same principal component analysis as the AS assay. See Cappuccio et al., 2022. The first two principal components for both assays were fitted by a support vector machine with a linear kernel. Based on the linear separation of samples, accuracy, positive percent agreement and negative percent agreement were calculated. To accommodate for the sample size differences between the gene expression and AS assay, the gene expression-profiled samples were down-sampled to match the same number of positive and negative samples in the AS assay for 10,000 times. P-values were computed as the frequency that the downsampled gene expression accuracies were equal or greater than the observed accuracy in the AS assay.

### Example 11—Characterization of Alternatively Spliced Exons

Protein domains for each exon were annotated by Pfam and downloaded from UCSC table browser (genome.ucsc.edu). The clan information (v32.0) for each protein domain was accessed from the Pfam ftp server (ftp.ebi.ac.uk/pub/databases/Pfam/). Alternative spliced exons were annotated on a per-splice site basis, where each alternative splicing event (across all four types of alternative splicing the present disclosure analyzed) had four key splice sites that uniquely identified an event. Annotated domains for all four splice sites per event were assigned to that splicing event, and subsequently merged into the clan wherever applicable. Only protein domains with at least 1 observation were analyzed.

RNA-binding proteins binding sites were profiled by eCLIP assays from ENCODE. eCLIP peaks were downloaded from ENCODE data portal (www.encodeproject.org), and IDR peaks were used as a set of high-confidence peaks for the RBP enrichment analysis. Similar to the domain analysis, the alternative splicing events were annotated to RBP binding sites on a per splice-site basis. Peaks that overlapped within ±250 bp to a splice site were annotated to the AS event. Annotated RBP binding sites for all four splice sites per event were assigned to that splicing event.

Fisher exact test was employed to test the enrichment of protein domains and RBP binding sites. Foreground was defined as the significantly differentially spliced events, and background was defined as all tested events. To increase statistical power, different types of significant DAS events were pooled from all disease stages, and analyzed for the enrichment of various protein domains and RBP binding sites. Subsequently, the ratios of protein domains, or RBP binding sites, were compared between foreground and background and P-values were multiple-testing corrected by FDR.

### Example 12—Model-Based Statistical Analysis of the Variance Stabilizing Effects from Alternative Splicing Biomarkers

The variance stabilizing effects as a result of measuring alternative-splicing ratios as biomarkers was explored using a simple statistical model. The variance was analyzed by the partitioning of technical and biological variabilities in a probe-based assay, to see how these variances could cancel out when taking the ratio as in alternative splicing.

In a typical probe-based targeted biomarker assay, let X_{α} be the raw probe value for the short isoform, and X_{b} be the raw probe value for the long isoform. The following additive model was used for the measured probe values:

$${X}_{α} = {μ}_{α} + {z}_{t} + {∈}_{α}$$

$${X}_{b} = {μ}_{b} + {z}_{t} + {∈}_{b}$$

where μₐ and μ_{b} are the expected values for the measured probes a and b, respectively. zₜ is the technical platform-induced random effect,

$${z}_{t} ∼ N ( O , {σ}_{t}^{2} ) .$$

∈ₖ is the probe-specific biological fluctuations for the probe k,

$${∈}_{κ} ∼ N ( O , {σ}_{κ}^{2} ) , k ∈ \{ a , b \}$$

The technical variabilities were assumed to independent of biological variabilities, and the probe-specific biological variabilities were assumed to be independent of each other:

$${z}_{r} ⊥ {∈}_{k} , ∀ k$$

$${∈}_{k} ⊥ {∈}_{j} , ∀ j \neq k$$

Under this model, the two probes follow a bivariate normal distribution:

$$( {X}_{a} , {X}_{b} ) ∼ BVN ( [ {μ}_{a} , {μ}_{b} ] , \begin{matrix}{σ}_{t}^{2} + {σ}_{a}^{2} & Cov ( {X}_{α} , {X}_{b} ) \\ Cov ( {X}_{α} , {X}_{b} ) & {σ}_{t}^{2} + {σ}_{b}^{2}\end{matrix} ] )$$

The covariance term can be found as:

$$Cov ( {X}_{α} , {X}_{b} ) = E ( {X}_{α} {X}_{b} ) - E ( {X}_{α} ) E ( {X}_{b} ) = E ( {z}_{t}^{2} ) = {σ}_{t}^{2} > 0$$

Because X_{α}, X_{b} are measured as Cycle Threshold (CT) values that correspond to log probe expression, the ratio is computed using their difference:

$$ψ = {X}_{a} - {X}_{b} ∼ N ( {μ}_{a} - {μ}_{b} , 2 {σ}_{t}^{2} + {σ}_{a}^{2} + {σ}_{b}^{2} - 2 Cov ( {X}_{α} , {X}_{b} ) )$$

Note, this is not a canonical definition of PSI in RNA-seq, because all skip and inclusion splice junctions are unbiasedly profiled in RNA-seq, while only one of the two inclusion junctions are profiled in a targeted probe-based assay. However, let r=exp(ψ), ∀r>0 there is a one-to-one mapping between the probe-based ψ and canonical RNA-seq-based ψ_{RNA}, i.e.,

$${ψ}_{RNA} = \frac{r}{r + 1} .$$

The variance term of the calculated ψ is of interest:

$$Var ( ψ ) = 2 {σ}_{t}^{2} + {σ}_{α}^{2} + {σ}_{b}^{2} - 2 Cov ( {X}_{α} , {X}_{b} ) = {σ}_{a}^{2} + {σ}_{b}^{2}$$

Therefore, the technical variance induced by zₜ is canceled out, the variance of PSI is the sum of biological variance from two probes that can't be further reduced.

When σₜ>σₐ, Var(ψ)<Var(X_{b}); similarly, when σₜ>σ_{b}, Var(ψ)<Var(Xₐ).

Overall, the alternative-splicing biomarker variation is smaller than the sum of measuring two probes, independently, i.e.

$${σ}_{a}^{2} + {σ}_{b}^{2} < {σ}_{a}^{2} + {σ}_{b}^{2} + 2 {σ}_{t}^{2} .$$

### CONCLUSION

The terminology used herein is for the purpose of describing particular cases and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated. The implementations were chosen and described in order to best explain the principles and their practical applications, to thereby enable others skilled in the art to best utilize the implementations and various implementations with various modifications as are suited to the particular use contemplated.

What is claimed:

1. A method for determining an infection status of a test subject, the method comprising:
at a computer system comprising one or more processors and a memory storing at least one program for execution by the one or more processors:
a) obtaining, in electronic form, a plurality of sequence reads from a biological sample of the test subject, wherein the plurality of sequence reads comprises at least 10,000 RNA sequence reads;
b) determining, for each respective alternative splicing event in a plurality of alternative splicing events a corresponding first abundance metric of the respective alternative splicing event, wherein:
each respective alternative splicing event in the plurality of alternative splicing events corresponds to a respective locus in a plurality of loci in a reference genome for the species of the test subject, and
the plurality of alternative splicing events comprises at least 10 alternative splicing events; and
c) receiving, responsive to inputting the corresponding first abundance metric for each respective alternative splicing event in the plurality of alternative splicing events into a first model, a predicted infection status of the test subject as output from the first model.

2. The method of claim 1, wherein the corresponding first abundance metric of the respective alternative splicing event uses a mapping of each respective sequence read in the plurality of sequence reads to one or more reference splice junctions in a plurality of reference splice junctions.

3. The method of claim 1, wherein the corresponding first abundance metric uses a mapping of each respective sequence read in the plurality of sequence reads to one or more reference isoforms in a plurality of reference isoforms, wherein:

4. The method of claim 1, determining b) determines, for each respective alternative splicing event in the plurality of alternative splicing events:
(i) the corresponding first abundance metric of the respective alternative splicing event in the biological sample based on a mapping of each respective sequence read in the plurality of sequence reads to one or more reference splice junctions in a plurality of reference splice junctions, and
(ii) a corresponding second abundance metric of the respective alternative splicing event in the biological sample based on a mapping of each respective sequence read in the plurality of sequence reads to one or more reference isoforms in a plurality of reference isoforms; and
the receiving c) further inputs the corresponding second abundance metric for each respective alternative splicing event in the plurality of alternative splicing events into the first model, to obtain the predicted infection status of the test subject as output from the first model.

5. The method of claim 4, wherein the first and second abundance metric for a respective alternative splicing event in the plurality of alternative splicing events is inputted into the first model as a mathematical combination.

6. The method of claim 4, wherein the first and second abundance metric for a respective alternative splicing event in the plurality of alternative splicing events are each separately inputted into the first model.

7. The method of claim 1, wherein the biological sample is whole blood.

8. The method of any one of claims 1-7, wherein the biological sample comprises a plurality of mRNA molecules and the obtaining the plurality of sequence reads further comprises sequencing the plurality of mRNA molecules using RNA sequencing.

9. The method of claim 8, wherein all or a portion of the plurality of mRNA molecules is derived from the test subject.

10. The method of claim 8 or 9, wherein the biological sample further comprises nucleic acid molecules derived from a pathogen.

11. The method of any one of claims 1-10, wherein the infection status of the test subject is for a SARS-CoV-2 infection.

12. The method of any one of claims 1-11, wherein the plurality of sequence reads comprises at least 100,000, at least 1×10⁶, or at least 1×10⁷ sequence reads.

13. The method of any one of claims 4-6, for a respective alternative splicing event in the plurality of alternative splicing events, the first abundance metric is a percent or proportion spliced in metric determined according to the equation:
$$\frac{\frac{inclusion count}{2}}{\frac{inclusion count}{2} + skip count}$$
wherein:
inclusion count is a count of inclusion splice junctions for a first intervening sequence corresponding to the respective alternative splicing event, each respective inclusion splice junction for the first intervening sequence comprising a first nucleic acid sequence for a 5′ or a 3′ end of the first intervening sequence and a second nucleic acid sequence for an adjoining sequence that is 5′ or 3′ of the first intervening sequence, and
skip count is a count of exclusion splice junctions for the first intervening sequence corresponding to the respective alternative splicing event, each respective exclusion splice junction excluding all or a portion of the first intervening sequence.

14. The method of any one of claims 1-14, wherein the determining b) further comprises aligning each respective sequence read in the plurality of sequence reads to a first reference sequence comprising the plurality of reference splice junctions.

15. The method of claim 14, wherein the first reference sequence is a reference human genome.

16. The method of any one of claims 1-15, wherein the first abundance metric is determined using an RNA sequencing mapping algorithm.

17. The method of any one of claims 4-6, wherein, for a respective alternative splicing event in the plurality of alternative splicing events, the second abundance metric is a percent or proportion spliced in metric determined according to the equation:
$$\frac{\sum inclusion isoform TPM}{\sum all relevant isoform TPM}$$
wherein:
inclusion isoform TPM is a count of transcript isoforms in the biological sample comprising a first intervening sequence corresponding to the respective alternative splicing event, measured in transcripts per million, and
all relevant isoform TPM is a count of transcript isoforms spanning the first intervening sequence corresponding to the respective alternative splicing event, measured in transcripts per million.

18. The method of any one of claims 1-18, wherein the determining b) further comprises aligning each respective sequence read in the plurality of sequence reads to a second reference sequence comprising the plurality of reference isoforms.

19. The method of claim 18, wherein the second reference sequence is a reference human transcriptome and the aligning comprises a pseudo-alignment.

20. The method of any one of claims 4-6, wherein the second abundance metric is determined using a differential splicing analysis algorithm.

21. The method of any one of claims 1-20, wherein each respective alternative splicing event in the plurality of alternative splicing events is a skipped exon, an alternative 5′ splice site, an alternative 3′ splice site, or a retained intron in the respective locus in the plurality of loci that corresponds to the respective alternative splicing event.

22. The method of any one of claims 1-21, wherein the plurality of alternative splicing events comprises at least 10, at least 20, at least 50, at least 100, or at least 500 alternative splicing events.

23. The method of any one of claims 1-22, wherein the plurality of alternative splicing events is no more than 2000, no more than 1000, no more than 500, no more than 100, or no more than 50 alternative splicing events.

24. The method of any one of claims 1-23, wherein the plurality of alternative splicing events consists of from 100 alternative splicing events to 600 alternative splicing events.

25. The method of any one of claims 1-24, wherein the plurality of alternative splicing events consists of from 10 alternative splicing events to 50 alternative splicing events.

26. The method of any one of claims 1-25, wherein each respective locus in the plurality of loci is a gene in a plurality of genes.

27. The method of claim 26, wherein the plurality of alternative splicing events is for determining an infection status of a SARS-CoV-2 infection, and the plurality of genes comprises one or more of IGLL5, LST1, GALNS, EPSTI1, LILRB2, RIN2, PALM2AKAP2, HMGN2, TUBA8, SNHG32, KIF22, ATP6V0B, SESN3, LRRK, U91328.1, IQSEC1, RPS3A, KY, PHOSPHO1, RILP, MRPS22, and ZFYVE26.

28. The method of any one of claims 1-27, wherein the plurality of alternative splicing events is selected from the group consisting of: skipped exon IGLL5, retained intron LST1, skipped exon GALNS, skipped exon EPSTI1, retained intron LILRB2, skipped exon RIN2, skipped exon PALM2AKAP2, retained intron HMGN2, alternative 5′ splice site TUBA8, skipped exon SNHG32, alternative 3′ splice site KIF22, alternative 5′ splice site ATP6V0B, skipped exon SESN3, alternative 3′ splice site LST1, alternative 3′ splice site LRRK, skipped exon U91328.1, alternative 3′ splice site IQSEC1, skipped exon RPS3A, alternative 5′ splice site KY, alternative 3′ splice site PHOSPHO1, skipped exon RILP, retained intron MRPS22, skipped exon ZFYVE26, skipped exon PHOSPHO1, and alternative 3′ splice site LILRB2.

29. The method of any one of claims 1-28, further comprising, prior to the determining b), selecting the plurality of alternative splicing events by:
obtaining a first plurality of training samples, wherein:
each respective training sample in the first plurality of training samples (i) corresponds to a respective training subject in a first plurality of training subjects and (ii) comprises a corresponding infection status,
each respective training sample in a first subset of the first plurality of training samples comprises a first infection status, and
each respective training sample in a second subset of the first plurality of training samples comprises a second infection status;
determining, for each respective training sample in the first plurality of training samples, for each respective candidate event in a plurality of candidate events, at least a third abundance metric of the respective candidate event in the respective training sample, thereby obtaining at least a plurality of first abundance metrics for the first plurality of training samples;
receiving, responsive to inputting at least the plurality of third abundance metrics into a second model:
for each respective candidate event in the plurality of candidate events, (i) a corresponding coefficient of effect between the respective candidate event and the corresponding infection status of each respective training sample in the first plurality of training samples and (ii) a measure of significance for the corresponding coefficient of effect; and
evaluating, for each respective candidate event in the plurality of candidate events, the (i) corresponding coefficient of effect or (ii) measure of significance against one or more selection criteria, thereby selecting the plurality of alternative splicing events.

30. The method of claim 29, wherein the first plurality of training subjects comprises a first subset of healthy subjects and a second subset of disease subjects.

31. The method of claim 30, wherein:
the first infection status is negative for infection,
the second infection status is positive for infection, and
each respective training sample in the second subset of training samples is obtained from the second subset of disease subjects.

32. The method of claim 30 or 31, wherein, for each respective training sample in the second subset of training samples, the corresponding infection status is selected from the group consisting of pre-infection, first-infection, mid-infection, and post-infection.

33. The method of any one of claims 29-32, wherein each respective training sample in the first subset of training samples is obtained from the first subset of healthy subjects or the second subset of disease subjects.

34. The method of any one of claims 29-33, wherein, for each respective training sample in the first plurality of training samples, the corresponding infection status is determined by polymerase chain reaction, immunoglobulin G antibody testing, or immunoglobulin M antibody testing.

35. The method of any one of claims 29-34, wherein, for each respective training sample in the first plurality of training samples, for each respective candidate event in the plurality of candidate events, the corresponding third abundance metric is determined based on a mapping of each respective sequence read, in a plurality of sequence reads for the respective training sample, to one or more reference splice junctions in a plurality of reference splice junctions.

36. The method of any one of claims 29-35, further comprising:
for each respective training sample in the first plurality of training samples, for each respective candidate event in the plurality of candidate events, determining a fourth abundance metric of the respective candidate event in the respective training sample,
thereby obtaining a plurality of fourth abundance metrics for the first plurality of training samples, and wherein
the receiving further comprises inputting the plurality of fourth abundance metrics, with the plurality of third abundance metrics, into the second model.

37. The method of any one of claims 29-36, wherein the second model is a regression model and the corresponding coefficient of effect is a regression coefficient.

38. The method of claim 37, wherein the regression model is a linear mixed model.

39. The method of any one of claims 29-38, wherein the measure of significance is a false discovery rate.

40. The method of any one of claims 29-39, wherein the one or more selection criteria comprises a threshold false discovery rate of less than 0.05, less than 0.01, or less than 0.001.

41. The method of any one of claims 35-40, wherein, for each respective candidate event in the plurality of candidate events, the corresponding coefficient of effect is determined as regression coefficient β, according to the equation:
$$logit ( {ψ}_{ijl} ) = {μ}_{I} + α {Sex}_{j} + β {Disease}_{j} + {P}_{ij} + {δ}_{i} 1 ( {ψ}_{ijl} ∈ {Ψ}_{JCT} ) + {\sum }_{k} {γ}_{k} {PC}_{kj} + {ε}_{ij}$$
wherein:
ψᵢⱼₗ is an inclusion level for alternative splicing event i in the RNA-seq sample j measured by approach l,
l is the third abundance metric or the fourth abundance metric, wherein the third abundance metric comprises exon-exon splice junction counts and the fourth abundance metric comprises isoform ratios,
μ_{I} is a baseline inclusion level for alternative splicing event i,
Sexⱼ is an annotated sex for sample j with regression coefficient α,
Diseaseⱼ is an annotated disease stage for sample j with regression coefficient β,
Pᵢⱼ is a random effect for sample j to account for covariance among multiple RNA sequencing samples derived from the same subject,
δᵢ quantifies a difference between measurement approaches for alternative splicing event i if ψᵢⱼₗ is measured by counting exon-exon splice junctions ψᵢⱼₗ∈ψ_{JCT} as compared to isoform ratios,
1(⋅) is an indicator function, and
γₖ is a coefficient for each of k principal components for sample j PCₖⱼ.

42. The method of any one of claims 1-42, further comprising filtering the plurality of alternative splicing events using a forward selection procedure comprising:
obtaining a ranked sequence of alternative splicing events by ranking the plurality of alternative splicing events by their respective coefficients of effect;
initializing a filtered subset of alternative splicing events with the highest ranked alternative splicing event in the ranked sequence of alternative splicing events; and
performing a plurality of iterations, each respective iteration in the plurality of iterations comprising, for each respective alternative splicing event that is the next highest ranked alternative splicing event in the ranked sequence of alternative splicing events:
obtaining a respective evaluation set of alternative splicing events comprising the respective alternative splicing event and the filtered subset of alternative splicing events,
for each respective validation subject in a plurality of validation subjects:
(i) for each respective alternative splicing event in the evaluation set of alternative splicing events, determining at least a corresponding fifth abundance metric for the respective alternative splicing event in a biological sample of the respective validation subject, and
(ii) receiving, responsive to inputting the corresponding fifth abundance metric for each respective alternative splicing event in the evaluation set of alternative splicing events into the first model, a predicted infection status of the respective validation subject as output from the first model, and
using the predicted infection status for each respective validation subject in the plurality of validation subjects to determine a corresponding evaluation metric for the respective evaluation set of alternative splicing events, wherein:
when the corresponding evaluation metric satisfies a filtering criterion, adding the respective alternative splicing event to the filtered subset of alternative splicing events and performing a subsequent iteration in the plurality of iterations, and
when the corresponding evaluation metric fails to satisfy the filtering criterion, ending the plurality of iterations thereby obtaining the filtered subset of alternative splicing events.

43. The method of claim 42, further comprising, for each respective validation subject in the plurality of validation subjects, for each respective alternative splicing event in the evaluation set of alternative splicing events:
determining a corresponding sixth abundance metric for the respective alternative splicing event in the biological sample of the respective validation subject, wherein:
the (ii) receiving further comprises inputting the corresponding sixth abundance metric, with the corresponding first abundance metric, into the first model.

44. The method of claim 42 or 43, wherein, for a respective iteration in the plurality of iterations:
the filtering criterion is satisfied when the corresponding evaluation metric exceeds an evaluation metric for the iteration immediately prior to the respective iteration, and
the filtering criterion is not satisfied when the corresponding evaluation metric does not exceed the evaluation metric for the iteration immediately prior to the respective iteration.

45. The method of any one of claims 42-44, wherein the evaluation metric is selected from the group consisting of accuracy, positive percent agreement, and negative percent agreement.

46. The method of any one of claims 1-45, wherein the first model is a logistic regression model.

47. The method of any one of claims 1-45, wherein the first model is selected from the group consisting of: a neural network, a support vector machine, a Naive Bayes model, a nearest neighbor model, a boosted trees model, a random forest model, a decision tree, or a clustering model.

48. The method of any one of claims 1-47, wherein the infection status of the test subject is a likelihood that the test subject has an infection.

49. The method of any one of claims 1-47, wherein the infection status of the test subject is a likelihood that the test subject is pre-infection, first-infection, mid-infection, or post-infection.

50. The method of any one of claims 1-47, wherein the infection status of the test subject is a binary indication as to whether or not test subject has an infection.

51. The method of any one of claims 1-47, wherein the infection status of the test subject is a binary indication as to whether or not the test subject has a pre-infection, a first-infection, a mid-infection, or a post-infection.

52. The method of any one of claims 1-51, further comprising, prior to the receiving c), training the first model by a procedure comprising:
determining, for each respective training sample in a second plurality of training samples, for each respective alternative splicing event in the plurality of alternative splicing events, at least the first abundance metric of the respective alternative splicing event in the respective training sample;
receiving, for each respective training sample in the second plurality of training samples, responsive to inputting at least the first abundance metric of each respective alternative splicing event in the plurality of alternative splicing events into the first model, a corresponding predicted infection status of the respective training sample as output from the first model, wherein the first model comprises a plurality of parameters;
applying a respective difference to a loss function to obtain a respective output of the loss function, wherein the respective difference is between, for each respective training sample in the second plurality of training samples, (i) the corresponding predicted infection status and (ii) the corresponding measured infection status; and
using the respective output of the loss function to adjust one or more parameters in the plurality of parameters of the first model, thereby training the first model.

53. The method of claim 52, wherein:
each respective training sample in the second plurality of training samples (i) corresponds to a respective training subject in a second plurality of training subjects and (ii) comprises a corresponding measured infection status,
each respective training sample in a first subset of the second plurality of training samples has a first measured infection status, and
each respective training sample in a second subset of the second plurality of training samples has a second measured infection status.

54. The method of claim 52 or 53, wherein the second plurality of training subjects comprises a first subset of healthy subjects and a second subset of disease subjects.

55. The method of claim 54, wherein:
the first measured infection status is negative for infection,
the second measured infection status is positive for infection, and
each respective training sample in the second subset of training samples is obtained from the second subset of disease subjects.

56. The method of claim 54, where the first measured infection status is selected from the group consisting of pre-infection, first-infection, mid-infection, and post-infection.

57. The method of any one of claims 52-56, wherein, for each respective training sample in the second plurality of training samples, the corresponding measured infection status is determined by polymerase chain reaction, immunoglobulin G antibody testing, or immunoglobulin M antibody testing.

58. The method of any one of claims 52-57, wherein, for each respective training sample in the second plurality of training samples, for each respective alternative splicing event in the plurality of alternative splicing events, the corresponding first abundance metric is determined based on a mapping of each respective sequence read, in a plurality of sequence reads for the respective training sample, to one or more reference splice junctions in a plurality of reference splice junctions.

59. The method of any one of claims 52-58, further comprising:
for each respective training sample in the second plurality of training samples, for each respective alternative splicing event in the plurality of alternative splicing events:
determining a second abundance metric of the respective alternative splicing event in the respective training sample, wherein:
the receiving further comprises inputting the second abundance metric of each respective alternative splicing event in the plurality of alternative splicing events into the first model.

60. The method of any one of claims 1-59, wherein the infection status of the test subject is for a bacterial infection, a viral infection, a fungal infection, a parasitic infection, sepsis, tuberculosis, a respiratory infection, a gastrointestinal infection, a urinary tract infection, or a combination thereof.

61. The method of any one of claims 1-59, wherein the infection status of the test subject is for an influenza infection, a human immunodeficiency viral infection, COVID-19, or a combination thereof.

62. The method of any one of claims 1-61, wherein the predicted infection status of the test subject provided as output from the first model is part of a host-based response assay.

63. A computer system for determining an infection status of a test subject, the computer system comprising:
one or more processors; and
memory addressable by the one or more processors, the memory storing at least one program for execution by the one or more processors, the at least one program comprising instructions for:
a) obtaining, in electronic form, a plurality of sequence reads from a biological sample of the test subject, wherein the plurality of sequence reads comprises at least 10,000 RNA sequence reads;
b) determining, for each respective alternative splicing event in a plurality of alternative splicing events a corresponding first abundance metric of the respective alternative splicing event, wherein:
each respective alternative splicing event in the plurality of alternative splicing events corresponds to a respective locus in a plurality of loci in a reference genome for the species of the test subject, and
the plurality of alternative splicing events comprises at least 10 alternative splicing events; and
c) receiving, responsive to inputting the corresponding first abundance metric for each respective alternative splicing event in the plurality of alternative splicing events into a first model, a predicted infection status of the test subject as output from the first model.

64. The computer system of claim 63, wherein the corresponding first abundance metric of the respective alternative splicing event uses a mapping of each respective sequence read in the plurality of sequence reads to one or more reference splice junctions in a plurality of reference splice junctions.

65. The computer system of claim 63, wherein the corresponding first abundance metric uses a mapping of each respective sequence read in the plurality of sequence reads to one or more reference isoforms in a plurality of reference isoforms, wherein:

66. The computer system of claim 63, determining b) determines, for each respective alternative splicing event in the plurality of alternative splicing events:
(i) the corresponding first abundance metric of the respective alternative splicing event in the biological sample based on a mapping of each respective sequence read in the plurality of sequence reads to one or more reference splice junctions in a plurality of reference splice junctions, and
(ii) a corresponding second abundance metric of the respective alternative splicing event in the biological sample based on a mapping of each respective sequence read in the plurality of sequence reads to one or more reference isoforms in a plurality of reference isoforms; and
the receiving c) further inputs the corresponding second abundance metric for each respective alternative splicing event in the plurality of alternative splicing events into the first model, to obtain the predicted infection status of the test subject as output from the first model.

67. The computer system of claim 63, wherein the first and second abundance metric for a respective alternative splicing event in the plurality of alternative splicing events is inputted into the first model as a mathematical combination.

68. The computer system of claim 63, wherein the first and second abundance metric for a respective alternative splicing event in the plurality of alternative splicing events are each separately inputted into the first model.

69. The computer system of claim 63, wherein the biological sample is whole blood.

70. The computer system of any one of claims 63-69, wherein the biological sample comprises a plurality of mRNA molecules and the obtaining the plurality of sequence reads further comprises sequencing the plurality of mRNA molecules using RNA sequencing.

71. The computer system of claim 70, wherein all or a portion of the plurality of mRNA molecules is derived from the test subject.

72. The computer system of claim 69 or 70, wherein the biological sample further comprises nucleic acid molecules derived from a pathogen.

73. The computer system of any one of claims 63-72, wherein the infection status of the test subject is for a SARS-CoV-2 infection.

74. The computer system of any one of claims 63-73, wherein the plurality of sequence reads comprises at least 100,000, at least 1×10⁶, or at least 1×10⁷ sequence reads.

75. The computer system of any one of claims 66-68, for a respective alternative splicing event in the plurality of alternative splicing events, the first abundance metric is a percent or proportion spliced in metric determined according to the equation:
$$\frac{\frac{inclusion count}{2}}{\frac{inclusion count}{2} + skip count}$$
wherein:
inclusion count is a count of inclusion splice junctions for a first intervening sequence corresponding to the respective alternative splicing event, each respective inclusion splice junction for the first intervening sequence comprising a first nucleic acid sequence for a 5′ or a 3′ end of the first intervening sequence and a second nucleic acid sequence for an adjoining sequence that is 5′ or 3′ of the first intervening sequence, and
skip count is a count of exclusion splice junctions for the first intervening sequence corresponding to the respective alternative splicing event, each respective exclusion splice junction excluding all or a portion of the first intervening sequence.

76. The computer system of any one of claims 63-75, wherein the determining b) further comprises aligning each respective sequence read in the plurality of sequence reads to a first reference sequence comprising the plurality of reference splice junctions.

77. The computer system of claim 76, wherein the first reference sequence is a reference human genome.

78. The computer system of any one of claims 63-77, wherein the first abundance metric is determined using an RNA sequencing mapping algorithm.

79. The computer system of any one of claims 66-68, wherein, for a respective alternative splicing event in the plurality of alternative splicing events, the corresponding second abundance metric is a percent or proportion spliced in metric determined according to the equation:
$$\frac{\sum inclusion isoform TPM}{\sum all relevant isoform TPM}$$
wherein:
inclusion isoform TPM is a count of transcript isoforms in the biological sample comprising a first intervening sequence corresponding to the respective alternative splicing event, measured in transcripts per million, and
all relevant isoform TPM is a count of transcript isoforms spanning the first intervening sequence corresponding to the respective alternative splicing event, measured in transcripts per million.

80. The computer system of any one of claims 63-79, wherein the determining b) further comprises aligning each respective sequence read in the plurality of sequence reads to a second reference sequence comprising the plurality of reference isoforms.

81. The computer system of claim 80, wherein the second reference sequence is a reference human transcriptome and the aligning comprises a pseudo-alignment.

82. The computer system of any one of claims 66-68, wherein the second abundance metric is determined using a differential splicing analysis algorithm.

83. The computer system of any one of claims 63-82, wherein each respective alternative splicing event in the plurality of alternative splicing events is a skipped exon, an alternative 5′ splice site, an alternative 3′ splice site, or a retained intron in the respective locus in the plurality of loci that corresponds to the respective alternative splicing event.

84. The computer system of any one of claims 63-83, wherein the plurality of alternative splicing events comprises at least 10, at least 20, at least 50, at least 100, or at least 500 alternative splicing events.

85. The computer system of any one of claims 63-84, wherein the plurality of alternative splicing events is no more than 2000, no more than 1000, no more than 500, no more than 100, or no more than 50 alternative splicing events.

86. The computer system of any one of claims 63-85, wherein the plurality of alternative splicing events consists of from 100 alternative splicing events to 600 alternative splicing events.

87. The computer system of any one of claims 63-86, wherein the plurality of alternative splicing events consists of from 10 alternative splicing events to 50 alternative splicing events.

88. The computer system of any one of claims 63-88, wherein each respective locus in the plurality of loci is a gene in a plurality of genes.

89. The computer system of claim 88, wherein the plurality of alternative splicing events is for determining an infection status of a SARS-CoV-2 infection, and the plurality of genes comprises one or more of IGLL5, LST1, GALNS, EPSTI1, LILRB2, RIN2, PALM2AKAP2, HMGN2, TUBA8, SNHG32, KIF22, ATP6V0B, SESN3, LRRK, U91328.1, IQSEC1, RPS3A, KY, PHOSPHO1, RILP, MRPS22, and ZFYVE26.

90. The computer system of any one of claims 63-89, wherein the plurality of alternative splicing events is selected from the group consisting of: skipped exon IGLL5, retained intron LST1, skipped exon GALNS, skipped exon EPSTI1, retained intron LILRB2, skipped exon RIN2, skipped exon PALM2AKAP2, retained intron HMGN2, alternative 5′ splice site TUBA8, skipped exon SNHG32, alternative 3′ splice site KIF22, alternative 5′ splice site ATP6V0B, skipped exon SESN3, alternative 3′ splice site LST1, alternative 3′ splice site LRRK, skipped exon U91328.1, alternative 3′ splice site IQSEC1, skipped exon RPS3A, alternative 5′ splice site KY, alternative 3′ splice site PHOSPHO1, skipped exon RILP, retained intron MRPS22, skipped exon ZFYVE26, skipped exon PHOSPHO1, and alternative 3′ splice site LILRB2.

91. The computer system of any one of claims 63-90, further comprising, prior to the determining b), selecting the plurality of alternative splicing events by:
obtaining a first plurality of training samples, wherein:
each respective training sample in the first plurality of training samples (i) corresponds to a respective training subject in a first plurality of training subjects and (ii) comprises a corresponding infection status,
each respective training sample in a first subset of the first plurality of training samples comprises a first infection status, and
each respective training sample in a second subset of the first plurality of training samples comprises a second infection status;
determining, for each respective training sample in the first plurality of training samples, for each respective candidate event in a plurality of candidate events, at least a third abundance metric of the respective candidate event in the respective training sample, thereby obtaining at least a plurality of first abundance metrics for the first plurality of training samples;
receiving, responsive to inputting at least the plurality of third abundance metrics into a second model:
for each respective candidate event in the plurality of candidate events, (i) a corresponding coefficient of effect between the respective candidate event and the corresponding infection status of each respective training sample in the first plurality of training samples and (ii) a measure of significance for the corresponding coefficient of effect; and
evaluating, for each respective candidate event in the plurality of candidate events, the (i) corresponding coefficient of effect or (ii) measure of significance against one or more selection criteria, thereby selecting the plurality of alternative splicing events.

92. The computer system of claim 91, wherein the first plurality of training subjects comprises a first subset of healthy subjects and a second subset of disease subjects.

93. The computer system of claim 92, wherein:
the first infection status is negative for infection,
the second infection status is positive for infection, and
each respective training sample in the second subset of training samples is obtained from the second subset of disease subjects.

94. The computer system of claim 92 or 93, wherein, for each respective training sample in the second subset of training samples, the corresponding infection status is selected from the group consisting of pre-infection, first-infection, mid-infection, and post-infection.

95. The computer system of any one of claims 92-94, wherein each respective training sample in the first subset of training samples is obtained from the first subset of healthy subjects or the second subset of disease subjects.

96. The computer system of any one of claims 92-95, wherein, for each respective training sample in the first plurality of training samples, the corresponding infection status is determined by polymerase chain reaction, immunoglobulin G antibody testing, or immunoglobulin M antibody testing.

97. The computer system of any one of claims 92-96, wherein, for each respective training sample in the first plurality of training samples, for each respective candidate event in the plurality of candidate events, the corresponding third abundance metric is determined based on a mapping of each respective sequence read, in a plurality of sequence reads for the respective training sample, to one or more reference splice junctions in a plurality of reference splice junctions.

98. A non-transitory computer readable storage medium, wherein the non-transitory computer readable storage medium stores instructions, which when executed by a computer system, cause the computer system to perform a method for determining an infection status of a test subject, the method comprising:
a) obtaining, in electronic form, a plurality of sequence reads from a biological sample of the test subject, wherein the plurality of sequence reads comprises at least 10,000 RNA sequence reads;
b) determining, for each respective alternative splicing event in a plurality of alternative splicing events a corresponding first abundance metric of the respective alternative splicing event, wherein:
each respective alternative splicing event in the plurality of alternative splicing events corresponds to a respective locus in a plurality of loci in a reference genome for the species of the test subject, and
the plurality of alternative splicing events comprises at least 10 alternative splicing events; and
c) receiving, responsive to inputting the corresponding first abundance metric for each respective alternative splicing event in the plurality of alternative splicing events into a first model, a predicted infection status of the test subject as output from the first model.

99. A method for determining a COVID-19 status of a test subject, the method comprising:
at a computer system comprising one or more processors and a memory storing at least one program for execution by the one or more processors:
a) obtaining, in electronic form, a plurality of sequence reads from a biological sample of the test subject;
b) determining, for each respective alternative splicing event in a plurality of alternative splicing events a corresponding first abundance metric of the respective alternative splicing event, wherein:
each respective alternative splicing event in the plurality of alternative splicing events corresponds to a respective locus in a plurality of loci in a reference genome for the species of the test subject, and
the plurality of alternative splicing events consists of between 2 and 27 splicing events listed in Table 2; and
c) receiving, responsive to inputting the corresponding first abundance metric for each respective alternative splicing event in the plurality of alternative splicing events into a first model, a predicted infection status of the test subject as output from the first model.

100. A method for determining a COVID-19 status of a test subject, the method comprising:
at a computer system comprising one or more processors and a memory storing at least one program for execution by the one or more processors:
a) obtaining, in electronic form, a plurality of sequence reads from a biological sample of the test subject;
b) determining, for each respective alternative splicing event in a plurality of alternative splicing events a corresponding first abundance metric of the respective alternative splicing event, wherein:
each respective alternative splicing event in the plurality of alternative splicing events corresponds to a respective locus in a plurality of loci in a reference genome for the species of the test subject, and
the plurality of alternative splicing events consists of between 2 and 50 splicing events listed in Table 4; and
c) receiving, responsive to inputting the corresponding first abundance metric for each respective alternative splicing event in the plurality of alternative splicing events into a first model, a predicted infection status of the test subject as output from the first model.
